(19) United States
(12) Patent Application Publication
(10) Pub. No.: US 2014/0314752 A1
(43) Pub. Date: Oct. 23, 2014

(54) METHODS FOR TREATING CANCER USING TOR KINASE INHIBITOR COMBINATION THERAPY

(71) Applicant: Signal Pharmaceuticals, LLC, San Diego, CA (US)

(72) Inventors: ANTONIA LOPEZ-GIRONA, San Diego, CA (US); KRISTEN MAE HEGE, Burlingame, CA (US); RAJESH CHOPRA, Summit, NJ (US)

(73) Assignee: Signal Pharmaceuticals, LLC, San Diego, CA (US)

(21) Appl. No.: 14/254,019

(22) Filed: Apr. 16, 2014

Related U.S. Application Data
(60) Provisional application No. 61/908,859, filed on Nov. 26, 2013.
(60) Provisional application No. 61/813,094, filed on Apr. 17, 2013.

(51) Int. Cl. A61K 31/4985; A61K 45/06; A61K 39/395

(52) U.S. Cl. CPC A61K 31/4985; A61K 39/39558; A61K 45/06
USPC 424/133.1; 424/174.1; 514/249

(57) ABSTRACT

Provided herein are methods for treating or preventing a cancer, comprising administering an effective amount of a TOR kinase inhibitor and an effective amount of an IMiD® immunomodulatory drug to a patient having a cancer.

This application claims the benefit of U.S. Provisional Application No. 61/813,094, filed Apr. 17, 2013 and U.S. Provisional Application No. 61/908,859, filed Nov. 26, 2013, the entire contents of which are incorporated herein by reference.

### 1. FIELD

Provided herein are methods for treating or preventing a cancer, comprising administering an effective amount of a TOR kinase inhibitor and an effective amount of an IMiD® immunomodulatory drug to a patient having a cancer.

### 2. BACKGROUND

The connection between abnormal protein phosphorylation and the cause or consequence of diseases has been known for over 20 years. Accordingly, protein kinases have become a very important group of drug targets. See Cohen, Nature, 1:309-315 (2002). Various protein kinase inhibitors have been used clinically in the treatment of a wide variety of diseases, such as cancer and chronic inflammatory diseases, including diabetes and stroke. See Cohen, Eur. J. Biochem., 268:5001-5010 (2001), Protein Kinase Inhibitors for the Treatment of Disease: The Promise and the Problems, Handbook of Experimental Pharmacology, Springer Berlin Heidelberg, 167 (2005).

The protein kinases are a large and diverse family of enzymes that catalyze protein phosphorylation and play a critical role in cellular signaling. Protein kinases may exert positive or negative regulatory effects, depending upon their target protein. Protein kinases are involved in specific signaling pathways which regulate cell functions such as, but not limited to, metabolism, cell cycle progression, cell adhesion, vascular function, apoptosis, and angiogenesis. Malfunctions of cellular signaling have been associated with many diseases, the most characterized of which include cancer and diabetes. The regulation of signal transduction by cytokines and the association of signal molecules with protooncogenes and tumor suppressor genes have been well documented. Similarly, the connection between diabetes and related conditions, and deregulated levels of protein kinases, has been demonstrated. See e.g., Sridhar et al. Pharmaceutical Research, 17(11):1345-1353 (2000). Viral infections and the conditions related thereto have also been associated with the regulation of protein kinases. Park et al. Cell 101 (7): 777-787 (2000).

Because protein kinases regulate nearly every cellular process, including metabolism, cell proliferation, cell differentiation, and cell survival, they are attractive targets for therapeutic intervention for various disease states. For example, cell-cycle control and angiogenesis, in which protein kinases play a pivotal role are cellular processes associated with numerous disease conditions such as but not limited to cancer, inflammatory diseases, abnormal angiogenesis and diseases related thereto, atherosclerosis, macular degeneration, diabetes, obesity, and pain.

Protein kinases have become attractive targets for the treatment of cancers. Fabbro et al., Pharmacology & Therapeutics 93:79-98 (2002). It has been proposed that the involvement of protein kinases in the development of human malignancies may occur by: (1) genomic rearrangements (e.g., BCR-ABL in chronic myelogenous leukemia), (2) mutations leading to constitutively active kinase activity, such as acute myelogenous leukemia and gastrointestinal tumors, (3) deregulation of kinase activity by activation of oncogenes or loss of tumor suppressor functions, such as in cancers with oncogenic RAS, (4) deregulation of kinase activity by over-expression, as in the case of EGFR and (5) ectopic expression of growth factors that can contribute to the development and maintenance of the neoplastic phenotype. Fabbro et al., Pharmacology & Therapeutics 93:79-98 (2002).

The elucidation of the intricacy of protein kinase pathways and the complexity of the relationship and interaction among and between the various protein kinases and kinase pathways highlights the importance of developing pharmaceutical agents capable of acting as protein kinase modulators, regulators or inhibitors that have beneficial activity on multiple kinases or multiple kinase pathways. Accordingly, there remains a need for new kinase modulators.

The protein named mTOR (mammalian target of rapamycin), which is also called FRAP, RAFTI or RAPTI), is a 2549-amino acid Ser/Thr protein kinase, that has been shown to be one of the most critical proteins in the mTOR/PI3K/Akt pathway that regulates cell growth and proliferation. Georgakis and Younes Expert Rev. Anticancer Ther. 6(1):131-140 (2006). mTOR exists within two complexes, mTORC1 and mTORC2. While mTORC1 is sensitive to rapamycin analogs (such as temsirolimus or everolimus), mTORC2 is largely rapamycin-insensitive. Notably, rapamycin is not a TOR kinase inhibitor. Several mTOR inhibitors have been or are being evaluated in clinical trials for the treatment of cancer. Temsirolimus was approved for use in renal cell carcinoma in 2007 and sirolimus was approved in 1999 for the prophylaxis of renal transplant rejection. Everolimus was approved in 2009 for renal cell carcinoma patients that have progressed on vascular endothelial growth factor receptor inhibitors, in 2010 for subependymal giant cell astrocytoma (SEGA) associated with tuberous sclerosis (TS) in patients who require therapy but are not candidates for surgical resection, and in 2011 for progressive neuroendocrine tumors of pancreatic origin (PNET) in patients with unresectable, locally advanced or metastatic disease. There remains a need for TOR kinase inhibitors that inhibit both mTORC1 and mTORC2 complexes.

DNA-dependent protein kinase (DNA-PK) is a serine/threonine kinase involved in the repair of DNA double strand breaks (DSBs). DSBs are considered to be the most lethal DNA lesion and occur endogenously or in response to ionizing radiation and chemotherapeutics (for review see Jackson, S. P., Bartek, J. The DNA-damage response in human biology and disease. Nature Rev 2009; 461:1071-1078). If left unrepaired, DSBs will lead to cell cycle arrest and/or cell death (Hoeijmakers, J. H. J. Genome maintenance mechanisms for preventing cancer. Nature 2001; 411: 366-374; van Gent, D. C., Hoeijmakers, J. H., Kanaar, R. Chromosomal stability and the DNA double-stranded break connection. Nat Rev Genet 2001; 2: 196-206). In response to the insult, cells have developed complex mechanisms to repair such breaks and these mechanisms may form the basis of therapeutic resistance. There are two major pathways used to repair DSBs, non-homologous end joining (NHEJ) and homologous recombination (HR). NHEJ brings broken ends of the DNA together and rejoins them without reference to a second template (Collis, S. J., DeWeese, T. L., Jeggo P. A., Parker, A. R. The life and death of DNA-PK. Oncogene 2005; 24: 949-961). In contrast, HR is dependent on the proximity of the sister chromatid which provides a template to mediate faithful repair (Takata, M., Sasaki, M. S., Sonoda, E., Morrison, C., Hashimoto, M., Utsumi, H., et al. Homologous recombination and non-homologous end joining pathways of DNA double-strand break repair have overlapping roles in the maintenance of chromosomal integrity in vertebrate cells. EMBO J 1998; 17: 5497-5508; Haber, J. E. Partners and pathways repairing a double-strand break. Trends Genet 2000; 16: 259-264). NHEJ repairs the majority of DSBs. In NHEJ, DSBs are recognized by the Ku protein that binds and then activates the catalytic subunit of DNA-PK. This leads to recruitment and activation of end-processing enzymes, polymerases and DNA ligase IV (Collis, S. J., DeWeese, T. L., Jeggo P. A., Parker, A. R. The life and death of DNA-PK. Oncogene 2005; 24: 949-961). NHEJ is primarily controlled by DNA-PK and thus inhibition of DNA-PK is an attractive approach to modulating the repair response to exogenously induced DSBs. Cells deficient in components of the NHEJ pathway are defective in DSB repair and highly sensitive to ionizing radiation and topoisomerase poisons (reviewed by Smith, G. C. M., Jackson, S. P. The DNA-dependent protein kinase. Genes Dev 1999; 13: 916-934; Jeggo, P. A., Caldecott, K., Pidsley, S., Banks, G. R. Sensitivity of Chinese hamster ovary mutants defective in DNA double strand break repair to topoisomerase II inhibitors. Cancer Res 1989; 49: 7057-7063). A DNA-PK inhibitor has been reported to have the same effect of sensitizing cancer cells to therapeutically induced DSBs (Smith, G. C. M., Jackson, S. P. The DNA-dependent protein kinase. Genes Dev 1999; 13: 916-934).

The mechanism of action of IMiD® immunomodulatory drugs is varied and complex. IMiD® immunomodulatory drugs are known to bind directly to cereblon, a component of the E3 ubiquitin ligase complex. These complexes regulate protein homeostasis. Cereblon mediates IMiD® immunomodulatory drugs tumorcidal effects, as well as certain immunomodulatory activities in T cells resulting in enhanced production of cytokine IL-2, which is important for immune cell proliferation and generation of immune responses.

IMiD® immunomodulatory drugs have immunomodulatory effects through CD4+ and CD8+ T-cell costimulation, Tregs suppression, Th1 cytokine production, NK and NKT cell activation and antibody-dependent cellular toxicity. These compounds interfere with the tumor micro-environment through anti-angiogenic actions, anti-inflammatory properties, downregulation of adhesion molecules and anti-osteogenic properties, mediated by TNFα, VEGF and βFGF secreted by BMSC, IL-6, MIP1-α and RANK, among other cytokines. The direct anti-tumor effects result from anti-proliferative activity mediated through inhibitin of cyclin-dependent kinase, change in ERG and SPARC, down regulation of NFκβ and variable inhibition of caspase 3, 8 and 9. While working through similar mechanism of action, each IMiD compound can be distinguished by unique activity and potency profiles.

Citation or identification of any reference in Section 2 of this application is not to be construed as an admission that the reference is prior art to the present application.

### 3. SUMMARY

Provided herein are methods for treating or preventing a cancer, comprising administering an effective amount of a TOR kinase inhibitor and an effective amount of an IMiD® immunomodulatory drug to a patient having a cancer, for example a hematological cancer, as described herein.

In certain embodiments, provided herein are methods for achieving an International Workshop on Chronic Lymphocytic Leukemia (IWCLL) response definition of complete response, partial response or stable disease in a patient having chronic lymphocytic leukemia, comprising administering an effective amount of a TOR kinase inhibitor in combination with an IMiD® immunomodulatory drug to said patient. In certain embodiments, provided herein are methods for achieving a National Cancer Institute-Sponsored Working Group on Chronic Lymphocytic Leukemia (NCI-WG CLL) response definition of complete response, partial response or stable disease in a patient having leukemia, comprising administering an effective amount of a TOR kinase inhibitor in combination with an IMiD® immunomodulatory drug to said patient. In certain embodiments, provided herein are methods for achieving an International Workshop Criteria (IWC) for non-Hodgkin's lymphoma of complete response, partial response or stable disease in a patient having non-Hodgkin's lymphoma, comprising administering an effective amount of a TOR kinase inhibitor in combination with an IMiD® immunomodulatory drug to said patient. In certain embodiments, provided herein are methods for achieving an International Uniform Response Criteria (IURC) for multiple myeloma of complete response, partial response or stable disease in a patient having multiple myeloma, comprising administering an effective amount of a TOR kinase inhibitor in combination with an IMiD® immunomodulatory drug to said patient. In certain embodiments, provided herein are methods for achieving a Response Evaluation Criteria in Solid Tumors (for example, RECIST 1.1) of complete response, partial response or stable disease in a patient having a solid tumor, comprising administering an effective amount of a TOR kinase inhibitor in combination with an IMiD® immunomodulatory drug to said patient. In certain embodiments, provided herein are methods for achieving a Prostate Cancer Working Group 2 (PCWG2) Criteria of complete response, partial response or stable disease in a patient having prostate cancer, comprising administering an effective amount of a TOR kinase inhibitor in combination with an IMiD® immunomodulatory drug to said patient. In certain embodiments, provided herein are methods for achieving a Responses Assessment for Neuro-Oncology (RANO) Working Group for glioblastoma multiforme of complete response, partial response or stable disease in a patient having glioblastoma multiforme, comprising administering an effective amount of a TOR kinase inhibitor in combination with an IMiD® immunomodulatory drug to said patient.

In certain embodiments, provided herein are methods for increasing survival without cancer progression of a patient having a cancer, comprising administering an effective amount of a TOR kinase inhibitor in combination with an effective amount of an IMiD® immunomodulatory drug to said patient.

In certain embodiments, the TOR kinase inhibitor is a compound as described herein. In some embodiments, the IMiD® immunomodulatory drug is a compound as described herein.

The present embodiments can be understood more fully by reference to the detailed description and examples, which are intended to exemplify non-limiting embodiments.

### 4. BRIEF DESCRIPTION OF THE DRAWINGS

FIG. 1A depicts the effect of Compound 1 when used in combination with lenalidomide on the acquisition of resistance in Multiple Myeloma cells. H929 cells were continuously treated with lenalidomide, Compound 1 or a combination of lenalidomide with Compound 1. Cell viability was assessed by propidium iodine staining and flow cytometry. FIG. 1B depicts the effect of Compound 2 when used in combination with lenalidomide on the acquisition of resistance in Multiple Myeloma cells. H929 cells were continuously treated with lenalidomide, Compound 2 or a combination of lenalidomide with Compound 2. Cell viability was assessed by propidium iodine staining and flow cytometry

FIG. 2 depicts the effects of Compound 1 on HepG2 colony formation. HepG2 cells were plated in agar and incubated with Compound 1 for 8 days before colonies were counted. Data were calculated as the percentage of control relative to the cells treated with DMSO only=100% control. Each data point represents the mean of n=3 experiments in triplicate. ***p<0.001 vs DMSO control by one way ANOVA followed by Dunnett's post test.

FIG. 3 depicts the effects of Compound 1 on SK-Hep-1 colony formation. SK-HEP-1 cells were plated in agar and incubated with Compound 1 for 8-10 days before colonies were counted. Data were calculated as the percentage of control relative to the cells treated with DMSO only=100% control. Each data point represents the mean of n=3 experiments in triplicate. ***p<0.001 vs DMSO control by one way ANOVA followed by Dunnett's post test.

FIG. 4 depicts the effects of Compound 1 plus lenalidomide on HepG2 Colony Formation. HepG2 cells were plated in agar and incubated with compound for 8 days before colonies were counted. Data were calculated as the percentage of control relative to the cells treated with DMSO only=100% control. Each data point represents the mean of n=3 experiments in triplicate. ***p<0.001, **p<0.01 vs theoretical additivity by unpaired t test.

FIG. 5 depicts the effects of Compound 1 plus lenalidomide on SK-Hep-1 colony formation. SK-Hep-1 cells were plated in agar and incubated with compound for 8 days before colonies were counted. Data were calculated as the percentage of control relative to the cells treated with DMSO only=100% control. Each data point represents the mean of n=3 experiments in triplicate. *p<0.05 vs theoretical additivity by unpaired t test.

### 5. DETAILED DESCRIPTION

### 5.1 Definitions

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxylamine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₈)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl and the like. The double bond of an alkenyl group can be unconjugated or conjugated to another unsaturated group. An alkenyl group can be unsubstituted or substituted.

A “cycloalkyl” group is a saturated, or partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, thiazolyl, pyrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3H-imidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclylalkyl group can be substituted or unsubstituted. Heterocyclyl groups encompass unsaturated, partially saturated and saturated ring systems, such as, for example, imidazolyl, imidazolinyl and imidazolidinyl groups. The phrase heterocyclyl includes fused ring species, including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridin-2(3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

A “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocyclylalkyl groups include but are not limited to 4-ethyl-morpholinyl, 4-propylmorpholinyl, furan-2-yl methyl, furan-3-yl methyl, pyridine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

A “hydroxyalkyl” group is an alkyl group as described above substituted with one or more hydroxy groups.

An “alkoxy” group is —O-(alkyl), wherein alkyl is defined above.

An “alkoxyalkyl” group is -(alkyl)-O-(alkyl), wherein alkyl is defined above.

An “amine” group is a radical of the formula: —NH₂.

A “hydroxylamine” group is a radical of the formula: —N(R^{#})OH or —NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula: —N(R^{#})O-alkyl or —NHO-alkyl, wherein R^{#} is as defined above.

An “aralkoxyamine” group is a radical of the formula: —N(R^{#})O-aryl or —NHO-aryl, wherein R^{#} is as defined above.

An “alkylamine” group is a radical of the formula: —NH-alkyl or —N(alkyl)₂, wherein each alkyl is independently as defined above.

An “aminocarbonyl” group is a radical of the formula: —C(═O)N(R^{#})₂, —C(═O)NH(R^{#}) or —C(═O)NH₂, wherein each R^{#} is as defined above.

An “acylamino” group is a radical of the formula: —NHC(═O)(R^{#}) or —N(alkyl)C(═O)(R^{#}), wherein each alkyl and R^{#} are independently as defined above.

An “O(alkyl)aminocarbonyl” group is a radical of the formula: —O(alkyl)C(═O)N(R^{#})₂, —O(alkyl)C(═O)NH(R^{#}) or —O(alkyl)C(═O)NH₂, wherein each R^{#} is independently as defined above.

An “N-oxide” group is a radical of the formula: —N⁺—O⁻.

A “carboxy” group is a radical of the formula: —C(═O)OH.

A “ketone” group is a radical of the formula: —C(═O)(R^{#}), wherein R^{#} is as defined above.

An “aldehyde” group is a radical of the formula: —CH(═O).

An “ester” group is a radical of the formula: —C(═O)O(R^{#}) or —OC(═O)(R^{#}), wherein R^{#} is as defined above.

A “urea” group is a radical of the formula: —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or —NHC(═O)NH₂^{#}, wherein each alkyl and R^{#} are independently as defined above.

An “imine” group is a radical of the formula: —N═C(R^{#})₂ or —C(R^{#})═N(R^{#}), wherein each R^{#} is independently as defined above.

An “imide” group is a radical of the formula: —C(═O)N(R#)C(═O)(R^{#}) or —N((C═O)(R^{#}))₂, wherein each R^{#} is independently as defined above.

A “urethane” group is a radical of the formula: —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula: —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})═NH, or —NHC(R^{#})═NH, wherein each R^{#} is independently as defined above.

A “guanidine” group is a radical of the formula: —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R^{#})₂)₂, —N═C(NH(R^{#}))₂, or —N═C(NH₂)₂, wherein each R^{#} is independently as defined above.

A “enamine” group is a radical of the formula: —N(R^{#})C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R^{#})₂, —C(R^{#})═C(R^{#})(N(R^{#})₂), —C(R^{#})═C(R^{#})(NH(R^{#})) or —C(R^{#})═C(R^{#})(NH₂), wherein each R^{#} is independently as defined above.

An “oxime” group is a radical of the formula: —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula: —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula: —N(R^{#})N(R^{#})₂, —NHN(R^{#})₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula: —C(═N—N(R^{#})₂)(R^{#})₂, —C(═N—NH(R^{#}))(R^{#})₂, —C(═N—NH₂)(R^{#})₂, —N(R^{#})(N═C(R^{#})₂), or —NH(N═C(R^{#})₂), wherein each R^{#} is independently as defined above.

An “azide” group is a radical of the formula: —N₃.

An “isocyanate” group is a radical of the formula: —N═C═O.

An “isothiocyanate” group is a radical of the formula: —N═C═S.

A “cyanate” group is a radical of the formula: —OCN.

A “thiocyanate” group is a radical of the formula: —SCN.

A “thioether” group is a radical of the formula; —S(R^{#}), wherein R^{#} is as defined above.

A “thiocarbonyl” group is a radical of the formula: —C(═S)(R^{#}), wherein R^{#} is as defined above.

A “sulfinyl” group is a radical of the formula: —S(═O)(R^{#}), wherein R^{#} is as defined above.

A “sulfone” group is a radical of the formula: —S(═O)₂(R^{#}), wherein R^{#} is as defined above.

A “sulfonylamino” group is a radical of the formula: —NHSO₂(R^{#}) or —N(alkyl)SO₂(R^{#}), wherein each alkyl and R^{#} are defined above.

A “sulfonamide” group is a radical of the formula: —S(═O)₂N(R^{#})₂, or —S(═O)₂NH(R^{#}), or —S(═O)₂NH₂, wherein each R^{#} is independently as defined above.

A “phosphonate” group is a radical of the formula: —P(═O)(O(R^{#}))₂, —P(═O)(OH)₂, —OP(═O)(O(R^{#})(R^{#}), or —OP(═O)(OH)(R^{#}), wherein each R^{#} is independently as defined above.

A “phosphine” group is a radical of the formula: —P(R^{#})₂, wherein each R^{#} is independently as defined above

When the groups described herein, with the exception of alkyl group are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxylamine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein and unless otherwise indicated, the term “clathrate” means a TOR kinase inhibitor or an IMiD® immunomodulatory drug, or a salt thereof, in the form of a crystal lattice that contains spaces (e.g., channels) that have a guest molecule (e.g., a solvent or water) trapped within or a crystal lattice wherein a TOR kinase inhibitor or an IMiD® immunomodulatory drug is a guest molecule.

As used herein and unless otherwise indicated, the term “solvate” means a TOR kinase inhibitor or an IMiD® immunomodulatory drug, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a TOR kinase inhibitor or an IMiD® immunomodulatory drug, or a salt thereof, that further includes a stoichiometric or non-stoichiometric amount of water bound by non-covalent intermolecular forces.

As used herein and unless otherwise indicated, the term “prodrug” means a TOR kinase inhibitor or an IMiD® immunomodulatory drug derivative that can hydrolyze, oxidize, or otherwise react under biological conditions (in vitro or in vivo) to provide an active compound, particularly a TOR kinase inhibitor or an IMiD® immunomodulatory drug. Examples of prodrugs include, but are not limited to, derivatives and metabolites of a TOR kinase inhibitor or an IMiD® immunomodulatory drug that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the terms “stereoisomer,” “stereomerically pure” or “optically pure” mean one stereoisomer of a TOR kinase inhibitor or an IMiD® immunomodulatory drug that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The TOR kinase inhibitors or IMiD® immunomodulatory drugs can have chiral centers and can occur as racemates, individual enantiomers or diastereomers, and mixtures thereof. All such isomeric forms are included within the embodiments disclosed herein, including mixtures thereof. The use of stereomerically pure forms of such TOR kinase inhibitors or IMiD® immunomodulatory drugs, as well as the use of mixtures of those forms are encompassed by the embodiments disclosed herein. For example, mixtures comprising equal or unequal amounts of the enantiomers of a particular TOR kinase inhibitor or an IMiD® immunomodulatory drug may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

It should also be noted the TOR kinase inhibitors or IMiD® immunomodulatory drugs can include E and Z isomers, or a mixture thereof, and cis and trans isomers or a mixture thereof. In certain embodiments, the TOR kinase inhibitors or IMiD® immunomodulatory drugs are isolated as either the cis or trans isomer. In other embodiments, the TOR kinase inhibitors or IMiD® immunomodulatory drugs are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

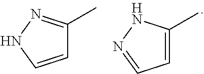

As readily understood by one skilled in the art, a wide variety of functional groups and other structures may exhibit tautomerism and all tautomers of the TOR kinase inhibitors or IMiD® immunomodulatory drugs are within the scope of the present invention.

It should also be noted the TOR kinase inhibitors or IMiD® immunomodulatory drugs can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the TOR kinase inhibitors or IMiD® immunomodulatory drugs as described herein, whether radioactive or not, are intended to be encompassed within the scope of the embodiments provided herein. In some embodiments, there are provided isotopologues of the TOR kinase inhibitors or IMiD® immunomodulatory drugs, for example, the isotopologues are deuterium, carbon-13, or nitrogen-15 enriched TOR kinase inhibitors or IMiD® immunomodulatory drugs.

It should be noted that if there is a discrepancy between a depicted structure and a name for that structure, the depicted structure is to be accorded more weight.

“Treating” as used herein, means an alleviation, in whole or in part, of a cancer or a symptom associated with a cancer, or slowing, or halting of further progression or worsening of those symptoms.

“Preventing” as used herein, means the prevention of the onset, recurrence or spread, in whole or in part, of a cancer, or a symptom thereof.

The term “effective amount” in connection with an TOR kinase inhibitor or an IMiD® immunomodulatory drug means an amount alone or in combination capable of alleviating, in whole or in part, a symptom associated with a cancer, or slowing or halting further progression or worsening of those symptoms, or treating or preventing a cancer in a subject having or at risk for having a cancer. The effective amount of the TOR kinase inhibitor or an IMiD® immunomodulatory drug, for example in a pharmaceutical composition, may be at a level that will exercise the desired effect; for example, about 0.005 mg/kg of a subject's body weight to about 100 mg/kg of a patient's body weight in unit dosage for both oral and parenteral administration.

The term “cancer” includes, but is not limited to, hematological or blood borne tumors and solid tumors. Blood borne tumors include lymphomas, leukemias and myelomas. Lymphomas and leukemias are malignancies arising among white blood cells. The term “cancer” also refers to any of various malignant neoplasms characterized by the proliferation of cells that can invade surrounding tissue and metastasize to new body sites. Both benign and malignant tumors are classified according to the type of tissue in which they are found. For example, fibromas are neoplasms of fibrous connective tissue, and melanomas are abnormal growths of pigment (melanin) cells. Malignant tumors originating from epithelial tissue, e.g., in skin, bronchi, and stomach, are termed carcinomas. Malignancies of epithelial glandular tissue such as are found in the breast, prostate, and colon, are known as adenocarcinomas. Malignant growths of connective tissue, e.g., muscle, cartilage, lymph tissue, and bone, are called sarcomas. Through the process of metastasis, tumor cell migration to other areas of the body establishes neoplasms in areas away from the site of initial appearance. Bone tissues are one of the most favored sites of metastases of malignant tumors, occurring in about 30% of all cancer cases. Among malignant tumors, cancers of the lung, breast, prostate or the like are particularly known to be likely to metastasize to bone.

In the context of neoplasm, cancer, tumor growth or tumor cell growth, inhibition may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, is referred to herein as prevention or chemoprevention. In this context, the term “prevention” includes either preventing the onset of clinically evident neoplasia altogether or preventing the onset of a preclinically evident stage of neoplasia in individuals at risk. Also intended to be encompassed by this definition is the prevention of transformation into malignant cells or to arrest or reverse the progression of premalignant cells to malignant cells. This includes prophylactic treatment of those at risk of developing the neoplasia.

The term “refractory B-cell non-Hodgkin's lymphoma” as used herein is defined as B-cell non-Hodgkin's lymphoma which was treated with an anti-CD-20 antibody-containing regimen, for example rituximab-containing regimen, (i) without achieving at least a partial response to therapy or (ii) which progressed within 6 months of treatment.

The term “relapsed B-cell non-Hodgkin's lymphoma” as used herein is defined as B-cell non-Hodgkin's lymphoma which progressed after ≧6 months post-treatment with an anti-CD-20 antibody-containing regimen, for example rituximab-containing regimen, after achieving partial response or complete response to therapy.

A person of ordinary skill will appreciate that diseases characterized as “B-cell lymphoma” exist as a continuum of diseases or disorders. While the continuum of B-cell lymphomas is sometimes discussed in terms of “aggressive” B-cell lymphomas or “indolent” B-cell lymphomas, a person of ordinary skill will appreciate that a B-cell lymphoma characterized as indolent may progress and become an aggressive B-cell lymphoma. Conversely, an aggressive form of B-cell lymphoma may be downgraded to an indolent or stable form of B-cell lymphoma. Reference is made to indolent and aggressive B-cell lymphomas as generally understood by a person skilled in the art with the recognition that such characterizations are inherently dynamic and depend on the particular circumstances of the individual.

As used herein, and unless otherwise specified, the term “in combination with” includes the administration of two or more therapeutic agents simultaneously, concurrently, or sequentially within no specific time limits unless otherwise indicated. In one embodiment, a TOR kinase inhibitor is administered in combination with an IMiD® immunomodulatory drug. In one embodiment, a TOR kinase inhibitor is administered in combination with an IMiD® immunomodulatory drug and further in combination with an anti-CD20 antibody, for example, rituximab (Rituxan®, Biogen Idec/Genentech or MabThera®, Hoffmann-La Roche) In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, or any combination thereof. For example, in one embodiment, the first agent can be administered prior to the second therapeutic agent, for e.g. 1 week. In another, the first agent can be administered prior to (for example 1 day prior) and then concomitant with the second therapeutic agent.

The terms “patient” and “subject” as used herein include an animal, including, but not limited to, an animal such as a cow, monkey, horse, sheep, pig, chicken, turkey, quail, cat, dog, mouse, rat, rabbit or guinea pig, in one embodiment a mammal, in another embodiment a human. In one embodiment, a “patient” or “subject” is a human having a cancer.

In the context of a cancer, inhibition may be assessed by inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others. OS as used herein means the time from randomization until death from any cause, and is measured in the intent-to-treat population. TTP as used herein means the time from randomization until objective tumor progression; TTP does not include deaths. As used herein, PFS means the time from randomization until objective tumor progression or death. In one embodiment, PFS rates will be computed using the Kaplan-Meier estimates. In the extreme, complete inhibition, is referred to herein as prevention or chemoprevention. In this context, the term “prevention” includes either preventing the onset of clinically evident advanced cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

In certain embodiments, the treatment of lymphoma may be assessed by the International Workshop Criteria (IWC) for non-Hodgkin lymphoma (NHL) (see Cheson B D, Pfistner B, Juweid, M E, et. al. Revised Response Criteria for Malignant Lymphoma. J. Clin. Oncol: 2007: (25) 579-586), using the response and endpoint definitions shown below:

| Response | Definition | Nodal Masses | Spleen, liver | Bone Marrow |
|---|---|---|---|---|
| CR | Disappearance | (a) FDG-avid or PET | Not | Infiltrate cleared |
| | of all evidence | positive prior to therapy; | palpable, | on repeat biopsy; if |
| | of disease | mass of any size permitted | nodules | indeterminate by |
| | | if PET negative | disappeared | morphology, |
| | | (b) Variably FDG-avid or | | immunohistochemistry |
| | | PET negative; regression | | should be negative |
| | | to normal size on CT | | |
| PR | Regression of | ≧50% decrease in SPD of | ≧50% | Irrelevant if |
| | measurable | up to 6 largest dominant | decrease in | positive prior to |
| | disease and no | masses; no increase in size | SPD of | therapy; cell type |
| | new sites | of other nodes | nodules (for | should be specified |
| | | (a) FDG-avid or PET | single | |
| | | positive prior to therapy; | nodule in | |
| | | one or more PET positive | greatest | |
| | | at previously involved site | transverse | |
| | | (b) Variably FDG-avid or | diameter); | |
| | | PET negative; regression | no increase | |
| | | on CT | in size of | |
| | | | liver or | |
| | | | spleen | |
| SD | Failure to | (a) FDG-avid or PET | | |
| | attain CR/PR | positive prior to therapy; | | |
| | or PD | PET positive at prior sites | | |
| | | of disease and no new | | |
| | | sites on CT or PET | | |
| | | (b) Variably FDG-avid or | | |
| | | PET negative; no change | | |
| | | in size of previous lesions | | |
| | | on CT | | |
| PD or | Any new | Appearance of a new | ≧50% | New or recurrent |
| relapsed | lesion or | lesion(s) ≧1.5 cm in any | increase | involvement |
| disease | increase by ≧50% | axis, ≧50% increase in | from nadir in | |
| | of | SPD of more than one | the SPD of | |
| | previously | node, | any previous | |
| | involved sites | or ≧50% increase in | lesions | |
| | from nadir | longest diameter of a | | |
| | | previously identifed node | | |
| | | ≧1 cm in short axis | | |
| | | Lesions PET positive if | | |
| | | FDG-avid lymphoma or | | |
| | | PET positive prior to | | |
| | | therapy | | |

Abbreviations:
CR, complete remission;
FDG, [¹⁸F]fluorodeoxyglucose;
PET, positron emission tomography;
CT, computed tomography;
PR, partial remission;
SPD, sum of the product of the diameters;
SD, stable disease;
PD, progressive disease.

| | | | Measured |
|---|---|---|---|
| End point | Patients | Definition | from |
| Primary | | | |
| Overall survival | All | Death as a result of any cause | Entry onto |
| | | | study |
| Progression-free | All | Disease progression or death as a result of | Entry onto |
| survival | | any cause | study |
| Secondary | | | |
| Event-free | All | Failure of treatment or death as result of | Entry onto |
| survival | | any cause | study |
| Time to | All | Time to progression or death as a result of | Entry onto |
| progression | | lymphoma | study |
| Disease-free | In CR | Time to relapse or death as a result of | Documentation |
| survival | | lymphoma or acute toxicity of treatment | of response |
| Response duration | In CR | Time to relapse or progression | Documentation |
| | or PR | | of response |
| Lymphoma- | All | Time to death as a result of lymphoma | Entry onto |
| specific survival | | | study |
| Time to next | All | Time to new treatment | End of primary |
| treatment | | | treatment |

Abbreviations:
CR: complete remission;
PR: partial remission.

In one embodiment, the end point for lymphoma is evidence of clinical benefit. Clinical benefit may reflect improvement in quality of life, or reduction in patient symptoms, transfusion requirements, frequent infections, or other parameters. Time to reappearance or progression of lymphoma-related symptoms can also be used in this end point.

In certain embodiments, the treatment of CLL may be assessed by the International Workshop Guidelines for CLL (see Hallek M, Cheson B D, Catovsky D, et al. Guidelines for the diagnosis and treatment of chronic lymphocytic leukemia: a report from the International Workshop on Chronic Lymphocytic Leukemia updating the National Cancer Institute-Working Group 1996 guidelines. Blood, 2008; (111) 12: 5446-5456) using the response and endpoint definitions shown therein and in particular:

| Parameter | CR | PR | PD |
|---|---|---|---|
| Group A | | | |
| Lymphadenopathy^{†} | None >1.5 cm | Decrease ≧50% | Increase ≧50% |
| Hepatomegaly | None | Decrease ≧50% | Increase ≧50% |
| Splenomegaly | None | Decrease ≧50% | Increase ≧50% |
| Blood lymphocytes | <4000/μL | Decrease ≧50% | Increase ≧50% |
| | | from baseline | over baseline |
| Marrow‡ | Normocellular, <30% | 50% reduction in | |
| | lymphocytes, no B- | marrow infiltrate, or | |
| | lymphoid nodules. | B-lymphoid nodules | |
| | Hypocellular marrow | | |
| | defines CRi (5.1.6). | | |
| Group B | | | |
| Platelet count | >100 000/μL | >100 000/μL or | Decrease of ≧50% |
| | | increase ≧50% over | from |
| | | baseline | baseline |
| | | | secondary to |
| | | | CLL |
| Hemoglobin | >11.0 g/dL | >11 g/dL or | Decrease of >2 g/dL |
| | | increase ≧50% over | from |
| | | baseline | baseline |
| | | | secondary to |
| | | | CLL |
| Neutrophils^{‡} | >1500/μL | >1500/μL or >50% | |
| | | improvement | |
| | | over baseline | |

Group A criteria define the tumor load;
Group B criteria define the function of the hematopoietic system (or marrow).
CR (complete remission): all of the criteria have to be met, and patients have to lack disease-related constitutional symptoms;
PR (partial remission): at least two of the criteria of group A plus one of the criteria of group B have to be met;
SD is absence of progressive disease (PD) and failure to achieve at least a PR;
PD: at least one of the above criteria of group A or group B has to be met.
Sum of the products of multiple lymph nodes (as evaluated by CT scans in clinical trials, or by physical examination in general practice). These parameters are irrelevant for some response categories.

In certain embodiments, the treatment of multiple myeloma may be assessed by the International Uniform Response Criteria for Multiple Myeloma (IURC) (see Durie B G M, Harousseau J-L, Miguel J S, et al. International uniform response criteria for multiple myeloma. Leukemia, 2006; (10) 10:1-7), using the response and endpoint definitions shown below:

| Response Subcategory | Response Criteria^{a} |
|---|---|
| sCR | CR as defined below plus |
| | Normal FLC ratio and |
| | Absence of clonal cells in bone marrow^{b} by |
| | immunohistochemistry or |
| | immunofluorescence^{c} |
| CR | Negative immunofixation on the serum and urine and |
| | Disappearance of any soft tissue plasmacytomas and |
| | <5% plasma cells in bone marrow^{b} |
| VGPR | Serum and urine M-protein detectable by |
| | immunofixation but not on electrophoresis or 90% or |
| | greater reduction in serum M-protein plus urine |
| | M-protein level <100 mg per 24 h |
| PR | ≧50% reduction of serum M-protein and reduction in |
| | 24-h urinary M-protein by≧90% or to <200 mg per 24 h |
| | If the serum and urine M-protein are unmeasurable,^{d} a |
| | ≧50% decrease in the difference between involved and |
| | uninvolved FLC levels is required in place of the M- |
| | protein criteria |
| | If serum and urine M-protein are unmeasurable, and |
| | serum free light assay is also unmeasurable, ≧50% |
| | reduction in plasma cells is required in place of |
| | M-protein, provided baseline bone marrow plasma cell |
| | percentage was ≧30% |
| | In addition to the above listed criteria, if present at |
| | baseline, a ≧50% reduction in the size of soft tissue |
| | plasmacytomas is also required |
| SD (not recommended for use as | Not meeting criteria for CR, VGPR, PR or progressive |
| an indicator of response; stability | disease |
| of disease is best described by | |
| providing the time to progression | |
| estimates) | |

Abbreviations:
CR, complete response;
FLC, free light chain;
PR, partial response;
SD, stable disease;
sCR, stringent complete response;
VGPR, very good partial response;
^{a}All response categories require two consecutive assessments made at anytime before the institution of any new therapy; all categories also require no known evidence of progressive or new bone lesions if radiographic studies were performed. Radiographic studies are not required to satisfy these response requirements;
^{b}Confirmation with repeat bone marrow biopsy not needed;
^{c}Presence/absence of clonal cells is based upon the κ/λ ratio. An abnormal κ/λ ratio by immunohistochemistry and/or immunofluorescence requires a minimum of 100 plasma cells for analysis. An abnormal ratio reflecting presence of an abnormal clone is κ/λ of >4:1 or <1:2.
^{d}Measurable disease defined by at least one of the following measurements: Bone marrow plasma cells ≧30%; Serum M-protein ≧1 g/dl (≧10 gm/l)[10 g/l]; Urine M-protein ≧200 mg/24 h; Serum FLC assay: Involved FLC level ≧10 mg/dl (≧100 mg/l); provided serum FLC ratio is abnormal.

In certain embodiments, the treatment of a cancer may be assessed by Response Evaluation Criteria in Solid Tumors (RECIST 1.1) (see Thereasse P., et al. New Guidelines to Evaluate the Response to Treatment in Solid Tumors. J. of the National Cancer Institute; 2000; (92) 205-216 and Eisenhauer E. A., Therasse P., Bogaerts J., et al. New response evaluation criteria in solid tumours: Revised RECIST guideline (version 1.1). European J. Cancer; 2009; (45) 228-247). Overall responses for all possible combinations of tumor responses in target and non-target lesions with our without the appearance of new lesions are as follows:

| Target lesions | Non-target lesions | New lesions | Overall response |
|---|---|---|---|
| CR | CR | No | CR |
| CR | Incomplete | No | PR |
| | response/SD | | |
| PR | Non-PD | No | PR |
| SD | Non-PD | No | SD |
| PD | Any | Yes or no | PD |
| Any | PD | Yes or no | PD |
| Any | Any | Yes | PD |

CR = complete response,
PR = partial response;
SD = stable disease; and
PD = progressive disease.

With respect to the evaluation of target lesions, complete response (CR) is the disappearance of all target lesions, partial response (PR) is at least a 30% decrease in the sum of the longest diameter of target lesions, taking as reference the baseline sum longest diameter, progressive disease (PD) is at least a 20% increase in the sum of the longest diameter of target lesions, taking as reference the smallest sum longest diameter recorded since the treatment started or the appearance of one or more new lesions and stable disease (SD) is neither sufficient shrinkage to qualify for partial response nor sufficient increase to qualify for progressive disease, taking as reference the smallest sum longest diameter since the treatment started.

With respect to the evaluation of non-target lesions, complete response (CR) is the disappearance of all non-target lesions and normalization of tumor marker level; incomplete response/stable disease (SD) is the persistence of one or more non-target lesion(s) and/or the maintenance of tumor marker level above the normal limits, and progressive disease (PD) is the appearance of one or more new lesions and/or unequivocal progression of existing non-target lesions.

The procedures, conventions, and definitions described below provide guidance for implementing the recommendations from the Response Assessment for Neuro-Oncology (RANO) Working Group regarding response criteria for high-grade gliomas (Wen P., Macdonald, D R., Reardon, D A., et al. Updated response assessment criteria for highgrade gliomas: Response assessment in neuro-oncology working group. J Clin Oncol 2010; 28: 1963-1972). Primary modifications to the RANO criteria for Criteria for Time Point Responses (TPR) can include the addition of operational conventions for defining changes in glucocorticoid dose, and the removal of subjects' clinical deterioration component to focus on objective radiologic assessments. The baseline MRI scan is defined as the assessment performed at the end of the post-surgery rest period, prior to re-initiating compound treatment. The baseline MRI is used as the reference for assessing complete response (CR) and partial response (PR). Whereas, the smallest SPD (sum of the products of perpendicular diameters) obtained either at baseline or at subsequent assessments will be designated the nadir assessment and utilized as the reference for determining progression. For the 5 days preceding any protocol-defined MRI scan, subjects receive either no glucocorticoids or are on a stable dose of glucocorticoids. A stable dose is defined as the same daily dose for the 5 consecutive days preceding the MRI scan. If the prescribed glucocorticoid dose is changed in the 5 days before the baseline scan, a new baseline scan is required with glucocorticoid use meeting the criteria described above. The following definitions will be used.

Measurable Lesions: Measurable lesions are contrast-enhancing lesions that can be measured bidimensionally. A measurement is made of the maximal enhancing tumor diameter (also known as the longest diameter, LD). The greatest perpendicular diameter is measured on the same image. The cross hairs of bidimensional measurements should cross and the product of these diameters will be calculated.

Minimal Diameter: T1-weighted image in which the sections are 5 mm with 1 mm skip. The minimal LD of a measurable lesion is set as 5 mm by 5 mm. Larger diameters may be required for inclusion and/or designation as target lesions. After baseline, target lesions that become smaller than the minimum requirement for measurement or become no longer amenable to bidimensional measurement will be recorded at the default value of 5 mm for each diameter below 5 mm. Lesions that disappear will be recorded as 0 mm by 0 mm.

Multicentric Lesions: Lesions that are considered multicentric (as opposed to continuous) are lesions where there is normal intervening brain tissue between the two (or more) lesions. For multicentric lesions that are discrete foci of enhancement, the approach is to separately measure each enhancing lesion that meets the inclusion criteria. If there is no normal brain tissue between two (or more) lesions, they will be considered the same lesion.

Nonmeasurable Lesions: All lesions that do not meet the criteria for measurable disease as defined above will be considered non-measurable lesions, as well as all nonenhancing and other truly nonmeasurable lesions. Nonmeasurable lesions include foci of enhancement that are less than the specified smallest diameter (ie., less than 5 mm by 5 mm), nonenhancing lesions (eg., as seen on T1-weighted post-contrast, T2-weighted, or fluid-attenuated inversion recovery (FLAIR) images), hemorrhagic or predominantly cystic or necrotic lesions, and leptomeningeal tumor. Hemorrhagic lesions often have intrinsic T1-weighted hyperintensity that could be misinterpreted as enhancing tumor, and for this reason, the pre-contrast T1-weighted image may be examined to exclude baseline or interval sub-acute hemorrhage.

At baseline, lesions will be classified as follows: Target lesions: Up to 5 measurable lesions can be selected as target lesions with each measuring at least 10 mm by 5 mm, representative of the subject's disease; Non-target lesions: All other lesions, including all nonmeasurable lesions (including mass effects and T2/FLAIR findings) and any measurable lesion not selected as a target lesion. At baseline, target lesions are to be measured as described in the definition for measurable lesions and the SPD of all target lesions is to be determined. The presence of all other lesions is to be documented. At all post-treatment evaluations, the baseline classification of lesions as target and non-target lesions will be maintained and lesions will be documented and described in a consistent fashion over time (eg., recorded in the same order on source documents and eCRFs). All measurable and nonmeasurable lesions must be assessed using the same technique as at baseline (e.g., subjects should be imaged on the same MRI scanner or at least with the same magnet strength) for the duration of the study to reduce difficulties in interpreting changes. At each evaluation, target lesions will be measured and the SPD calculated. Non-target lesions will be assessed qualitatively and new lesions, if any, will be documented separately. At each evaluation, a time point response will be determined for target lesions, non-target lesions, and new lesion. Tumor progression can be established even if only a subset of lesions is assessed. However, unless progression is observed, objective status (stable disease, PR or CR) can only be determined when all lesions are assessed.

Confirmation assessments for overall time point responses of CR and PR will be performed at the next scheduled assessment, but confirmation may not occur if scans have an interval of <28 days. Best response, incorporating confirmation requirements, will be derived from the series of time points.

In certain embodiments, treatment of a cancer may be assessed by the inhibition of phosphorylation of S6RP, 4E-BP1, AKT and/or DNA-PK in circulating blood and/or tumor cells, and/or skin biopsies or tumor biopsies/aspirates, before, during and/or after treatment with a TOR kinase inhibitor. For example, the inhibition of phosphorylation of S6RP, 4E-BP1, AKT and/or DNA-PK is assessed in B-cells, T-cells and/or monocytes. In other embodiments, treatment of a cancer may be assessed by the inhibition of DNA-dependent protein kinase (DNA-PK) activity in skin samples and/or tumor biopsies/aspirates, such as by assessment of the amount of pDNA-PK 52056 as a biomarker for DNA damage pathways, before, during, and/or after TOR kinase inhibitor treatment. In one embodiment, the skin sample is irradiated by UV light.

In the extreme, complete inhibition, is referred to herein as prevention or chemoprevention. In this context, the term “prevention” includes either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

As used herein, the term “antibody”, or grammatical variations thereof (i.e., antibodies), refers to polypeptide(s) capable of binding to an epitope. In some embodiments, an antibody is a full-length antibody. In some embodiments, an antibody is less than full length (i.e., an antibody fragment) but includes at least one binding site. In some such embodiments, the binding site comprises at least one, and preferably at least two sequences with structure of antibody variable regions. In some embodiments, the term “antibody” encompasses any protein having a binding domain which is homologous or largely homologous to an immunoglobulin-binding domain. In particular embodiments, the term “antibody” encompasses polypeptides having a binding domain that shows at least 99% identity with an immunoglobulin-binding domain. In some embodiments, the antibody is any protein having a binding domain that shows at least 70%, at least 80%, at least 85%, at least 90% or at least 95% identity with an immunoglobulin-binding domain. Antibody polypeptides in accordance with the present invention may be prepared by any available means, including, for example, isolation from a natural source or antibody library, recombinant production in or with a host system, chemical synthesis, etc., or combinations thereof. In some embodiments, an antibody is monoclonal or polyclonal. In some embodiments, an antibody may be a member of any immunoglobulin class, including any of the human classes IgG, IgM, IgA, IgD and IgE. In certain embodiments, an antibody is a member of the IgG immunoglobulin class. In some embodiments, the term “antibody” refers to any derivative of an antibody that possesses the ability to bind to an epitope of interest. In some embodiments, an antibody fragment comprises multiple chains that are linked together, for example, by disulfide linkages. In some embodiments, an antibody is a human antibody. In some embodiments, an antibody is a humanized antibody. In some embodiments, humanized antibodies include chimeric immunoglobulins, immunoglobulin chains or antibody fragments (Fv, Fab, Fab′, F(ab′)₂ or other antigen binding subsequences of antibodies) that contain minimal sequence derived from non-human immunoglobulin. In some embodiments, humanized antibodies are human immunoglobulin (recipient antibody) in which residues from a complementary-determining region (CDR) of the recipient are replaced by residues from a CDR of a non-human species (donor antibody) such as mouse, rat or rabbit having the desired specificity, affinity and capacity. In particular embodiments, antibodies for use in the present invention bind to particular epitopes of CD20. In some embodiments, epitopes of CD20 to which anti-CD20 antibodies bind include, for example, 170ANPS173 (Binder et al., Blood 2006, 108(6): 1975-1978), FMC7 (Deans et al., Blood 2008, 111(4): 2492), Rp5-L and Rp15-C (mimotopes of CD20) (Perosa et al., J. Immunol. 2009, 182:416-423), 182YCYSI185 (Binder et al., Blood 2006, 108(6): 1975-1978) and WEWTI (a mimic of 182YCYSI185) (Binder et al., Blood 2006, 108(6): 1975-1978). In some embodiments, an anti-CD20 antibody has a binding affinity (Kd) for an epitope of CD20 of less than 12 nM, less than 11 nM, less than 10 nM, less than 9 nM, less than 8 nM, less than 7 nM, less than 6 nM, less than 5 nM, less than 4 nM, less than 3 nM, less than 2 nM or less than 1 nM.

As used herein, the term “biosimilar” (for example, of an approved reference product/biological drug, such as a protein therapeutic, antibody, etc.) refers to a biologic product that is similar to the reference product based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is approved and intended to be used and for which approval is sought (e.g., that there are no clinically meaningful differences between the biological product and the reference product in terms of the safety, purity, and potency of the product).

In some embodiments, the biosimilar biological product and reference product utilizes the same mechanism or mechanisms of action for the condition or conditions of use prescribed, recommended, or suggested in the proposed labeling, but only to the extent the mechanism or mechanisms of action are known for the reference product. In some embodiments, the condition or conditions of use prescribed, recommended, or suggested in the labeling proposed for the biological product have been previously approved for the reference product. In some embodiments, the route of administration, the dosage form, and/or the strength of the biological product are the same as those of the reference product. In some embodiments, the facility in which the biological product is manufactured, processed, packed, or held meets standards designed to assure that the biological product continues to be safe, pure, and potent. The reference product may be approved in at least one of the U.S., Europe, or Japan. A biosimilar can be for example, a presently known antibody having the same primary amino acid sequence as a marketed antibody, but may be made in different cell types or by different production, purification or formulation methods.

### 5.2 TOR Kinase Inhibitors

The compounds provided herein are generally referred to as “TOR kinase inhibitor(s).” one aspect, the TOR kinase inhibitors do not include rapamycin or rapamycin analogs (rapalogs).

In one embodiment, the TOR kinase inhibitors include compounds having the following formula (I):

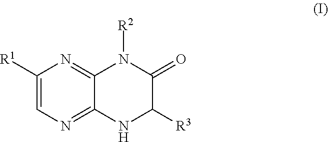

and pharmaceutically acceptable salts, clathrates, solvates, stereoisomers, tautomers, metabolites, isotopologues and prodrugs thereof, wherein:

R¹ is substituted or unsubstituted C₁₋₈ alkyl, substituted or unsubstituted aryl, substituted or unsubstituted cycloalkyl, substituted or unsubstituted heterocyclyl, or substituted or unsubstituted heterocyclylalkyl;

R² is H, substituted or unsubstituted C₁₋₈ alkyl, substituted or unsubstituted cycloalkyl, substituted or unsubstituted heterocyclyl, substituted or unsubstituted heterocyclylalkyl, substituted or unsubstituted aralkyl, or substituted or unsubstituted cycloalkylalkyl;

R³ is H, or a substituted or unsubstituted C₁₋₈ alkyl,

wherein in certain embodiments, the TOR kinase inhibitors do not include 7-(4-hydroxyphenyl)-1-(3-methoxybenzyl)-3,4-dihydropyrazino[2,3-b]pyrazin-2(1H)-one, depicted below:

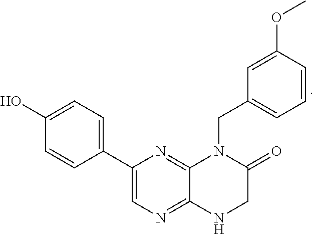 [SMILES: COc1cccc(CN2C(=O)CNc3ncc(-c4ccc(O)cc4)nc32)c1]

In some embodiments of compounds of formula (I), R¹ is substituted or unsubstituted aryl or substituted or unsubstituted heteroaryl. For example, R¹ is phenyl, pyridyl, pyrimidyl, benzimidazolyl, 1H-pyrrolo[2,3-b]pyridyl, indazolyl, indolyl, 1H-imidazo[4,5-b]pyridyl, 1H-imidazo[4,5-b]pyridin-2(3H)-onyl, 3H-imidazo[4,5-b]pyridyl, or pyrazolyl, each optionally substituted. In some embodiments, R¹ is phenyl substituted with one or more substituents independently selected from the group consisting of substituted or unsubstituted C₁₋₈ alkyl (for example, methyl), substituted or unsubstituted heterocyclyl (for example, a substituted or unsubstituted triazolyl or pyrazolyl), aminocarbonyl, halogen (for example, fluorine), cyano, hydroxyalkyl and hydroxy. In other embodiments, R¹ is pyridyl substituted with one or more substituents independently selected from the group consisting of substituted or unsubstituted C₁₋₈ alkyl (for example, methyl), substituted or unsubstituted heterocyclyl (for example, a substituted or unsubstituted triazolyl), halogen, aminocarbonyl, cyano, hydroxyalkyl (for example, hydroxypropyl), —OR, and —NR₂, wherein each R is independently H, or a substituted or unsubstituted C₁₋₄ alkyl. In some embodiments, R¹ is 1H-pyrrolo[2,3-b]pyridyl or benzimidazolyl, optionally substituted with one or more substituents independently selected from the group consisting of substituted or unsubstituted C₁₋₈ alkyl, and —NR₂, wherein R is independently H, or a substituted or unsubstituted C₁₋₄ alkyl.

In some embodiments, R¹ is

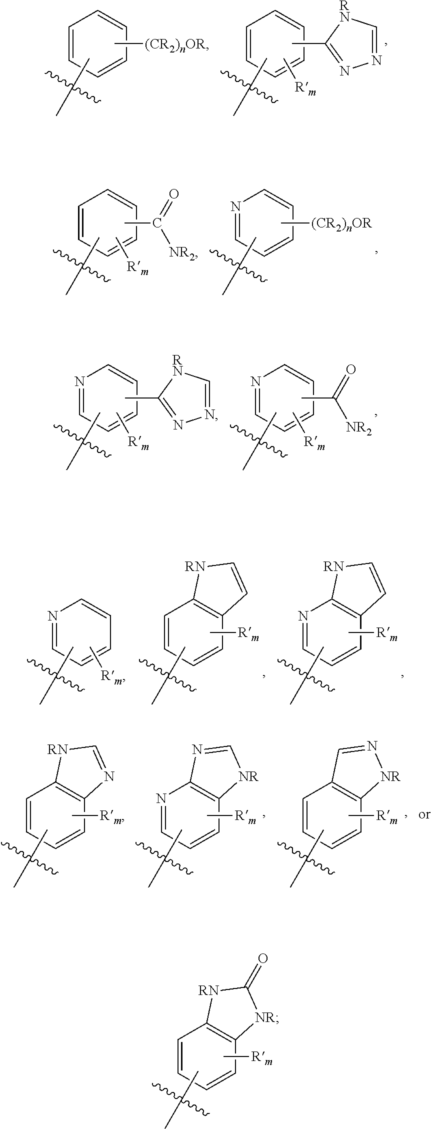

wherein R is at each occurrence independently H, or a substituted or unsubstituted C₁₋₄ alkyl (for example, methyl); R′ is at each occurrence independently a substituted or unsubstituted C₁₋₄ alkyl (for example, methyl), halogen (for example, fluoro), cyano, —OR, or —NR₂; m is 0-3; and n is 0-3. It will be understood by those skilled in the art that any of the substituents R′ may be attached to any suitable atom of any of the rings in the fused ring systems.

In some embodiments of compounds of formula (I), R¹ is

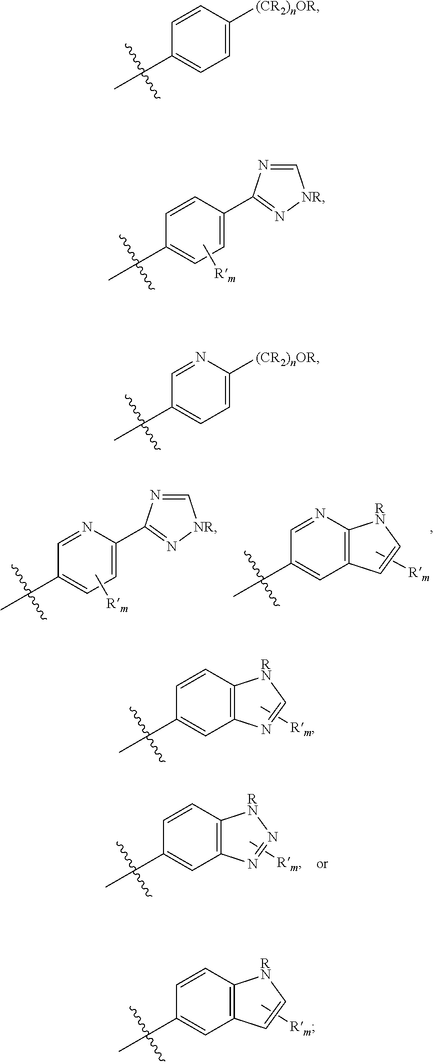

wherein R is at each occurrence independently H, or a substituted or unsubstituted C₁₋₄ alkyl; R′ is at each occurrence independently a substituted or unsubstituted C₁₋₄ alkyl, halogen, cyano, —OR or —NR₂; m is 0-3; and n is 0-3.

In some embodiments of compounds of formula (I), R² is H, substituted or unsubstituted C₁₋₈ alkyl, substituted or unsubstituted cycloalkyl, substituted or unsubstituted heterocyclyl, substituted or unsubstituted C₁₋₄ alkyl-heterocyclyl, substituted or unsubstituted C₁₋₄ alkyl-aryl, or substituted or unsubstituted C₁₋₄ alkyl-cycloalkyl. For example, R² is H, methyl, ethyl, n-propyl, isopropyl, n-butyl, sec-butyl, isobutyl, tert-butyl, n-pentyl, isopentyl, cyclopentyl, cyclohexyl, tetrahydrofuranyl, tetrahydropyranyl, (C₁₋₄ alkyl)-phenyl, (C₁₋₄ alkyl)-cyclopropyl, (C₁₋₄ alkyl)-cyclobutyl, (C₁₋₄ alkyl)-cyclopentyl, (C₁₋₄ alkyl)-cyclohexyl, (C₁₋₄ alkyl)-pyrrolidyl, (C₁₋₄ alkyl)-piperidyl, (C₁₋₄ alkyl)-piperazinyl, (C₁₋₄ alkyl)-morpholinyl, (C₁₋₄ alkyl)-tetrahydrofuranyl, or (C₁₋₄ alkyl)-tetrahydropyranyl, each optionally substituted.

In other embodiments, R² is H, C₁₋₄ alkyl, (C₁₋₄alkyl)(OR),

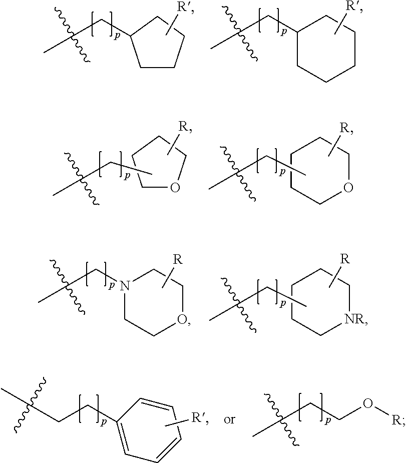

wherein R is at each occurrence independently H, or a substituted or unsubstituted C₁₋₄ alkyl (for example, methyl); R′ is at each occurrence independently H, —OR, cyano, or a substituted or unsubstituted C₁₋₄ alkyl (for example, methyl); and p is 0-3.

In other embodiments of compounds of formula (I), R² is H, C₁₋₄ alkyl, (C₁₋₄alkyl)(OR),

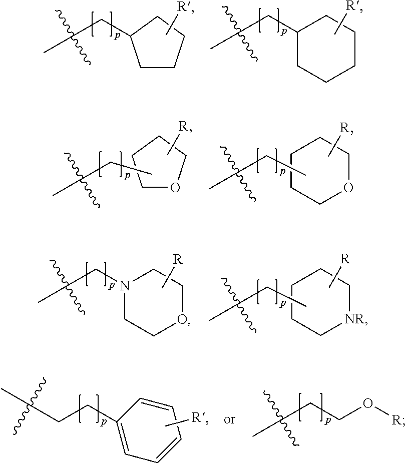

wherein R is at each occurrence independently H, or a substituted or unsubstituted C₁₋₂ alkyl; R′ is at each occurrence independently H, —OR, cyano, or a substituted or unsubstituted C₁₋₂ alkyl; and p is 0-1.

In other embodiments of compounds of formula (I), R³ is H.

In some such embodiments described herein, R¹ is substituted or unsubstituted aryl, or substituted or unsubstituted heteroaryl. For example, R¹ is phenyl, pyridyl, pyrimidyl, benzimidazolyl, 1H-pyrrolo[2,3-b]pyridyl, indazolyl, indolyl, 1H-imidazo[4,5-b]pyridine, pyridyl, 1H-imidazo[4,5-b]pyridin-2(3H)-onyl, 3H-imidazo[4,5-b]pyridyl, or pyrazolyl, each optionally substituted. In some embodiments, R¹ is phenyl substituted with one or more substituents independently selected from the group consisting of substituted or unsubstituted C₁₋₈ alkyl, substituted or unsubstituted heterocyclyl, aminocarbonyl, halogen, cyano, hydroxyalkyl and hydroxy. In others, R¹ is pyridyl substituted with one or more substituents independently selected from the group consisting of C₁₋₈ alkyl, substituted or unsubstituted heterocyclyl, halogen, aminocarbonyl, cyano, hydroxyalkyl, —OR, and —NR₂, wherein each R is independently H, or a substituted or unsubstituted C₁₋₄ alkyl. In still others, R¹ is 1H-pyrrolo[2,3-b]pyridyl or benzimidazolyl, optionally substituted with one or more substituents independently selected from the group consisting of substituted or unsubstituted C₁₋₈ alkyl, and —NR₂, wherein R is independently H, or a substituted or unsubstituted C₁₋₄ alkyl.

In certain embodiments, the compounds of formula (I) have an R¹ group set forth herein and an R² group set forth herein.

In some embodiments of compounds of formula (I), the compound inhibits TOR kinase. In other embodiments of compounds of formula (I), the compound inhibits DNA-PK. In certain embodiments of compounds of formula (I), the compound inhibits both TOR kinase and DNA-PK.

In some embodiments of compounds of formula (I), the compound at a concentration of 10 μM inhibits TOR kinase, DNA-PK, PI3K, or a combination thereof by at least about 50%. Compounds of formula (I) may be shown to be inhibitors of the kinases above in any suitable assay system.

Representative TOR kinase inhibitors of formula (I) include compounds from Table A.

TABLE A
7-(5-fluoro-2-methyl-4-(1H-1,2,4-triazol-3-yl)phenyl)-1-((trans-4-
methoxycyclohexyl)methyl)-3,4-dihydropyrazino[2,3-b]pyrazin-2(1H)-one;
7-(6-(1H-1,2,4-triazol-3-yl)pyridin-3-yl)-1-(cis-4-methoxycyclohexyl)-3,4-
dihydropyrazino[2,3-b]pyrazin-2(1H)-one;
7-(1H-pyrrolo[2,3-b]pyridin-3-yl)-1-(2-(tetrahydro-2H-pyran-4-yl)ethyl)-3,4-
dihydropyrazino[2,3-b]pyrazin-2(1H)-one;
7-(5-fluoro-2-methyl-4-(1H-1,2,4-triazol-3-yl)phenyl)-1-((cis-4-
methoxycyclohexyl)methyl)-3,4-dihydropyrazino[2,3-b]pyrazin-2(1H)-one;
1-ethyl-7-(1H-pyrrolo[3,2-b]pyridin-5-yl)-3,4-dihydropyrazino[2,3-b]pyrazin-2(1H)-one;
7-(6-(1H-1,2,4-triazol-3-yl)pyridin-3-yl)-1-((cis-4-methoxycyclohexyl)methyl)-3,4-
dihydropyrazino[2,3-b]pyrazin-2(1H)-one;
7-(1H-benzo[d]imidazol-4-yl)-1-(2-(tetrahydro-2H-pyran-4-yl)ethyl)-3,4-
dihydropyrazino[2,3-b]pyrazin-2(1H)-one;
7-(1H-pyrrolo[2,3-b]pyridin-4-yl)-1-(2-(tetrahydro-2H-pyran-4-yl)ethyl)-3,4-
dihydropyrazino[2,3-b]pyrazin-2(1H)-one;
7-(6-(1H-1,2,4-triazol-3-yl)pyridin-3-yl)-1-((trans-4-methoxycyclohexyl)methyl)-3,4-
dihydropyrazino[2,3-b]pyrazin-2(1H)-one;
7-(6-(1H-1,2,4-triazol-3-yl)pyridin-3-yl)-1-((trans-4-hydroxycyclohexyl)methyl)-3,4-
dihydropyrazino[2,3-b]pyrazin-2(1H)-one;
7-(6-(1H-1,2,4-triazol-3-yl)pyridin-3-yl)-1-(cis-4-hydroxycyclohexyl)-3,4-
dihydropyrazino[2,3-b]pyrazin-2(1H)-one;
7-(5-fluoro-2-methyl-4-(1H-1,2,4-triazol-3-yl)phenyl)-1-(cis-4-hydroxycyclohexyl)-3,4-
dihydropyrazino[2,3-b]pyrazin-2(1H)-one;
7-(6-(1H-1,2,4-triazol-3-yl)pyridin-3-yl)-1-(tetrahydro-2H-pyran-4-yl)-3,4-
dihydropyrazino[2,3-b]pyrazin-2(1H)-one;
7-(6-(1H-1,2,4-triazol-3-yl)pyridin-3-yl)-1-(2-methoxyethyl)-3,4-dihydropyrazino[2,3-
b]pyrazin-2(1H)-one;
7-(6-(1H-1,2,4-triazol-3-yl)pyridin-3-yl)-1-ethyl-3,4-dihydropyrazino[2,3-b]pyrazin-2(1H)-
one;
7-(5-fluoro-2-methyl-4-(1H-1,2,4-triazol-3-yl)phenyl)-1-((cis-4-
hydroxycyclohexyl)methyl)-3,4-dihydropyrazino[2,3-b]pyrazin-2(1H)-one;
7-(5-fluoro-2-methyl-4-(1H-1,2,4-triazol-3-yl)phenyl)-1-(tetrahydro-2H-pyran-4-yl)-3,4-
dihydropyrazino[2,3-b]pyrazin-2(1H)-one;
7-(1H-indol-4-yl)-1-(2-(tetrahydro-2H-pyran-4-yl)ethyl)-3,4-dihydropyrazino[2,3-
b]pyrazin-2(1H)-one;
7-(5-fluoro-2-methyl-4-(1H-1,2,4-triazol-3-yl)phenyl)-1-((trans-4-
hydroxycyclohexyl)methyl)-3,4-dihydropyrazino[2,3-b]pyrazin-2(1H)-one;
7-(6-(1H-1,2,4-triazol-3-yl)pyridin-3-yl)-1-((cis-4-hydroxycyclohexyl)methyl)-3,4-
dihydropyrazino[2,3-b]pyrazin-2(1H)-one;
7-(6-(1H-1,2,4-triazol-3-yl)pyridin-3-yl)-1-(trans-4-hydroxycyclohexyl)-3,4-
dihydropyrazino[2,3-b]pyrazin-2(1H)-one;
7-(6-(1H-1,2,4-triazol-3-yl)pyridin-3-yl)-1-(trans-4-methoxycyclohexyl)-3,4-
dihydropyrazino[2,3-b]pyrazin-2(1H)-one;
7-(6-(1H-1,2,4-triazol-3-yl)pyridin-3-yl)-1-isopropyl-3,4-dihydropyrazino[2,3-b]pyrazin-
2(1H)-one;
7-(5-fluoro-2-methyl-4-(1H-1,2,4-triazol-3-yl)phenyl)-1-(trans-4-methoxycyclohexyl)-3,4-
dihydropyrazino[2,3-b]pyrazin-2(1H)-one;
7-(5-fluoro-2-methyl-4-(1H-1,2,4-triazol-3-yl)phenyl)-1-(trans-4-hydroxycyclohexyl)-3,4-
dihydropyrazino[2,3-b]pyrazin-2(1H)-one;
7-(5-fluoro-2-methyl-4-(1H-1,2,4-triazol-3-yl)phenyl)-1-(2-methoxyethyl)-3,4-
dihydropyrazino[2,3-b]pyrazin-2(1H)-one;
7-(5-fluoro-2-methyl-4-(1H-1,2,4-triazol-3-yl)phenyl)-1-isopropyl-3,4-dihydropyrazino[2,3-
b]pyrazin-2(1H)-one;
1-ethyl-7-(5-fluoro-2-methyl-4-(1H-1,2,4-triazol-3-yl)phenyl)-3,4-dihydropyrazino[2,3-
b]pyrazin-2(1H)-one;
7-(2-hydroxypyridin-4-yl)-1-(2-(tetrahydro-2H-pyran-4-yl)ethyl)-3,4-dihydropyrazino[2,3-
b]pyrazin-2(1H)-one;
1-isopropyl-7-(4-methyl-6-(1H-1,2,4-triazol-3-yl)pyridin-3-yl)-3,4-dihydropyrazino[2,3-
b]pyrazin-2(1H)-one;
5-(8-isopropyl-7-oxo-5,6,7,8-tetrahydropyrazino[2,3-b]pyrazin-2-yl)-4-methylpicolinamide;
7-(1H-indazol-4-yl)-1-(2-(tetrahydro-2H-pyran-4-yl)ethyl)-3,4-dihydropyrazino[2,3-
b]pyrazin-2(1H)-one;
7-(2-aminopyrimidin-5-yl)-1-(2-(tetrahydro-2H-pyran-4-yl)ethyl)-3,4-dihydropyrazino[2,3-
b]pyrazin-2(1H)-one;
7-(2-aminopyridin-4-yl)-1-(2-(tetrahydro-2H-pyran-4-yl)ethyl)-3,4-dihydropyrazino[2,3-
b]pyrazin-2(1H)-one;
7-(6-(methylamino)pyridin-3-yl)-1-(2-(tetrahydro-2H-pyran-4-yl)ethyl)-3,4-
dihydropyrazino[2,3-b]pyrazin-2(1H)-one;
7-(6-hydroxypyridin-3-yl)-1-(2-(tetrahydro-2H-pyran-4-yl)ethyl)-3,4-dihydropyrazino[2,3-
b]pyrazin-2(1H)-one;
7-(4-(1H-pyrazol-3-yl)phenyl)-1-(2-methoxyethyl)-3,4-dihydropyrazino[2,3-b]pyrazin-
2(1H)-one;
7-(pyridin-3-yl)-1-(2-(tetrahydro-2H-pyran-4-yl)ethyl)-3,4-dihydropyrazino[2,3-b]pyrazin-
2(1H)-one;
7-(1H-indazol-4-yl)-1-(2-methoxyethyl)-3,4-dihydropyrazino[2,3-b]pyrazin-2(1H)-one;
7-(1H-indazol-6-yl)-1-(2-methoxyethyl)-3,4-dihydropyrazino[2,3-b]pyrazin-2(1H)-one;
7-(pyrimidin-5-yl)-1-(2-(tetrahydro-2H-pyran-4-yl)ethyl)-3,4-dihydropyrazino[2,3-
b]pyrazin-2(1H)-one;
7-(6-methoxypyridin-3-yl)-1-(2-(tetrahydro-2H-pyran-4-yl)ethyl)-3,4-dihydropyrazino[2,3-
b]pyrazin-2(1H)-one;
1-(2-methoxyethyl)-7-(1H-pyrrolo[2,3-b]pyridin-5-yl)-3,4-dihydropyrazino[2,3-b]pyrazin-
2(1H)-one;
1-ethyl-7-(1H-pyrrolo[2,3-b]pyridin-5-yl)-3,4-dihydropyrazino[2,3-b]pyrazin-2(1H)-one;
1-ethyl-7-(1H-indazol-4-yl)-3,4-dihydropyrazino[2,3-b]pyrazin-2(1H)-one;
7-(pyridin-4-yl)-1-(2-(tetrahydro-2H-pyran-4-yl)ethyl)-3,4-dihydropyrazino[2,3-b]pyrazin-
2(1H)-one;
7-(6-aminopyridin-3-yl)-1-(2-(tetrahydro-2H-pyran-4-yl)ethyl)-3,4-dihydropyrazino[2,3-
b]pyrazin-2(1H)-one;
1-methyl-7-(2-methyl-6-(4H-1,2,4-triazol-3-yl)pyridin-3-yl)-3,4-dihydropyrazino[2,3-
b]pyrazin-2(1H)-one;
2-(2-hydroxypropan-2-yl)-5-(8-(trans-4-methoxycyclohexyl)-7-oxo-5,6,7,8-
tetrahydropyrazino[2,3-b]pyrazin-2-yl)pyridine 1-oxide;
4-methyl-5-(7-oxo-8-((tetrahydro-2H-pyran-4-yl)methyl)-5,6,7,8-tetrahydropyrazino[2,3-
b]pyrazin-2-yl)picolinamide;
5-(8-((cis-4-methoxycyclohexyl)methyl)-7-oxo-5,6,7,8-tetrahydropyrazino[2,3-b]pyrazin-2-
yl)-4-methylpicolinamide;
7-(1H-pyrazol-4-yl)-1-(2-(tetrahydro-2H-pyran-4-yl)ethyl)-3,4-dihydropyrazino[2,3-
b]pyrazin-2(1H)-one;
1-(trans-4-methoxycyclohexyl)-7-(4-methyl-6-(1H-1,2,4-triazol-3-yl)pyridin-3-yl)-3,4-
dihydropyrazino[2,3-b]pyrazin-2(1H)-one;
3-((7-(2-methyl-6-(4H-1,2,4-triazol-3-yl)pyridin-3-yl)-2-oxo-3,4-dihydropyrazino[2,3-
b]pyrazin-1(2H)-yl)methyl)benzonitrile;
1-((trans-4-methoxycyclohexyl)methyl)-7-(4-methyl-6-(1H-1,2,4-triazol-3-yl)pyridin-3-yl)-
3,4-dihydropyrazino[2,3-b]pyrazin-2(1H)-one;
3-(7-oxo-8-(2-(tetrahydro-2H-pyran-4-yl)ethyl)-5,6,7,8-tetrahydropyrazino[2,3-b]pyrazin-2-
yl)benzamide;
5-(8-((trans-4-methoxycyclohexyl)methyl)-7-oxo-5,6,7,8-tetrahydropyrazino[2,3-b]pyrazin-
2-yl)-4-methylpicolinamide;
3-((7-(6-(2-hydroxypropan-2-yl)pyridin-3-yl)-2-oxo-3,4-dihydropyrazino[2,3-b]pyrazin-
1(2H)-yl)methyl)benzonitrile;
7-(6-(2-hydroxypropan-2-yl)pyridin-3-yl)-1-((1R,3R)-3-methoxycyclopentyl)-3,4-
dihydropyrazino[2,3-b]pyrazin-2(1H)-one;
7-(6-(2-hydroxypropan-2-yl)pyridin-3-yl)-1-((1S,3R)-3-methoxycyclopentyl)-3,4-
dihydropyrazino[2,3-b]pyrazin-2(1H)-one;
7-(6-(2-hydroxypropan-2-yl)pyridin-3-yl)-1-((1S,3S)-3-methoxycyclopentyl)-3,4-
dihydropyrazino[2,3-b]pyrazin-2(1H)-one;
7-(6-(2-hydroxypropan-2-yl)pyridin-3-yl)-1-((1R,3S)-3-methoxycyclopentyl)-3,4-
dihydropyrazino[2,3-b]pyrazin-2(1H)-one;
7-(1H-indazol-6-yl)-1-(2-(tetrahydro-2H-pyran-4-yl)ethyl)-3,4-dihydropyrazino[2,3-
b]pyrazin-2(1H)-one;
7-(2-methyl-6-(4H-1,2,4-triazol-3-yl)pyridin-3-yl)-1-(2-morpholinoethyl)-3,4-
dihydropyrazino[2,3-b]pyrazin-2(1H)-one;
1-(trans-4-hydroxycyclohexyl)-7-(2-methyl-6-(4H-1,2,4-triazol-3-yl)pyridin-3-yl)-3,4-
dihydropyrazino[2,3-b]pyrazin-2(1H)-one;
1-(cis-4-hydroxycyclohexyl)-7-(2-methyl-6-(4H-1,2,4-triazol-3-yl)pyridin-3-yl)-3,4-
dihydropyrazino[2,3-b]pyrazin-2(1H)-one;
7-(6-(2-hydroxypropan-2-yl)pyridin-3-yl)-1-(2-morpholinoethyl)-3,4-dihydropyrazino[2,3-
b]pyrazin-2(1H)-one;
1-isopropyl-7-(2-methyl-6-(4H-1,2,4-triazol-3-yl)pyridin-3-yl)-3,4-dihydropyrazino[2,3-
b]pyrazin-2(1H)-one;
7-(1H-imidazo[4,5-b]pyridin-6-yl)-1-(2-(tetrahydro-2H-pyran-4-yl)ethyl)-3,4-
dihydropyrazino[2,3-b]pyrazin-2(1H)-one;
1-((cis-4-methoxycyclohexyl)methyl)-7-(2-methyl-6-(1H-1,2,4-triazol-3-yl)pyridin-3-yl)-
3,4-dihydropyrazino[2,3-b]pyrazin-2(1H)-one;
1-(trans-4-hydroxycyclohexyl)-7-(6-(2-hydroxypropan-2-yl)pyridin-3-yl)-3,4-
dihydropyrazino[2,3-b]pyrazin-2(1H)-one;
1-(cis-4-hydroxycyclohexyl)-7-(6-(2-hydroxypropan-2-yl)pyridin-3-yl)-3,4-
dihydropyrazino[2,3-b]pyrazin-2(1H)-one;
4-(7-oxo-8-(2-(tetrahydro-2H-pyran-4-yl)ethyl)-5,6,7,8-tetrahydropyrazino[2,3-b]pyrazin-2-
yl)benzamide;
7-(1H-indazol-5-yl)-1-(2-(tetrahydro-2H-pyran-4-yl)ethyl)-3,4-dihydropyrazino[2,3-
b]pyrazin-2(1H)-one;
7-(1H-pyrrolo[2,3-b]pyridin-5-yl)-1-(2-(tetrahydro-2H-pyran-4-yl)ethyl)-3,4-
dihydropyrazino[2,3-b]pyrazin-2(1H)-one;
7-(2-methyl-6-(4H-1,2,4-triazol-3-yl)pyridin-3-yl)-1-(tetrahydro-2H-pyran-4-yl)-3,4-
dihydropyrazino[2,3-b]pyrazin-2(1H)-one;
1-((1S,3R)-3-methoxycyclopentyl)-7-(2-methyl-6-(4H-1,2,4-triazol-3-yl)pyridin-3-yl)-3,4-
dihydropyrazino[2,3-b]pyrazin-2(1H)-one;
1-((1R,3R)-3-methoxycyclopentyl)-7-(2-methyl-6-(4H-1,2,4-triazol-3-yl)pyridin-3-yl)-3,4-
dihydropyrazino[2,3-b]pyrazin-2(1H)-one;
1-((1R,3S)-3-methoxycyclopentyl)-7-(2-methyl-6-(4H-1,2,4-triazol-3-yl)pyridin-3-yl)-3,4-
dihydropyrazino[2,3-b]pyrazin-2(1H)-one;
1-((1S,3S)-3-methoxycyclopentyl)-7-(2-methyl-6-(4H-1,2,4-triazol-3-yl)pyridin-3-yl)-3,4-
dihydropyrazino[2,3-b]pyrazin-2(1H)-one;
7-(1H-indol-5-yl)-1-(2-(tetrahydro-2H-pyran-4-yl)ethyl)-3,4-dihydropyrazino[2,3-
b]pyrazin-2(1H)-one;
1-ethyl-7-(2-methyl-6-(4H-1,2,4-triazol-3-yl)pyridin-3-yl)-3,4-dihydropyrazino[2,3-
b]pyrazin-2(1H)-one;
7-(1H-indol-6-yl)-1-(2-(tetrahydro-2H-pyran-4-yl)ethyl)-3,4-dihydropyrazino[2,3-
b]pyrazin-2(1H)-one;
7-(4-(2-hydroxypropan-2-yl)phenyl)-1-(trans-4-methoxycyclohexyl)-3,4-
dihydropyrazino[2,3-b]pyrazin-2(1H)-one;
7-(6-(2-hydroxypropan-2-yl)pyridin-3-yl)-1-(tetrahydro-2H-pyran-4-yl)-3,4-
dihydropyrazino[2,3-b]pyrazin-2(1H)-one;
1-((trans-4-methoxycyclohexyl)methyl)-7-(2-methyl-6-(1H-1,2,4-triazol-3-yl)pyridin-3-yl)-
3,4-dihydropyrazino[2,3-b]pyrazin-2(1H)-one;
7-(6-(2-hydroxypropan-2-yl)pyridin-3-yl)-1-((cis-4-methoxycyclohexyl)methyl)-3,4-
dihydropyrazino[2,3-b]pyrazin-2(1H)-one;
1-(2-methoxyethyl)-7-(4-methyl-2-(methylamino)-1H-benzo[d]imidazol-6-yl)-3,4-
dihydropyrazino[2,3-b]pyrazin-2(1H)-one;
7-(7-methyl-2-oxo-2,3-dihydro-1H-benzo[d]imidazol-5-yl)-1-((tetrahydro-2H-pyran-4-
yl)methyl)-3,4-dihydropyrazino[2,3-b]pyrazin-2(1H)-one;
7-(2-methyl-4-(4H-1,2,4-triazol-3-yl)phenyl)-3,4-dihydropyrazino[2,3-b]pyrazin-2(1H)-one;
1-(2-methoxyethyl)-7-(4-methyl-6-(1H-1,2,4-triazol-3-yl)pyridin-3-yl)-3,4-
dihydropyrazino[2,3-b]pyrazin-2(1H)-one;
1-benzyl-7-(2-methyl-4-(4H-1,2,4-triazol-3-yl)phenyl)-3,4-dihydropyrazino[2,3-b]pyrazin-
2(1H)-one;
7-(3-fluoro-4-(4H-1,2,4-triazol-3-yl)phenyl)-1-(2-methoxyethyl)-3,4-dihydropyrazino[2,3-
b]pyrazin-2(1H)-one;
7-(3-fluoro-4-(4H-1,2,4-triazol-3-yl)phenyl)-1-(2-(tetrahydro-2H-pyran-4-yl)ethyl)-3,4-
dihydropyrazino[2,3-b]pyrazin-2(1H)-one;
7-(3-fluoro-2-methyl-4-(1H-1,2,4-triazol-3-yl)phenyl)-1-(2-methoxyethyl)-3,4-
dihydropyrazino[2,3-b]pyrazin-2(1H)-one;
1-(trans-4-methoxycyclohexyl)-7-(2-methyl-6-(4H-1,2,4-triazol-3-yl)pyridin-3-yl)-3,4-
dihydropyrazino[2,3-b]pyrazin-2(1H)-one;
7-(6-(2-hydroxypropan-2-yl)pyridin-3-yl)-1-(trans-4-methoxycyclohexyl)-3,4-
dihydropyrazino[2,3-b]pyrazin-2(1H)-one;
7-(5-fluoro-2-methyl-4-(4H-1,2,4-triazol-3-yl)phenyl)-1-(2-(tetrahydro-2H-pyran-4-
yl)ethyl)-3,4-dihydropyrazino[2,3-b]pyrazin-2(1H)-one;
7-(3-fluoro-2-methyl-4-(1H-1,2,4-triazol-3-yl)phenyl)-1-(2-(tetrahydro-2H-pyran-4-
yl)ethyl)-3,4-dihydropyrazino[2,3-b]pyrazin-2(1H)-one;
1-(2-methoxyethyl)-7-(2-methyl-6-(4H-1,2,4-triazol-3-yl)pyridin-3-yl)-3,4-
dihydropyrazino[2,3-b]pyrazin-2(1H)-one;
7-(6-(2-hydroxypropan-2-yl)pyridin-3-yl)-1-((trans-4-methoxycyclohexyl)methyl)-3,4-
dihydropyrazino[2,3-b]pyrazin-2(1H)-one;
1-(cyclopentylmethyl)-7-(6-(2-hydroxypropan-2-yl)pyridin-3-yl)-3,4-dihydropyrazino[2,3-
b]pyrazin-2(1H)-one;
7-(4-(2-hydroxypropan-2-yl)phenyl)-1-(2-methoxyethyl)-3,4-dihydropyrazino[2,3-
b]pyrazin-2(1H)-one;
(S)-7-(6-(1-hydroxyethyl)pyridin-3-yl)-1-(2-(tetrahydro-2H-pyran-4-yl)ethyl)-3,4-
dihydropyrazino[2,3-b]pyrazin-2(1H)-one;
(R)-7-(6-(1-hydroxyethyl)pyridin-3-yl)-1-(2-(tetrahydro-2H-pyran-4-yl)ethyl)-3,4-
dihydropyrazino[2,3-b]pyrazin-2(1H)-one;
7-(2-methyl-6-(4H-1,2,4-triazol-3-yl)pyridin-3-yl)-1-((tetrahydro-2H-pyran-4-yl)methyl)-
3,4-dihydropyrazino[2,3-b]pyrazin-2(1H)-one;
7-(4-(2-hydroxypropan-2-yl)phenyl)-1-(2-(tetrahydro-2H-pyran-4-yl)ethyl)-3,4-
dihydropyrazino[2,3-b]pyrazin-2(1H)-one;
7-(6-(2-hydroxypropan-2-yl)pyridin-3-yl)-1-(4-(trifluoromethyl)benzyl)-3,4-
dihydropyrazino[2,3-b]pyrazin-2(1H)-one;
7-(6-(2-hydroxypropan-2-yl)pyridin-3-yl)-1-(3-(trifluoromethyl)benzyl)-3,4-
dihydropyrazino[2,3-b]pyrazin-2(1H)-one;
7-(6-(2-hydroxypropan-2-yl)pyridin-3-yl)-1-(3-methoxypropyl)-3,4-dihydropyrazino[2,3-
b]pyrazin-2(1H)-one;
7-(4-methyl-6-(1H-1,2,4-triazol-3-yl)pyridin-3-yl)-1-(2-(tetrahydro-2H-pyran-4-yl)ethyl)-
3,4-dihydropyrazino[2,3-b]pyrazin-2(1H)-one;
7-(6-(2-hydroxypropan-2-yl)pyridin-3-yl)-1-(2-methoxyethyl)-3,4-dihydropyrazino[2,3-
b]pyrazin-2(1H)-one;
7-(6-(2-hydroxypropan-2-yl)pyridin-3-yl)-1-((tetrahydro-2H-pyran-4-yl)methyl)-3,4-
dihydropyrazino[2,3-b]pyrazin-2(1H)-one;
7-(4-methyl-2-(methylamino)-1H-benzo[d]imidazol-6-yl)-1-((tetrahydro-2H-pyran-4-
yl)methyl)-3,4-dihydropyrazino[2,3-b]pyrazin-2(1H)-one;
7-(2-amino-4-methyl-1H-benzo[d]imidazol-6-yl)-1-((tetrahydro-2H-pyran-4-yl)methyl)-3,4-
dihydropyrazino[2,3-b]pyrazin-2(1H)-one;
7-(2-methyl-6-(4H-1,2,4-triazol-3-yl)pyridin-3-yl)-1-(2-(tetrahydro-2H-pyran-4-yl)ethyl)-
3,4-dihydropyrazino[2,3-b]pyrazin-2(1H)-one;
(R)-7-(6-(2-hydroxypropan-2-yl)pyridin-3-yl)-3-methyl-1-(2-(tetrahydro-2H-pyran-4-
yl)ethyl)-3,4-dihydropyrazino[2,3-b]pyrazin-2(1H)-one;
(S)-7-(6-(2-hydroxypropan-2-yl)pyridin-3-yl)-3-methyl-1-(2-(tetrahydro-2H-pyran-4-
yl)ethyl)-3,4-dihydropyrazino[2,3-b]pyrazin-2(1H)-one;
7-(6-(2-hydroxypropan-2-yl)pyridin-3-yl)-3,3-dimethyl-1-(2-(tetrahydro-2H-pyran-4-
yl)ethyl)-3,4-dihydropyrazino[2,3-b]pyrazin-2(1H)-one;
7-(2-amino-4-methyl-1H-benzo[d]imidazol-6-yl)-1-(2-(tetrahydro-2H-pyran-4-yl)ethyl)-3,4-
dihydropyrazino[2,3-b]pyrazin-2(1H)-one;
7-(6-(2-hydroxypropan-2-yl)pyridin-3-yl)-1-(2-(tetrahydro-2H-pyran-4-yl)ethyl)-3,4-
dihydropyrazino[2,3-b]pyrazin-2(1H)-one;
7-(2-methyl-4-(1H-1,2,4-triazol-3-yl)phenyl)-1-(2-(tetrahydro-2H-pyran-4-yl)ethyl)-3,4-
dihydropyrazino[2,3-b]pyrazin-2(1H)-one;
7-(4-(1H-1,2,4-triazol-5-yl)phenyl)-1-(2-(tetrahydro-2H-pyran-4-yl)ethyl)-3,4-
dihydropyrazino[2,3-b]pyrazin-2(1H)-one;
1-(1-hydroxypropan-2-yl)-7-(2-methyl-6-(1H-1,2,4-triazol-3-yl)pyridin-3-yl)-3,4-
dihydropyrazino[2,3-b]pyrazin-2(1H)-one; and
1-(2-hydroxyethyl)-7-(2-methyl-6-(1H-1,2,4-triazol-3-yl)pyridin-3-yl)-3,4-
dihydropyrazino[2,3-b]pyrazin-2(1H)-one,
and pharmaceutically acceptable salts, clathrates, solvates, stereoisomers, tautomers,
metabolites, isotopologues and prodrugs thereof.

### 5.3 Methods for Making TOR Kinase Inhibitors

The TOR kinase inhibitors can be obtained via standard, well-known synthetic methodology, see e.g., March, J. Advanced Organic Chemistry; Reactions Mechanisms, and Structure, 4th ed., 1992. Starting materials useful for preparing compounds of formula (III) and intermediates therefore, are commercially available or can be prepared from commercially available materials using known synthetic methods and reagents.

Particular methods for preparing compounds of formula (I) are disclosed in U.S. Pat. No. 8,110,578, issued Feb. 7, 2012, and U.S. Pat. No. 8,569,494, issued Oct. 29, 2013, each incorporated by reference herein in their entirety.

### 5.4 IMID® Immunomodulatory Drugs

As used herein and unless otherwise indicated, the term “IMiD® immunomodulatory drug(s)” (Celgene Corporation) encompasses certain small organic molecules that inhibit LPS induced monocyte TNF-α, IL-1β, IL-12, IL-6, MIP-1α, MCP-1, GM-CSF, G-CSF, and COX-2 production. Specific IMiD® immunomodulatory drugs are discussed below.

TNF-α is an inflammatory cytokine produced by macrophages and monocytes during acute inflammation. TNF-α is responsible for a diverse range of signaling events within cells. Without being limited by a particular theory, one of the biological effects exerted by the IMiD® immunomodulatory drugs provided herein is the reduction of myeloid cell TNF-α production. IMiD® immunomodulatory drugs provided herein may enhance the degradation of TNF-α mRNA.

Further, without being limited by theory, IMiD® immunomodulatory drugs provided herein may also be potent co-stimulators of T cells and increase cell proliferation dramatically in a dose dependent manner. IMiD® immunomodulatory drugs provided herein may also have a greater co-stimulatory effect on the CD8+ T cell subset than on the CD4+ T cell subset. In addition, the IMiD® immunomodulatory drugs preferably have anti-inflammatory properties against myeloid cell responses, yet efficiently co-stimulate T cells to produce greater amounts of IL-2, IFN-γ, and to enhance T cell proliferation and CD8+ T cell cytotoxic activity. Further, without being limited by a particular theory, IMiD® immunomodulatory drugs provided herein may be capable of acting both indirectly through cytokine activation and directly on Natural Killer (“NK”) cells and Natural Killer T (“NKT”) cells, and increase the NK cells' ability to produce beneficial cytokines such as, but not limited to, IFN-γ, and to enhance NK and NKT cell cytotoxic activity.

Specific examples of IMiD® immunomodulatory drugs include cyano and carboxy derivatives of substituted styrenes such as those disclosed in U.S. Pat. No. 5,929,117; 1-oxo-2-(2,6-dioxo-3-fluoropiperidin-3-yl)isoindolines and 1,3-dioxo-2-(2,6-dioxo-3-fluoropiperidine-3-yl)isoindolines such as those described in U.S. Pat. Nos. 5,874,448 and 5,955,476; the tetra substituted 2-(2,6-dioxopiperidin-3-yl)-1-oxoisoindolines described in U.S. Pat. No. 5,798,368; 1-oxo and 1,3-dioxo-2-(2,6-dioxopiperidin-3-yl)isoindolines (e.g., 4-methyl derivatives of thalidomide), substituted 2-(2,6-dioxopiperidin-3-yl)phthalimides and substituted 2-(2,6-dioxopiperidin-3-yl)-1-oxoisoindoles including, but not limited to, those disclosed in U.S. Pat. Nos. 5,635,517, 6,281,230, 6,316,471, 6,403,613, 6,476,052 and 6,555,554; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; and isoindole-imide compounds such as those described in U.S. Pat. No. 7,091,353. Further specific examples of IMiD® immunomodulatory drugs include isoindolines such as those described in U.S. Pat. Nos. 7,405,237 and 7,816,393. The entireties of each of the patents and patent applications identified herein are incorporated herein by reference. IMiD® immunomodulatory drugs do not include thalidomide.

Various IMiD® immunomodulatory drugs provided herein contain one or more chiral centers, and can exist as racemic mixtures of enantiomers or mixtures of diastereomers. Provided herein are the use of stereomerically pure forms of such compounds, as well as the use of mixtures of those forms. For example, mixtures comprising equal or unequal amounts of the enantiomers of a particular IMiD® immunomodulatory drugs provided herein may be used in methods and compositions provided herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

Preferred IMiD® immunomodulatory drugs provided herein include, but are not limited to, 1-oxo- and 1,3 dioxo-2-(2,6-dioxopiperidin-3-yl)isoindolines substituted with amino in the benzo ring as described in U.S. Pat. No. 5,635,517 which is incorporated herein by reference. These compounds have the structure I:

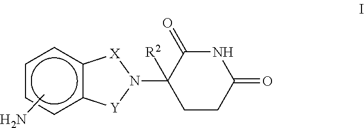

in which one of X and Y is C═O, the other of X and Y is C═O or CH₂, and R² is hydrogen or lower alkyl, in particular methyl.

Specific IMiD® immunomodulatory drugs include, but are not limited to:

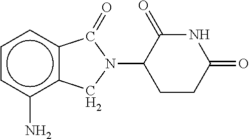

1-oxo-2-(2,6-dioxopiperidin-3-yl)-4-aminoisoindoline;

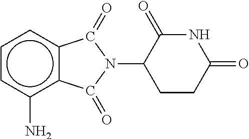

1,3-dioxo-2-(2,6-dioxopiperidin-3-yl)-4-aminoisoindoline; and

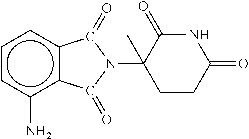

1,3-dioxo-2-(3-methyl-2,6-dioxopiperidin-3-yl)-4-aminoisoindoline,

and optically pure isomers thereof. The compounds can be obtained via standard, synthetic methods (see e.g., U.S. Pat. No. 5,635,517, incorporated herein by reference). The compounds are also available from Celgene Corporation, Warren, N.J.

Other specific IMiD® immunomodulatory drugs provided herein belong to a class of substituted 2-(2,6-dioxopiperidin-3-yl)phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference.

Representative compounds are of formula:

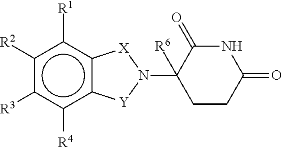

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzyl, or halo;

provided that R⁶ is other than hydrogen if X and Y are C═O and (i) each of R¹, R², R³, and R⁴ is fluoro or (ii) one of R¹, R², R³, or R⁴ is amino.

Compounds representative of this class are of the formulas:

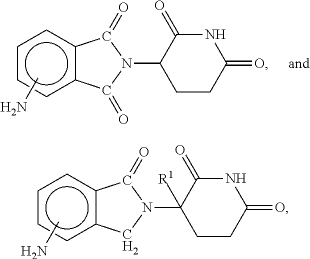

wherein R¹ is hydrogen or methyl. In a separate embodiment, provided herein is the use of enantiomerically pure forms (e.g. optically pure (R) or (S) enantiomers) of these compounds.

Still other specific IMiD® immunomodulatory drugs provided herein belong to a class of isoindole-imides disclosed in U.S. Pat. No. 7,091,353, which is incorporated herein by reference. Representative compounds are of formula II:

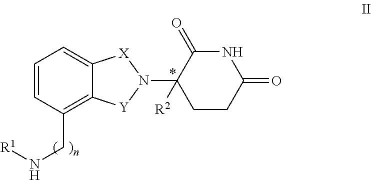

and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;

n is 0 or 1; and

* represents a chiral-carbon center.

In specific compounds of formula II, when n is 0 then R¹ is (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(O)OR⁴, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;

R² is H or (C₁-C₈)alkyl; and

R³ is (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₅-C₈)alkyl-N(R⁶)₂; (C₀-C₈)alkyl-NH—C(O)O—R⁵; (C₁-C₈)alkyl-OR⁵, (C₁-C₈)alkyl-C(O)OR⁵, (C₁-C₈)alkyl-O(CO)R⁵, or C(O)OR⁵; and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃, CH₂CH₂OCH₃, or

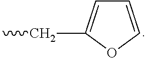

In another embodiment of the compounds of formula II, R¹ is

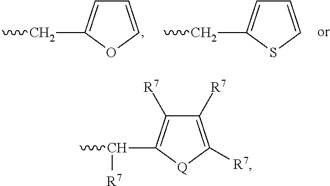

wherein Q is O or S, and each occurrence of R⁷ is independently H, (C₁-C₈)alkyl, (C₃-C₇)cycloalkyl, (C₂-C₈)alkenyl, (C₂-C₈)alkynyl, benzyl, aryl, halogen, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵, or adjacent occurrences of R⁷ can be taken together to form a bicyclic alkyl or aryl ring.

In other specific compounds of formula II, R¹ is C(O)R³.

In other specific compounds of formula II, R³ is (C₀-C₄)alkyl-(C₂-C₅)heteroaryl, (C₁-C₈)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Further examples of the compounds in this class include, but are not limited to: [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl}cyclopropyl-carboxamide; 2-chloro-N-{(2-(2,6-dioxo(3-piperidyl))-1,3-dioxoisoindolin-4-yl)methyl}acetamide; N-(2-(2,6-dioxo(3-piperidyl))-1,3-dioxoisoindolin-4-yl)-3-pyridylcarboxamide; 3-{1-oxo-4-(benzylamino)isoindolin-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl}propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl}heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl}methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl}(butylamino)carboxamide; N-{[2-(2,6-dioxo(3-piperidyl))-1,3-dioxoisoindolin-4-yl]methyl}(octylamino)carboxamide; and N-{[2-(2,6-dioxo(3-piperidyl))-1,3-dioxoisoindolin-4-yl]methyl}(benzylamino)carboxamide.

Still other specific IMiD® immunomodulatory drugs provided herein belong to a class of isoindole-imides disclosed in U.S. Pat. No. 6,555,554, International Publication No. WO 98/54170, and U.S. Pat. No. 6,395,754, each of which is incorporated herein by reference. Representative compounds are of formula III:

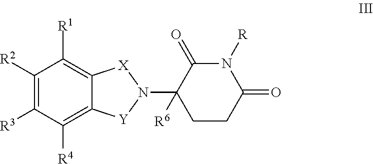

and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷—CHR¹⁰—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;

R¹⁰ is hydrogen, alkyl of to 8 carbon atoms, or phenyl; and

* represents a chiral-carbon center.

Other representative compounds are of formula:

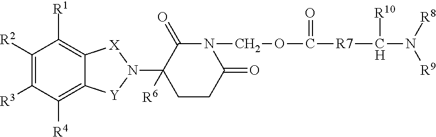

wherein:

one of X and Y is C═O and the other of X and Y is C═O or CH₂;

(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzo, chloro, or fluoro;

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S—, or —NH—; and

R¹⁰ is hydrogen, alkyl of to 8 carbon atoms, or phenyl.

Other representative compounds are of formula:

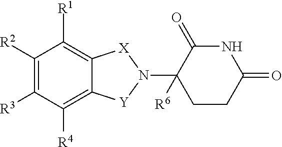

in which

one of X and Y is C═O and the other of X and Y is C═O or CH₂;

each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and

R⁶ is hydrogen, alkyl of 1 to 8 carbon atoms, benzo, chloro, or fluoro.

Other representative compounds are of formula:

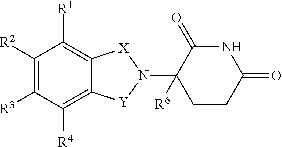

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen, alkyl of 1 to 8 carbon atoms, or)CO—R⁷—CH(R¹⁰)NR⁸R⁹ in which each of R⁷, R⁸, R⁹, and R¹⁰ is as herein defined; and

R⁶ is alkyl of 1 to 8 carbon atoms, benzo, chloro, or fluoro.

Specific examples of the compounds are of formula:

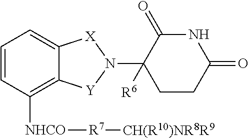

in which:

one of X and Y is C═O and the other of X and Y is C═O or CH₂;

R⁶ is hydrogen, alkyl of 1 to 8 carbon atoms, benzyl, chloro, or fluoro;

R⁷ is m-phenylene, p-phenylene or —(CₙH₂ₙ)— in which n has a value of 0 to 4; each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and

R¹⁰ is hydrogen, alkyl of 1 to 8 carbon atoms, or phenyl.

Other specific IMiD® immunomodulatory drugs provided herein include, but are not limited to, 1-oxo-2-(2,6-dioxo-3-fluoropiperidin-3-yl)isoindolines and 1,3-dioxo-2-(2,6-dioxo-3-fluoropiperidine-3-yl)isoindolines such as those described in U.S. Pat. Nos. 5,874,448 and 5,955,476, each of which is incorporated herein by reference. Representative compounds are of formula:

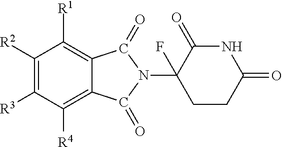

wherein:

Y is oxygen or H² and

each of R¹, R², R³, and R⁴, independently of the others, is hydrogen, halo, alkyl of 1 to 4 carbon atoms, alkoxy of 1 to 4 carbon atoms, or amino.

Other specific IMiD® immunomodulatory drugs provided herein include, but are not limited to, the tetra substituted 2-(2,6-dioxopiperidin-3-yl)-1-oxoisoindolines described in U.S. Pat. No. 5,798,368, which is incorporated herein by reference. Representative compounds are of formula:

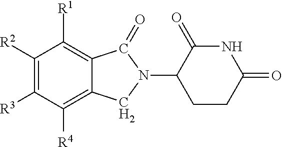

wherein each of R¹, R², R³, and R⁴, independently of the others, is halo, alkyl of 1 to 4 carbon atoms, or alkoxy of 1 to 4 carbon atoms.

Other specific IMiD® immunomodulatory drugs provided herein include, but are not limited to, 1-oxo and 1,3-dioxo-2-(2,6-dioxopiperidin-3-yl)isoindolines disclosed in U.S. Pat. No. 6,403,613, which is incorporated herein by reference. Representative compounds are of formula:

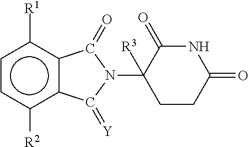

in which

Y is oxygen or H₂,

a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and

R³ is hydrogen, alkyl, or benzyl.

Specific examples of the compounds are of formula:

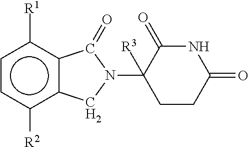

wherein

a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and

R³ is hydrogen, alkyl of from 1 to 4 carbon atoms, or benzyl. Specific examples include, but are not limited to, 1-oxo-2-(2,6-dioxopiperidin-3-yl)-4-methylisoindoline.

Other representative compounds are of formula:

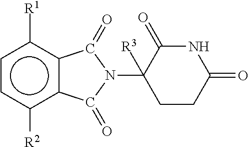

wherein:

a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and

R³ is hydrogen, alkyl of from 1 to 4 carbon atoms, or benzyl.

Other specific IMiD® immunomodulatory drugs provided herein include, but are not limited to, 1-oxo and 1,3-dioxoisoindolines substituted in the 4- or 5-position of the indoline ring described in U.S. Pat. No. 6,380,239 and U.S. Pat. No. 7,244,759, which are incorporated herein by reference. Representative compounds are of formula:

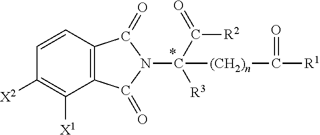

in which the carbon atom designated C* constitutes a center of chirality (when n is not zero and R¹ is not the same as R²); one of X¹ and X² is amino, nitro, alkyl of one to six carbons, or NH—Z, and the other of X¹ or X² is hydrogen; each of R¹ and R² independent of the other, is hydroxy or NH—Z; R³ is hydrogen, alkyl of one to six carbons, halo, or haloalkyl; Z is hydrogen, aryl, alkyl of one to six carbons, formyl, or acyl of one to six carbons; and n has a value of 0, 1, or 2; provided that if X¹ is amino, and n is 1 or 2, then R¹ and R² are not both hydroxy; and the salts thereof.

Further representative compounds are of formula:

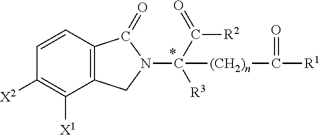

in which the carbon atom designated C* constitutes a center of chirality when n is not zero and R¹ is not R²; one of X¹ and X² is amino, nitro, alkyl of one to six carbons, or NH—Z, and the other of X¹ or X² is hydrogen; each of R¹ and R² independent of the other, is hydroxy or NH—Z; R³ is alkyl of one to six carbons, halo, or hydrogen; Z is hydrogen, aryl or an alkyl or acyl of one to six carbons; and n has a value of 0, 1, or 2.

Specific examples include, but are not limited to, 2-(4-amino-1-oxo-1,3-dihydro-isoindol-2-yl)-4-carbamoyl-butyric acid and 4-(4-amino-1-oxo-1,3-dihydro-isoindol-2-yl)-4-carbamoyl-butyric acid, which have the following structures, respectively, and pharmaceutically acceptable salts, solvates, prodrugs, and stereoisomers thereof:

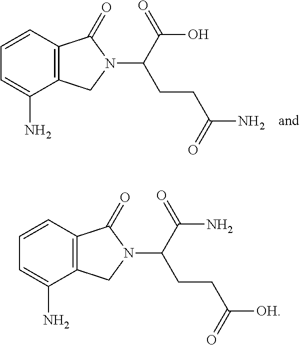 [SMILES: NC(=O)CCC(C(=O)O)N1Cc2c(N)cccc2C1=O ; NC(=O)C(CCC(=O)O)N1Cc2c(N)cccc2C1=O]

Other representative compounds are of formula:

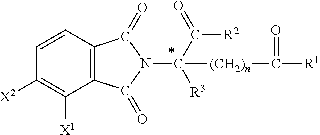

in which the carbon atom designated C* constitutes a center of chirality when n is not zero and R¹ is not R²; one of X¹ and X² is amino, nitro, alkyl of one to six carbons, or NH—Z, and the other of X¹ or X² is hydrogen; each of R¹ and R² independent of the other, is hydroxy or NH—Z; R³ is alkyl of one to six carbons, halo, or hydrogen; Z is hydrogen, aryl, or an alkyl or acyl of one to six carbons; and n has a value of 0, 1, or 2; and the salts thereof.

Specific examples include, but are not limited to, 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl}-4-phenylcarbamoyl-butyric acid, and 2-{4-[(furan-2-yl-methyl)-amino]-1,3-dioxo-1,3-dihydro-isoindol-2-yl}-pentanedioic acid, which have the following structures, respectively, and pharmaceutically acceptable salts, solvate, prodrugs, and stereoisomers thereof:

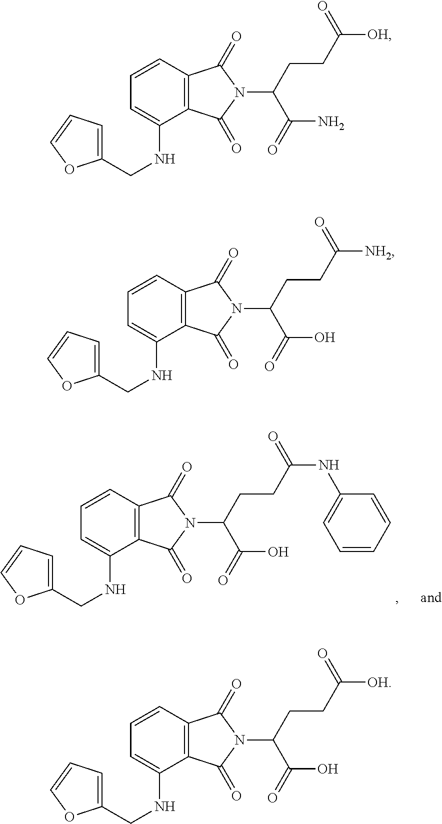 [SMILES: NC(=O)C(CCC(=O)O)N1C(=O)c2cccc(NCc3ccco3)c2C1=O ; NC(=O)CCC(C(=O)O)N1C(=O)c2cccc(NCc3ccco3)c2C1=O ; O=C(CCC(C(=O)O)N1C(=O)c2cccc(NCc3ccco3)c2C1=O)Nc1ccccc1 ; O=C(O)CCC(C(=O)O)N1C(=O)c2cccc(NCc3ccco3)c2C1=O]

Other specific examples of the compounds are of formula:

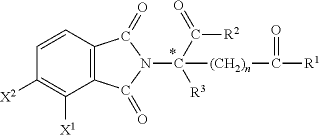

wherein:

one of X¹ and X² is nitro, or NH—Z, and the other of X¹ or X² is hydrogen;

each of R¹ and R², independent of the other, is hydroxy or NH—Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Other representative compounds are of formula:

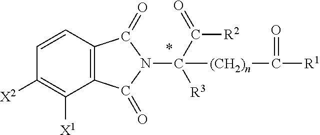

wherein:

one of X¹ and X² is alkyl of one to six carbons;

each of R¹ and R², independent of the other, is hydroxy or NH—Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Still other specific IMiD® immunomodulatory drugs provided herein include, but are not limited to, isoindoline-1-one and isoindoline-1,3-dione substituted in the 2-position with 2,6-dioxo-3-hydroxypiperidin-5-yl described in U.S. Pat. No. 6,458,810, which is incorporated herein by reference. Representative compounds are of formula:

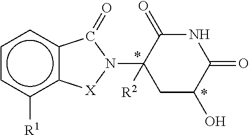

wherein:

the carbon atoms designated * constitute centers of chirality;

X is —C(O)— or —CH₂—;

R¹ is alkyl of 1 to 8 carbon atoms or —NHR³;

R² is hydrogen, alkyl of 1 to 8 carbon atoms, or halogen; and

R³ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms,

benzyl, unsubstituted or substituted with alkyl of 1 to 8 carbon atoms, alkoxy of 1 to 8 carbon atoms, halo, amino, or alkylamino of 1 to 4 carbon atoms, or —COR⁴ in which

R⁴ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms, or

benzyl, unsubstituted or substituted with alkyl of 1 to 8 carbon atoms, alkoxy of 1 to 8 carbon atoms, halo, amino, or alkylamino of 1 to 4 carbon atoms.

Still other specific IMiD® immunomodulatory drugs provided herein belong to a class of isoindole-imides disclosed in U.S. Patent Application Publication No. US 2007/0049618, the entirety of which is incorporated herein by reference. Representative compounds are of formula IV:

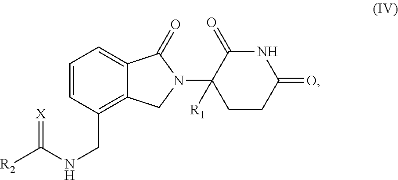

and pharmaceutically acceptable salts, solvates, stereoisomers, and prodrugs thereof,
wherein:

### X is O or S;

R₁ is H or methyl;

### R₂ is:

(C₂-C₆)alkyl, excluding cycloalkyl; (C₄-C₆)cycloalkyl; (C₁-C₄)alkoxy;
(C₁-C₆)alkyl, substituted with (C₁-C₄)alkoxy;
(C₀-C₁)alkyl-phenyl, wherein the phenyl is optionally substituted with one or more of halogen, (C₁-C₄)alkoxy, (C₁-C₄)alkyl, or cyano;
(C₀-C₁)alkyl- (5 to 6 membered heteroaryl), wherein the heteroaryl is optionally substituted with one or more of (C₁-C₄)alkyl or halogen; or
(C₀-C₃)alkyl-NR₃R₄;
   R₃ and R₄ are each independently:
H; (C₁-C₆)alkyl; (C₃-C₆)cycloalkyl;
(C₀-C₁)alkyl-(C₆-C₁₀)aryl, wherein the aryl is optionally substituted with one or more of (C₁-C₄)alkoxy, halogen, methyl, cyano, or —O—CH₂—O—;
(C₀-C₁)alkyl- (5 to 10 membered heteroaryl), wherein the heteroaryl is substituted with one or more of (C₁-C₄)alkoxy, halogen, or methyl; or C(O)R₅; and
   R₅ is (C₁-C₄)alkoxy or (C₁-C₂)alkyl-O—(C₁-C₂)alkyl;
   with the proviso that if one of R₃ and R₄ is H, then the other is not ethyl.

In one embodiment, X is O. In another embodiment, X is S. In another embodiment, R² is phenyl, optionally substituted with one or more halogen.

In another embodiment, R² is NHR⁴. In a specific embodiment, R⁴ is (C₆-C₁₀)aryl or 5 to 10 membered heteroaryl, both optionally substituted with one or more of (C₁-C₄)alkoxy, halogen, and methyl. In particular, the aryl or heteroaryl is phenyl, pyridyl, or naphthyl.

Examples of compounds of formula (IV) include, but are not limited to, those listed in Table B, below:

TABLE B
Compounds of Formula IV

| No. | Structure | Name |
|---|---|---|
| 1 | 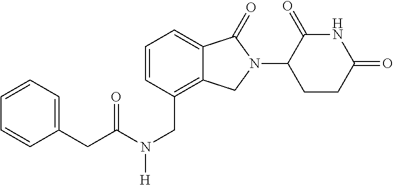 [SMILES: O=C(Cc1ccccc1)NCc1cccc2c1CN(C1CCC(=O)NC1=O)C2=O] | N-[2-(2,6-Dioxo-piperidin- 3-yl)-1-oxo2,3-dihydro-1H- isoindol-4-ylmethyl]-2- phenyl-acetamide |
| 2 | 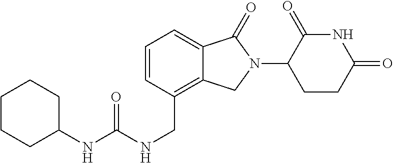 [SMILES: O=C1CCC(N2Cc3c(CNC(=O)NC4CCCCC4)cccc3C2=O)C(=O)N1] | 1-Cyclohexyl-3-[2-(2,6- dioxo-piperidin-3-yl)-1- oxo-2,3-dihydro-1H- isoindol-4-ylmethyl]-urea |
| 3 | 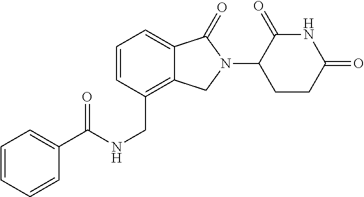 [SMILES: O=C1CCC(N2Cc3c(CNC(=O)c4ccccc4)cccc3C2=O)C(=O)N1] | N-[2-(2,6-Dioxo-piperidin- 3-yl)-1-oxo-2,3-dihydro- 1H-isoindol-4-ylmethyl]- benzamide |
| 4 | 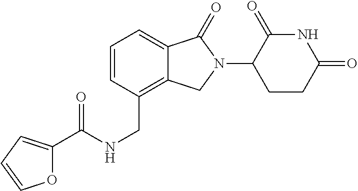 [SMILES: O=C1CCC(N2Cc3c(CNC(=O)c4ccco4)cccc3C2=O)C(=O)N1] | Furan-2-carboxylic acid [2- (2,6-dioxo-piperidin-3-yl)- 1-oxo-2,3-dihydro-1H- isoindol-4-ylmethyl]-amide |
| 5 | 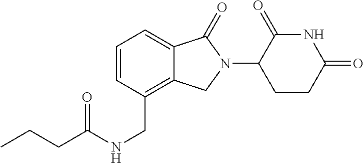 [SMILES: CCCC(=O)NCc1cccc2c1CN(C1CCC(=O)NC1=O)C2=O] | N-[2-(2,6-Dioxo-piperidin- 3-yl)-1-oxo-2,3-dihydro- 1H-isoindol-4-ylmethyl]- butyramide |
| 6 | 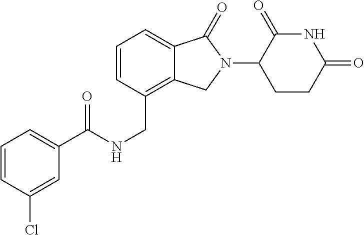 [SMILES: O=C1CCC(N2Cc3c(CNC(=O)c4cccc(Cl)c4)cccc3C2=O)C(=O)N1] | 3-Chloro-N-[2-(2,6-dioxo- piperidin-3-yl)-1-oxo-2,3- dihydro-1H-sioindol-4- ylmethyl]-benzamide |
| 7 | 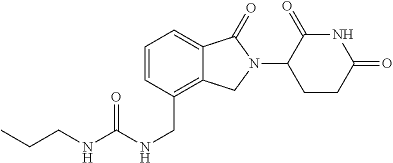 [SMILES: CCCNC(=O)NCc1cccc2c1CN(C1CCC(=O)NC1=O)C2=O] | 1-[2-(2,6-Dioxo-piperidin- 3-yl)-1-oxo-2,3-dihydro- 1H-isoindol-4-ylmethyl]-3- propyl-urea |
| 8 | 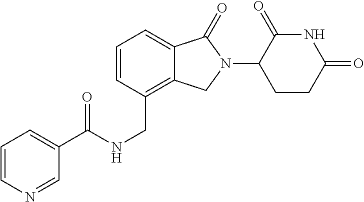 [SMILES: O=C1CCC(N2Cc3c(CNC(=O)c4cccnc4)cccc3C2=O)C(=O)N1] | N-[2-(2,6-Dioxo-piperidin- 3-yl)-1-oxo-2,3-dihydro- 1H-isoindol-4-ylmethyl]- nicotinamide |
| 9 | 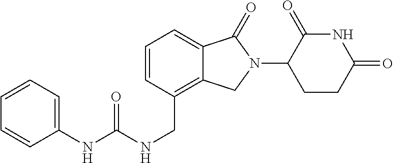 [SMILES: O=C1CCC(N2Cc3c(CNC(=O)Nc4ccccc4)cccc3C2=O)C(=O)N1] | 1-[2-(2,6-Dioxo-piperidin- 3-yl)-1-oxo-2,3-dihydro- 1H-isoindol-4-ylmethyl]-3- phenyl-urea |
| 10 | 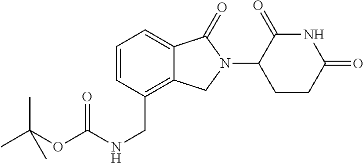 [SMILES: CC(C)(C)OC(=O)NCc1cccc2c1CN(C1CCC(=O)NC1=O)C2=O] | [2-(2,6-Dioxo-piperidin-3- yl)-1-oxo-2,3-dihydro-1H- isoindol-4-ylmethyl]- carbamic acid tert-butyl ester |
| 11 | 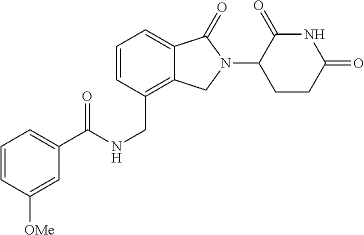 [SMILES: COc1cccc(C(=O)NCc2cccc3c2CN(C2CCC(=O)NC2=O)C3=O)c1] | N-[2-(2,6-Dioxo-piperidin- 3-yl)-1-oxo-2,3-dihydro- 1H-isoindol-4-ylmethyl]-3- methoxy-benzamide |
| 12 | 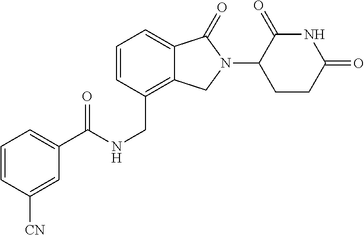 [SMILES: N#Cc1cccc(C(=O)NCc2cccc3c2CN(C2CCC(=O)NC2=O)C3=O)c1] | 3-Cyano-N-[2-(2,6-dioxo- piperidin-3-yl)-1-oxo-2,3- dihydro-1H-isoindol-4- ylmethyl]-benzamide |
| 13 | 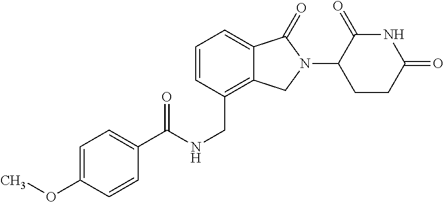 [SMILES: COc1ccc(C(=O)NCc2cccc3c2CN(C2CCC(=O)NC2=O)C3=O)cc1] | N-[2-(2,6-Dioxo-piperidin- 3-yl)-1-oxo-2,3-dihydro- 1H-isoindol-4-ylmethyl]-4- methoxy-benzamide |
| 14 | 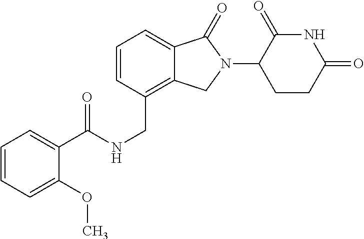 [SMILES: COc1ccccc1C(=O)NCc1cccc2c1CN(C1CCC(=O)NC1=O)C2=O] | N-[2-(2,6-Dioxo-piperidin- 3-yl)-1-oxo-2,3-dihydro- 1H-isoindol-4-ylmethyl]-2- methoxy-benzamide |
| 15 | 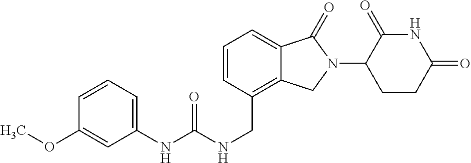 [SMILES: COc1cccc(NC(=O)NCc2cccc3c2CN(C2CCC(=O)NC2=O)C3=O)c1] | 1-[2-(2,6-Dioxo-pipderidin- 3-yl)-1-oxo-2,3-dihydro- 1H-isoindol-4-ylmethyl]-3- (3-methoxy-phenyl)-urea |
| 16 | 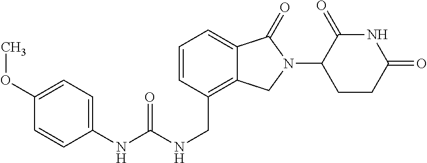 [SMILES: COc1ccc(NC(=O)NCc2cccc3c2CN(C2CCC(=O)NC2=O)C3=O)cc1] | 1-[2-(2,6-Dioxo-piperidin- 3-yl)-1-oxo-2,3-dihydro- 1H-isoindol-4-ylmethyl]-3- (4-methoxy-phenyl)-urea |
| 17 | 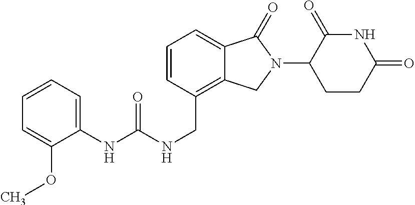 [SMILES: COc1ccccc1NC(=O)NCc1cccc2c1CN(C1CCC(=O)NC1=O)C2=O] | 1-[2-(2,6-Dioxo-piperidin- 3-yl)-1-oxo-2,3-dihydro- 1H-isoindol-4-ylmethyl]-3- (2-methoxy-phenyl)-urea |
| 18 | 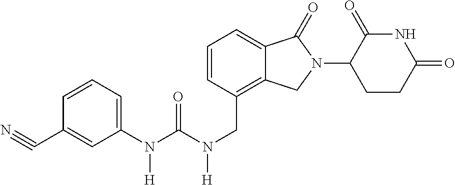 [SMILES: N#Cc1cccc(NC(=O)NCc2cccc3c2CN(C2CCC(=O)NC2=O)C3=O)c1] | 1-(3-Cyano-phenyl)-3-[2- (2,6-dioxo-piperidin-3-yl)- 1-oxo-2,3-dihydro-1H- isoindol-4-ylmethyl]-urea |
| 19 | 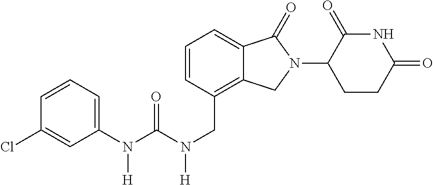 [SMILES: O=C1CCC(N2Cc3c(CNC(=O)Nc4cccc(Cl)c4)cccc3C2=O)C(=O)N1] | 1-(3-Chloro-phenyl)-3-[2- (2,6-dioxo-piperidin-3-yl)- 1-oxo-2,3-dihydro-1H- isoindol-4-ylmethyl]-urea |
| 20 | 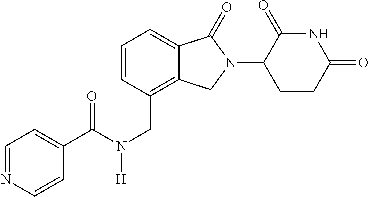 [SMILES: O=C1CCC(N2Cc3c(CNC(=O)c4ccncc4)cccc3C2=O)C(=O)N1] | N-[2-(2,6-Dioxo-piperidin- 3-yl)-1-oxo-2,3-dihydro- 1H-isoindol-4-ylmethyl]- isonicotinamide |
| 21 | 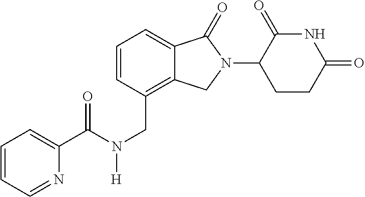 [SMILES: O=C1CCC(N2Cc3c(CNC(=O)c4ccccn4)cccc3C2=O)C(=O)N1] | Pyridine-2-carboxylic acid [2-(2,6-dioxo-piperidin-3- yl)-1-oxo-2,3-dihydro-1H- isoindol-4-ylmethyl]-amide |
| 22 | 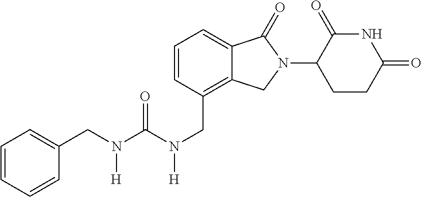 [SMILES: O=C1CCC(N2Cc3c(CNC(=O)NCc4ccccc4)cccc3C2=O)C(=O)N1] | 1-Benzyl-3-[2-(2,6-dioxo- piperidin-3-yl)-1-oxo-2,3- dihydro-1H-isoindol-4- ylmethyl]-urea |
| 23 | 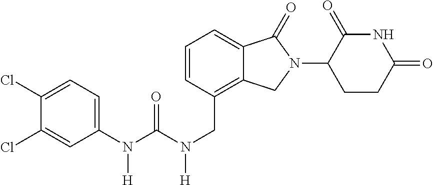 [SMILES: O=C1CCC(N2Cc3c(CNC(=O)Nc4ccc(Cl)c(Cl)c4)cccc3C2=O)C(=O)N1] | 1-(3,4-Dichloro-phenyl)-3- [2-(2,6-dioxo-piperidin-3- yl)-1-oxo-2,3-dihydro-1H- isoindol-4-ylmethyl]-urea |
| 24 | 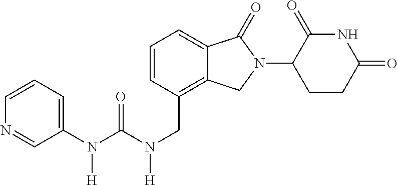 [SMILES: O=C1CCC(N2Cc3c(CNC(=O)Nc4cccnc4)cccc3C2=O)C(=O)N1] | 1-[2-(2,6-Dioxo-piperidin- 3-yl)-1-oxo-2,3-dihydro- 1H-isoindol-4-ylmehyl]-3- pyridin-3-yl-urea |
| 25 | 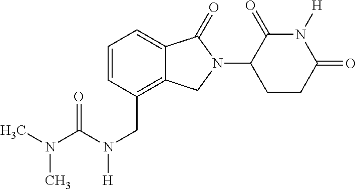 [SMILES: CN(C)C(=O)NCc1cccc2c1CN(C1CCC(=O)NC1=O)C2=O] | 3-[2-(2,6-Dioxo-piperidin- 3-yl)-1-oxo-2,3-dihydro- 1H-isoindol-4-ylmethyl]- 1,1-dimethyl-urea |
| 26 | 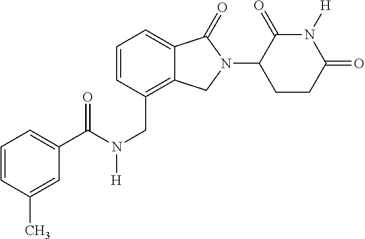 [SMILES: Cc1cccc(C(=O)NCc2cccc3c2CN(C2CCC(=O)NC2=O)C3=O)c1] | N-[2-(2,6-Dioxo-piperidin- 3-yl)-1-oxo-2,3-dihydro- 1H-isoindol-4-ylmethyl]-3- methyl-benzamide |
| 27 | 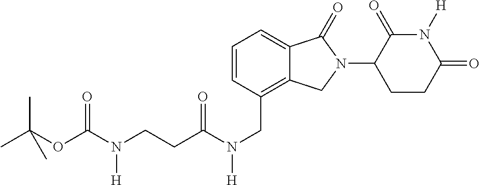 [SMILES: CC(C)(C)OC(=O)NCCC(=O)NCc1cccc2c1CN(C1CCC(=O)NC1=O)C2=O] | (2-{[2-(2,6-Dioxo- piperidin-3-yl)-1-oxo-2,3- dihydro-1H-isoindol-4- ylmethyl]-carbamoyl}- ethyl)-carbamic acid t-butyl ester |
| 28 | 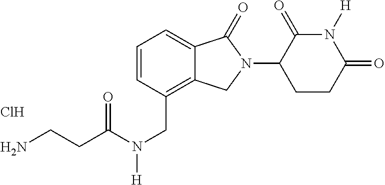 [SMILES: Cl ; NCCC(=O)NCc1cccc2c1CN(C1CCC(=O)NC1=O)C2=O] | 3-Amino-N-[2-(2,6-dioxo- piperidin-3-yl)-1-oxo-2,3- dihydro-1H-isoindol-4- ylmethyl]-propionamide Hydrochloride |
| 29 | 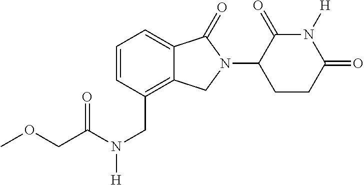 [SMILES: COCC(=O)NCc1cccc2c1CN(C1CCC(=O)NC1=O)C2=O] | N-[2-(2,6-Dioxo-piperidin- 3-yl)-1-oxo-2,3-dihydro- 1H-isoindol-4-ylmethyl]-2- methoxy-acetamide |
| 30 | 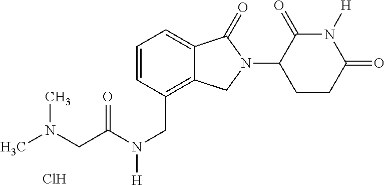 [SMILES: CN(C)CC(=O)NCc1cccc2c1CN(C1CCC(=O)NC1=O)C2=O ; Cl] | 2-Dimethylamino-N-[2- (2,6-dioxo-piperidin-3-yl)- 1-oxo-2,3-dihydro-1H- isoindol-4-ylmethyl]- acetamide Hydrochloride |
| 31 | 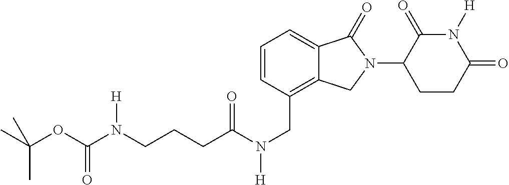 [SMILES: CC(C)(C)OC(=O)NCCCC(=O)NCc1cccc2c1CN(C1CCC(=O)NC1=O)C2=O] | (3-{[2-(2,6-Dioxo- piperidin-3-yl)-1-oxo-2,3- dihydro-1H-isoindol-4- ylmethyl]-carbamoyl}- propyl)-carbamic acid t- butyl ester |
| 32 | 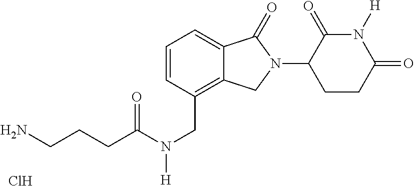 [SMILES: NCCCC(=O)NCc1cccc2c1CN(C1CCC(=O)NC1=O)C2=O ; Cl] | 4-Amino-[2-(2,6-dioxo- piperidin-3-yl)-1-oxo-2,3- dihydro-1H-isoindol-4- ylmethyl]-butyramide hydrochloride |
| 33 | 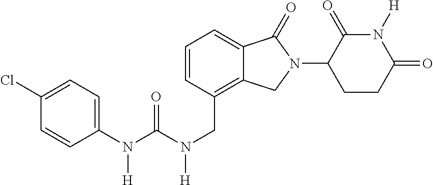 [SMILES: O=C1CCC(N2Cc3c(CNC(=O)Nc4ccc(Cl)cc4)cccc3C2=O)C(=O)N1] | 1-(4-Chloro-phenyl)-3-[2- (2,6-dioxo-piperidin-3-yl)- 1-oxo-2,3-dihydro-1H- isoindol-4-ylmethyl]-urea |
| 34 | 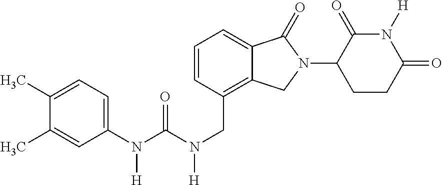 [SMILES: Cc1ccc(NC(=O)NCc2cccc3c2CN(C2CCC(=O)NC2=O)C3=O)cc1C] | 1-(3,4-Dimethyl-phenyl)-3- [2-(2,6-dioxo-piperidin-3- yl)-1-oxo-2,3-dihydro-1H- isoindol-4-ylmethyl]-urea |
| 35 | 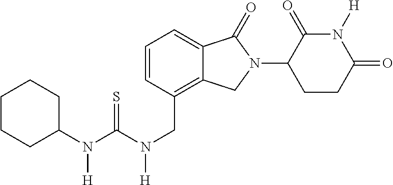 [SMILES: O=C1CCC(N2Cc3c(CNC(=S)NC4CCCCC4)cccc3C2=O)C(=O)N1] | 1-Cyclohexyl-3-[2-(2,6- dioxo-piperidin-3-yl)-1- oxo-2,3-dihydro-1H- isoindol-4-ylmethyl]- thiourea |
| 36 | 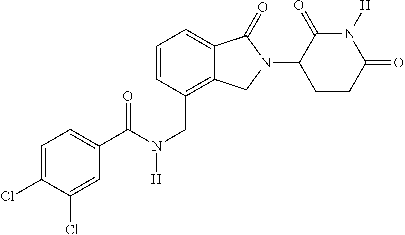 [SMILES: O=C1CCC(N2Cc3c(CNC(=O)c4ccc(Cl)c(Cl)c4)cccc3C2=O)C(=O)N1] | 3,4-Dichloro-N-[2-(2,6- dioxo-piperidin-3-yl)-1- oxo-2,3-dihydro-1H- isoindol-4-ylmethyl]- benzamide |
| 37 | 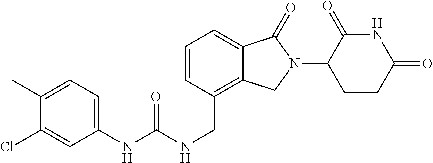 [SMILES: Cc1ccc(NC(=O)NCc2cccc3c2CN(C2CCC(=O)NC2=O)C3=O)cc1Cl] | 1-(3-Chloro-4- methylphenyl)-3-[2-(2,6- dioxo-piperidin-3-yl)-1- oxo-2,3-dihydro-1H- isoindol-4-ylmethyl]urea |
| 38 | 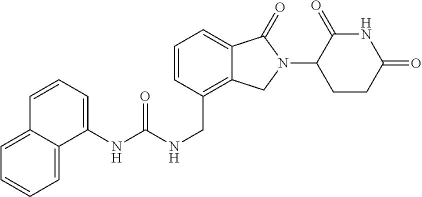 [SMILES: O=C1CCC(N2Cc3c(CNC(=O)Nc4cccc5ccccc45)cccc3C2=O)C(=O)N1] | 1-[2-(2,6-Dioxopiperidin-3- yl)-1-oxo-2,3-dihydro-1H- isoindol-4-ylmethyl]-3- naphthalen-1-yl-urea |
| 39 | 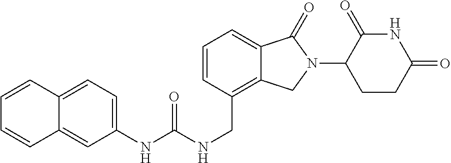 [SMILES: O=C1CCC(N2Cc3c(CNC(=O)Nc4ccc5ccccc5c4)cccc3C2=O)C(=O)N1] | 1-[2-(2,6-Dioxopiperidin-3- yl)-1-oxo-2,3-dihydro-1H- isoindol-4-ylmethyl]-3- naphthalen-2-yl-urea |

Still other representative compounds are of formula V:

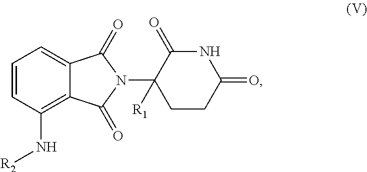

and pharmaceutically acceptable salts, solvates, stereoisomers, and prodrugs thereof,
wherein:
R₁ is H or methyl; and

### R₂ is:

(C₆-C₁₀)aryl, optionally substituted with one or more of: (C₁-C₈)alkyl, optionally substituted with NH₂, NH(CH₃), or N(CH₃)₂; (C₁-C₄)alkoxy, optionally substituted with NH₂, NH(CH₃), N(CH₃)₂, or 3 to 6 membered heterocycloalkyl; (C₃-C₆)cycloalkyl; (C₅-C₁₀)aryloxy; hydroxy; NH₂; NH(CH₃); N(CH₃)₂; —CH₂—CH₂—CH₂—; halogen; or —O—CH₂—O—;
(C₃-C₆)alkyl, optionally substituted with one or more of (C₁-C₄)alkoxy;
(C₁-C₂)alkyl, optionally substituted with carboxyl;
(C₁-C₆)alkyl-(C₃-C₆)cycloalkyl; or
5 to 10 membered heterocycle;
   with the proviso that if R₂ is pentyl, then R₁ is methyl.

In one embodiment, R₂ is phenyl, optionally substituted with one or more of (C₁-C₄)alkoxy or —O—CH₂—O—. In another embodiment, R₂ is phenyl substituted with one or more (C₁-C₄)alkoxy, substituted with N(CH₃)₂. In another embodiment, R₂ is (C₃-C₆)alkyl, optionally substituted with one or more of (C₁-C₄)alkoxy.

Examples of compounds of formula (V) include, but are not limited to, those listed in Table C, below:

TABLE C
Compounds of Formula V

| No. | Structure | Name |
|---|---|---|
| 40 | 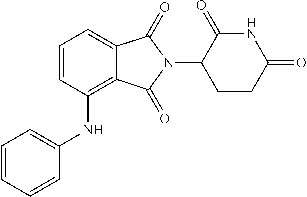 [SMILES: O=C1CCC(N2C(=O)c3cccc(Nc4ccccc4)c3C2=O)C(=O)N1] | 2-(2,6-Dioxopiperidin-3-yl)-4- phenylaminoisoindole-1,3- dione |
| 41 | 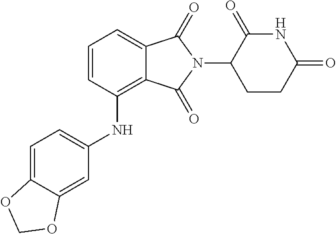 [SMILES: O=C1CCC(N2C(=O)c3cccc(Nc4ccc5c(c4)OCO5)c3C2=O)C(=O)N1] | 2-(2,6-Dioxopiperidin-3-yl)-4- (3,4-methylenedioxy- phenylamino)isoindole- 1,3-dione |
| 42 | 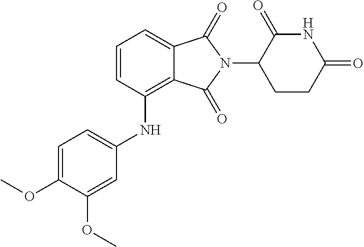 [SMILES: COc1ccc(Nc2cccc3c2C(=O)N(C2CCC(=O)NC2=O)C3=O)cc1OC] | 2-(2,6-Dioxopiperidin-3-yl)-4- (3,4-dimethoxy- phenylamino)isoindole- 1,3-dione |
| 43 | 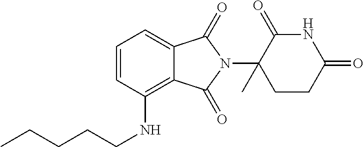 [SMILES: CCCCCNc1cccc2c1C(=O)N(C1(C)CCC(=O)NC1=O)C2=O] | 2-(3-Methyl-2,6- dioxopiperidin-3-yl)-4- pentylaminoisoindole-1,3- dione |
| 44 | 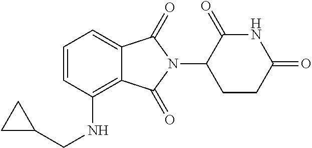 [SMILES: O=C1CCC(N2C(=O)c3cccc(NCC4CC4)c3C2=O)C(=O)N1] | 4-(Cyclopropylmethylamino)- 2-(2,6-dioxopiperidin-3- yl)isoindole-1,3-dione |
| 45 | 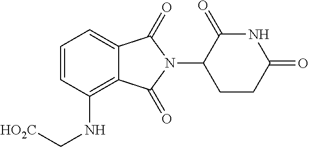 [SMILES: O=C(O)CNc1cccc2c1C(=O)N(C1CCC(=O)NC1=O)C2=O] | [2-(2,6-Dioxopiperidin-3-yl)- 1,3-dioxo-2,3-dihydro-1H- isoindol-4-yl-amino]acetic acid |
| 46 | 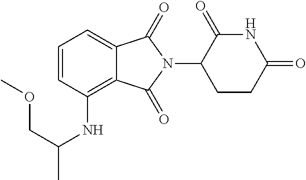 [SMILES: COCC(C)Nc1cccc2c1C(=O)N(C1CCC(=O)NC1=O)C2=O] | 2-(2,6-Dioxopiperidin-3-yl)-4- (2-methoxy-1- methylethylamino)isoindole- 1,3-dione |
| 47 | 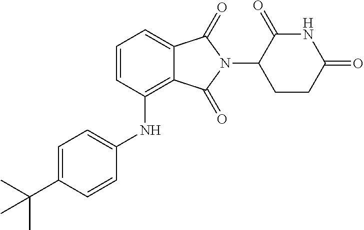 [SMILES: CC(C)(C)c1ccc(Nc2cccc3c2C(=O)N(C2CCC(=O)NC2=O)C3=O)cc1] | 4-(4-tert-Butylphenylamino)- 2-(2,6-dioxopiperidin-3- yl)isoindole-1,3-dione |
| 48 | 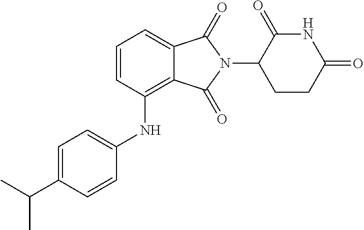 [SMILES: CC(C)c1ccc(Nc2cccc3c2C(=O)N(C2CCC(=O)NC2=O)C3=O)cc1] | 4-(4-Isopropylphenylamino)-2- (2,6-dioxopiperidin-3- yl)isoindole-1,3-dione |
| 49 | 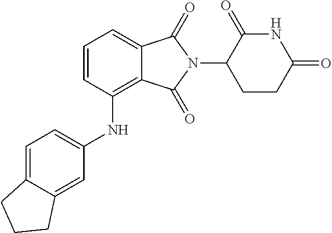 [SMILES: O=C1CCC(N2C(=O)c3cccc(Nc4ccc5c(c4)CCC5)c3C2=O)C(=O)N1] | 2-(2,6-Dioxo-piperidin-3-yl)- 4-(indan-5-ylamino)-isoindole- 1,3-dione |
| 50 | 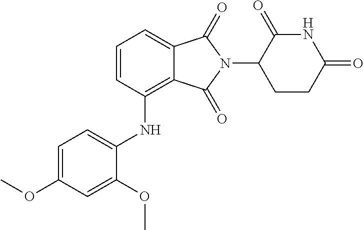 [SMILES: COc1ccc(Nc2cccc3c2C(=O)N(C2CCC(=O)NC2=O)C3=O)c(OC)c1] | 4-(2,4- Dimethoxyphenylamino)-2- (2,6-dioxopiperidin-3- yl)isoindole-1,3-dione |
| 51 | 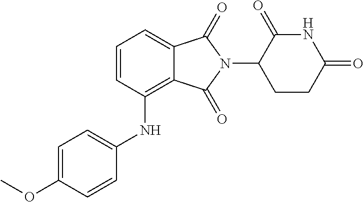 [SMILES: COc1ccc(Nc2cccc3c2C(=O)N(C2CCC(=O)NC2=O)C3=O)cc1] | 2-(2,6-Dioxopiperidin-3-yl)-4- (4-methoxyphenylamino) isoindole-1,3-dione |
| 52 | 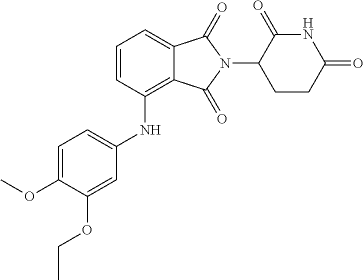 [SMILES: CCOc1cc(Nc2cccc3c2C(=O)N(C2CCC(=O)NC2=O)C3=O)ccc1OC] | 2-(2,6-Dioxopiperidin-3-yl)-4- (3-ethoxy-4- methoxyphenylamino)- isoindole-1,3-dione |
| 53 | 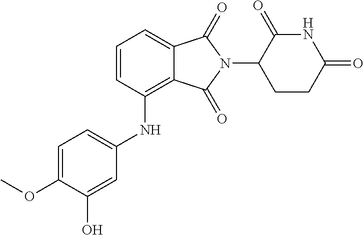 [SMILES: COc1ccc(Nc2cccc3c2C(=O)N(C2CCC(=O)NC2=O)C3=O)cc1O] | 2-(2,6-Dioxopiperidin-3-yl)-4- (3-hydroxy-4- methoxyphenylamino)- isoindole-1,3-dione |
| 54 | 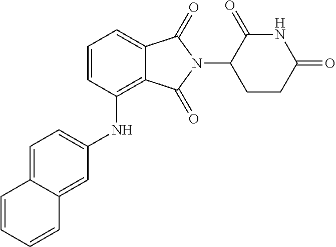 [SMILES: O=C1CCC(N2C(=O)c3cccc(Nc4ccc5ccccc5c4)c3C2=O)C(=O)N1] | i. 2-(2,6-Dioxopiperidin- 3-yl)-4-(naphthalen-2- ylamino) isoindole-1,3-dione |
| 55 | 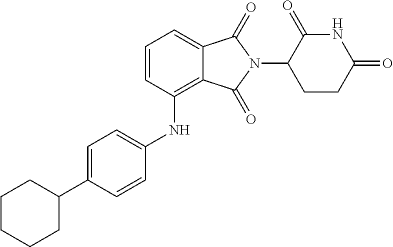 [SMILES: O=C1CCC(N2C(=O)c3cccc(Nc4ccc(C5CCCCC5)cc4)c3C2=O)C(=O)N1] | 4-(4-Cyclohexylphenylamino)- 2-(2,6-dioxopiperidin-3- yl)isoindole-1,3-dione |
| 56 | 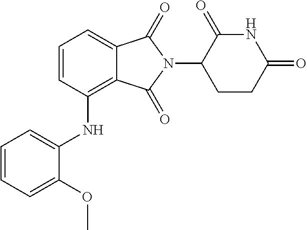 [SMILES: COc1ccccc1Nc1cccc2c1C(=O)N(C1CCC(=O)NC1=O)C2=O] | 4-(2-Methoxyphenylamino)-2- (2,6-dioxopiperidin-3- yl)isoindole-1,3-dione |
| 57 | 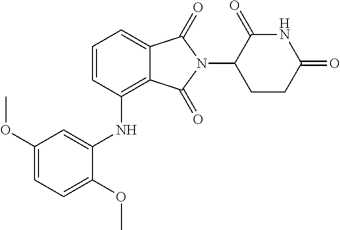 [SMILES: COc1ccc(OC)c(Nc2cccc3c2C(=O)N(C2CCC(=O)NC2=O)C3=O)c1] | 4-(2,5- Dimethoxyphenylamino)-2- (2,6-dioxopiperidin-3- yl)isoindole-1,3-dione |
| 58 | 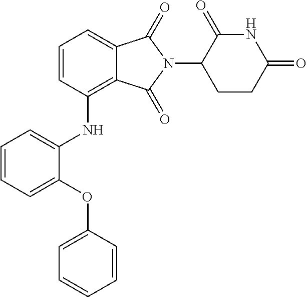 [SMILES: O=C1CCC(N2C(=O)c3cccc(Nc4ccccc4Oc4ccccc4)c3C2=O)C(=O)N1] | 4-(2-Phenoxyphenylamino)-2- (2,6-dioxopiperidin-3-yl) isoindole-1,3-dione |
| 59 | 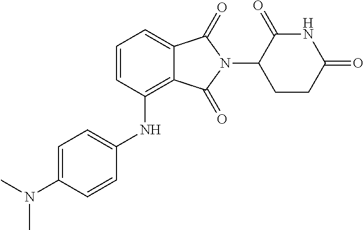 [SMILES: CN(C)c1ccc(Nc2cccc3c2C(=O)N(C2CCC(=O)NC2=O)C3=O)cc1] | 4-(4- Dimethylaminophenylamino)- 2-(2,6-dioxopiperidin-3- yl)isoindole-1,3-dione |
| 60 | 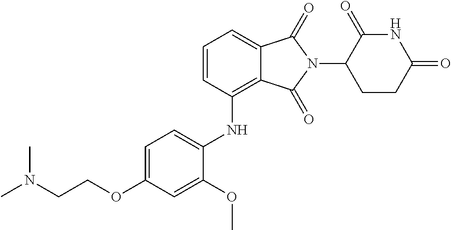 [SMILES: COc1cc(OCCN(C)C)ccc1Nc1cccc2c1C(=O)N(C1CCC(=O)NC1=O)C2=O] | 4-[4-(2- Dimethylaminoethoxy)-2- methoxyphenylamino]-2-(2,6- dioxo-piperidin-3-yl)- isoindole-1,3-dione |
| 61 | 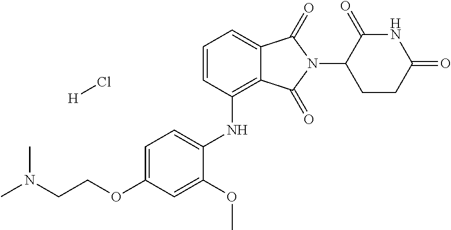 [SMILES: COc1cc(OCCN(C)C)ccc1Nc1cccc2c1C(=O)N(C1CCC(=O)NC1=O)C2=O ; Cl] | 4-[4-(2- Dimethylaminoethoxy)-2- methoxyphenylamino]-2-(2,6- dioxo-piperidin-3-yl)- isoindole-1,3-dione hydrochloride |
| 62 | 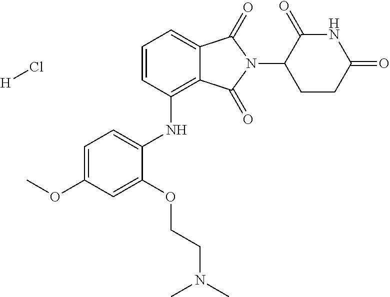 [SMILES: Cl ; COc1ccc(Nc2cccc3c2C(=O)N(C2CCC(=O)NC2=O)C3=O)c(OCCN(C)C)c1] | 4-[2-(2-Dimethylamino- ethoxy)-4-methoxyphenyl- amino]-2-(2,6-dioxopiperidin- 3-yl)isoindole-1,3-dione hydrochloride |
| 63 | 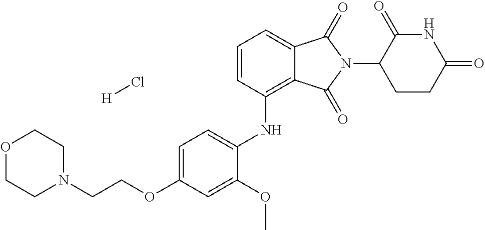 [SMILES: COc1cc(OCCN2CCOCC2)ccc1Nc1cccc2c1C(=O)N(C1CCC(=O)NC1=O)C2=O ; Cl] | 2-(2,6-Dioxopiperidin-3-yl)-4- [2-methoxy-4-(2-morpholin-4- ylethoxy)phenylamino]- isoindole-1,3-dione hydrochloride |
| 64 | 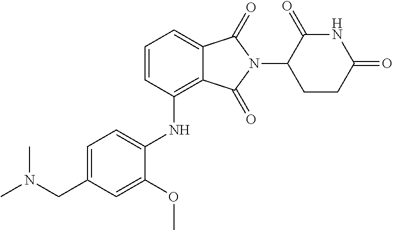 [SMILES: COc1cc(CN(C)C)ccc1Nc1cccc2c1C(=O)N(C1CCC(=O)NC1=O)C2=O] | 4-(4-Dimethylaminomethyl-2- methoxyphenylamino)-2-(2,6- dioxopiperidin-3-yl)isoindole- 1,3-dione |
| 65 | 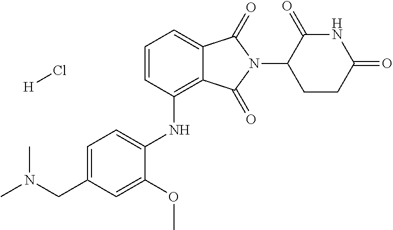 [SMILES: COc1cc(CN(C)C)ccc1Nc1cccc2c1C(=O)N(C1CCC(=O)NC1=O)C2=O ; Cl] | 4-(4-Dimethylaminomethyl-2- methoxyphenylamino)-2-(2,6- dioxopiperidin-3-yl)isoindole- 1,3-dione hydrochloride |
| 66 | 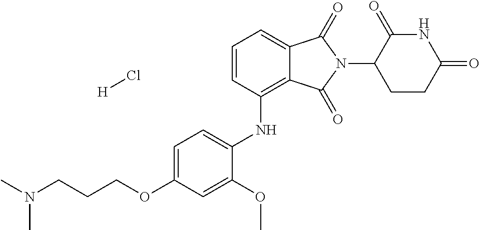 [SMILES: COc1cc(OCCCN(C)C)ccc1Nc1cccc2c1C(=O)N(C1CCC(=O)NC1=O)C2=O ; Cl] | 4-[4-(3- Dimethylaminopropoxy)-2- methoxyphenylamino]-2-(2,6- dioxopiperidin-3-yl)isoindole- 1,3-dione hydrochloride |
| 67 | 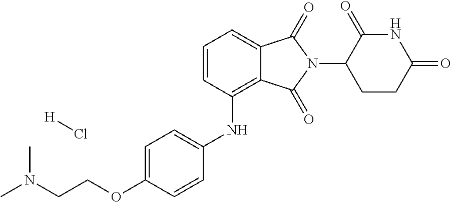 | 4-[4-(2-Dimethylamino- ethoxy)-phenylamino]-2-(2,6- dioxo-piperidin-3-yl)- isoindole-1,3-dione |
| 68 | 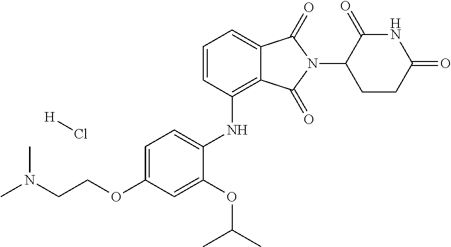 | 4-[4-(2-Dimethylamino- ethoxy)-2-isopropoxy- phenylamino]-2-(2,6-dioxo- piperidin-3-yl)-isoindole-1,3- dione |
| 69 | 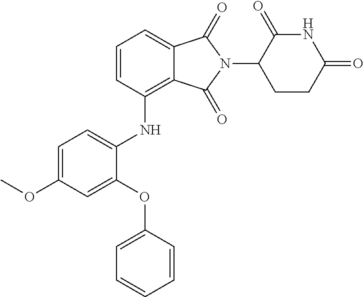 [SMILES: COc1ccc(Nc2cccc3c2C(=O)N(C2CCC(=O)NC2=O)C3=O)c(Oc2ccccc2)c1] | 2-(2,6-Dioxo-piperidin-3-yl)- 4-(4-methoxy-2-phenoxy- phenylamino)-isoindole-1,3- dione |
| 70 | 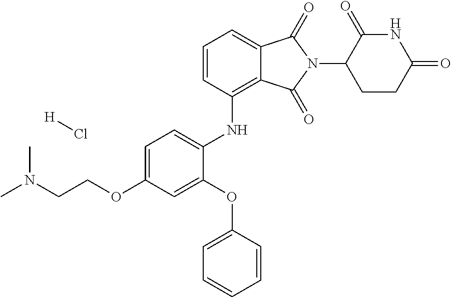 | 4-[4-(2-Dimethylamino- ethoxy)-2-phenoxy- phenylamino]-2-(2,6-dioxo- piperidin-3-yl)-isoindole-1,3- dione |
| 71 | 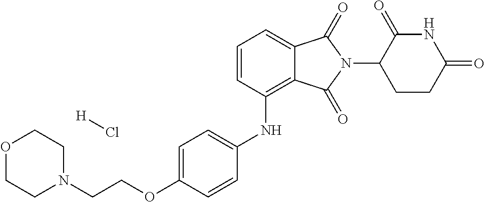 | 2-(2,6-Dioxo-piperidin-3-yl)- 4-[4-(2-morpholin-4-yl- ethoxy)-phenylamino]- isoindole-1,3-dione |
| 72 | 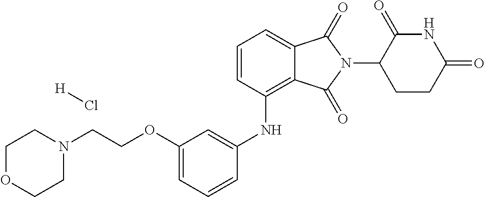 | 2-(2,6-Dioxo-piperidin-3-yl)- 4-[3-(2-morpholin-4-yl- ethoxy)-phenylamino]- isoindole-1,3-dione |
| 73 | 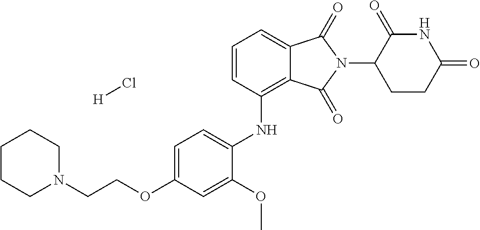 [SMILES: COc1cc(OCCN2CCCCC2)ccc1Nc1cccc2c1C(=O)N(C1CCC(=O)NC1=O)C2=O ; Cl] | 2-(2,6-Dioxo-piperidin-3-yl)- 4-[2-methoxy-4-(2-piperidin- 1-yl-ethoxy)-phenylamino]- isoindole-1,3-dione |
| 74 | 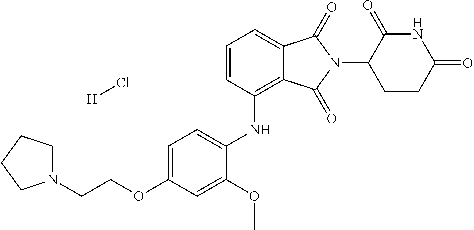 [SMILES: COc1cc(OCCN2CCCC2)ccc1Nc1cccc2c1C(=O)N(C1CCC(=O)NC1=O)C2=O ; Cl] | 2-(2,6-Dioxo-piperidin-3-yl)- 4-[2-methoxy-4-(2-pyrrolidin- 1-yl-ethoxy)-phenylamino]- isoindole-1,3-dione |
| 75 | 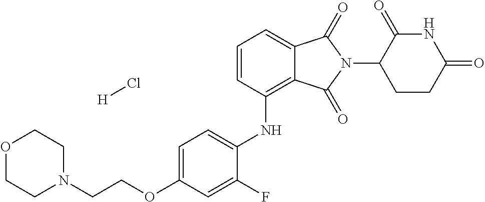 [SMILES: O=C1CCC(N2C(=O)c3cccc(Nc4ccc(OCCN5CCOCC5)cc4F)c3C2=O)C(=O)N1 ; Cl] | 2-(2,6-Dioxo-piperidin-3-yl)- 4-[2-fluoro-4-(2-morpholin-4- yl-ethoxy)-phenylamino]- isoindole-1,3-dione |
| 76 | 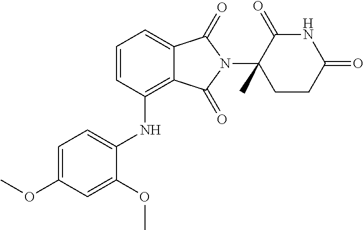 [SMILES: COc1ccc(Nc2cccc3c2C(=O)N([C@@]2(C)CCC(=O)NC2=O)C3=O)c(OC)c1] | 4-(2,4-Dimethoxy- phenylamino)-2-[(3S)-3- methyl-2,6-dioxo-piperidin-3- yl]-isoindole-1,3-dione |
| 77 | 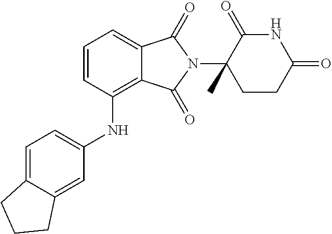 [SMILES: C[C@]1(N2C(=O)c3cccc(Nc4ccc5c(c4)CCC5)c3C2=O)CCC(=O)NC1=O] | 4-(Indan-5-ylamino)-2-[(3S)- 3-methyl-2,6-dioxo-piperidin- 3-yl]-isoindole-1,3-dione |
| 78 | 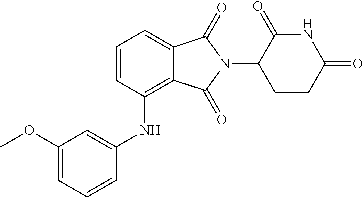 [SMILES: COc1cccc(Nc2cccc3c2C(=O)N(C2CCC(=O)NC2=O)C3=O)c1] | 2-(2,6-Dioxo-piperidin-3-yl)- 4-(3-methoxy-phenylamino)- isoindole-1,3-dione |

Still other representative compounds are of formula VI:

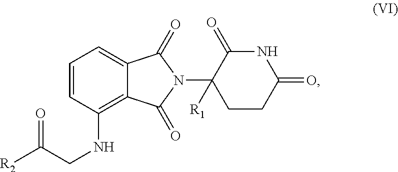

and pharmaceutically acceptable salts, solvates, stereoisomers, and prodrugs thereof,
wherein:
R₁ is H or methyl; and
R₂ is: amino, optionally substituted with one or more of (C₁-C₆)alkyl, (C₃-C₆)cycloalkyl, or phenyl; 3 to 6 membered heterocycloalkyl; or (C₁-C₄)alkoxy.

In one specific embodiment, R₂ is —NH(CH₃) or —N(CH₃)₂. In another embodiment, R₂ is (C₃-C₆)cycloalkyl.

Examples of compounds of formula (VI) include, but are not limited to, those listed in Table D, below:

TABLE D
Compounds of Formula VI

| No. | Structure | Name |
|---|---|---|
| 79 | 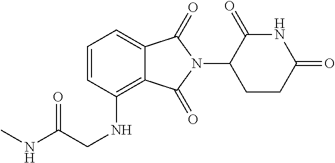 [SMILES: CNC(=O)CNc1cccc2c1C(=O)N(C1CCC(=O)NC1=O)C2=O] | 2-[2-(2,6-Dioxopiperidin-3-yl)-1,3- dioxo-2,3-dihydro-1H-isoindol-4- ylamino]-N-methylacetamide |
| 80 | 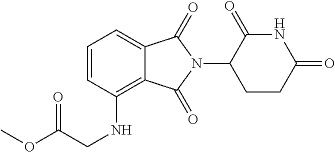 [SMILES: COC(=O)CNc1cccc2c1C(=O)N(C1CCC(=O)NC1=O)C2=O] | [2-(2,6-Dioxopiperidin-3-yl)-1,3- dioxo-2,3-dihydro-1H-isoindol-4- ylamino]acetic acid methyl ester |
| 81 | 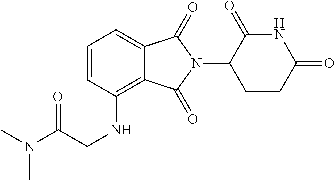 [SMILES: CN(C)C(=O)CNc1cccc2c1C(=O)N(C1CCC(=O)NC1=O)C2=O] | 2-[2-(2,6-Dioxopiperidin-3-yl)-1,3- dioxo-2,3-dihydro-1H-isoindol-4- ylamino]-N-methylacetamide |
| 82 | 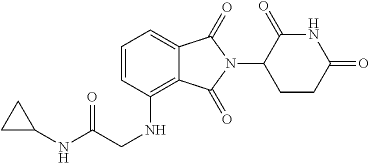 [SMILES: O=C1CCC(N2C(=O)c3cccc(NCC(=O)NC4CC4)c3C2=O)C(=O)N1] | N-Cyclopropyl-2-[2-(2,6- dioxopiperidin-3-yl)-1,3-dioxo- 2,3-dihydro-1H-isoindol-4- ylamino]acetamide |
| 83 | 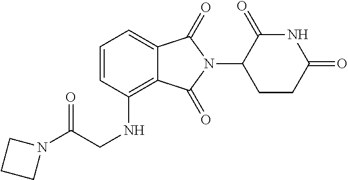 [SMILES: O=C1CCC(N2C(=O)c3cccc(NCC(=O)N4CCC4)c3C2=O)C(=O)N1] | 4-(2-(Azetidin-1-yl)-2- oxoethylamino)-2-(2,6-dioxo piperidin-3-yl)isoindoline-1,3- dione |
| 84 | 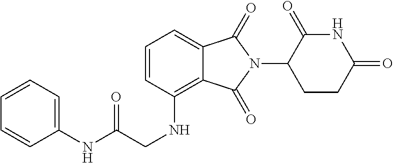 [SMILES: O=C1CCC(N2C(=O)c3cccc(NCC(=O)Nc4ccccc4)c3C2=O)C(=O)N1] | 2-[2-(2,6-Dioxopiperidin-3-yl)-1,3- dioxo-2,3-dihydro-1H-isoindol-4- ylamino]-N-phenyl-acetamide |

Still other representative compounds are of formula VII:

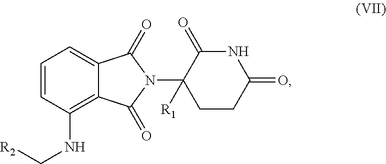

and pharmaceutically acceptable salts, solvates, stereoisomers, and prodrugs thereof,
wherein R₁ is H or methyl; and R₂ is 5 to 6 membered heteroaryl;
with the proviso that if R₂ is furan or thiophene, then R₁ is methyl; and
with the proviso that if R₂ is pyridine, then the pyridine is not connected to the core at the 3 position.

In one specific embodiment, R₂ is not pyridine.

Examples of compounds of formula VII include, but are not limited to, those listed in Table E, below:

TABLE E
Compounds of Formula VII

| No. | Structure | Name |
|---|---|---|
| 85 | 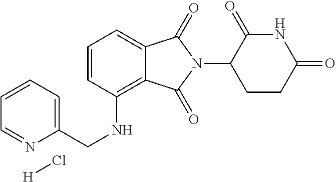 | 2-(2,6-Dioxopiperidin-3-yl)-4- [(pyridin-2-yl- methyl)amino]isoindole-1,3-dione hydrochloride |
| 86 | 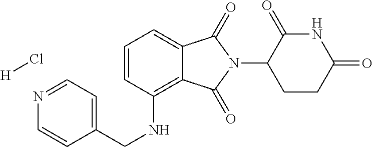 | 2-(2,6-Dioxopiperidin-3-yl)-4- [(pyridin-4-yl- methyl)amino]isoindole-1,3-dione hydrochloride |
| 87 | 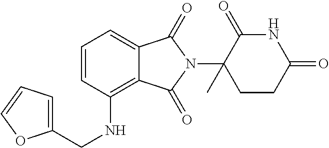 [SMILES: CC1(N2C(=O)c3cccc(NCc4ccco4)c3C2=O)CCC(=O)NC1=O] | 4-[(Furan-2-ylmethyl)amino]-2- (3 -methyl-2,6-dioxopiperidin-3- yl)isoindole-1,3-dione |

Still other representative compounds are of formula VIII:

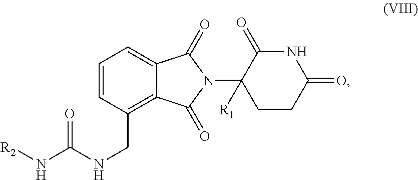

and pharmaceutically acceptable salts, solvates, stereoisomers, and prodrugs thereof:
wherein:
R₁ is H or methyl; and

### R₂ is:

H; methyl; ethyl;
phenyl, substituted with one or more of (C₁-C₆)alkyl, halogen, (C₁-C₄)alkoxy, cyano, or —O—CH₂—O—;
naphthyl, optionally substituted with one or more of (C₁-C₆)alkyl, halogen, (C₁-C₄)alkoxy, or cyano; or
5 to 10 membered heteroaryl, optionally substituted with one or more of (C₁-C₆)alkyl, halogen, (C₁-C₄)alkoxy, or cyano;
   with the proviso that if R₂ is ethyl, then R₁ is methyl; and
   with the proviso that if R₂ is pyridine, then the pyridine is not connected to the core at the 3 position.

In one specific embodiment, R₂ is phenyl, optionally substituted with one or more of methyl, halogen, (C₁-C₄)alkoxy, cyano, and —O—CH₂—O—. In another embodiment, R₂ is naphthyl. In another embodiment, R₂ is not pyridine.

Examples of compounds of formula (VIII) include, but are not limited to, those listed in Table F, below:

TABLE F
Compounds of Formula VIII

| No. | Structure | Name |
|---|---|---|
| 88 | 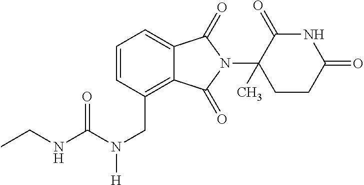 [SMILES: CCNC(=O)NCc1cccc2c1C(=O)N(C1(C)CCC(=O)NC1=O)C2=O] | 1-Ethyl-3-[2-(3-methyl- 2,6-dioxo-piperidin-3-yl)- 1,3-dioxo-2,3-dihydro-1H- isoindol-4-ylmethyl]-urea |
| 89 | 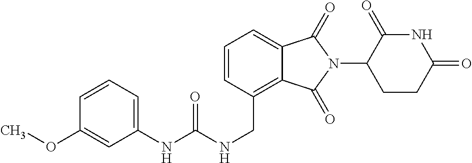 [SMILES: COc1cccc(NC(=O)NCc2cccc3c2C(=O)N(C2CCC(=O)NC2=O)C3=O)c1] | 1-[2-(2,6-Dioxo-piperidin- 3-yl)-1,3-dioxo-2,3- dihydro-1H-isoindol-4- ylmethyl]-3-(3-methoxy- phenyl)-urea |
| 90 | 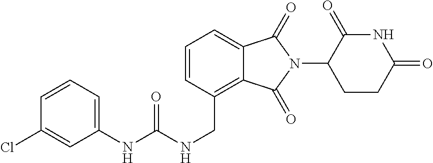 [SMILES: O=C1CCC(N2C(=O)c3cccc(CNC(=O)Nc4cccc(Cl)c4)c3C2=O)C(=O)N1] | 1-(3-Chloro-phenyl)-3-[2- (2,6-dioxo-piperidin-3-yl)- 1,3-dioxo-2,3-dihydro-1H- isoindol-4-ylmethyl]-urea |
| 91 | 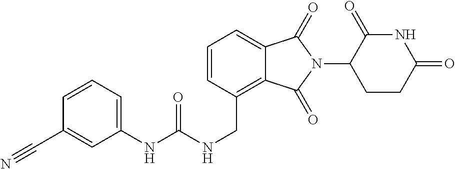 [SMILES: N#Cc1cccc(NC(=O)NCc2cccc3c2C(=O)N(C2CCC(=O)NC2=O)C3=O)c1] | 1-(3-Cyano-phenyl)-3-[2- (2,6-dioxo-piperidin-3-yl)- 1,3-dioxo-2,3-dihydro-1H- isoindol-4-ylmethyl]-urea |
| 92 | 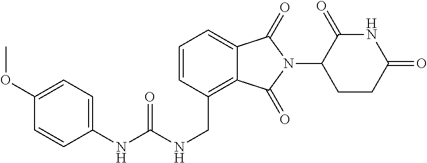 [SMILES: COc1ccc(NC(=O)NCc2cccc3c2C(=O)N(C2CCC(=O)NC2=O)C3=O)cc1] | 1-[2-(2,6-Dioxo-piperidin- 3-yl)-1,3-dioxo-2,3- dihydro-1H-isoindol-4- ylmethyl]-3-(4-methoxy- phenyl)-urea |
| 93 | 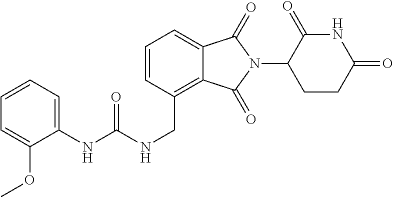 [SMILES: COc1ccccc1NC(=O)NCc1cccc2c1C(=O)N(C1CCC(=O)NC1=O)C2=O] | 1-[2-(2,6-Dioxo-piperidin- 3-yl)-1,3-dioxo-2,3- dihydro-1H-isoindol-4- ylmethyl]-3-(2-methoxy- phenyl)-urea |
| 94 | 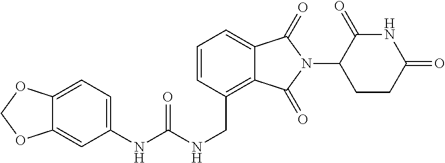 [SMILES: O=C1CCC(N2C(=O)c3cccc(CNC(=O)Nc4ccc5c(c4)OCO5)c3C2=O)C(=O)N1] | 1-(3,4- Methylenedioxyphenyl)- 3-[2-(2,6-dioxopiperidin- 3-yl)-1,3-dioxo-2,3- dihydro-1H-isoindol-4- ylmethyl]urea |
| 95 | 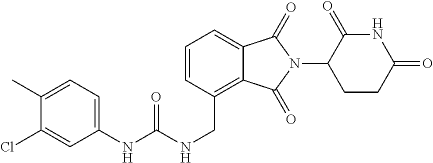 [SMILES: Cc1ccc(NC(=O)NCc2cccc3c2C(=O)N(C2CCC(=O)NC2=O)C3=O)cc1Cl] | 1-(3-Chloro-4- methylphenyl)-3-[2-(2,6- dioxopiperidin-3-yl)-1,3- dioxo-2,3-dihydro-1H- isoindol-4-ylmethyl]urea |
| 96 | 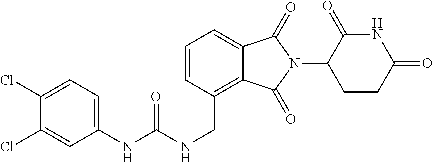 [SMILES: O=C1CCC(N2C(=O)c3cccc(CNC(=O)Nc4ccc(Cl)c(Cl)c4)c3C2=O)C(=O)N1] | 1-(3,4-dichlorophenyl)-3- [2-(2,6-dioxopiperidin-3- yl)-1,3-dioxo-2,3-dihydro- 1H-isoindol-4- ylmethyl]urea |
| 97 | 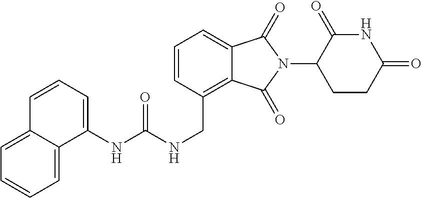 [SMILES: O=C1CCC(N2C(=O)c3cccc(CNC(=O)Nc4cccc5ccccc45)c3C2=O)C(=O)N1] | 1-[2-(2,6-Dioxopiperidin- 3-yl)-1,3-dioxo-2,3- dihydro-1H-isoindol-4- ylmethyl]-3-naphthalen-1- yl-urea |
| 98 | 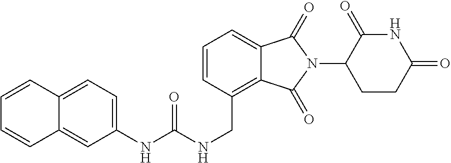 [SMILES: O=C1CCC(N2C(=O)c3cccc(CNC(=O)Nc4ccc5ccccc5c4)c3C2=O)C(=O)N1] | 1-[2-(2,6-Dioxopiperidin- 3-yl)-1,3-dioxo-2,3- dihydro-1H-isoindol-4- ylmethyl]-3-naphthalen-2- yl-urea |
| 99 | 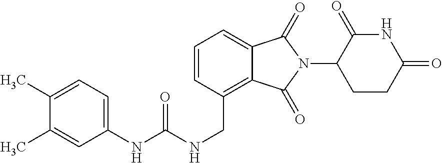 [SMILES: Cc1ccc(NC(=O)NCc2cccc3c2C(=O)N(C2CCC(=O)NC2=O)C3=O)cc1C] | 1-(3,4-Dimethyl-phenyl)-3- [2-(2,6-dioxo-piperidin-3- yl)-1,3-dioxo-2,3-dihydro- 1H-isoindol-4-ylmethyl]- urea |
| 100 | 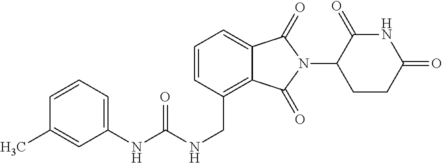 [SMILES: Cc1cccc(NC(=O)NCc2cccc3c2C(=O)N(C2CCC(=O)NC2=O)C3=O)c1] | 1-[2-(2,6-Dioxo-piperidin- 3-yl)-1,3-dioxo-2,3- dihydro-1H-isoindol-4- ylmethyl]-3-m-tolyl-urea |
| 101 | 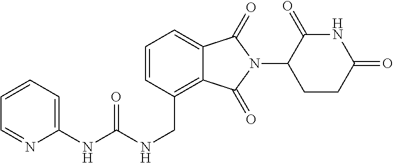 [SMILES: O=C1CCC(N2C(=O)c3cccc(CNC(=O)Nc4ccccn4)c3C2=O)C(=O)N1] | 1-[2-(2,6-Dioxo-piperidin- 3-yl)-1,3-dioxo-2,3- dihydro-1H-isoindol-4- ylmethyl]-3-pyridin-2-yl- urea |
| 102 | 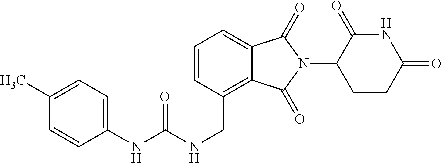 [SMILES: Cc1ccc(NC(=O)NCc2cccc3c2C(=O)N(C2CCC(=O)NC2=O)C3=O)cc1] | 1-[2-(2,6-Dioxo-piperidin- 3-yl)-1,3-dioxo-2,3- dihydro-1H-isoindol-4- ylmethyl]-3-p-tolyl-urea |
| 103 | 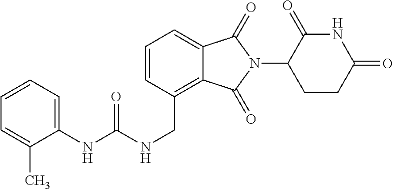 [SMILES: Cc1ccccc1NC(=O)NCc1cccc2c1C(=O)N(C1CCC(=O)NC1=O)C2=O] | 1-[2-(2,6-Dioxo-piperidin- 3-yl)-1,3-dioxo-2,3- dihydro-1H-isoindol-4- ylmethyl]-3-o-tolyl-urea |
| 104 | 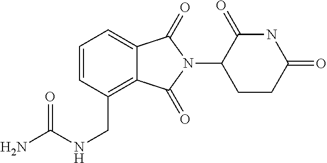 [SMILES: NC(=O)NCc1cccc2c1C(=O)N(C1CCC(=O)NC1=O)C2=O] | [2-(2,6-Dioxo-piperidin-3- yl)-1,3-dioxo-2,3-dihydro- 1H-isoindol-4-ylmethyl]- urea |

Still other representative compounds are of formula (IX):

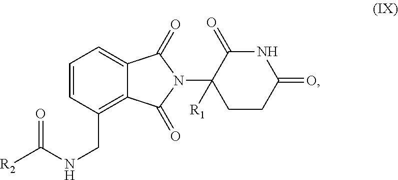

and pharmaceutically acceptable salts, solvates, stereoisomers, and prodrugs thereof,
wherein:
R₁ is H or methyl; and

### R₂ is:

N(CH₃)₂;
(C₀-C₁)alkyl-(C₆-C₁₀)aryl, substituted with one or more of: methyl, itself optionally substituted with one or more halogen; (C₁-C₄)alkoxy, itself optionally substituted with one or more halogen; or halogen;
(C₀-C₁)alkyl- (5 to 10 membered heteroaryl), optionally substituted with one or more of (C₁-C₄)alkyl, (C₁-C₄)alkoxy, or halogen; or
(5 to 6 membered heteroaryl)-phenyl, wherein the heteroaryl and phenyl are each independently optionally substituted with one or more of (C₁-C₄)alkyl or (C₁-C₄)alkoxy;
   with the proviso that R₂ is not unsubstituted pyridine, furan, or thiophene.

In one specific embodiment, R₂ is phenyl, substituted with one or more of methyl, (C₁-C₄)alkoxy, and halogen. In another embodiment, R₂ is pyrazine, pyrimidine, quinoxaline, or isoquinoline, optionally substituted with one or more of (C₁-C₄)alkyl and halogen. In another embodiment, R₂ is 5 membered heteroaryl, substituted with one of more (C₁-C₄)alkyl.

Examples of compounds of formula (IX) include, but are not limited to, those listed in Table G, below:

TABLE G
Compounds of Formula IX

| No. | Structure | Name |
|---|---|---|
| 105 | 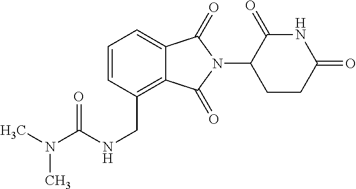 [SMILES: CN(C)C(=O)NCc1cccc2c1C(=O)N(C1CCC(=O)NC1=O)C2=O] | 3-[2-(2,6-Dioxo-piperidin- 3-yl)-1,3-dioxo-2,3- dihydro-1H-isoindol-4- ylmethyl]-1,1-dimethyl- urea |
| 106 | 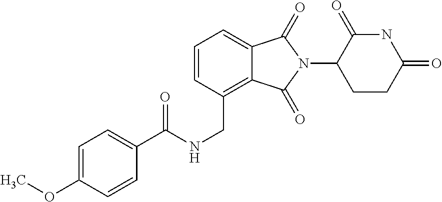 [SMILES: COc1ccc(C(=O)NCc2cccc3c2C(=O)N(C2CCC(=O)NC2=O)C3=O)cc1] | N-[2-(2,6-Dioxo-piperidin- 3-yl)-1,3-dioxo-2,3- dihydro-1H-isoindol-4- ylmethyl]-4-methoxy- benzamide |
| 107 | 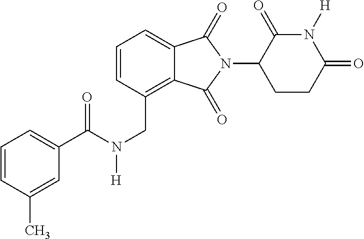 [SMILES: Cc1cccc(C(=O)NCc2cccc3c2C(=O)N(C2CCC(=O)NC2=O)C3=O)c1] | N-[2-(2,6-Dioxo-piperidin- 3-yl)-1,3-dioxo-2,3- dihydro-1H-isoindol-4- ylmethyl]-3-methyl- benzamide |
| 108 | 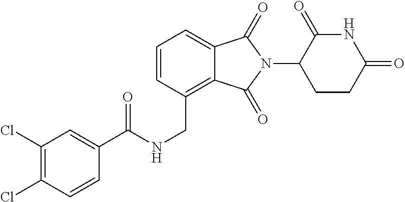 [SMILES: O=C1CCC(N2C(=O)c3cccc(CNC(=O)c4ccc(Cl)c(Cl)c4)c3C2=O)C(=O)N1] | 3,4-Dichloro-N-[2-(2,6- dioxopiperidin-3-yl)-1,3- dioxo-2,3-dihydro-1H- isoindol-4-yl- methyl]benzamide |
| 109 | 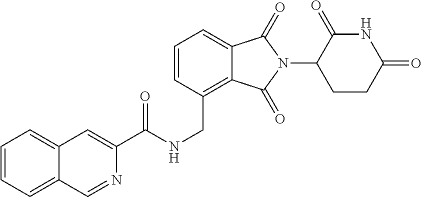 [SMILES: O=C1CCC(N2C(=O)c3cccc(CNC(=O)c4cc5ccccc5cn4)c3C2=O)C(=O)N1] | Isoquinoline-3-carboxylic acid [2-(2,6- dioxopiperidin-3-yl)-1,3- dioxo-2,3-dihydro-1H- isoindol-4-ylmethyl]amide |
| 110 | 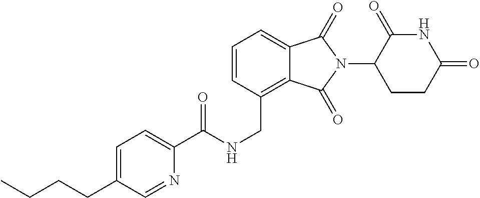 [SMILES: CCCCc1ccc(C(=O)NCc2cccc3c2C(=O)N(C2CCC(=O)NC2=O)C3=O)nc1] | 5-Butylpyridine-2- carboxylic acid [2-(2,6- dioxopiperidin-3-yl)-1,3- dioxo-2,3-dihydro-1H- isoindol-4-ylmethyl]amide |
| 111 | 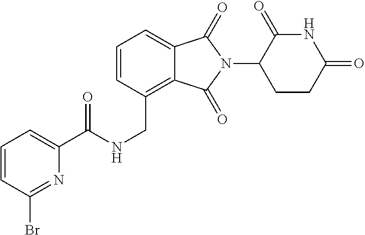 [SMILES: O=C1CCC(N2C(=O)c3cccc(CNC(=O)c4cccc(Br)n4)c3C2=O)C(=O)N1] | 6-Bromopyridine-2- carboxylic acid [2-(2,6- dioxopiperidin-3-yl)-1,3- dioxo-2,3-dihydro-1H- isoindol-4-ylmethyl]amide |
| 112 | 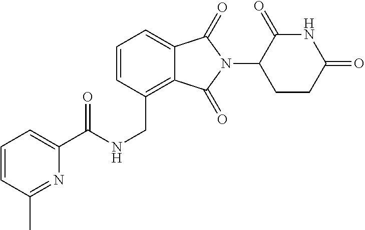 [SMILES: Cc1cccc(C(=O)NCc2cccc3c2C(=O)N(C2CCC(=O)NC2=O)C3=O)n1] | 6-Methylpyridine-2- carboxylic acid [2-(2,6- dioxopiperidin-3-yl)-1,3- dioxo-2,3-dihydro-1H- isoindol-4-ylmethyl]amide |
| 113 | 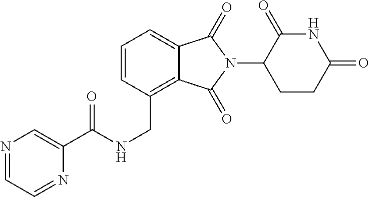 [SMILES: O=C1CCC(N2C(=O)c3cccc(CNC(=O)c4cnccn4)c3C2=O)C(=O)N1] | Pyrazine-2-carboxylic acid [2-(2,6-dioxopiperidin-3- yl)-1,3-dioxo-2,3-dihydro- 1H-isoindol-4-yl- methyl]amide |
| 114 | 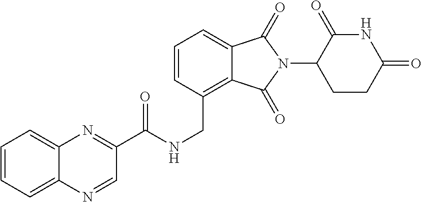 [SMILES: O=C1CCC(N2C(=O)c3cccc(CNC(=O)c4cnc5ccccc5n4)c3C2=O)C(=O)N1] | Quinoxaline-2-carboxylic acid [2-(2,6- dioxopiperidin-3-yl)-1,3- dioxo-2,3-dihydro-1H- isoindol-4-ylmethyl]amide |
| 115 | 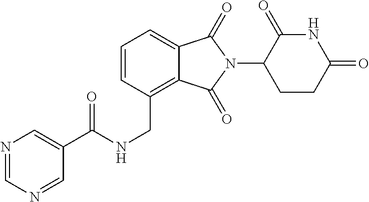 [SMILES: O=C1CCC(N2C(=O)c3cccc(CNC(=O)c4cncnc4)c3C2=O)C(=O)N1] | Pyrimidine-5-carboxylic acid [2-(2,6- dioxopiperidin-3-yl)-1,3- dioxo-2,3-dihydro-1H- isoindol-4-ylmethyl]amide |
| 116 | 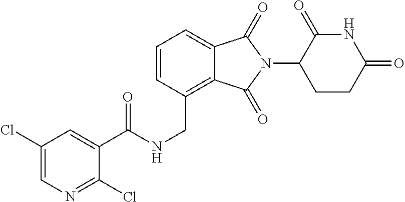 [SMILES: O=C1CCC(N2C(=O)c3cccc(CNC(=O)c4cc(Cl)cnc4Cl)c3C2=O)C(=O)N1] | 2,5-Dichloro-N-[2-(2,6- dioxopiperidin-3-yl)-1,3- dioxo-2,3-dihydro-1H- isoindol-4-yl- methyl]nicotinamide |
| 117 | 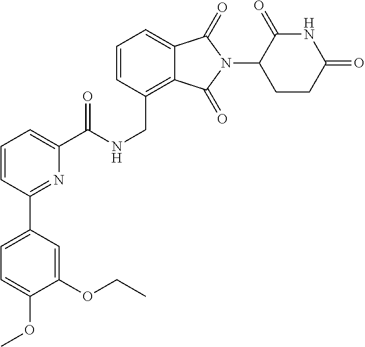 [SMILES: CCOc1cc(-c2cccc(C(=O)NCc3cccc4c3C(=O)N(C3CCC(=O)NC3=O)C4=O)n2)ccc1OC] | 6-(3-Ethoxy-4- methoxyphenyl)pyridine-2- carboxylic acid [2-(2,6- dioxopiperidin-3-yl)-1,3- dioxo-2,3-dihydro-1H- isoindol-4-ylmethyl]amide |
| 118 | 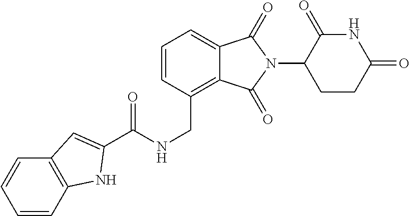 [SMILES: O=C1CCC(N2C(=O)c3cccc(CNC(=O)c4cc5ccccc5[nH]4)c3C2=O)C(=O)N1] | 1H-Indole-2-carboxylic acid [2-(2,6-dioxo- piperidin-3-yl)-1,3-dioxo- 2,3-dihydro-1H-isoindol-4- ylmethyl]-amide |
| 119 | 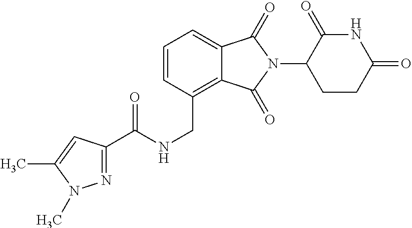 [SMILES: Cc1cc(C(=O)NCc2cccc3c2C(=O)N(C2CCC(=O)NC2=O)C3=O)nn1C] | 1,5-Dimethyl-1H-pyrazole- 3-carboxylic acid [2-(2,6- dioxo-piperidin-3-yl)-1,3- dioxo-2,3-dihydro-1H- isoindol-4-ylmethyl]-amide |
| 120 | 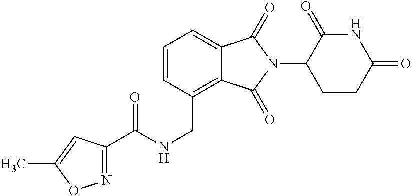 [SMILES: Cc1cc(C(=O)NCc2cccc3c2C(=O)N(C2CCC(=O)NC2=O)C3=O)no1] | 5-Methyl-isoxazole-3- carboxylic acid [2-(2,6- dioxo-piperidin-3-yl)-1,3- dioxo-2,3-dihydro-1H- isoindol-4-ylmethyl]-amide |
| 121 | 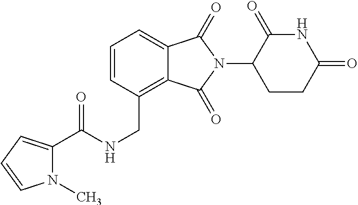 [SMILES: Cn1cccc1C(=O)NCc1cccc2c1C(=O)N(C1CCC(=O)NC1=O)C2=O] | 1-Methyl-1H-pyrrole-2- carboxylic acid [2-(2,6- dioxo-piperidin-3-yl)-1,3- dioxo-2,3-dihydro-1H- isoindol-4-ylmethyl]-amide |
| 122 | 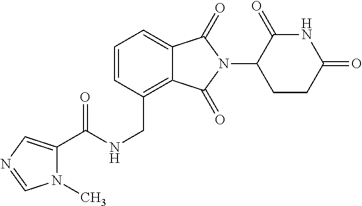 [SMILES: Cn1cncc1C(=O)NCc1cccc2c1C(=O)N(C1CCC(=O)NC1=O)C2=O] | 3-Methyl-3H-imidazole-4- carboxylic acid [2-(2,6- dioxo-piperidin-3-yl)-1,3- dioxo-2,3-dihydro-1H- isoindol-4-ylmethyl]-amide |
| 123 | 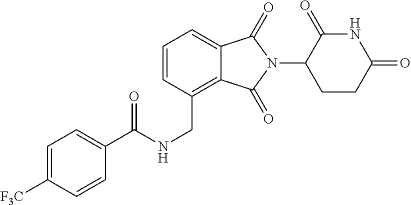 [SMILES: O=C1CCC(N2C(=O)c3cccc(CNC(=O)c4ccc(C(F)(F)F)cc4)c3C2=O)C(=O)N1] | N-[2-(2,6-dioxo-piperidin- 3-yl)-1,3-dioxo-2,3- dihydro-1H-isoindol-4- ylmethyl]-4- trifluoromethyl-benzamide |
| 124 | 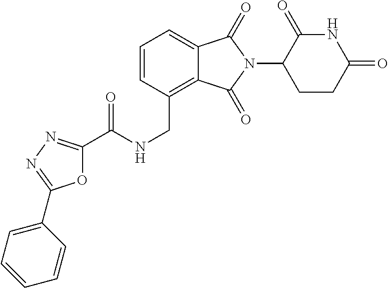 [SMILES: O=C1CCC(N2C(=O)c3cccc(CNC(=O)c4nnc(-c5ccccc5)o4)c3C2=O)C(=O)N1] | 5-Phenyl- [1,3,4]oxadiazole-2- carboxylic acid [2-(2,6- dioxo-piperidin-3-yl)-1,3- dioxo-2,3-dihydro-1H- isoindol-4-ylmethylamide |
| 125 | 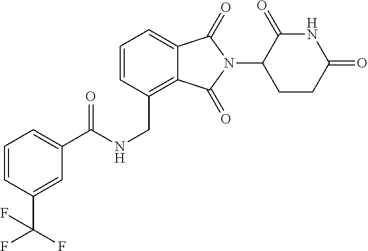 [SMILES: O=C1CCC(N2C(=O)c3cccc(CNC(=O)c4cccc(C(F)(F)F)c4)c3C2=O)C(=O)N1] | N-[2-(2,6-Dioxo-piperidin- 3-yl)-1,3-dioxo-2,3- dihydro-1H-isoindol-4- ylmethyl]-3- trifluoromethyl-benzamide |
| 126 | 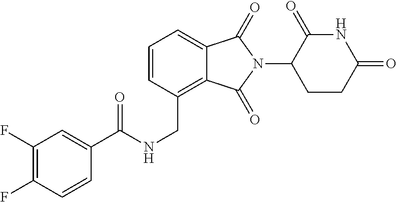 [SMILES: O=C1CCC(N2C(=O)c3cccc(CNC(=O)c4ccc(F)c(F)c4)c3C2=O)C(=O)N1] | N-[2-(2,6-Dioxo-piperidin- 3-yl)-1,3-dioxo-2,3- dihydro-1H-isoindol-4- ylmethyl]-3,4-difluoro- benzamide |
| 127 | 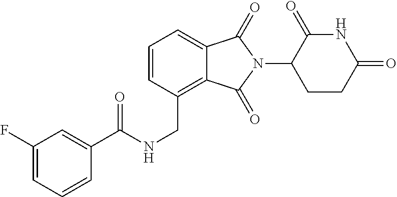 [SMILES: O=C1CCC(N2C(=O)c3cccc(CNC(=O)c4cccc(F)c4)c3C2=O)C(=O)N1] | N-[2-(2,6-Dioxo-piperidin- 3-yl)-1,3-dioxo-2,3- dihydro-1H-isoindol-4- ylmethyl]-3-fluoro- benzamide |
| 128 | 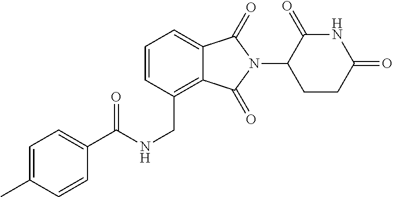 [SMILES: Cc1ccc(C(=O)NCc2cccc3c2C(=O)N(C2CCC(=O)NC2=O)C3=O)cc1] | N-[2-(2,6-Dioxo-piperidin- 3-yl)-1,3-dioxo-2,3- dihydro-1H-isoindol-4- ylmethyl]-4-methyl- benzamide |
| 129 | 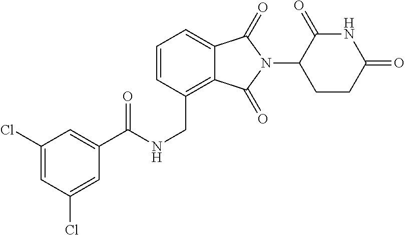 [SMILES: O=C1CCC(N2C(=O)c3cccc(CNC(=O)c4cc(Cl)cc(Cl)c4)c3C2=O)C(=O)N1] | 3,5-Dichloro-N-[2-(2,6- dioxo-piperidin-3-yl)-1,3- dioxo-2,3-dihydro-1H- isoindol-4-ylmethyl]- benzamide |
| 130 | 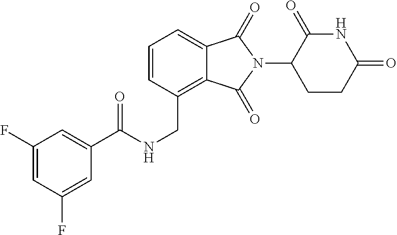 [SMILES: O=C1CCC(N2C(=O)c3cccc(CNC(=O)c4cc(F)cc(F)c4)c3C2=O)C(=O)N1] | N-[2-(2,6-Dioxo-piperidin- 3-yl)-1,3-dioxo-2,3- dihydro-1H-isoindol-4- ylmethyl]-3,5-difluoro- benzamide |
| 131 | 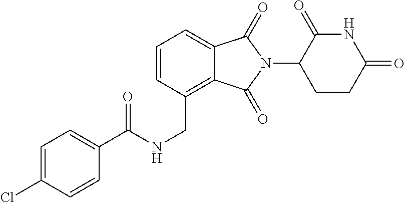 [SMILES: O=C1CCC(N2C(=O)c3cccc(CNC(=O)c4ccc(Cl)cc4)c3C2=O)C(=O)N1] | 4-Chloro-N-[2-(2,6-dioxo- piperidin-3-yl)-1,3-dioxo- 2,3-dihydro-1H-isoindol-4- ylmethyl]-benzamide |
| 132 | 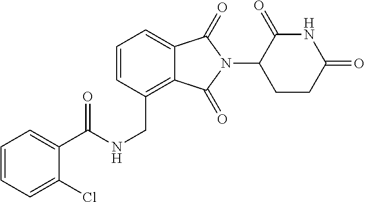 [SMILES: O=C1CCC(N2C(=O)c3cccc(CNC(=O)c4ccccc4Cl)c3C2=O)C(=O)N1] | 2-Chloro-N-[2-(2,6-dioxo- piperidin-3-yl)-1,3-dioxo- 2,3-dihydro-1H-isoindol-4- ylmethyl]-benzamide |
| 133 | 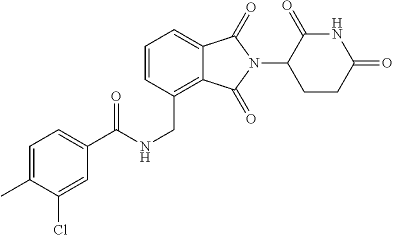 [SMILES: Cc1ccc(C(=O)NCc2cccc3c2C(=O)N(C2CCC(=O)NC2=O)C3=O)cc1Cl] | 3-Chloro-N-[2-(2,6-dioxo- piperidin-3-yl)-1,3-dioxo- 2,3-dihydro-1H-isoindol-4- ylmethyl]-4-methyl- benzamide |
| 134 | 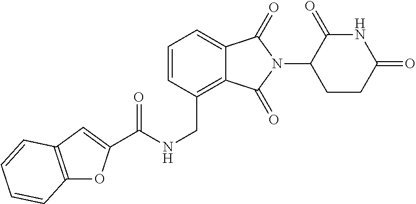 [SMILES: O=C1CCC(N2C(=O)c3cccc(CNC(=O)c4cc5ccccc5o4)c3C2=O)C(=O)N1] | Benzofuran-2-carboxylic acid [2-(2,6-dioxo- piperidin-3-yl)-1,3-dioxo- 2,3-dihydro-1H-isoindol-4- ylmethyl]-amide |
| 135 | 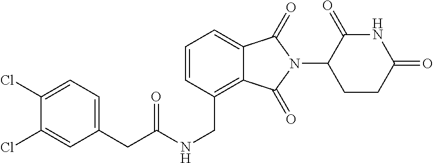 [SMILES: O=C(Cc1ccc(Cl)c(Cl)c1)NCc1cccc2c1C(=O)N(C1CCC(=O)NC1=O)C2=O] | 2-(3,4-Dichloro-phenyl)-N- [2-(2,6-dioxo-piperidin-3- yl)-1,3-dioxo-2,3-dihydro- 1H-isoindol-4-ylmethyl]- acetamide |
| 136 | 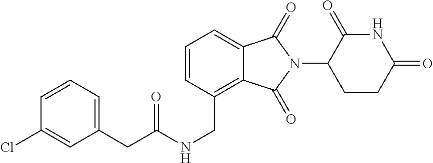 [SMILES: O=C(Cc1cccc(Cl)c1)NCc1cccc2c1C(=O)N(C1CCC(=O)NC1=O)C2=O] | 2-(3-Chloro-phenyl)-N-[2- (2,6-dioxo-piperidin-3-yl)- 1,3-dioxo-2,3-dihydro-1H- isoindol-4-ylmethyl]- acetamide |
| 137 | 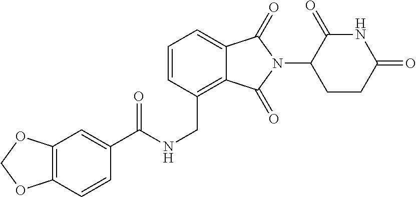 [SMILES: O=C1CCC(N2C(=O)c3cccc(CNC(=O)c4ccc5c(c4)OCO5)c3C2=O)C(=O)N1] | Benzo[1,3]dioxole-5- carboxylic acid [2-(2,6- dioxo-piperidin-3-yl)-1,3- dioxo-2,3-dihydro-1H- isoindol-4-ylmethyl]-amide |
| 138 | 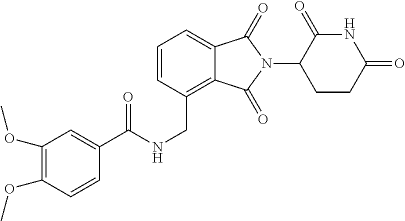 [SMILES: COc1ccc(C(=O)NCc2cccc3c2C(=O)N(C2CCC(=O)NC2=O)C3=O)cc1OC] | N-[2-(2,6-Dioxo-piperidin- 3-yl)-1,3-dioxo-2,3- dihydro-1H-isoindol-4-yl- methyl]-3,4-dimethoxy- benzamide |
| 139 | 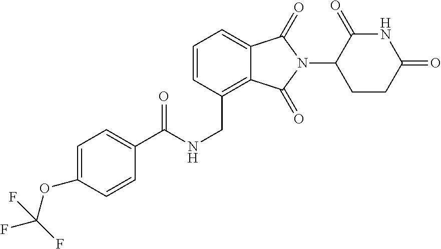 [SMILES: O=C1CCC(N2C(=O)c3cccc(CNC(=O)c4ccc(OC(F)(F)F)cc4)c3C2=O)C(=O)N1] | N-[2-(2,6-Dioxo-piperidin- 3-yl)-1,3-dioxo-2,3- dihydro-1H-isoindol-4- ylmethyl]-4- trifluoromethoxy- benzamide |
| 140 | 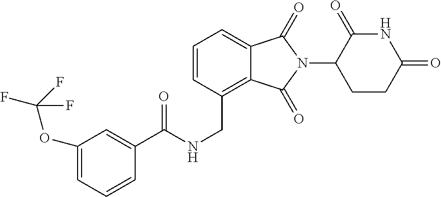 [SMILES: O=C1CCC(N2C(=O)c3cccc(CNC(=O)c4cccc(OC(F)(F)F)c4)c3C2=O)C(=O)N1] | N-[2-(2,6-Dioxo-piperidin- 3-yl)-1,3-dioxo-2,3- dihydro-1H-isoindol-4- ylmethyl]-3- trifluoromethoxy- benzamide |
| 141 | 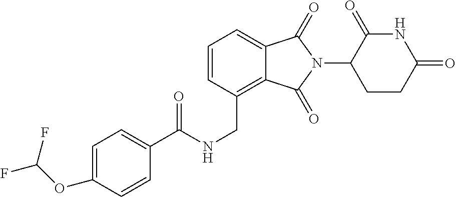 [SMILES: O=C1CCC(N2C(=O)c3cccc(CNC(=O)c4ccc(OC(F)F)cc4)c3C2=O)C(=O)N1] | 4-Difluoromethoxy-N-[2- (2,6-dioxo-piperidin-3-yl)- 1,3-dioxo-2,3-dihydro-1H- isoindol-4-ylmethyl]- benzamide |
| 142 | 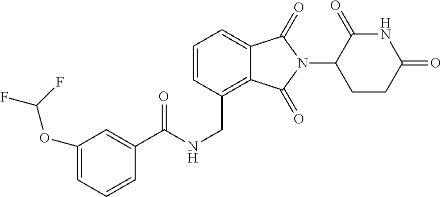 [SMILES: O=C1CCC(N2C(=O)c3cccc(CNC(=O)c4cccc(OC(F)F)c4)c3C2=O)C(=O)N1] | 3-Difluoromethoxy-N-[2- (2,6-dioxo-piperidin-3-yl)- 1,3-dioxo-2,3-dihydro-1H- isoindol-4-ylmethyl]- benzamide |
| 143 | 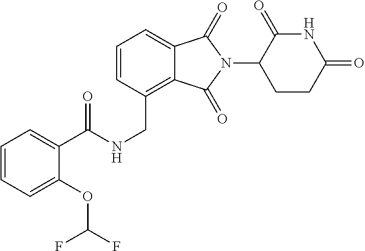 [SMILES: O=C1CCC(N2C(=O)c3cccc(CNC(=O)c4ccccc4OC(F)F)c3C2=O)C(=O)N1] | 2-Difluoromethoxy-N-[2- (2,6-dioxo-piperidin-3-yl)- 1,3-dioxo-2,3-dihydro-1H- isoindol-4-ylmethyl]- benzamide |
| 144 | 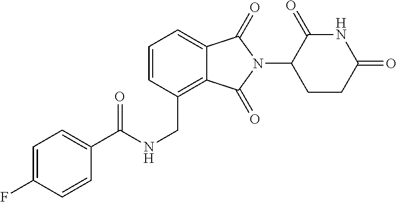 [SMILES: O=C1CCC(N2C(=O)c3cccc(CNC(=O)c4ccc(F)cc4)c3C2=O)C(=O)N1] | N-[2-(2,6-Dioxo-piperidin- 3-yl)-1,3-dioxo-2,3- dihydro-1H-isoindol-4- ylmethyl]-4-fluoro- benzamide |
| 145 | 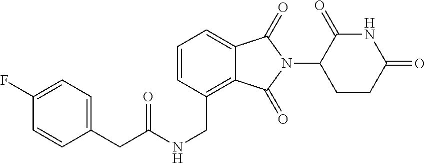 [SMILES: O=C(Cc1ccc(F)cc1)NCc1cccc2c1C(=O)N(C1CCC(=O)NC1=O)C2=O] | N-[2-(2,6-Dioxo-piperidin- 3-yl)-1,3-dioxo-2,3- dihydro-1H-isoindol-4- ylmethyl]-2-(4-fluoro- phenyl)-acetamide |
| 146 | 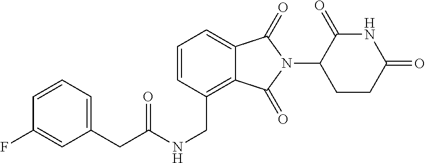 [SMILES: O=C(Cc1cccc(F)c1)NCc1cccc2c1C(=O)N(C1CCC(=O)NC1=O)C2=O] | N-[2-(2,6-Dioxo-piperidin- 3-yl)-1,3-dioxo-2,3- dihydro-1H-isoindol-4- ylmethyl]-2-(3-fluoro- phenyl)-acetamide |
| 147 | 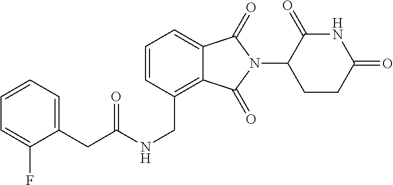 [SMILES: O=C(Cc1ccccc1F)NCc1cccc2c1C(=O)N(C1CCC(=O)NC1=O)C2=O] | N-[2-(2,6-Dioxo-piperidin- 3-yl)-1,3-dioxo-2,3- dihydro-1H-isoindol-4- ylmethyl]-2-(2-fluoro- phenyl)-acetamide |
| 148 | 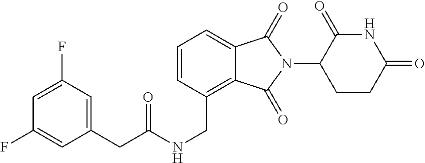 [SMILES: O=C(Cc1cc(F)cc(F)c1)NCc1cccc2c1C(=O)N(C1CCC(=O)NC1=O)C2=O] | 2-(3,5-Difluoro-phenyl)-N- [2-(2,6-dioxo-piperidin-3- yl)-1,3-dioxo-2,3-dihydro- 1H-isindol-4-ylmethyl]- acetamide |
| 149 | 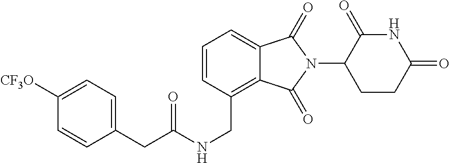 | N-[2-(2,6-Dioxo-piperidin- 3-yl)-1,3-dioxo-2,3- dihydro-1H-isoindol-4- ylmethyl]-2-(4- trifluoromethoxy-phenyl)- acetamide |
| 150 | 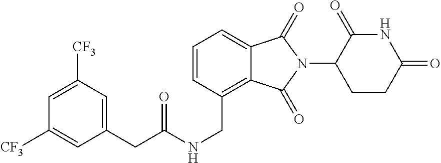 [SMILES: O=C(Cc1cc(C(F)(F)F)cc(C(F)(F)F)c1)NCc1cccc2c1C(=O)N(C1CCC(=O)NC1=O)C2=O] | 2-(3,5-Bis-trifluoromethyl- phenyl)-N-[2-(2,6-dioxo- piperidin-3-yl)-1,3-dioxo- 2,3-dihydro-1H-isoindol-4- ylmethyl]-acetamide |
| 151 | 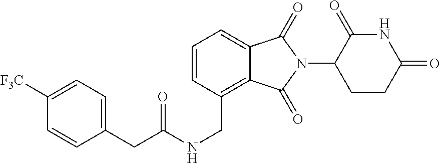 [SMILES: O=C(Cc1ccc(C(F)(F)F)cc1)NCc1cccc2c1C(=O)N(C1CCC(=O)NC1=O)C2=O] | (N-[2-(2,6-Dioxo- piperidin-3-yl)-1,3-dioxo- 2,3-dihydro-1H-isoindol-4- ylmethyl]-2-(4- trifluoromethyl-phenyl)- acetamide |
| 152 | 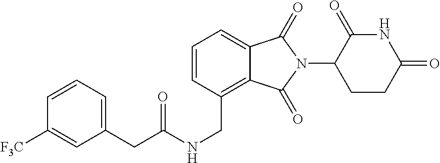 [SMILES: O=C(Cc1cccc(C(F)(F)F)c1)NCc1cccc2c1C(=O)N(C1CCC(=O)NC1=O)C2=O] | N-[2-(2,6-Dioxo-piperidin- 3-yl)-1,3-dioxo-2,3- dihydro-1H-isoindol-4- ylmethyl]-2-(3- trifluoromethyl-phenyl)- acetamide |
| 153 | 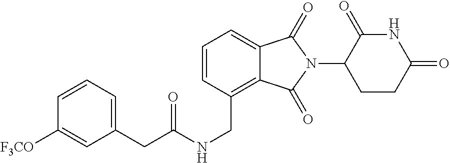 [SMILES: O=C(Cc1cccc(OC(F)(F)F)c1)NCc1cccc2c1C(=O)N(C1CCC(=O)NC1=O)C2=O] | N-[2-(2,6-Dioxo-piperidin- 3-yl)-1,3-dioxo-2,3- dihydro-1H-isoindol-4- ylmethyl]-2-(3- trifluoromethoxy-phenyl)- acetamide |
| 154 | 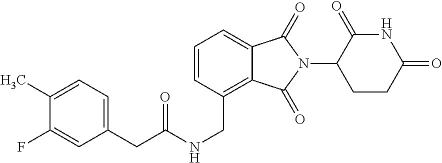 [SMILES: Cc1ccc(CC(=O)NCc2cccc3c2C(=O)N(C2CCC(=O)NC2=O)C3=O)cc1F] | N-[2-(2,6-Dioxo-piperidin- 3-yl)-1,3-dioxo-2,3- dihydro-1H-isoindol-4- ylmethyl]-2-(3-fluoro-4- methyl-phenyl)-acetamide |
| 155 | 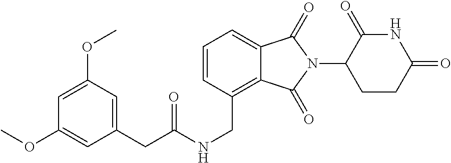 [SMILES: COc1cc(CC(=O)NCc2cccc3c2C(=O)N(C2CCC(=O)NC2=O)C3=O)cc(OC)c1] | 2-(3,5-Dimethoxy-phenyl)- N-[2-(2,6-dioxo-piperidin- 3-yl)-1,3-dioxo-2,3- dihydro-1H-isoindol-4- ylmethyl]-acetamide |
| 156 | 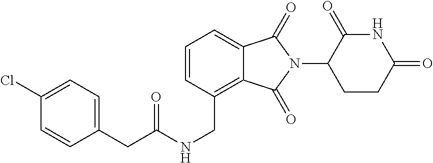 [SMILES: O=C(Cc1ccc(Cl)cc1)NCc1cccc2c1C(=O)N(C1CCC(=O)NC1=O)C2=O] | 2-(4-Chloro-phenyl)-N-[2- (2,6-dioxo-piperidin-3-yl)- 1,3-dioxo-2,3-dihydro-1H- isoindol-4-ylmethyl]- acetamide |
| 157 | 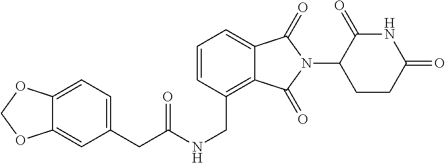 [SMILES: O=C(Cc1ccc2c(c1)OCO2)NCc1cccc2c1C(=O)N(C1CCC(=O)NC1=O)C2=O] | 2-Benzo[1,3]dioxo-5-yl-N- [2-(2,6-dioxo-piperidin-3- yl)-1,3-dioxo-2,3-dihydro- 1H-isoindol-4-ylmethyl]- acetamide |
| 158 | 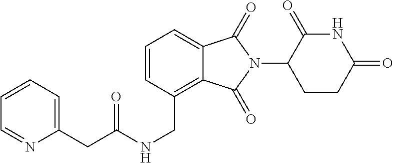 [SMILES: O=C(Cc1ccccn1)NCc1cccc2c1C(=O)N(C1CCC(=O)NC1=O)C2=O] | N-[2-(2,6-Dioxo-piperidin- 3-yl)-1,3-dioxo-2,3- dihydro-1H-isoindol-4- ylmethyl]-2-pyridinyl-2-yl- acetamide |
| 159 | 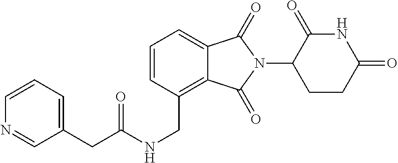 [SMILES: O=C(Cc1cccnc1)NCc1cccc2c1C(=O)N(C1CCC(=O)NC1=O)C2=O] | N-[2-(2,6-dioxo-piperidin- 3-yl)-1,3-dioxo-2,3- dihydro-1H-isoindol-4- ylmethyl]-2-pyridinyl-3-yl- acetamide |
| 160 | 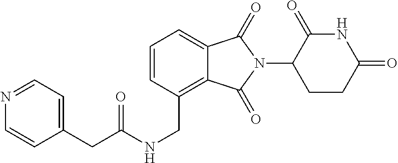 [SMILES: O=C(Cc1ccncc1)NCc1cccc2c1C(=O)N(C1CCC(=O)NC1=O)C2=O] | N-[2-(2,6-Dioxo-piperidin- 3-yl)-1,3-dioxo-2,3- dihydro-1H-isoindol-4- ylmethyl]-2-pyridin-4-yl- acetamide |
| 161 | 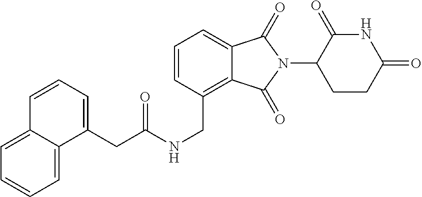 [SMILES: O=C(Cc1cccc2ccccc12)NCc1cccc2c1C(=O)N(C1CCC(=O)NC1=O)C2=O] | N-[2-(2,6-Dioxo-piperidin- 3-yl)-1,3-dioxo-2,3- dihydro-1H-isoindol-4- ylmethyl]-2-naphthalen-1- yl-acetamide |
| 162 | 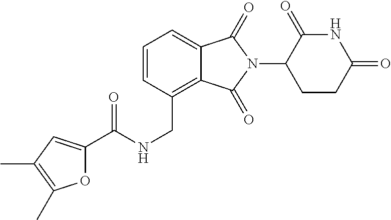 [SMILES: Cc1cc(C(=O)NCc2cccc3c2C(=O)N(C2CCC(=O)NC2=O)C3=O)oc1C] | 2-(4,5-Dimethyl-furan-2- yl)-N-[2-(2,6-dioxo- piperidin-3-yl)-1,3-dioxo- 2,3-dihydro-1H-isoindol-4- ylmethyl]-acetamide |
| 163 | 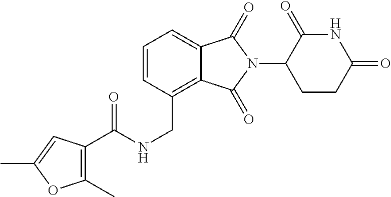 [SMILES: Cc1cc(C(=O)NCc2cccc3c2C(=O)N(C2CCC(=O)NC2=O)C3=O)c(C)o1] | 2-(2,5-Dimethyl-furan-3- yl)-N-[2-(2,6-dioxo- piperidin-3-yl)-1,3-dioxo- 2,3-dihydro-1H-isoindol-4- ylmethyl]-acetamide |
| 164 | 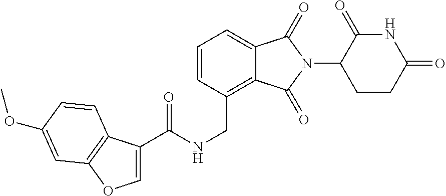 [SMILES: COc1ccc2c(C(=O)NCc3cccc4c3C(=O)N(C3CCC(=O)NC3=O)C4=O)coc2c1] | N-[2-(2,6-Dioxo-piperidin- 3-yl)-1,3-dioxo-2,3- dihydro-1H-isoindol-4- ylmethyl]2-(6-methoxy- benzofuran-3-yl)- acetamide |
| 165 | 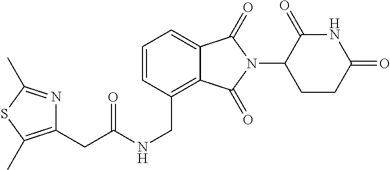 [SMILES: Cc1nc(CC(=O)NCc2cccc3c2C(=O)N(C2CCC(=O)NC2=O)C3=O)c(C)s1] | 2-{2,5-Dimethyl-1,3- thiazol-4-yl)-N-[2-(2,6- dioxo-piperidin-3-yl)-1,3- dioxo-2,3-dihydro-1H- isoindol-4-ylmethyl]- acetamide |
| 166 | 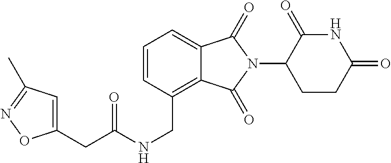 [SMILES: Cc1cc(CC(=O)NCc2cccc3c2C(=O)N(C2CCC(=O)NC2=O)C3=O)on1] | N-[2-(2,6-Dioxo-piperidin- 3-yl)-1,3-dioxo-2,3- dihydro-1H-isoindol-4- ylmethyl]-2-(3-methyl- isoxazol-5-yl)-acetamide |
| 167 | 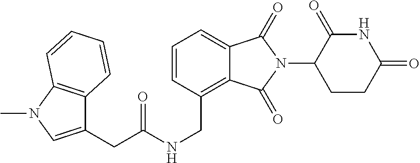 [SMILES: Cn1cc(CC(=O)NCc2cccc3c2C(=O)N(C2CCC(=O)NC2=O)C3=O)c2ccccc21] | N-[2-(2,6-Dioxo-piperidin- 3-yl)-1,3-dioxo-2,3- dihydro-1H-isoindol-4- ylmethyl]-2-(1-methyl-1H- indol-3-yl)-acetamide |
| 168 | 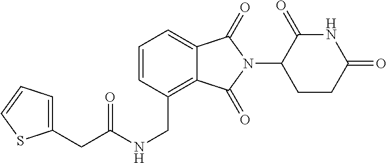 [SMILES: O=C(Cc1cccs1)NCc1cccc2c1C(=O)N(C1CCC(=O)NC1=O)C2=O] | N-[2-(2,6-Dioxo-piperidin- 3-yl)-1,3-dioxo-2,3- dihydro-1H-isoindol-4- ylmethyl]-2-thiophen-2-yl- acetamide |
| 169 | 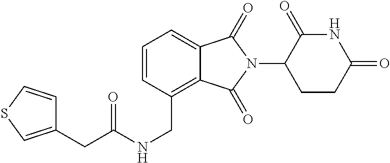 [SMILES: O=C(Cc1ccsc1)NCc1cccc2c1C(=O)N(C1CCC(=O)NC1=O)C2=O] | N-[2-(2,6-Dioxo-piperidin- 3-yl)-1,3-dioxo-2,3- dihydro-1H-isoindol-4- ylmethyl]-2-thiophen-2-yl- acetamide |
| 170 | 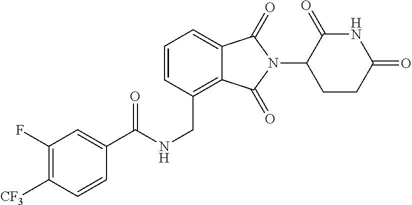 [SMILES: O=C1CCC(N2C(=O)c3cccc(CNC(=O)c4ccc(C(F)(F)F)c(F)c4)c3C2=O)C(=O)N1] | N-[2-(2,6-Dioxo-piperidin- 3-yl)-1,3-dioxo-2,3- dihydro-1H-isoindol-4- ylmethyl]-3-fluoro-4- trifluoromethyl-benzamide |
| 171 | 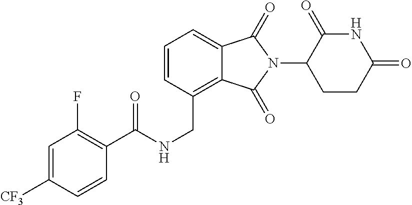 [SMILES: O=C1CCC(N2C(=O)c3cccc(CNC(=O)c4ccc(C(F)(F)F)cc4F)c3C2=O)C(=O)N1] | N-[2-(2,6-Dioxo-piperidin- 3-yl)-1,3-dioxo-2,3- dihydro-1H-isoindol-4- ylmethyl]-2-fluoro-4- trifluoromethyl-benzamide |
| 172 | 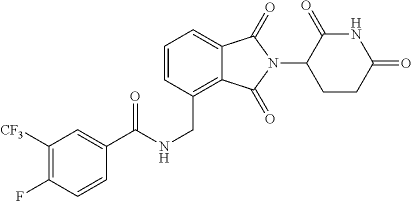 [SMILES: O=C1CCC(N2C(=O)c3cccc(CNC(=O)c4ccc(F)c(C(F)(F)F)c4)c3C2=O)C(=O)N1] | N-[2-(2,6-Dioxo-piperidin- 3-yl)-1,3-dioxo-2,3- dihydro-1H-isoindol-4- ylmethyl]-4-fluoro-3- trifluoromethyl-benzamide |
| 173 | 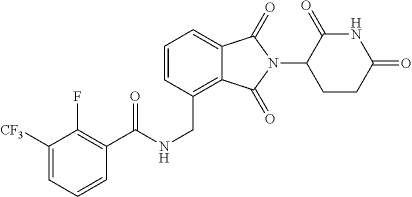 [SMILES: O=C1CCC(N2C(=O)c3cccc(CNC(=O)c4cccc(C(F)(F)F)c4F)c3C2=O)C(=O)N1] | N-[2-(2,6-Dioxo-piperidin- 3-yl)-1,3-dioxo-2,3- dihydro-1H-isoindol-4- ylmethyl]-2-fluoro-3- trifluoromethyl-benzamide |
| 174 | 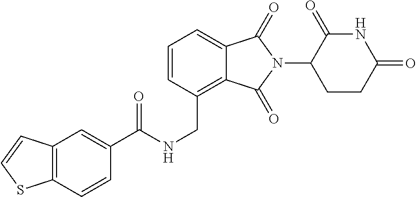 [SMILES: O=C1CCC(N2C(=O)c3cccc(CNC(=O)c4ccc5sccc5c4)c3C2=O)C(=O)N1] | Benzo[b]thiophene-5- carboxylic acid [2-(2,6- dioxo-piperidin-3-yl)-1,3- dioxo-2,3-dihydro-1H- isoindol-4-ylmethyl]-amide |
| 175 | 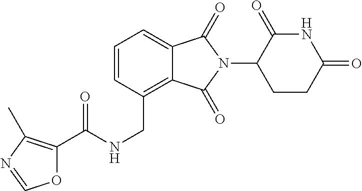 [SMILES: Cc1ncoc1C(=O)NCc1cccc2c1C(=O)N(C1CCC(=O)NC1=O)C2=O] | 4-Methyl-oxazole-5- carboxylic acid [2-(2,6- dioxo-piperidin-3-yl)-1,3- dioxo-2,3-dihydro-1H- isoindol-4-ylmethyl]-amide |
| 176 | 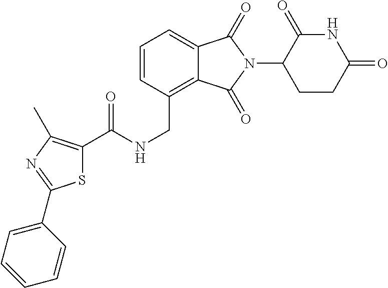 [SMILES: Cc1nc(-c2ccccc2)sc1C(=O)NCc1cccc2c1C(=O)N(C1CCC(=O)NC1=O)C2=O] | 4-Methyl-2-phenyl- thiazole-5-carboxylic acid [2-(2,6-dioxo-piperidin-3- yl)-1,3-dioxo-2,3-dihydro- 1H-isoindol-4- ylmethylamide |
| 177 | 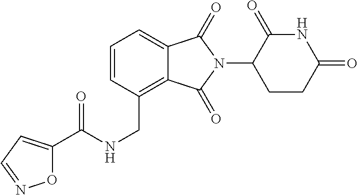 [SMILES: O=C1CCC(N2C(=O)c3cccc(CNC(=O)c4ccno4)c3C2=O)C(=O)N1] | Isoxazole-5-carboxylic acid [2-(2,6-dioxo- piperidin-3-yl)-1,3-dioxo- 2,3-dihydro-1H-isoindol-4- ylmethyl]-amide |
| 178 | 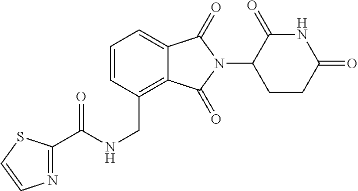 [SMILES: O=C1CCC(N2C(=O)c3cccc(CNC(=O)c4nccs4)c3C2=O)C(=O)N1] | Thiazole-2-carboxylic acid [2-(2,6-dioxo-piperidin-3- yl)-1,3-dioxo-2,3-dihydro- 1H-isoindol-4-ylmethyl]- amide |
| 179 | 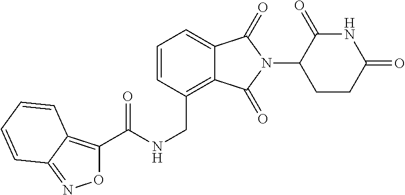 [SMILES: O=C1CCC(N2C(=O)c3cccc(CNC(=O)c4onc5ccccc45)c3C2=O)C(=O)N1] | Benzo[c]isoxazole-3- carboxylic acid [2-(2,6- dioxo-piperidin-3-yl)-1,3- dioxo-2,3-dihydro-1H- isoindol-4-ylmethyl]-amide |

Still other representative compounds are those listed in Table H, below, and pharmaceutically acceptable salts, solvates, stereoisomers, and prodrugs thereof.

TABLE H

| | | |
|---|---|---|
| 180 | 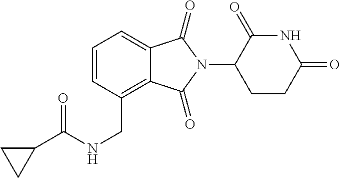 [SMILES: O=C1CCC(N2C(=O)c3cccc(CNC(=O)C4CC4)c3C2=O)C(=O)N1] | cyclopropanecarboxylic acid [2-(2,6-dioxo-piperidin-3- yl)-1,3-dioxo-2,3-dihydro- 1H-isoindol-4-ylmethyl]- amide |
| 181 | 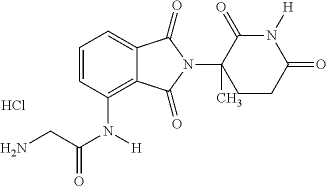 [SMILES: Cl ; CC1(N2C(=O)c3cccc(NC(=O)CN)c3C2=O)CCC(=O)NC1=O] | 2-amino-N-[2-(3-methyl-2,6- dioxo-piperidin-3-yl)-1,3- dioxo-2,3-dihydro-1H- isoindol-4-yl]-acetamide |
| 182 | 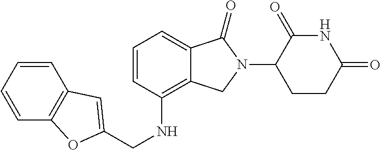 [SMILES: O=C1CCC(N2Cc3c(NCc4cc5ccccc5o4)cccc3C2=O)C(=O)N1] | 3-{4-[(Benzofuran-2- ylmethyl)-amino]-1-oxo-1,3- dihydro-isoindol-2-yl}- piperidine-2,6-dione |
| 183 | 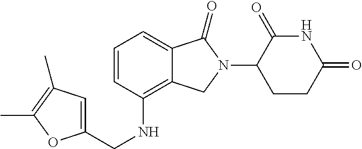 [SMILES: Cc1cc(CNc2cccc3c2CN(C2CCC(=O)NC2=O)C3=O)oc1C] | 3-{4-[(4,5-Dimethyl-furan-2- ylmethyl)-amino]-1-oxo-1,3- dihydro-isoindol-2-yl}- piperidine-2,6-dione |
| 184 | 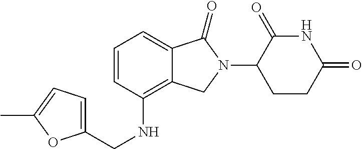 [SMILES: Cc1ccc(CNc2cccc3c2CN(C2CCC(=O)NC2=O)C3=O)o1] | 3-{4-[(5-Methyl-furan-2- ylmethyl)-amino]-1-oxo-1,3- dihydro-isoindol-2-yl}- piperidine-2,6-dione |

In specific embodiments, provided herein is a stereomerically pure (R) isomer and a stereomerically pure (S) isomer of the compounds listed above.

In specific embodiments, provided herein are a stereomerically pure (R) isomer and a stereomerically pure (S) isomer of 2-amino-N-[2-(3-methyl-2,6-dioxo-piperidin-3-yl)-1,3-dioxo-2,3-dihydro-1H-isoindol-4-yl]-acetamide, and a racemic mixture thereof.

Still other specific IMiD® immunomodulatory drugs provided herein belong to a class of N-methylaminomethyl isoindole compounds disclosed in U.S. Patent Application Publication No. US 2008/0214615, the entirety of which is incorporated herein by reference. Representative compounds are of formula X:

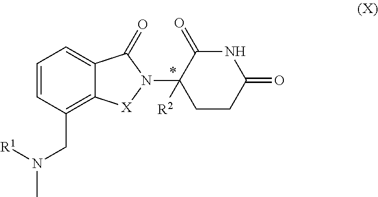

and pharmaceutically acceptable salts, solvates, stereoisomers, and prodrugs thereof,
wherein:
* denotes chiral center;
X is CH₂ or C═O;
R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₉)hetero aryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;
R² is H, CH₃, or (C₂-C₈)alkyl;
R³ and R^{3′} are independently
   (C₁-C₈)alkyl;
   (C₃-C₇)cycloalkyl;
   (C₂-C₈)alkenyl;
   (C₂-C₈)alkynyl;
   benzyl;
   (C₀-C₄)alkyl-(C₅-C₁₀)aryl, optionally substituted with one or more of:
      (C₁-C₆)alkyl, said alkyl itself optionally substituted with one or more halogen,
      (C₁-C₆)alkoxy, said alkoxy itself optionally substituted with one or more halogen,
      SCY₃, wherein Y is hydrogen or halogen,
      NZ₂, wherein Z is hydrogen or (C₁-C₆)alkyl
      (C₁-C₆)alkylenedioxy, or
      halogen;
   (C₀-C₄)alkyl-(C₁-C₆)heterocycloalkyl;
   (C₀-C₄)alkyl-(C₂-C₉)heteroaryl;
   (C₀-C₈)alkyl-N(R⁶)₂;
   (C₁-C₈)alkyl-OR⁵;
   (C₁-C₈)alkyl-C(O)OR⁵;
   (C₁-C₈)alkyl-O(CO)R⁵; or
   C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₉)heteroaryl;
R⁵ is (C₁-C₈)alkyl, (C₂-C₈)alkenyl, (C₂-C₈)alkynyl, benzyl, (C₅-C₁₀)aryl, or (C₂-C₉)heteroaryl;
   each occurrence of R⁶ is independently H, (C₁-C₈)alkyl, (C₂-C₈)alkenyl, (C₂-C₈)alkynyl, benzyl, (C₅-C₁₀)aryl, (C₂-C₉)heteroaryl, or (C₀-C₈)alkyl-C(O)O—R⁵, or
   two R⁶ groups can join to form a heterocycloalkyl group.

In one embodiment, X is C═O. In another embodiment, X is CH₂.

In one embodiment, R¹ is H. In another embodiment, R¹ is CH₃. In another embodiment, R¹ is (C₂-C₈)alkyl. In another embodiment, R¹ is (C₃-C₇)cycloalkyl. In another embodiment, R¹ is (C₂-C₈)alkenyl. In another embodiment, R¹ is (C₂-C₈)alkynyl. In another embodiment, R¹ is benzyl. In another embodiment, R¹ is aryl. In another embodiment, R¹ is (C₀-C₄)alkyl-(C₁-C₆)heterocycloalkyl. In another embodiment, R¹ is (C₀-C₄)alkyl-(C₂-C₉)heteroaryl. In another embodiment, R¹ is C(O)R³. In another embodiment, R¹ is C(S)R³. In another embodiment, R¹ is C(O)OR⁴. In another embodiment, R¹ is (C₁-C₈)alkyl-N(R⁶)₂. In another embodiment, R¹ is (C₁-C₈)alkyl-OR⁵. In another embodiment, R¹ is (C₁-C₈)alkyl-C(O)OR⁵. In another embodiment, R¹ is C(O)NHR³. In one embodiment, R¹ is C(O)NH—(C₀-C₄)alkyl-(C₅-C₁₀)aryl, wherein the aryl is optionally substituted as described herein below. In another embodiment, R¹ is C(S)NHR³. In another embodiment, R¹ is C(O)NR³R^{3′}. In another embodiment, R¹ is C(S)NR³R^{3′}. In another embodiment, R¹ is (C₁-C₈)alkyl-O(CO)R⁵.

In one embodiment, R² is H. In another embodiment, R² is (C₁-C₈)alkyl.

In one embodiment, R³ is (C₁-C₈)alkyl. In another embodiment, R³ is (C₃-C₇)cycloalkyl. In another embodiment, R³ is (C₂-C₈)alkenyl. In another embodiment, R³ is (C₂-C₈)alkynyl. In another embodiment, R³ is benzyl. In another embodiment, R³ is (C₀-C₄)alkyl-(C₅-C₁₀)aryl, optionally substituted with one or more of: (C₁-C₆)alkyl, said alkyl itself optionally substituted with one or more halogen; (C₁-C₆)alkoxy, said alkoxy itself optionally substituted with one or more halogen; SCY₃, wherein Y is hydrogen or halogen; NZ₂, wherein Z is hydrogen or (C₁-C₆)alkyl; (C₁-C₆)alkylenedioxy; or halogen. In another embodiment, R³ is (C₀-C₄)alkyl-(C₁-C₆)heterocycloalkyl. In another embodiment, R³ is (C₀-C₄)alkyl-(C₂-C₉)heteroaryl. In another embodiment, R³ is (C₀-C₈)alkyl-N(R⁶)₂. In another embodiment, R³ is (C₁-C₈)alkyl-OR⁵. In another embodiment, R³ is (C₁-C₈)alkyl-C(O)OR⁵. In another embodiment, R³ is (C₁-C₈)alkyl-O(CO)R⁵. In another embodiment, R³ is C(O)OR⁵.

In one embodiment, R^{3′} is (C₁-C₈)alkyl. In another embodiment, R^{3′} is (C₃-C₇)cycloalkyl. In another embodiment, R^{3′} is (C₂-C₈)alkenyl. In another embodiment, R^{3′} is (C₂-C₈)alkynyl. In another embodiment, R^{3′} is benzyl. In another embodiment, R^{3′} is aryl. In another embodiment, R^{3′} is (C₀-C₄)alkyl-(C₁-C₆)heterocycloalkyl. In another embodiment, R^{3′} is (C₀-C₄)alkyl-(C₂-C₉)heteroaryl. In another embodiment, R^{3′} is (C₀-C₈)alkyl-N(R⁶)₂. In another embodiment, R^{3′} is (C₁-C₈)alkyl-OR⁵. In another embodiment, R^{3′} is (C₁-C₈)alkyl-C(O)OR⁵. In another embodiment, R^{3′} is (C₁-C₈)alkyl-O(CO)R⁵. In another embodiment, R^{3′} is C(O)OR⁵.

In one embodiment, R⁴ is (C₁-C₈)alkyl. In another embodiment, R⁴ is (C₂-C₈)alkenyl. In another embodiment, R⁴ is (C₂-C₈)alkynyl. In another embodiment, R⁴ is (C₁-C₄)alkyl-OR⁵. In another embodiment, R⁴ is benzyl. In another embodiment, R⁴ is aryl. In another embodiment, R⁴ is (C₀-C₄)alkyl-(C₁-C₆)heterocycloalkyl. In another embodiment, R⁴ is (C₀-C₄)alkyl-(C₂-C₉)heteroaryl.

In one embodiment, R⁵ is (C₁-C₈)alkyl. In another embodiment, R⁵ is (C₂-C₈)alkenyl. In another embodiment, R⁵ is (C₂-C₈)alkynyl. In another embodiment, R⁵ is benzyl. In another embodiment, R⁵ is (C₅-C₁₀)aryl. In another embodiment, R⁵ is (C₂-C₉)heteroaryl.

In one embodiment, R⁶ is H. In another embodiment, R⁶ is (C₁-C₈)alkyl. In another embodiment, R⁶ is (C₂-C₈)alkenyl. In another embodiment, R⁶ is (C₂-C₈)alkynyl. In another embodiment, R⁶ is benzyl. In another embodiment, R⁶ is (C₅-C₁₀)aryl. In another embodiment, R⁶ is (C₂-C₉)heteroaryl. In another embodiment, R⁶ is or (C₀-C₈)alkyl-C(O)O—R⁵. In another embodiment, two R⁶ groups join to form a heterocycloalkyl group.

In other embodiments, provided herein are any combination of X, R¹, R², R³, R^{3′}, R⁴, R⁵, and/or R⁶ as set forth above.

In one embodiment, representative compounds are of formula:

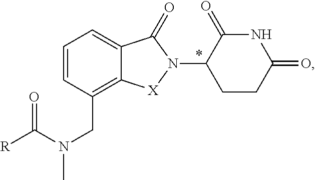

and pharmaceutically acceptable salts, solvates, stereoisomers, and prodrugs thereof,
wherein:
* denotes chiral center;

### X is CH₂ or C═O;

R is (C₁-C₆)alkyl; (C₁-C₆)alkoxy; amino; (C₁-C₆)alkyl-amino; dialkylamino, wherein each of the alkyl groups is independently (C₁-C₆)alkyl; (C₀-C₄)alkyl-(C₆-C₁₀)aryl, optionally substituted with one or more (C₁-C₆)alkyl, (C₁-C₆)alkoxy or halogen; 5 to 10 membered heteroaryl, optionally substituted with one or more (C₁-C₆)alkyl; —NHR′; or
(C₀-C₈)alkyl-N(R″)₂;

### R′ is:

(C₁-C₆)alkyl;
(C₀-C₄)alkyl-(C₆-C₁₀)aryl, optionally substituted with one or more of:
   (C₁-C₆)alkyl, said alkyl itself optionally substituted with one or more halogen,
   (C₁-C₆)alkoxy, said alkoxy itself optionally substituted with one or more halogen, (C₁-C₆)alkylenedioxy, or
   halogen; or
5 to 10 membered heteroaryl, optionally substituted with one or more (C₁-C₆)alkyl; and
   each occurrence of R″ is independently H, (C₁-C₈)alkyl, (C₂-C₈)alkenyl, (C₂-C₈)alkynyl, benzyl, (C₆-C₁₀)aryl, 5 to 10 membered heteroaryl, or (C₀-C₈)alkyl-C(O)O—(C₁-C₈)alkyl.

In one embodiment, X is C═O. In another embodiment, X is CH₂.

In one embodiment, R is (C₁-C₆)alkyl. In certain specific embodiments, R is methyl, ethyl, propyl, cyclopropyl, or hexyl.

In another embodiment, R is (C₁-C₆)alkoxy. In certain specific embodiments, R is t-butoxy.

In another embodiment, R is amino. In another embodiment, R is (C₁-C₆)alkyl-amino. In another embodiment, R is dialkylamino, wherein each of the alkyl groups is independently (C₁-C₆)alkyl. In certain specific embodiments, R is dimethylamino.

In another embodiment, R is (C₀-C₄)alkyl-(C₆-C₁₀)aryl, optionally substituted with one or more (C₁-C₆)alkyl, (C₁-C₆)alkoxy, or halogen. In certain specific embodiments, R is phenyl or —CH₂-phenyl, optionally substituted with one or more methyl and/or halogen.

In another embodiment, R is 5 to 10 membered heteroaryl, optionally substituted with one or more (C₁-C₆)alkyl. In certain specific embodiments, R is pyridyl or furanyl.

In another embodiment, R is —NHR′.

In one embodiment, R′ is (C₁-C₆)alkyl, optionally substituted with one or more halogen. In certain specific embodiments, R′ is methyl, ethyl, propyl, t-butyl, cyclohexyl, or trifluoromethyl.

In another embodiment, R′ is (C₀-C₄)alkyl-(C₆-C₁₀)aryl, optionally substituted with one or more (C₁-C₆)alkyl, (C₁-C₆)alkoxy, (C₁-C₆)alkylenedioxy or halogen. In certain specific embodiments, R′ is phenyl, optionally substituted with one or more of methyl, methoxy, and/or chloride. In another embodiment, R′ is naphthyl. In another embodiment, R′ is phenyl, substituted with (C₁-C₆)alkylenedioxy, specifically, methylenedioxy. In another embodiment, R′ is toluoyl.

In another embodiment, R′ is 5 to 10 membered heteroaryl, optionally substituted with one or more (C₁-C₆)alkyl. In certain specific embodiments, R′ is pyridyl or naphthyl.

In one embodiment, R is (C₀-C₈)alkyl-N(R″)₂.

In another embodiment, R″ is H. In another embodiment, R″ is (C₁-C₈)alkyl. In another embodiment, R″ is (C₂-C₈)alkenyl. In another embodiment, R″ is (C₂-C₈)alkynyl. In another embodiment, R″ is benzyl. In another embodiment, R″ is (C₆-C₁₀)aryl. In another embodiment, R″ is 5 to 10 membered heteroaryl. In another embodiment, R″ is (C₀-C₈)alkyl-C(O)O—(C₁-C₈)alkyl. In a specific embodiment, one of R″ is H and the other of R″ is (C₀-C₈)alkyl-C(O)O—(C₁-C₈)alkyl, in particular, —COO-isobutyl.

In other embodiments, provided herein are any combination of X, R, and/or R′ as set forth above.

Examples include, but are not limited to, those listed in Table I, below, or a pharmaceutically acceptable salt, solvate (e.g., hydrate), or stereoisomer thereof:

TABLE I

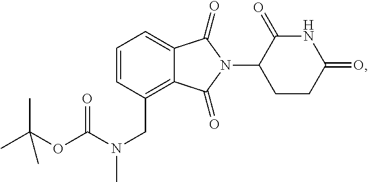 [SMILES: CN(Cc1cccc2c1C(=O)N(C1CCC(=O)NC1=O)C2=O)C(=O)OC(C)(C)C]

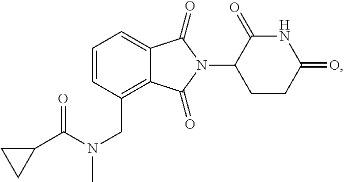 [SMILES: CN(Cc1cccc2c1C(=O)N(C1CCC(=O)NC1=O)C2=O)C(=O)C1CC1]

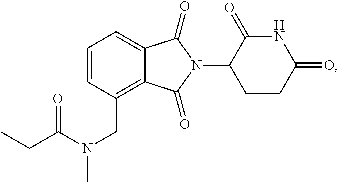 [SMILES: CCC(=O)N(C)Cc1cccc2c1C(=O)N(C1CCC(=O)NC1=O)C2=O]

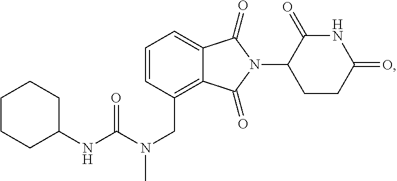 [SMILES: CN(Cc1cccc2c1C(=O)N(C1CCC(=O)NC1=O)C2=O)C(=O)NC1CCCCC1]

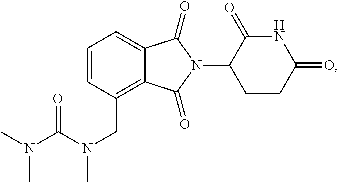 [SMILES: CN(C)C(=O)N(C)Cc1cccc2c1C(=O)N(C1CCC(=O)NC1=O)C2=O]

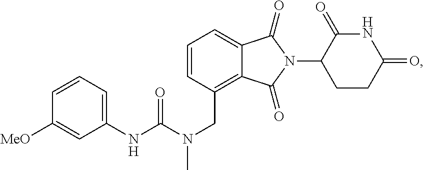 [SMILES: COc1cccc(NC(=O)N(C)Cc2cccc3c2C(=O)N(C2CCC(=O)NC2=O)C3=O)c1]

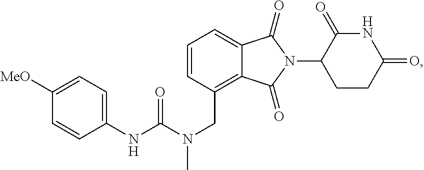 [SMILES: COc1ccc(NC(=O)N(C)Cc2cccc3c2C(=O)N(C2CCC(=O)NC2=O)C3=O)cc1]

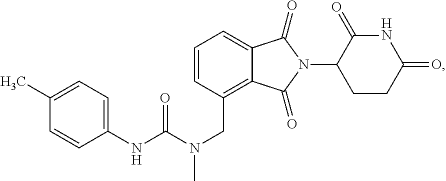 [SMILES: Cc1ccc(NC(=O)N(C)Cc2cccc3c2C(=O)N(C2CCC(=O)NC2=O)C3=O)cc1]

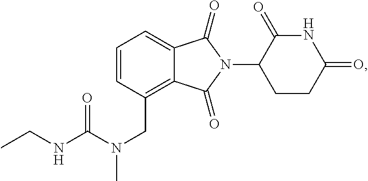 [SMILES: CCNC(=O)N(C)Cc1cccc2c1C(=O)N(C1CCC(=O)NC1=O)C2=O]

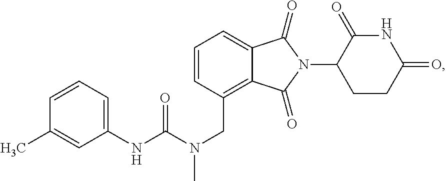 [SMILES: Cc1cccc(NC(=O)N(C)Cc2cccc3c2C(=O)N(C2CCC(=O)NC2=O)C3=O)c1]

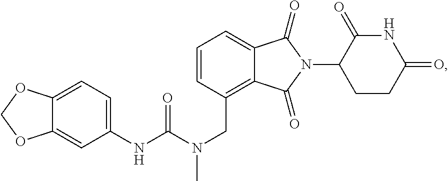 [SMILES: CN(Cc1cccc2c1C(=O)N(C1CCC(=O)NC1=O)C2=O)C(=O)Nc1ccc2c(c1)OCO2]

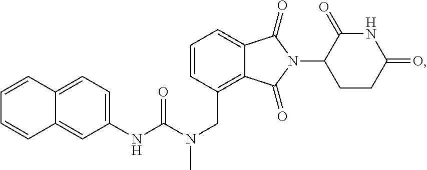 [SMILES: CN(Cc1cccc2c1C(=O)N(C1CCC(=O)NC1=O)C2=O)C(=O)Nc1ccc2ccccc2c1]

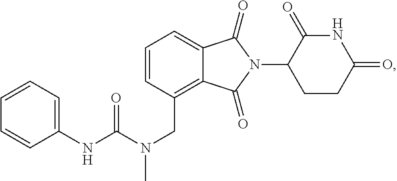 [SMILES: CN(Cc1cccc2c1C(=O)N(C1CCC(=O)NC1=O)C2=O)C(=O)Nc1ccccc1]

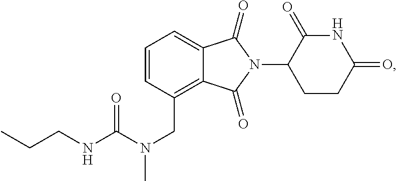 [SMILES: CCCNC(=O)N(C)Cc1cccc2c1C(=O)N(C1CCC(=O)NC1=O)C2=O]

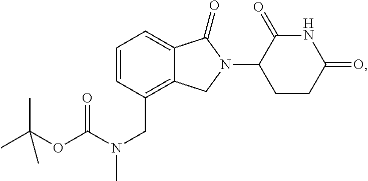 [SMILES: CN(Cc1cccc2c1CN(C1CCC(=O)NC1=O)C2=O)C(=O)OC(C)(C)C]

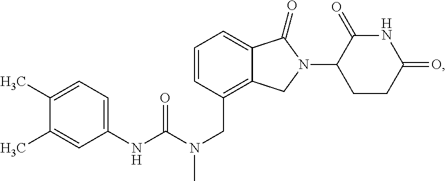 [SMILES: Cc1ccc(NC(=O)N(C)Cc2cccc3c2CN(C2CCC(=O)NC2=O)C3=O)cc1C]

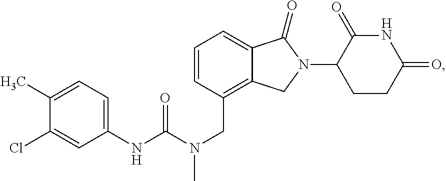 [SMILES: Cc1ccc(NC(=O)N(C)Cc2cccc3c2CN(C2CCC(=O)NC2=O)C3=O)cc1Cl]

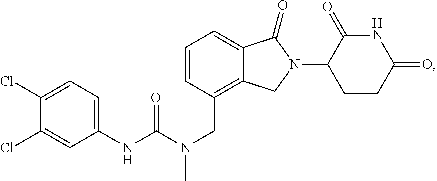 [SMILES: CN(Cc1cccc2c1CN(C1CCC(=O)NC1=O)C2=O)C(=O)Nc1ccc(Cl)c(Cl)c1]

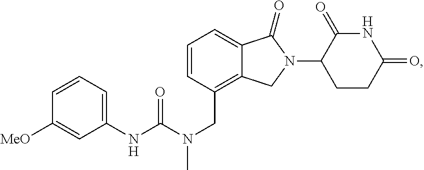 [SMILES: COc1cccc(NC(=O)N(C)Cc2cccc3c2CN(C2CCC(=O)NC2=O)C3=O)c1]

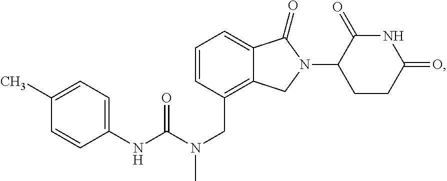 [SMILES: Cc1ccc(NC(=O)N(C)Cc2cccc3c2CN(C2CCC(=O)NC2=O)C3=O)cc1]

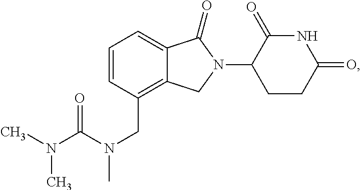 [SMILES: CN(C)C(=O)N(C)Cc1cccc2c1CN(C1CCC(=O)NC1=O)C2=O]

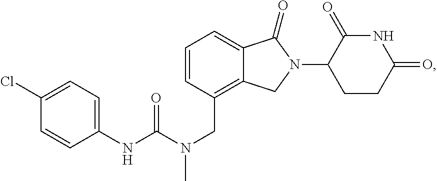 [SMILES: CN(Cc1cccc2c1CN(C1CCC(=O)NC1=O)C2=O)C(=O)Nc1ccc(Cl)cc1]

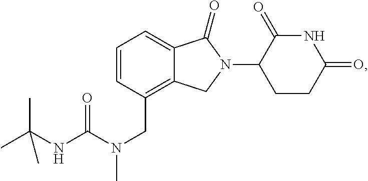 [SMILES: CN(Cc1cccc2c1CN(C1CCC(=O)NC1=O)C2=O)C(=O)NC(C)(C)C]

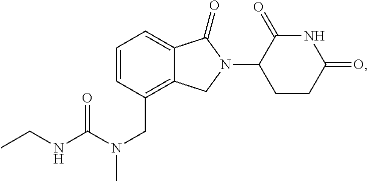 [SMILES: CCNC(=O)N(C)Cc1cccc2c1CN(C1CCC(=O)NC1=O)C2=O]

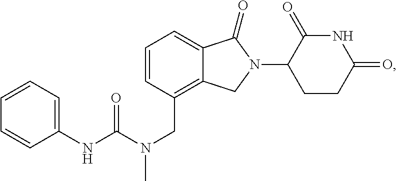 [SMILES: CN(Cc1cccc2c1CN(C1CCC(=O)NC1=O)C2=O)C(=O)Nc1ccccc1]

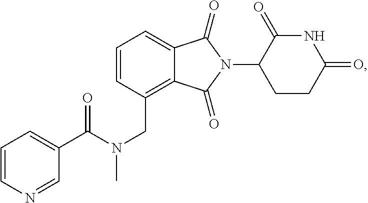 [SMILES: CN(Cc1cccc2c1C(=O)N(C1CCC(=O)NC1=O)C2=O)C(=O)c1cccnc1]

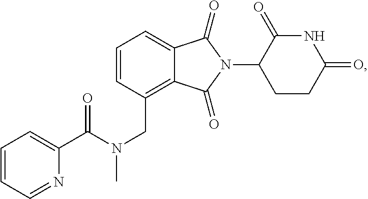 [SMILES: CN(Cc1cccc2c1C(=O)N(C1CCC(=O)NC1=O)C2=O)C(=O)c1ccccn1]

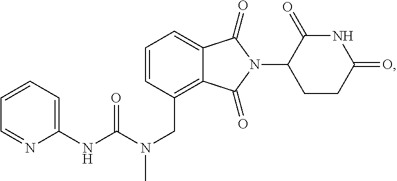 [SMILES: CN(Cc1cccc2c1C(=O)N(C1CCC(=O)NC1=O)C2=O)C(=O)Nc1ccccn1]

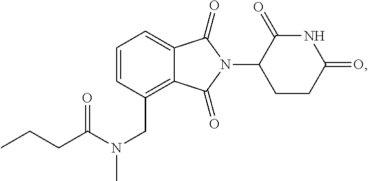 [SMILES: CCCC(=O)N(C)Cc1cccc2c1C(=O)N(C1CCC(=O)NC1=O)C2=O]

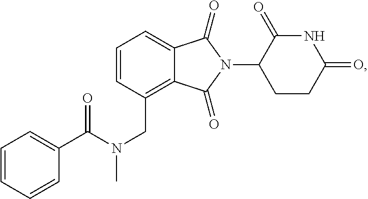 [SMILES: CN(Cc1cccc2c1C(=O)N(C1CCC(=O)NC1=O)C2=O)C(=O)c1ccccc1]

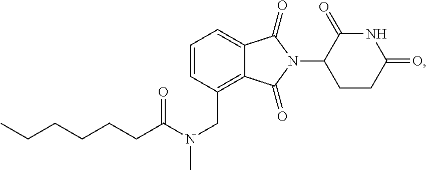 [SMILES: CCCCCCC(=O)N(C)Cc1cccc2c1C(=O)N(C1CCC(=O)NC1=O)C2=O]

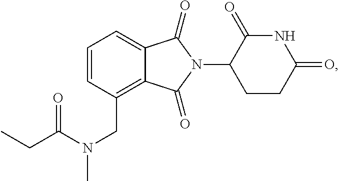 [SMILES: CCC(=O)N(C)Cc1cccc2c1C(=O)N(C1CCC(=O)NC1=O)C2=O]

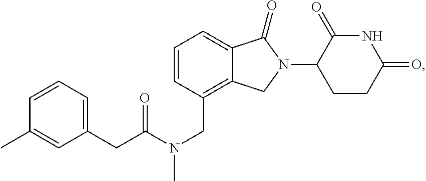 [SMILES: Cc1cccc(CC(=O)N(C)Cc2cccc3c2CN(C2CCC(=O)NC2=O)C3=O)c1]

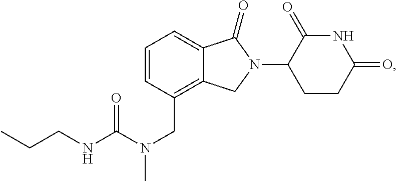 [SMILES: CCCNC(=O)N(C)Cc1cccc2c1CN(C1CCC(=O)NC1=O)C2=O]

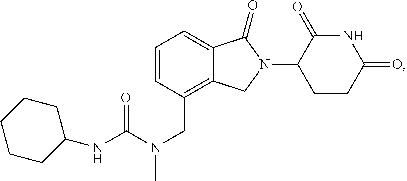 [SMILES: CN(Cc1cccc2c1CN(C1CCC(=O)NC1=O)C2=O)C(=O)NC1CCCCC1]

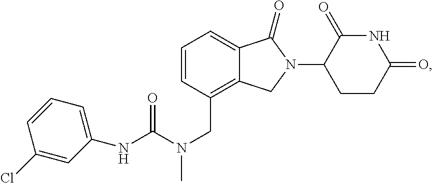 [SMILES: CN(Cc1cccc2c1CN(C1CCC(=O)NC1=O)C2=O)C(=O)Nc1cccc(Cl)c1]

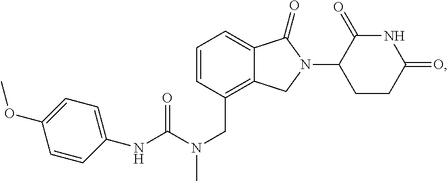 [SMILES: COc1ccc(NC(=O)N(C)Cc2cccc3c2CN(C2CCC(=O)NC2=O)C3=O)cc1]

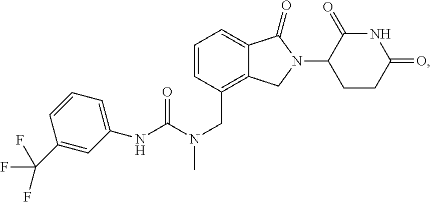 [SMILES: CN(Cc1cccc2c1CN(C1CCC(=O)NC1=O)C2=O)C(=O)Nc1cccc(C(F)(F)F)c1]

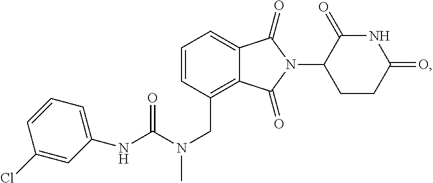 [SMILES: CN(Cc1cccc2c1C(=O)N(C1CCC(=O)NC1=O)C2=O)C(=O)Nc1cccc(Cl)c1]

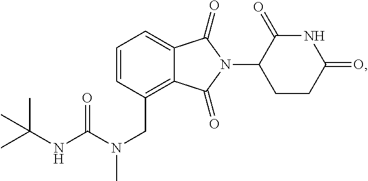 [SMILES: CN(Cc1cccc2c1C(=O)N(C1CCC(=O)NC1=O)C2=O)C(=O)NC(C)(C)C]

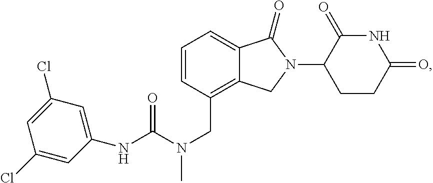 [SMILES: CN(Cc1cccc2c1CN(C1CCC(=O)NC1=O)C2=O)C(=O)Nc1cc(Cl)cc(Cl)c1]

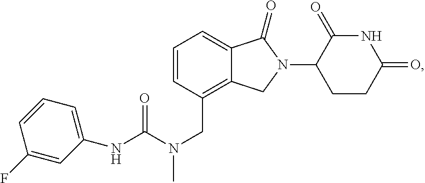 [SMILES: CN(Cc1cccc2c1CN(C1CCC(=O)NC1=O)C2=O)C(=O)Nc1cccc(F)c1]

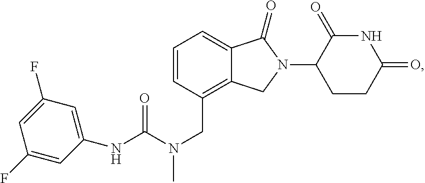 [SMILES: CN(Cc1cccc2c1CN(C1CCC(=O)NC1=O)C2=O)C(=O)Nc1cc(F)cc(F)c1]

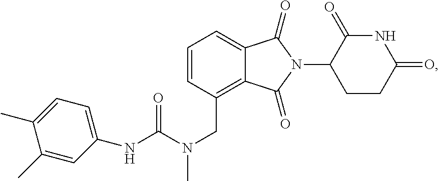 [SMILES: Cc1ccc(NC(=O)N(C)Cc2cccc3c2C(=O)N(C2CCC(=O)NC2=O)C3=O)cc1C]

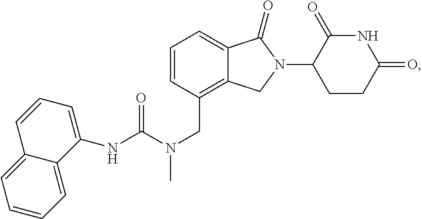 [SMILES: CN(Cc1cccc2c1CN(C1CCC(=O)NC1=O)C2=O)C(=O)Nc1cccc2ccccc12]

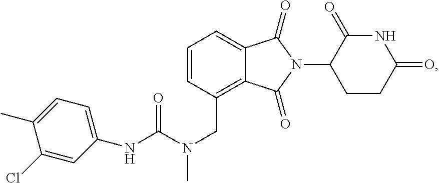 [SMILES: Cc1ccc(NC(=O)N(C)Cc2cccc3c2C(=O)N(C2CCC(=O)NC2=O)C3=O)cc1Cl]

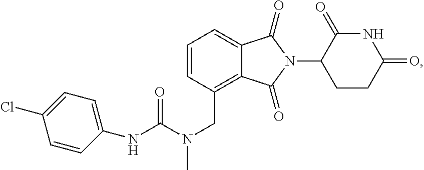 [SMILES: CN(Cc1cccc2c1C(=O)N(C1CCC(=O)NC1=O)C2=O)C(=O)Nc1ccc(Cl)cc1]

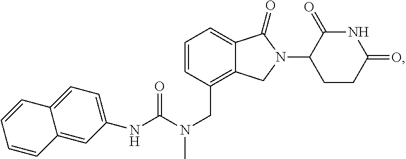 [SMILES: CN(Cc1cccc2c1CN(C1CCC(=O)NC1=O)C2=O)C(=O)Nc1ccc2ccccc2c1]

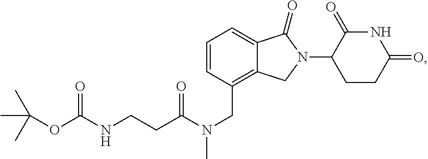 [SMILES: CN(Cc1cccc2c1CN(C1CCC(=O)NC1=O)C2=O)C(=O)CCNC(=O)OC(C)(C)C]

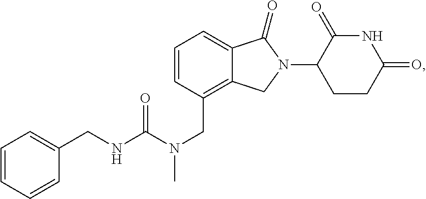 [SMILES: CN(Cc1cccc2c1CN(C1CCC(=O)NC1=O)C2=O)C(=O)NCc1ccccc1]

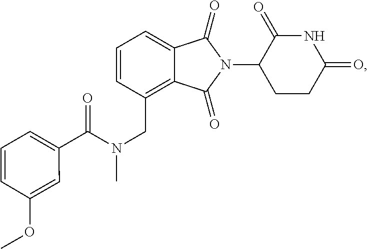 [SMILES: COc1cccc(C(=O)N(C)Cc2cccc3c2C(=O)N(C2CCC(=O)NC2=O)C3=O)c1]

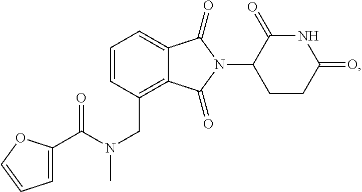 [SMILES: CN(Cc1cccc2c1C(=O)N(C1CCC(=O)NC1=O)C2=O)C(=O)c1ccco1]

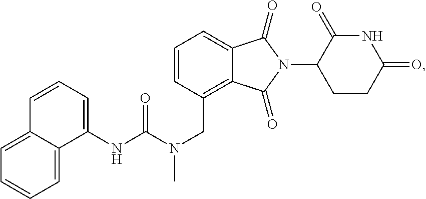 [SMILES: CN(Cc1cccc2c1C(=O)N(C1CCC(=O)NC1=O)C2=O)C(=O)Nc1cccc2ccccc12]

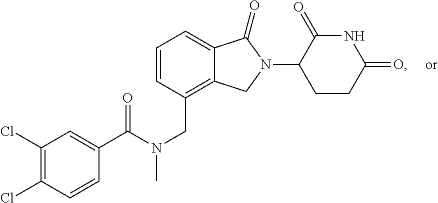 [SMILES: CN(Cc1cccc2c1CN(C1CCC(=O)NC1=O)C2=O)C(=O)c1ccc(Cl)c(Cl)c1]

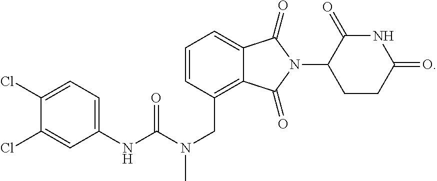 [SMILES: CN(Cc1cccc2c1C(=O)N(C1CCC(=O)NC1=O)C2=O)C(=O)Nc1ccc(Cl)c(Cl)c1]

Still other specific IMiD® immunomodulatory drugs provided herein belong to a class of 5-substituted isoindole compounds disclosed in U.S. Patent Application Publication No. US 2009/0142297, the entirety of which is incorporated herein by reference. Representative compounds are of formula XI:

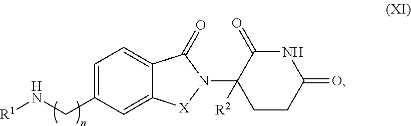

and pharmaceutically acceptable salts, solvates, stereoisomers, and prodrugs thereof,
wherein:
n is 0 or 1;

### X is CH₂, C═O, or C═S;

### R¹ is:

a) —(CH₂)ₘR³ or —CO(CH₂)ₘR³, wherein
   m is 0, 1, 2, or 3; and
   R³ is 5-10 membered aryl or heteroaryl, optionally substituted with one or more halogen;
b) —C═YR⁴, wherein
   Y is O or S; and
      R⁴ is: (C₁-C₁₀)alkyl; (C₁-C₁₀)alkoxy; (C₀-C₁₀)alkyl- (5 to 10 membered heteroaryl or heterocycle), said heteroaryl or heterocycle optionally substituted with one or more of (C₁-C₆)alkyl, halogen, oxo, (C₁-C₆)alkoxy, or —Z—(C₁-C₆)alkyl, wherein Z is S or SO₂, and wherein said (C₁-C₆)alkyl may be optionally substituted with one or more halogen; (C₀-C₁₀)alkyl- (5 to 10 membered aryl), said aryl optionally substituted with one or more of: halogen; (C₁-C₆)alkoxy, itself optionally substituted with one or more halogen; (C₁-C₆)alkyl, itself optionally substituted with one or more halogen; or —Z—(C₁-C₆)alkyl, wherein Z is S or SO₂, and wherein said (C₁-C₆)alkyl may be optionally substituted with one or more halogen; or (C₁-C₆)alkyl-CO—O—R¹², wherein R¹² is H or (C₁-C₆)alkyl; or
c) —C═ZNHR⁶, wherein

### Z is O or S; and

R⁶ is: (C₁-C₁₀)alkyl; (C₁-C₁₀)alkoxy; 5 to 10 membered aryl or heteroaryl, optionally substituted with one or more of: halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and
R² is H or (C₁-C₆)alkyl.

Representative compounds are of formula:

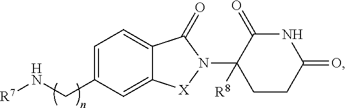

and pharmaceutically acceptable salts, solvates, stereoisomers, and prodrugs thereof,
wherein:
n is 0 or 1;

### X is CH₂ or C═O;

R⁷ is —(CH₂)ₘR⁹, wherein m is 0, 1, 2, or 3, and R⁹ is 5-10 membered aryl or heteroaryl, optionally substituted with one or more halogen; and
R⁸ is H or (C₁-C₆)alkyl.

In one embodiment, X is C═O. In another embodiment, X is CH₂.

In one embodiment, n is 0. In another embodiment, n is 1.

In one embodiment, m is 0. In another embodiment, m is 1. In another embodiment, m is 2. In another embodiment, m is 3.

In one embodiment, R⁹ is 5-10 membered aryl. In certain specific embodiments, R⁹ is phenyl, optionally substituted with one or more halogen.

In one embodiment, R⁹ is 5-10 membered heteroaryl. In certain specific embodiments, R⁹ is furyl or benzofuryl.

In one embodiment, R⁸ is H. In another embodiment, R⁸ is (C₁-C₆)alkyl. In certain specific embodiments, R⁸ is methyl.

All of the combinations of the above embodiments are encompassed by this invention.

Examples include, but are not limited to, those listed below, or a pharmaceutically acceptable salt, solvate (e.g., hydrate), prodrug or stereoisomer thereof:

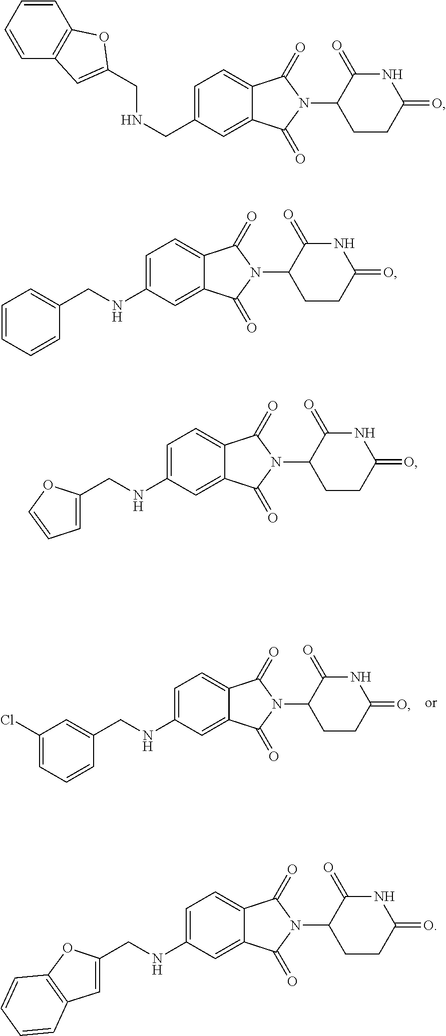 [SMILES: O=C1CCC(N2C(=O)c3ccc(CNCc4cc5ccccc5o4)cc3C2=O)C(=O)N1 ; O=C1CCC(N2C(=O)c3ccc(NCc4ccccc4)cc3C2=O)C(=O)N1 ; O=C1CCC(N2C(=O)c3ccc(NCc4ccco4)cc3C2=O)C(=O)N1 ; O=C1CCC(N2C(=O)c3ccc(NCc4cccc(Cl)c4)cc3C2=O)C(=O)N1 ; O=C1CCC(N2C(=O)c3ccc(NCc4cc5ccccc5o4)cc3C2=O)C(=O)N1]

Other representative compounds are of formula:

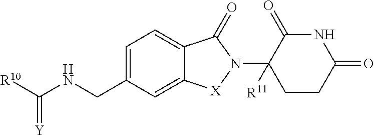

and pharmaceutically acceptable salts, solvates, stereoisomers, and prodrugs thereof,
wherein:

### X is CH₂ or C═O;

### Y is O or S;

R¹⁰ is: (C₁-C₁₀)alkyl; (C₁-C₁₀)alkoxy; (C₀-C₁₀)alkyl- (5 to 10 membered heteroaryl or heterocycle), said heteroaryl or heterocycle optionally substituted with one or more of: (C₁-C₆)alkyl, itself substituted with one or more halogen; halogen; oxo; (C₁-C₆)alkoxy, itself substituted with one or more halogen; or —Z—(C₁-C₆)alkyl, wherein Z is S or SO₂, and wherein said (C₁-C₆)alkyl may be optionally substituted with one or more halogen; (C₀-C₁₀)alkyl- (5 to 10 membered aryl), said aryl optionally substituted with one or more of: halogen; (C₁-C₆)alkoxy, itself optionally substituted with one or more halogen; (C₁-C₆)alkyl, itself optionally substituted with one or more halogen; or —Z—(C₁-C₆)alkyl, wherein Z is S or SO₂, and wherein said (C₁-C₆)alkyl may be optionally substituted with one or more halogen; or (C₁-C₆)alkyl-CO—O—R¹², wherein R¹² is H or (C₁-C₆)alkyl; and
R¹¹ is H or (C₁-C₆)alkyl.

In one embodiment, X is CH₂. In another embodiment, X is C═O.

In one embodiment, Y is O. In another embodiment, Y is S.

In one embodiment, R¹⁰ is (C₁-C₁₀)alkyl. In certain specific embodiments, R¹⁰ is (C₅-C₁₀)alkyl. In certain specific embodiments, R¹⁰ is pentyl or hexyl.

In one embodiment, R¹⁰ is (C₁-C₁₀)alkoxy. In certain specific embodiments, R¹⁰ is (C₅-C₁₀)alkoxy. In certain specific embodiments, R¹⁰ is pentyloxy or hexyloxy.

In one embodiment, R¹⁰ is 5 to 10 membered heteroaryl. In certain specific embodiments, R¹⁰ is thiophenyl or furyl.

In one embodiment, R¹⁰ is 5 to 10 membered aryl, optionally substituted with one or more halogen. In certain specific embodiments, R¹⁰ is phenyl, optionally substituted with one or more halogen.

In one embodiment, R¹⁰ is 5 to 10 membered aryl or heteroaryl, optionally substituted with (C₁-C₆)alkyl or (C₁-C₆)alkoxy, themselves optionally substituted with one or more halogen. In certain specific embodiments, R¹⁰ is phenyl substituted with (C₁-C₃)alkyl or (C₁-C₃)alkoxy, substituted with one or more halogen. In certain specific embodiments, R¹⁰ is phenyl substituted with methyl or methoxy, substituted with 1, 2, or 3 halogens.

In one embodiment, R¹⁰ is aryl or heteroaryl substituted with —S—(C₁-C₆)alkyl, wherein said alkyl itself optionally substituted with one or more halogen. In another embodiment, R¹⁰ is aryl or heteroaryl substituted with —SO₂—(C₁-C₆)alkyl, wherein said alkyl itself optionally substituted with one or more halogen.

In one embodiment, R¹⁰ is (C₁-C₆)alkyl-CO—O—R¹², and R¹² is (C₁-C₆)alkyl. In one specific embodiment, R¹⁰ is butyl-CO—O-tBu.

In one embodiment, R¹⁰ is (C₁-C₆)alkyl-CO—O—R¹², and R¹² is H. In one specific embodiment, R¹⁰ is butyl-COOH.

In one embodiment, R¹¹ is H. In another embodiment, R¹¹ is (C₁-C₆)alkyl. In certain specific embodiments, R¹¹ is methyl.

All of the combinations of the above embodiments are encompassed by this invention.

Examples include, but are not limited to, those listed in Table J, below, or a pharmaceutically acceptable salt, solvate (e.g., hydrate), or stereoisomer thereof:

TABLE J

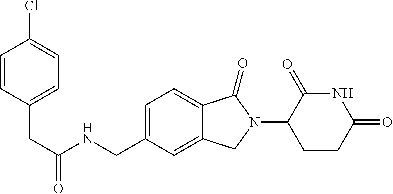 [SMILES: O=C(Cc1ccc(Cl)cc1)NCc1ccc2c(c1)CN(C1CCC(=O)NC1=O)C2=O]

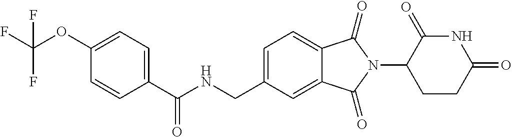 [SMILES: O=C1CCC(N2C(=O)c3ccc(CNC(=O)c4ccc(OC(F)(F)F)cc4)cc3C2=O)C(=O)N1]

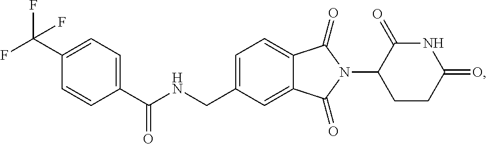 [SMILES: O=C1CCC(N2C(=O)c3ccc(CNC(=O)c4ccc(C(F)(F)F)cc4)cc3C2=O)C(=O)N1]

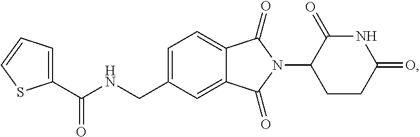 [SMILES: O=C1CCC(N2C(=O)c3ccc(CNC(=O)c4cccs4)cc3C2=O)C(=O)N1]

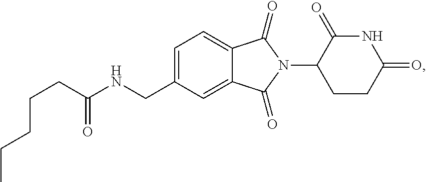 [SMILES: CCCCCC(=O)NCc1ccc2c(c1)C(=O)N(C1CCC(=O)NC1=O)C2=O]

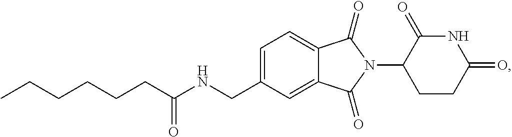 [SMILES: CCCCCCC(=O)NCc1ccc2c(c1)C(=O)N(C1CCC(=O)NC1=O)C2=O]

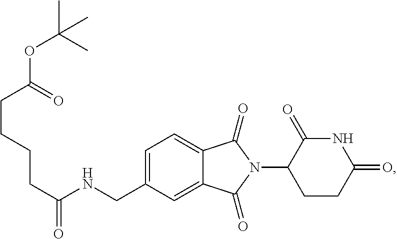 [SMILES: CC(C)(C)OC(=O)CCCCC(=O)NCc1ccc2c(c1)C(=O)N(C1CCC(=O)NC1=O)C2=O]

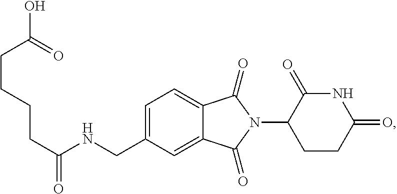 [SMILES: O=C(O)CCCCC(=O)NCc1ccc2c(c1)C(=O)N(C1CCC(=O)NC1=O)C2=O]

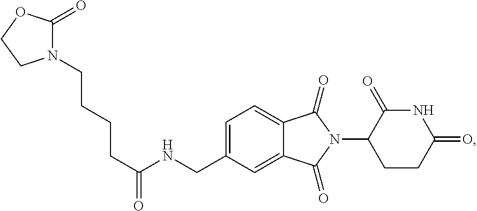 [SMILES: O=C(CCCCN1CCOC1=O)NCc1ccc2c(c1)C(=O)N(C1CCC(=O)NC1=O)C2=O]

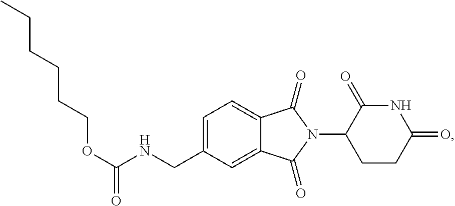 [SMILES: CCCCCCOC(=O)NCc1ccc2c(c1)C(=O)N(C1CCC(=O)NC1=O)C2=O]

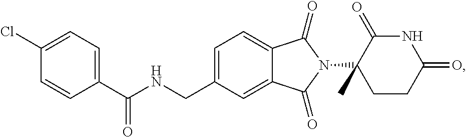

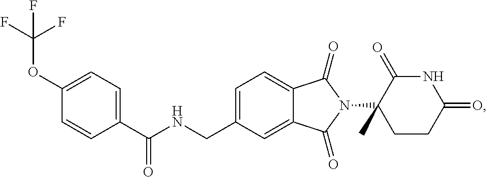

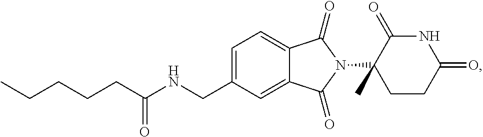

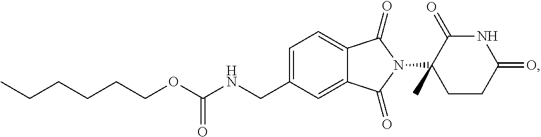

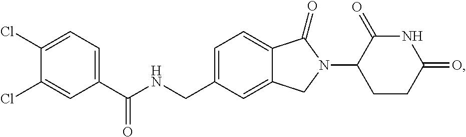 [SMILES: O=C1CCC(N2Cc3cc(CNC(=O)c4ccc(Cl)c(Cl)c4)ccc3C2=O)C(=O)N1]

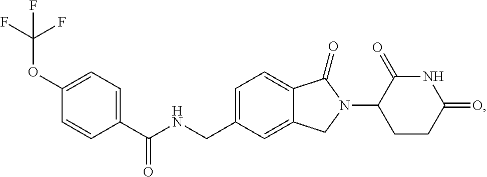 [SMILES: O=C1CCC(N2Cc3cc(CNC(=O)c4ccc(OC(F)(F)F)cc4)ccc3C2=O)C(=O)N1]

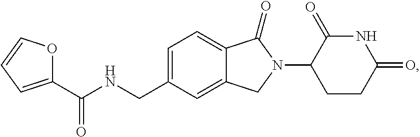 [SMILES: O=C1CCC(N2Cc3cc(CNC(=O)c4ccco4)ccc3C2=O)C(=O)N1]

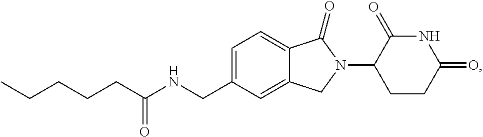 [SMILES: CCCCCC(=O)NCc1ccc2c(c1)CN(C1CCC(=O)NC1=O)C2=O]

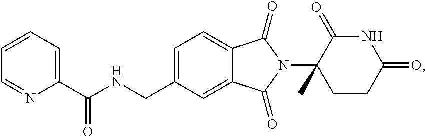 [SMILES: C[C@]1(N2C(=O)c3ccc(CNC(=O)c4ccccn4)cc3C2=O)CCC(=O)NC1=O]

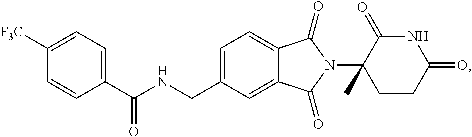 [SMILES: C[C@]1(N2C(=O)c3ccc(CNC(=O)c4ccc(C(F)(F)F)cc4)cc3C2=O)CCC(=O)NC1=O]

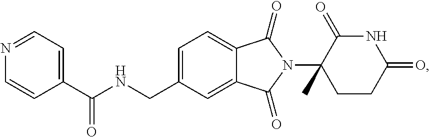 [SMILES: C[C@]1(N2C(=O)c3ccc(CNC(=O)c4ccncc4)cc3C2=O)CCC(=O)NC1=O]

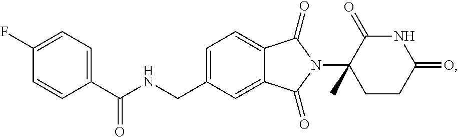 [SMILES: C[C@]1(N2C(=O)c3ccc(CNC(=O)c4ccc(F)cc4)cc3C2=O)CCC(=O)NC1=O]

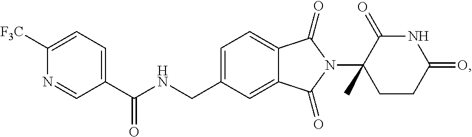 [SMILES: C[C@]1(N2C(=O)c3ccc(CNC(=O)c4ccc(C(F)(F)F)nc4)cc3C2=O)CCC(=O)NC1=O]

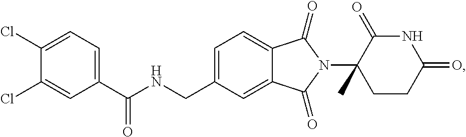 [SMILES: C[C@]1(N2C(=O)c3ccc(CNC(=O)c4ccc(Cl)c(Cl)c4)cc3C2=O)CCC(=O)NC1=O]

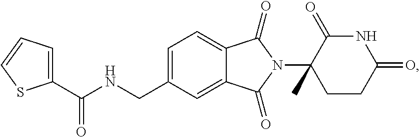 [SMILES: C[C@]1(N2C(=O)c3ccc(CNC(=O)c4cccs4)cc3C2=O)CCC(=O)NC1=O]

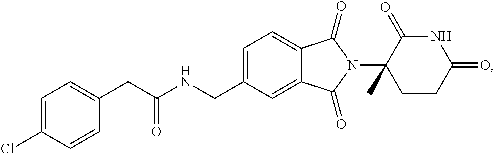 [SMILES: C[C@]1(N2C(=O)c3ccc(CNC(=O)Cc4ccc(Cl)cc4)cc3C2=O)CCC(=O)NC1=O]

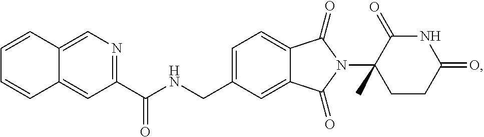 [SMILES: C[C@]1(N2C(=O)c3ccc(CNC(=O)c4cc5ccccc5cn4)cc3C2=O)CCC(=O)NC1=O]

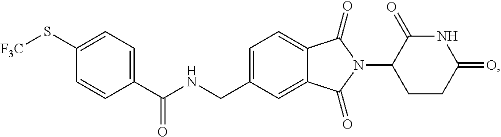 [SMILES: O=C1CCC(N2C(=O)c3ccc(CNC(=O)c4ccc(SC(F)(F)F)cc4)cc3C2=O)C(=O)N1]

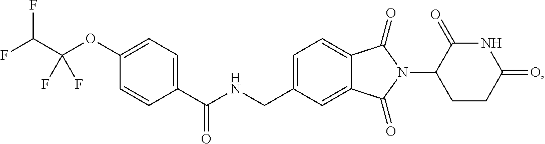 [SMILES: O=C1CCC(N2C(=O)c3ccc(CNC(=O)c4ccc(OC(F)(F)C(F)F)cc4)cc3C2=O)C(=O)N1]

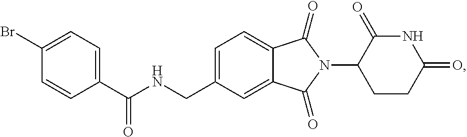 [SMILES: O=C1CCC(N2C(=O)c3ccc(CNC(=O)c4ccc(Br)cc4)cc3C2=O)C(=O)N1]

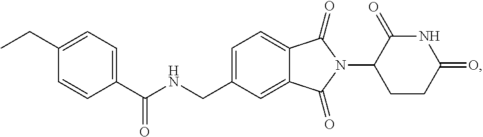 [SMILES: CCc1ccc(C(=O)NCc2ccc3c(c2)C(=O)N(C2CCC(=O)NC2=O)C3=O)cc1]

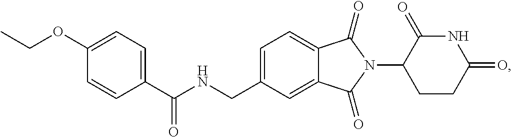 [SMILES: CCOc1ccc(C(=O)NCc2ccc3c(c2)C(=O)N(C2CCC(=O)NC2=O)C3=O)cc1]

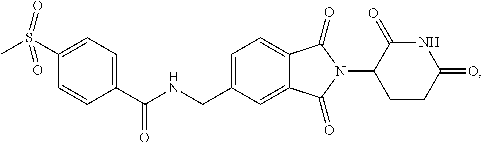 [SMILES: CS(=O)(=O)c1ccc(C(=O)NCc2ccc3c(c2)C(=O)N(C2CCC(=O)NC2=O)C3=O)cc1]

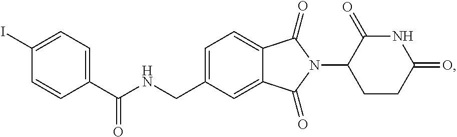 [SMILES: O=C1CCC(N2C(=O)c3ccc(CNC(=O)c4ccc(I)cc4)cc3C2=O)C(=O)N1]

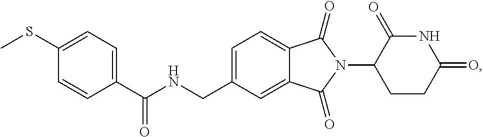 [SMILES: CSc1ccc(C(=O)NCc2ccc3c(c2)C(=O)N(C2CCC(=O)NC2=O)C3=O)cc1]

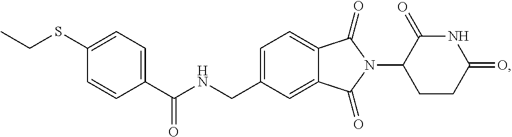 [SMILES: CCSc1ccc(C(=O)NCc2ccc3c(c2)C(=O)N(C2CCC(=O)NC2=O)C3=O)cc1]

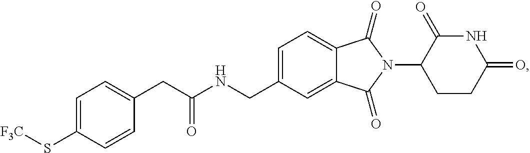 [SMILES: O=C(Cc1ccc(SC(F)(F)F)cc1)NCc1ccc2c(c1)C(=O)N(C1CCC(=O)NC1=O)C2=O]

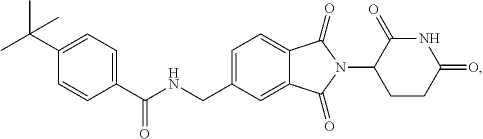 [SMILES: CC(C)(C)c1ccc(C(=O)NCc2ccc3c(c2)C(=O)N(C2CCC(=O)NC2=O)C3=O)cc1]

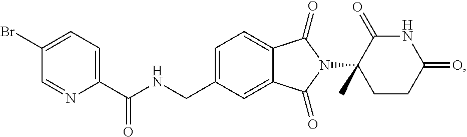 [SMILES: C[C@]1(N2C(=O)c3ccc(CNC(=O)c4ccc(Br)cn4)cc3C2=O)CCC(=O)NC1=O]

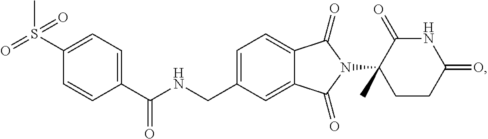 [SMILES: C[C@]1(N2C(=O)c3ccc(CNC(=O)c4ccc(S(C)(=O)=O)cc4)cc3C2=O)CCC(=O)NC1=O]

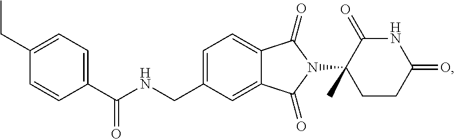 [SMILES: CCc1ccc(C(=O)NCc2ccc3c(c2)C(=O)N([C@@]2(C)CCC(=O)NC2=O)C3=O)cc1]

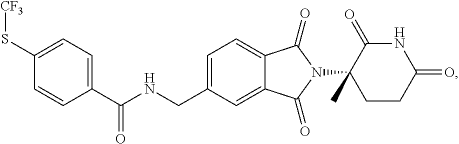 [SMILES: C[C@]1(N2C(=O)c3ccc(CNC(=O)c4ccc(SC(F)(F)F)cc4)cc3C2=O)CCC(=O)NC1=O]

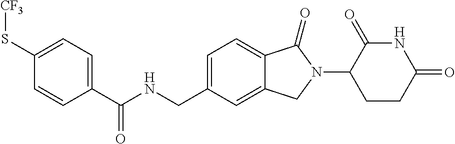 [SMILES: O=C1CCC(N2Cc3cc(CNC(=O)c4ccc(SC(F)(F)F)cc4)ccc3C2=O)C(=O)N1]

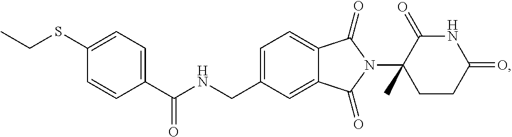 [SMILES: CCSc1ccc(C(=O)NCc2ccc3c(c2)C(=O)N([C@@]2(C)CCC(=O)NC2=O)C3=O)cc1]

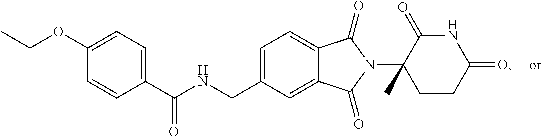 [SMILES: CCOc1ccc(C(=O)NCc2ccc3c(c2)C(=O)N([C@@]2(C)CCC(=O)NC2=O)C3=O)cc1]

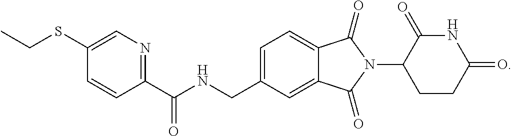 [SMILES: CCSc1ccc(C(=O)NCc2ccc3c(c2)C(=O)N(C2CCC(=O)NC2=O)C3=O)nc1]

Other examples include, but are not limited to, those listed in Table K, below, or a pharmaceutically acceptable salt, solvate (e.g., hydrate), or stereoisomer thereof:

TABLE K

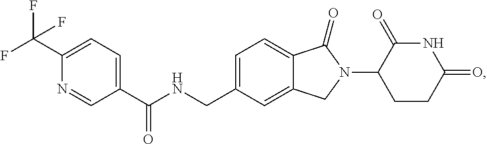 [SMILES: O=C1CCC(N2Cc3cc(CNC(=O)c4ccc(C(F)(F)F)nc4)ccc3C2=O)C(=O)N1]

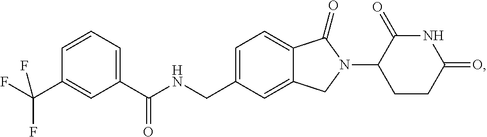 [SMILES: O=C1CCC(N2Cc3cc(CNC(=O)c4cccc(C(F)(F)F)c4)ccc3C2=O)C(=O)N1]

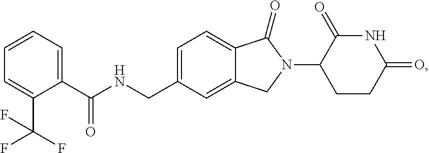 [SMILES: O=C1CCC(N2Cc3cc(CNC(=O)c4ccccc4C(F)(F)F)ccc3C2=O)C(=O)N1]

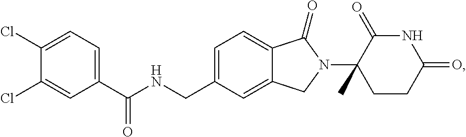 [SMILES: C[C@]1(N2Cc3cc(CNC(=O)c4ccc(Cl)c(Cl)c4)ccc3C2=O)CCC(=O)NC1=O]

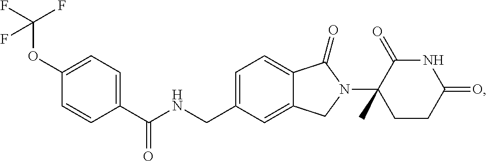 [SMILES: C[C@]1(N2Cc3cc(CNC(=O)c4ccc(OC(F)(F)F)cc4)ccc3C2=O)CCC(=O)NC1=O]

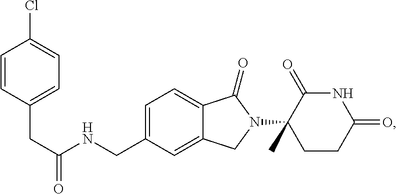 [SMILES: C[C@]1(N2Cc3cc(CNC(=O)Cc4ccc(Cl)cc4)ccc3C2=O)CCC(=O)NC1=O]

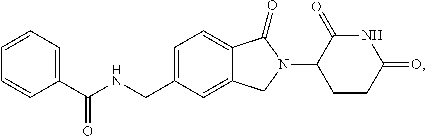 [SMILES: O=C1CCC(N2Cc3cc(CNC(=O)c4ccccc4)ccc3C2=O)C(=O)N1]

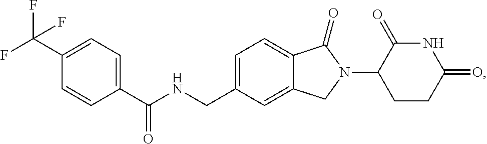 [SMILES: O=C1CCC(N2Cc3cc(CNC(=O)c4ccc(C(F)(F)F)cc4)ccc3C2=O)C(=O)N1]

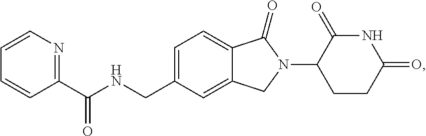 [SMILES: O=C1CCC(N2Cc3cc(CNC(=O)c4ccccn4)ccc3C2=O)C(=O)N1]

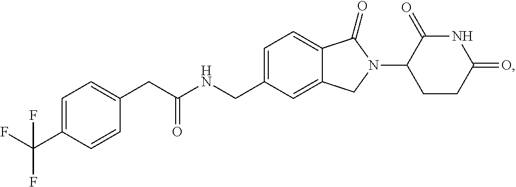 [SMILES: O=C(Cc1ccc(C(F)(F)F)cc1)NCc1ccc2c(c1)CN(C1CCC(=O)NC1=O)C2=O]

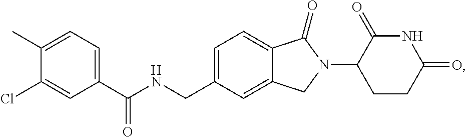 [SMILES: Cc1ccc(C(=O)NCc2ccc3c(c2)CN(C2CCC(=O)NC2=O)C3=O)cc1Cl]

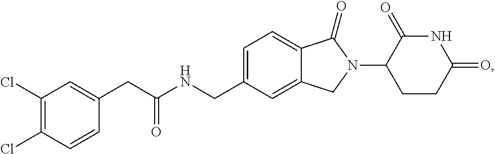 [SMILES: O=C(Cc1ccc(Cl)c(Cl)c1)NCc1ccc2c(c1)CN(C1CCC(=O)NC1=O)C2=O]

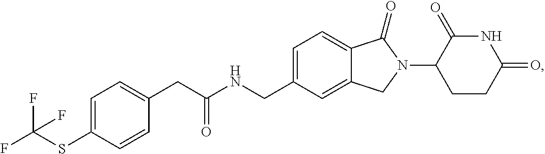 [SMILES: O=C(Cc1ccc(SC(F)(F)F)cc1)NCc1ccc2c(c1)CN(C1CCC(=O)NC1=O)C2=O]

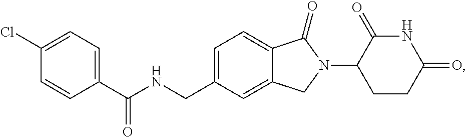 [SMILES: O=C1CCC(N2Cc3cc(CNC(=O)c4ccc(Cl)cc4)ccc3C2=O)C(=O)N1]

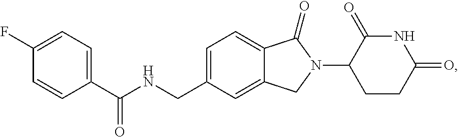 [SMILES: O=C1CCC(N2Cc3cc(CNC(=O)c4ccc(F)cc4)ccc3C2=O)C(=O)N1]

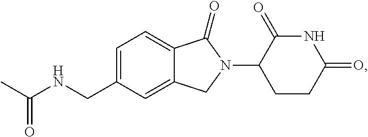 [SMILES: CC(=O)NCc1ccc2c(c1)CN(C1CCC(=O)NC1=O)C2=O]

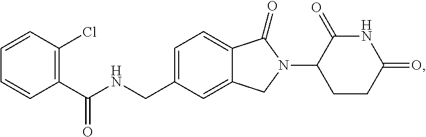 [SMILES: O=C1CCC(N2Cc3cc(CNC(=O)c4ccccc4Cl)ccc3C2=O)C(=O)N1]

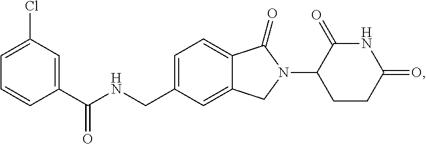 [SMILES: O=C1CCC(N2Cc3cc(CNC(=O)c4cccc(Cl)c4)ccc3C2=O)C(=O)N1]

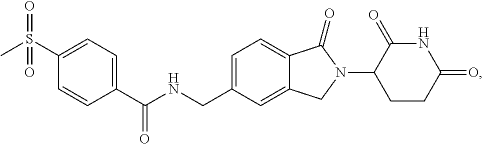 [SMILES: CS(=O)(=O)c1ccc(C(=O)NCc2ccc3c(c2)CN(C2CCC(=O)NC2=O)C3=O)cc1]

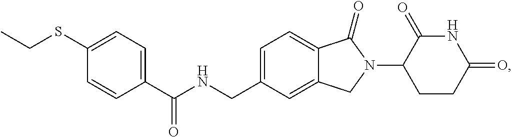 [SMILES: CCSc1ccc(C(=O)NCc2ccc3c(c2)CN(C2CCC(=O)NC2=O)C3=O)cc1]

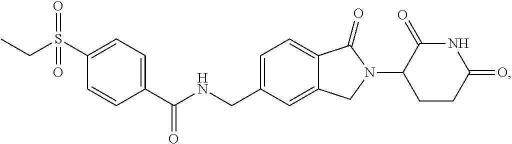 [SMILES: CCS(=O)(=O)c1ccc(C(=O)NCc2ccc3c(c2)CN(C2CCC(=O)NC2=O)C3=O)cc1]

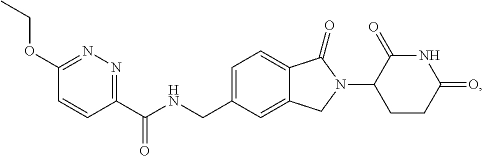 [SMILES: CCOc1ccc(C(=O)NCc2ccc3c(c2)CN(C2CCC(=O)NC2=O)C3=O)nn1]

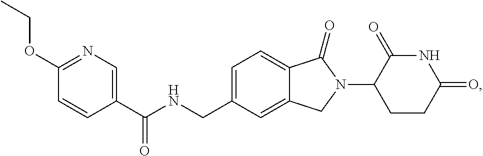 [SMILES: CCOc1ccc(C(=O)NCc2ccc3c(c2)CN(C2CCC(=O)NC2=O)C3=O)cn1]

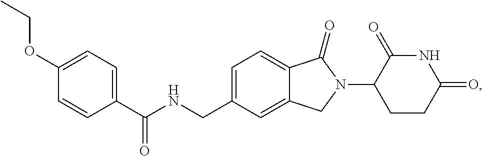 [SMILES: CCOc1ccc(C(=O)NCc2ccc3c(c2)CN(C2CCC(=O)NC2=O)C3=O)cc1]

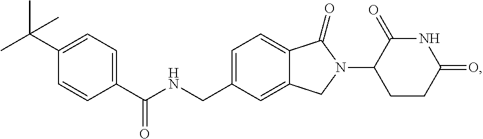 [SMILES: CC(C)(C)c1ccc(C(=O)NCc2ccc3c(c2)CN(C2CCC(=O)NC2=O)C3=O)cc1]

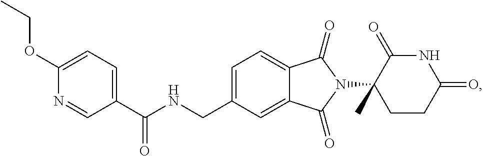 [SMILES: CCOc1ccc(C(=O)NCc2ccc3c(c2)C(=O)N([C@@]2(C)CCC(=O)NC2=O)C3=O)cn1]

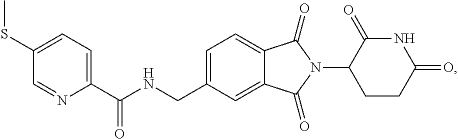 [SMILES: CSc1ccc(C(=O)NCc2ccc3c(c2)C(=O)N(C2CCC(=O)NC2=O)C3=O)nc1]

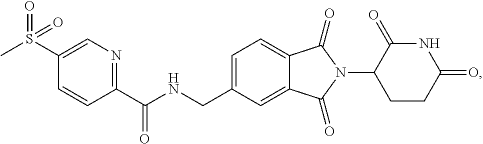 [SMILES: CS(=O)(=O)c1ccc(C(=O)NCc2ccc3c(c2)C(=O)N(C2CCC(=O)NC2=O)C3=O)nc1]

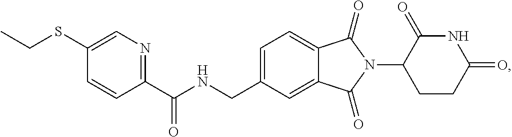 [SMILES: CCSc1ccc(C(=O)NCc2ccc3c(c2)C(=O)N(C2CCC(=O)NC2=O)C3=O)nc1]

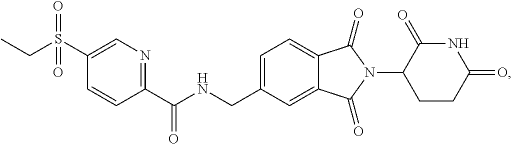 [SMILES: CCS(=O)(=O)c1ccc(C(=O)NCc2ccc3c(c2)C(=O)N(C2CCC(=O)NC2=O)C3=O)nc1]

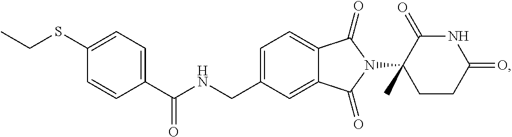 [SMILES: CCSc1ccc(C(=O)NCc2ccc3c(c2)C(=O)N([C@@]2(C)CCC(=O)NC2=O)C3=O)cc1]

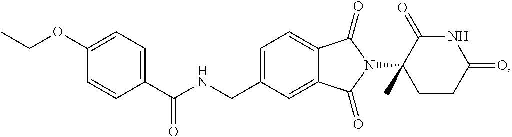 [SMILES: CCOc1ccc(C(=O)NCc2ccc3c(c2)C(=O)N([C@@]2(C)CCC(=O)NC2=O)C3=O)cc1]

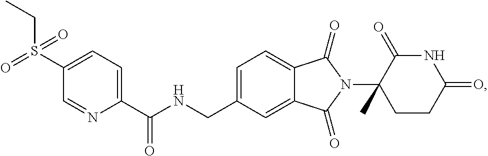 [SMILES: CCS(=O)(=O)c1ccc(C(=O)NCc2ccc3c(c2)C(=O)N([C@@]2(C)CCC(=O)NC2=O)C3=O)nc1]

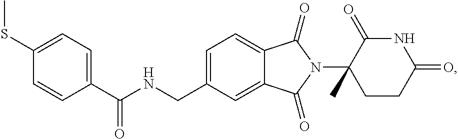 [SMILES: CSc1ccc(C(=O)NCc2ccc3c(c2)C(=O)N([C@@]2(C)CCC(=O)NC2=O)C3=O)cc1]

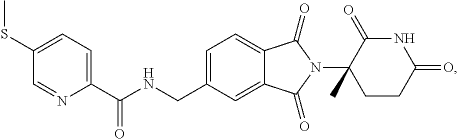 [SMILES: CSc1ccc(C(=O)NCc2ccc3c(c2)C(=O)N([C@@]2(C)CCC(=O)NC2=O)C3=O)nc1]

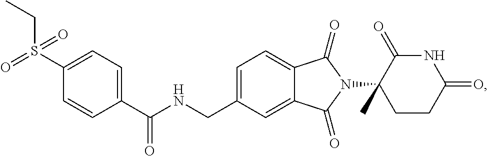 [SMILES: CCS(=O)(=O)c1ccc(C(=O)NCc2ccc3c(c2)C(=O)N([C@@]2(C)CCC(=O)NC2=O)C3=O)cc1]

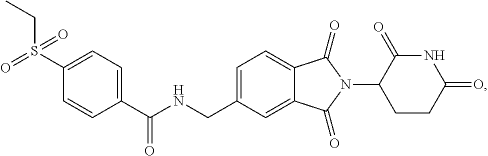 [SMILES: CCS(=O)(=O)c1ccc(C(=O)NCc2ccc3c(c2)C(=O)N(C2CCC(=O)NC2=O)C3=O)cc1]

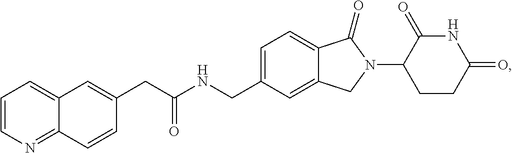 [SMILES: O=C(Cc1ccc2ncccc2c1)NCc1ccc2c(c1)CN(C1CCC(=O)NC1=O)C2=O]

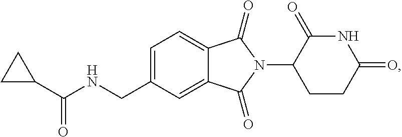 [SMILES: O=C1CCC(N2C(=O)c3ccc(CNC(=O)C4CC4)cc3C2=O)C(=O)N1]

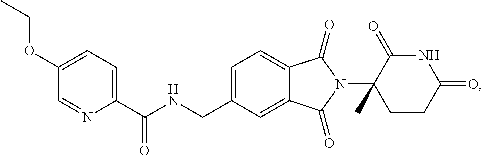 [SMILES: CCOc1ccc(C(=O)NCc2ccc3c(c2)C(=O)N([C@@]2(C)CCC(=O)NC2=O)C3=O)nc1]

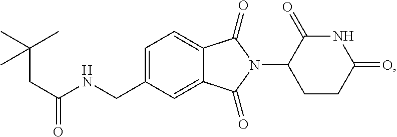 [SMILES: CC(C)(C)CC(=O)NCc1ccc2c(c1)C(=O)N(C1CCC(=O)NC1=O)C2=O]

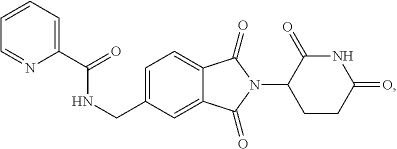 [SMILES: O=C1CCC(N2C(=O)c3ccc(CNC(=O)c4ccccn4)cc3C2=O)C(=O)N1]

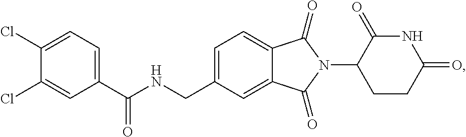 [SMILES: O=C1CCC(N2C(=O)c3ccc(CNC(=O)c4ccc(Cl)c(Cl)c4)cc3C2=O)C(=O)N1]

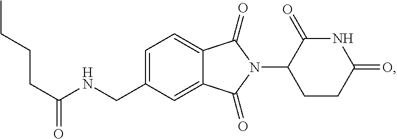 [SMILES: CCCCC(=O)NCc1ccc2c(c1)C(=O)N(C1CCC(=O)NC1=O)C2=O]

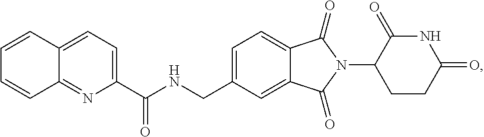 [SMILES: O=C1CCC(N2C(=O)c3ccc(CNC(=O)c4ccc5ccccc5n4)cc3C2=O)C(=O)N1]

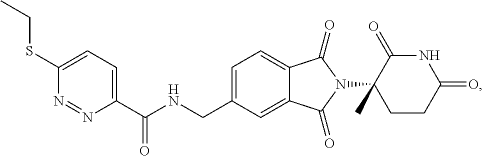 [SMILES: CCSc1ccc(C(=O)NCc2ccc3c(c2)C(=O)N([C@@]2(C)CCC(=O)NC2=O)C3=O)nn1]

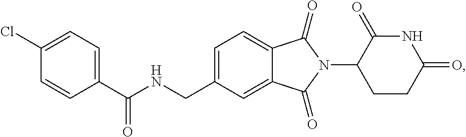 [SMILES: O=C1CCC(N2C(=O)c3ccc(CNC(=O)c4ccc(Cl)cc4)cc3C2=O)C(=O)N1]

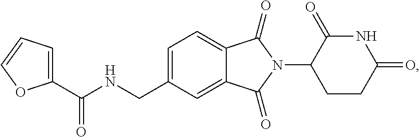 [SMILES: O=C1CCC(N2C(=O)c3ccc(CNC(=O)c4ccco4)cc3C2=O)C(=O)N1]

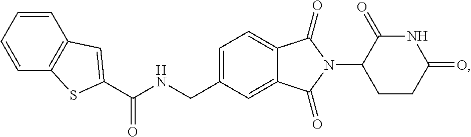 [SMILES: O=C1CCC(N2C(=O)c3ccc(CNC(=O)c4cc5ccccc5s4)cc3C2=O)C(=O)N1]

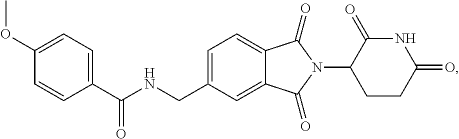 [SMILES: COc1ccc(C(=O)NCc2ccc3c(c2)C(=O)N(C2CCC(=O)NC2=O)C3=O)cc1]

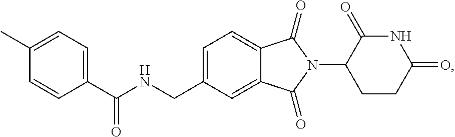 [SMILES: Cc1ccc(C(=O)NCc2ccc3c(c2)C(=O)N(C2CCC(=O)NC2=O)C3=O)cc1]

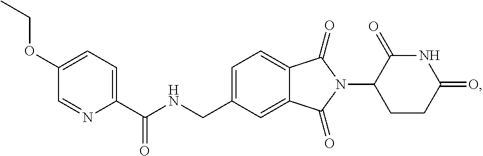 [SMILES: CCOc1ccc(C(=O)NCc2ccc3c(c2)C(=O)N(C2CCC(=O)NC2=O)C3=O)nc1]

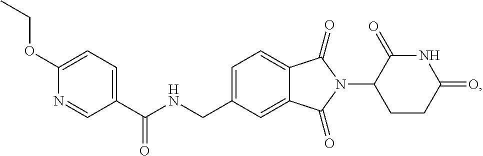 [SMILES: CCOc1ccc(C(=O)NCc2ccc3c(c2)C(=O)N(C2CCC(=O)NC2=O)C3=O)cn1]

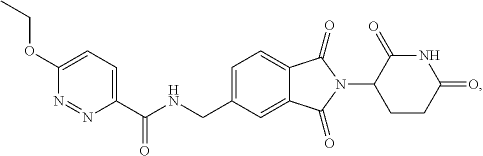 [SMILES: CCOc1ccc(C(=O)NCc2ccc3c(c2)C(=O)N(C2CCC(=O)NC2=O)C3=O)nn1]

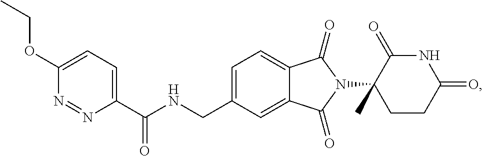 [SMILES: CCOc1ccc(C(=O)NCc2ccc3c(c2)C(=O)N([C@@]2(C)CCC(=O)NC2=O)C3=O)nn1]

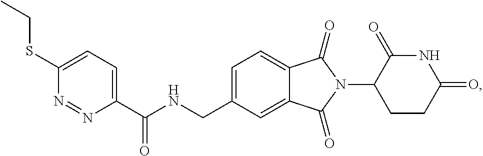 [SMILES: CCSc1ccc(C(=O)NCc2ccc3c(c2)C(=O)N(C2CCC(=O)NC2=O)C3=O)nn1]

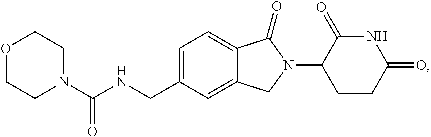 [SMILES: O=C1CCC(N2Cc3cc(CNC(=O)N4CCOCC4)ccc3C2=O)C(=O)N1]

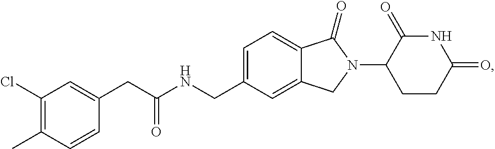 [SMILES: Cc1ccc(CC(=O)NCc2ccc3c(c2)CN(C2CCC(=O)NC2=O)C3=O)cc1Cl]

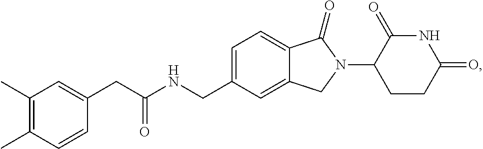 [SMILES: Cc1ccc(CC(=O)NCc2ccc3c(c2)CN(C2CCC(=O)NC2=O)C3=O)cc1C]

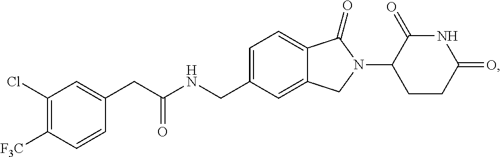 [SMILES: O=C(Cc1ccc(C(F)(F)F)c(Cl)c1)NCc1ccc2c(c1)CN(C1CCC(=O)NC1=O)C2=O]

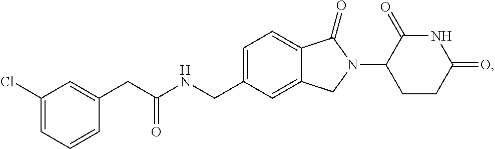 [SMILES: O=C(Cc1cccc(Cl)c1)NCc1ccc2c(c1)CN(C1CCC(=O)NC1=O)C2=O]

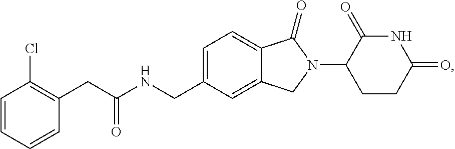 [SMILES: O=C(Cc1ccccc1Cl)NCc1ccc2c(c1)CN(C1CCC(=O)NC1=O)C2=O]

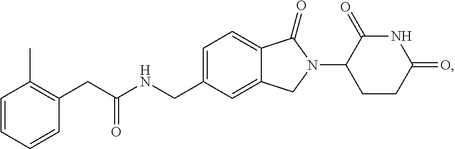 [SMILES: Cc1ccccc1CC(=O)NCc1ccc2c(c1)CN(C1CCC(=O)NC1=O)C2=O]

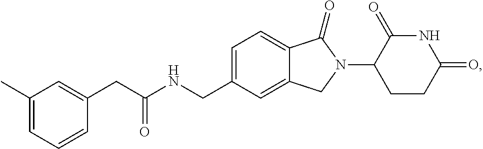 [SMILES: Cc1cccc(CC(=O)NCc2ccc3c(c2)CN(C2CCC(=O)NC2=O)C3=O)c1]

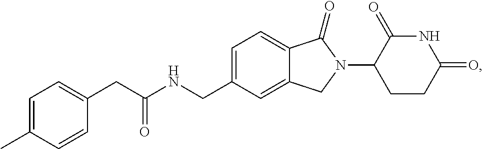 [SMILES: Cc1ccc(CC(=O)NCc2ccc3c(c2)CN(C2CCC(=O)NC2=O)C3=O)cc1]

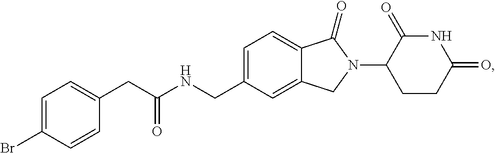 [SMILES: O=C(Cc1ccc(Br)cc1)NCc1ccc2c(c1)CN(C1CCC(=O)NC1=O)C2=O]

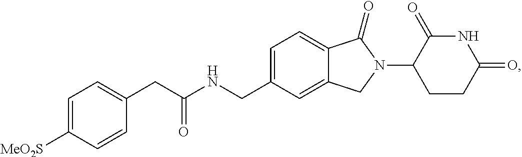 [SMILES: CS(=O)(=O)c1ccc(CC(=O)NCc2ccc3c(c2)CN(C2CCC(=O)NC2=O)C3=O)cc1]

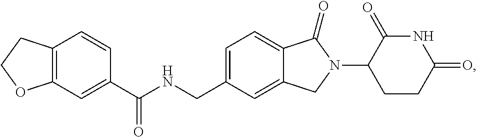 [SMILES: O=C1CCC(N2Cc3cc(CNC(=O)c4ccc5c(c4)OCC5)ccc3C2=O)C(=O)N1]

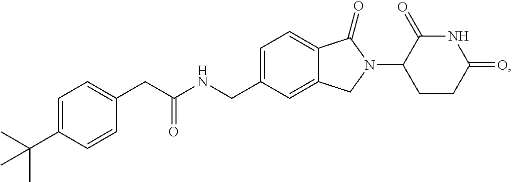 [SMILES: CC(C)(C)c1ccc(CC(=O)NCc2ccc3c(c2)CN(C2CCC(=O)NC2=O)C3=O)cc1]

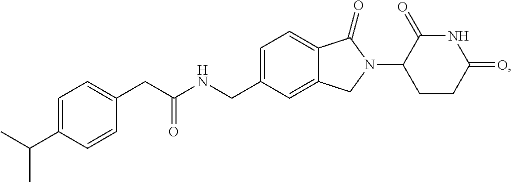 [SMILES: CC(C)c1ccc(CC(=O)NCc2ccc3c(c2)CN(C2CCC(=O)NC2=O)C3=O)cc1]

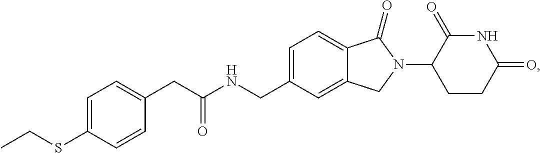 [SMILES: CCSc1ccc(CC(=O)NCc2ccc3c(c2)CN(C2CCC(=O)NC2=O)C3=O)cc1]

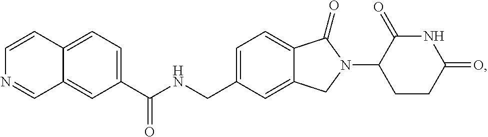 [SMILES: O=C1CCC(N2Cc3cc(CNC(=O)c4ccc5ccncc5c4)ccc3C2=O)C(=O)N1]

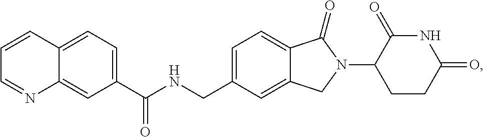 [SMILES: O=C1CCC(N2Cc3cc(CNC(=O)c4ccc5cccnc5c4)ccc3C2=O)C(=O)N1]

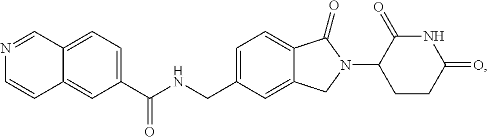 [SMILES: O=C1CCC(N2Cc3cc(CNC(=O)c4ccc5cnccc5c4)ccc3C2=O)C(=O)N1]

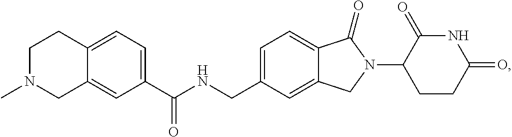 [SMILES: CN1CCc2ccc(C(=O)NCc3ccc4c(c3)CN(C3CCC(=O)NC3=O)C4=O)cc2C1]

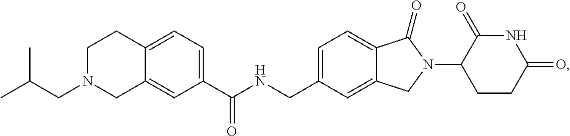 [SMILES: CC(C)CN1CCc2ccc(C(=O)NCc3ccc4c(c3)CN(C3CCC(=O)NC3=O)C4=O)cc2C1]

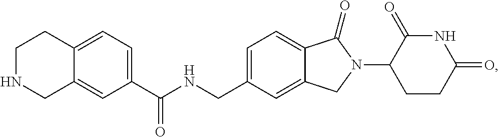 [SMILES: O=C1CCC(N2Cc3cc(CNC(=O)c4ccc5c(c4)CNCC5)ccc3C2=O)C(=O)N1]

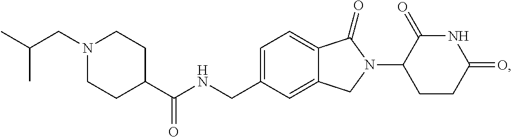 [SMILES: CC(C)CN1CCC(C(=O)NCc2ccc3c(c2)CN(C2CCC(=O)NC2=O)C3=O)CC1]

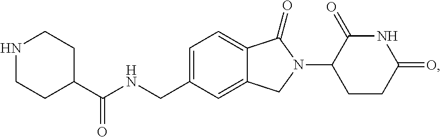 [SMILES: O=C1CCC(N2Cc3cc(CNC(=O)C4CCNCC4)ccc3C2=O)C(=O)N1]

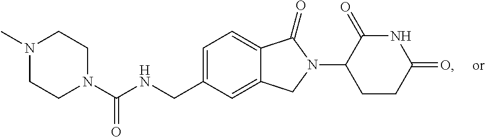 [SMILES: CN1CCN(C(=O)NCc2ccc3c(c2)CN(C2CCC(=O)NC2=O)C3=O)CC1]

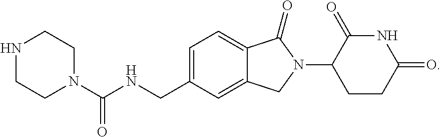 [SMILES: O=C1CCC(N2Cc3cc(CNC(=O)N4CCNCC4)ccc3C2=O)C(=O)N1]

In one embodiment, the immunomodulatory compound is:

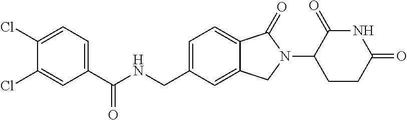 [SMILES: O=C1CCC(N2Cc3cc(CNC(=O)c4ccc(Cl)c(Cl)c4)ccc3C2=O)C(=O)N1]

or a pharmaceutically acceptable salt, solvate, prodrug, or stereoisomer thereof.

In one embodiment, the immunomodulatory compound is:

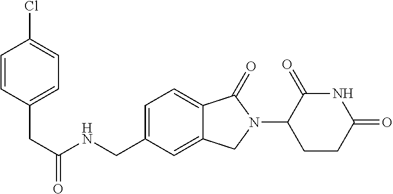 [SMILES: O=C(Cc1ccc(Cl)cc1)NCc1ccc2c(c1)CN(C1CCC(=O)NC1=O)C2=O]

or a pharmaceutically acceptable salt, solvate, prodrug, or stereoisomer thereof.

Still other representative compounds of formula:

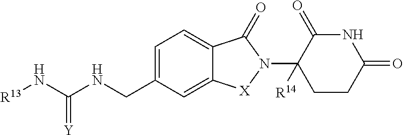

and pharmaceutically acceptable salts, solvates, stereoisomers, and prodrugs thereof,
wherein:

### X is CH₂ or C═O;

### Y is O or S;

R¹³ is: (C₁-C₁₀)alkyl; (C₁-C₁₀)alkoxy; 5 to 10 membered aryl or heteroaryl, optionally substituted with one or more of: halogen; cyano; (C₁-C₆)alkylenedioxy; (C₁-C₆)alkoxy, itself optionally substituted with one or more halogen; (C₁-C₆)alkyl, itself optionally substituted with one or more halogen; or (C₁-C₆)alkylthio, itself optionally substituted with one or more halogen; and
R¹⁴ is H or (C₁-C₆)alkyl.

In one embodiment, X is CH₂. In another embodiment, X is C═O.

In one embodiment, Y is O. In another embodiment, Y is S.

In one embodiment, R¹³ is (C₁-C₁₀)alkyl. In certain specific embodiments, R¹³ is (C₁-C₆)alkyl. In certain specific embodiments, R¹³ is propyl, butyl, pentyl, or hexyl.

In one embodiment, R¹³ is (C₁-C₁₀)alkoxy.

In one embodiment, R¹³ is 5 to 10 membered aryl or heteroaryl, optionally substituted with cyano. In certain specific embodiments, R¹³ is phenyl, optionally substituted with cyano.

In one embodiment, R¹³ is 5 to 10 membered aryl or heteroaryl, optionally substituted with (C₁-C₆)alkylenedioxy. In certain specific embodiments, R¹³ is phenyl, optionally substituted with methylenedioxy.

In one embodiment, R¹³ is 5 to 10 membered aryl or heteroaryl, optionally substituted with one or more halogen. In certain specific embodiments, R¹³ is phenyl, optionally substituted with one or more halogen.

In another embodiment, R¹³ is 5 to 10 membered aryl or heteroaryl, optionally substituted with (C₁-C₆)alkyl or (C₁-C₆)alkoxy, themselves optionally substituted with one or more halogens. In certain specific embodiments, R¹³ is phenyl, optionally substituted with methyl or methoxy, themselves optionally substituted with 1, 2, or 3 halogens.

In another embodiment, R¹³ is 5 to 10 membered aryl or heteroaryl, optionally substituted with (C₁-C₆)alkylthio, itself optionally substituted with one or more halogens.

In another embodiment, R¹⁴ is H. In another embodiment, R¹⁴ is (C₁-C₆)alkyl. In certain specific embodiments, R¹⁴ is methyl.

All of the combinations of the above embodiments are encompassed by this invention.

Examples include, but are not limited to, those listed in Table L, below, or a pharmaceutically acceptable salt, solvate (e.g., hydrate), prodrug or stereoisomer thereof:

TABLE L

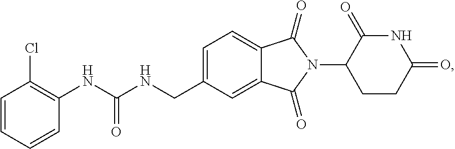 [SMILES: O=C1CCC(N2C(=O)c3ccc(CNC(=O)Nc4ccccc4Cl)cc3C2=O)C(=O)N1]

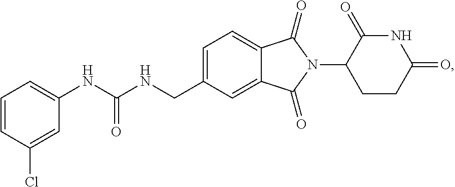 [SMILES: O=C1CCC(N2C(=O)c3ccc(CNC(=O)Nc4cccc(Cl)c4)cc3C2=O)C(=O)N1]

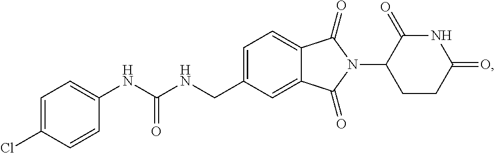 [SMILES: O=C1CCC(N2C(=O)c3ccc(CNC(=O)Nc4ccc(Cl)cc4)cc3C2=O)C(=O)N1]

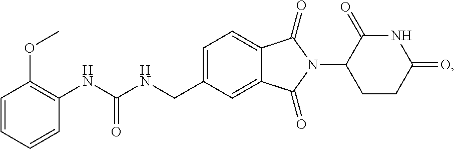 [SMILES: COc1ccccc1NC(=O)NCc1ccc2c(c1)C(=O)N(C1CCC(=O)NC1=O)C2=O]

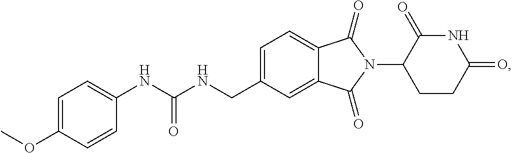 [SMILES: COc1ccc(NC(=O)NCc2ccc3c(c2)C(=O)N(C2CCC(=O)NC2=O)C3=O)cc1]

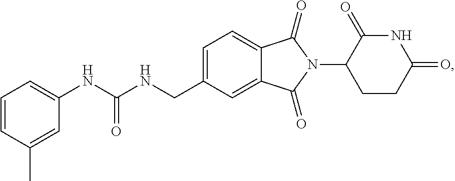 [SMILES: Cc1cccc(NC(=O)NCc2ccc3c(c2)C(=O)N(C2CCC(=O)NC2=O)C3=O)c1]

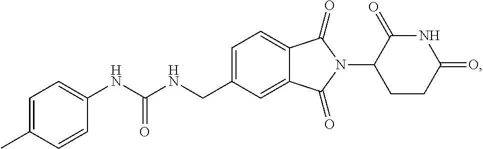 [SMILES: Cc1ccc(NC(=O)NCc2ccc3c(c2)C(=O)N(C2CCC(=O)NC2=O)C3=O)cc1]

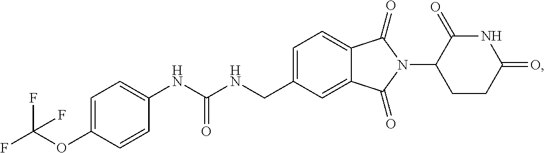 [SMILES: O=C1CCC(N2C(=O)c3ccc(CNC(=O)Nc4ccc(OC(F)(F)F)cc4)cc3C2=O)C(=O)N1]

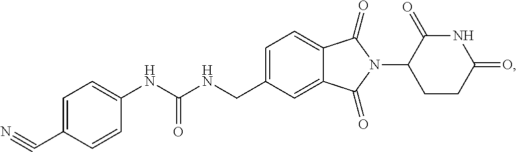 [SMILES: N#Cc1ccc(NC(=O)NCc2ccc3c(c2)C(=O)N(C2CCC(=O)NC2=O)C3=O)cc1]

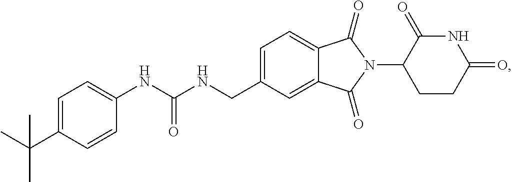 [SMILES: CC(C)(C)c1ccc(NC(=O)NCc2ccc3c(c2)C(=O)N(C2CCC(=O)NC2=O)C3=O)cc1]

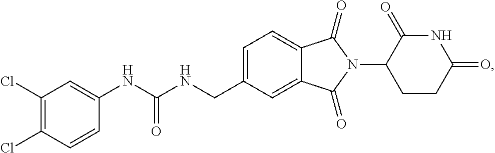 [SMILES: O=C1CCC(N2C(=O)c3ccc(CNC(=O)Nc4ccc(Cl)c(Cl)c4)cc3C2=O)C(=O)N1]

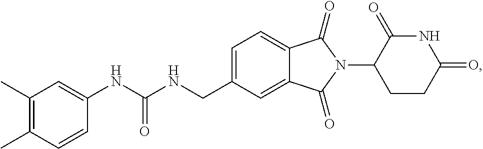 [SMILES: Cc1ccc(NC(=O)NCc2ccc3c(c2)C(=O)N(C2CCC(=O)NC2=O)C3=O)cc1C]

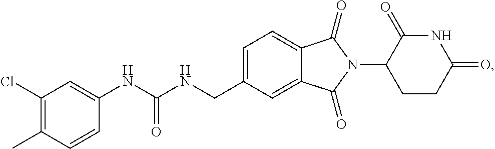 [SMILES: Cc1ccc(NC(=O)NCc2ccc3c(c2)C(=O)N(C2CCC(=O)NC2=O)C3=O)cc1Cl]

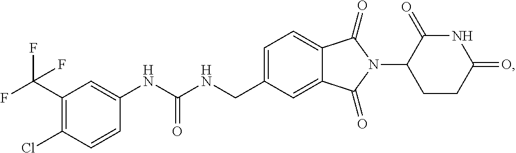 [SMILES: O=C1CCC(N2C(=O)c3ccc(CNC(=O)Nc4ccc(Cl)c(C(F)(F)F)c4)cc3C2=O)C(=O)N1]

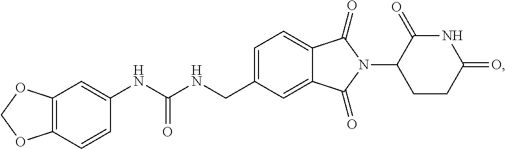 [SMILES: O=C1CCC(N2C(=O)c3ccc(CNC(=O)Nc4ccc5c(c4)OCO5)cc3C2=O)C(=O)N1]

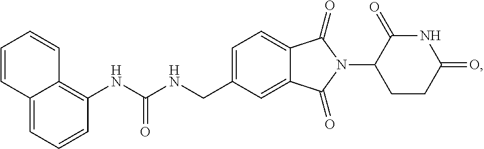 [SMILES: O=C1CCC(N2C(=O)c3ccc(CNC(=O)Nc4cccc5ccccc45)cc3C2=O)C(=O)N1]

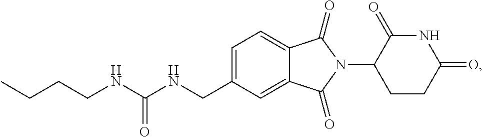 [SMILES: CCCCNC(=O)NCc1ccc2c(c1)C(=O)N(C1CCC(=O)NC1=O)C2=O]

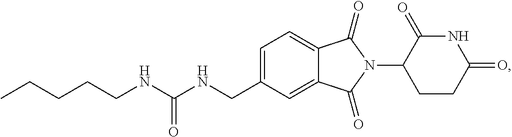 [SMILES: CCCCCNC(=O)NCc1ccc2c(c1)C(=O)N(C1CCC(=O)NC1=O)C2=O]

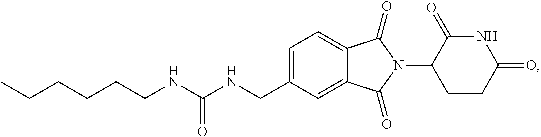 [SMILES: CCCCCCNC(=O)NCc1ccc2c(c1)C(=O)N(C1CCC(=O)NC1=O)C2=O]

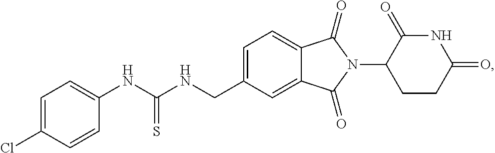 [SMILES: O=C1CCC(N2C(=O)c3ccc(CNC(=S)Nc4ccc(Cl)cc4)cc3C2=O)C(=O)N1]

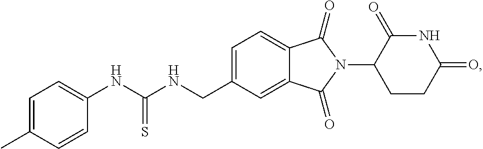 [SMILES: Cc1ccc(NC(=S)NCc2ccc3c(c2)C(=O)N(C2CCC(=O)NC2=O)C3=O)cc1]

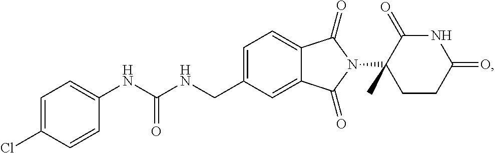

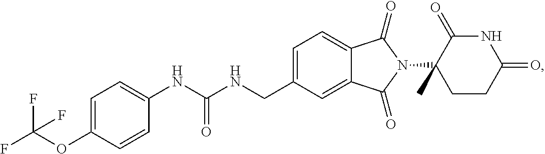

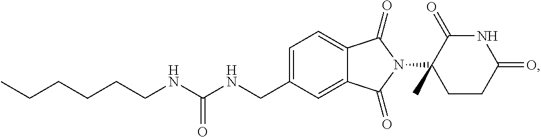

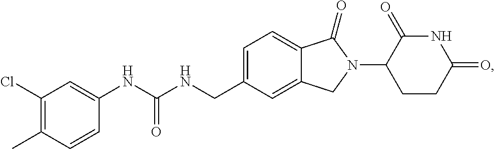 [SMILES: Cc1ccc(NC(=O)NCc2ccc3c(c2)CN(C2CCC(=O)NC2=O)C3=O)cc1Cl]

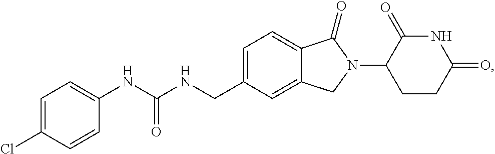 [SMILES: O=C1CCC(N2Cc3cc(CNC(=O)Nc4ccc(Cl)cc4)ccc3C2=O)C(=O)N1]

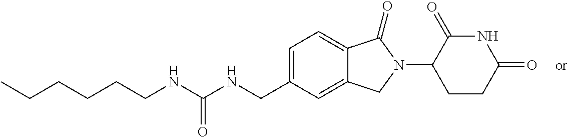 [SMILES: CCCCCCNC(=O)NCc1ccc2c(c1)CN(C1CCC(=O)NC1=O)C2=O]

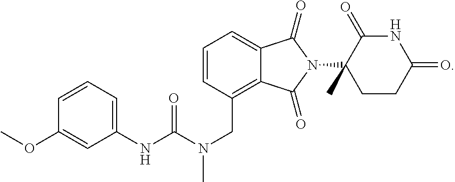

Other examples include, but are not limited to, those listed in Table M, below, or a pharmaceutically acceptable salt, solvate (e.g., hydrate), prodrug or stereoisomer thereof:

TABLE M

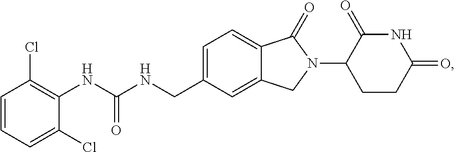 [SMILES: O=C1CCC(N2Cc3cc(CNC(=O)Nc4c(Cl)cccc4Cl)ccc3C2=O)C(=O)N1]

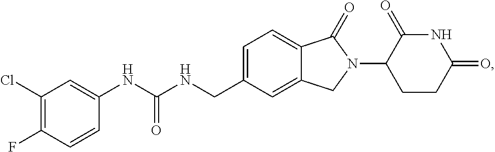 [SMILES: O=C1CCC(N2Cc3cc(CNC(=O)Nc4ccc(F)c(Cl)c4)ccc3C2=O)C(=O)N1]

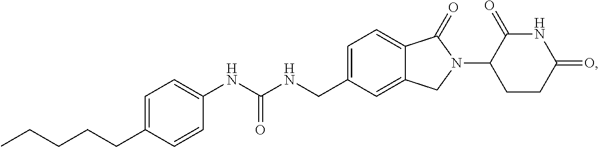 [SMILES: CCCCCc1ccc(NC(=O)NCc2ccc3c(c2)CN(C2CCC(=O)NC2=O)C3=O)cc1]

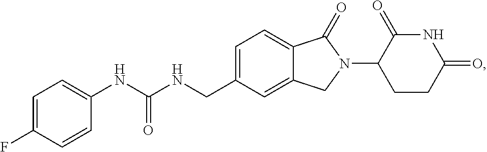 [SMILES: O=C1CCC(N2Cc3cc(CNC(=O)Nc4ccc(F)cc4)ccc3C2=O)C(=O)N1]

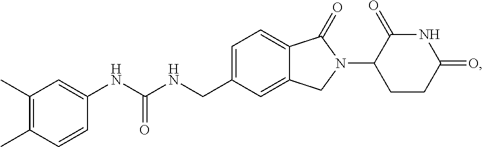 [SMILES: Cc1ccc(NC(=O)NCc2ccc3c(c2)CN(C2CCC(=O)NC2=O)C3=O)cc1C]

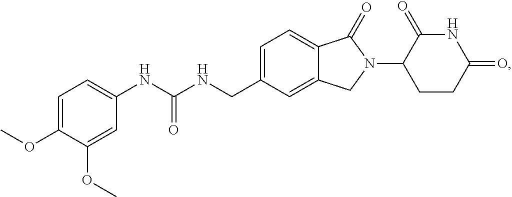 [SMILES: COc1ccc(NC(=O)NCc2ccc3c(c2)CN(C2CCC(=O)NC2=O)C3=O)cc1OC]

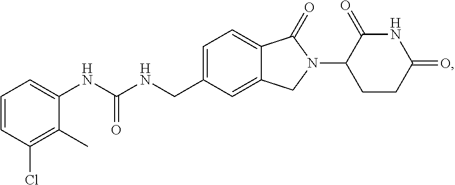 [SMILES: Cc1c(Cl)cccc1NC(=O)NCc1ccc2c(c1)CN(C1CCC(=O)NC1=O)C2=O]

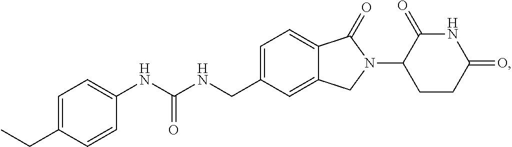 [SMILES: CCc1ccc(NC(=O)NCc2ccc3c(c2)CN(C2CCC(=O)NC2=O)C3=O)cc1]

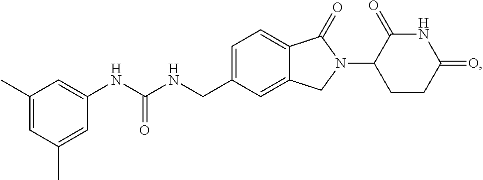 [SMILES: Cc1cc(C)cc(NC(=O)NCc2ccc3c(c2)CN(C2CCC(=O)NC2=O)C3=O)c1]

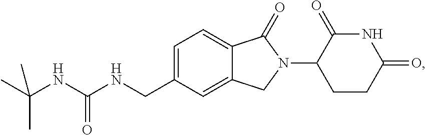 [SMILES: CC(C)(C)NC(=O)NCc1ccc2c(c1)CN(C1CCC(=O)NC1=O)C2=O]

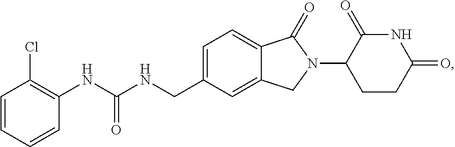 [SMILES: O=C1CCC(N2Cc3cc(CNC(=O)Nc4ccccc4Cl)ccc3C2=O)C(=O)N1]

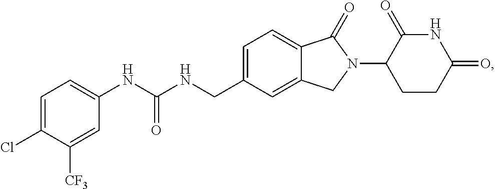 [SMILES: O=C1CCC(N2Cc3cc(CNC(=O)Nc4ccc(Cl)c(C(F)(F)F)c4)ccc3C2=O)C(=O)N1]

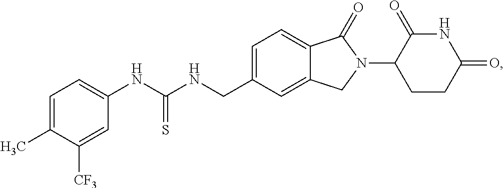 [SMILES: Cc1ccc(NC(=S)NCc2ccc3c(c2)CN(C2CCC(=O)NC2=O)C3=O)cc1C(F)(F)F]

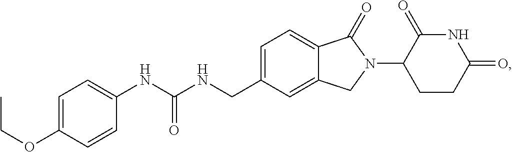 [SMILES: CCOc1ccc(NC(=O)NCc2ccc3c(c2)CN(C2CCC(=O)NC2=O)C3=O)cc1]

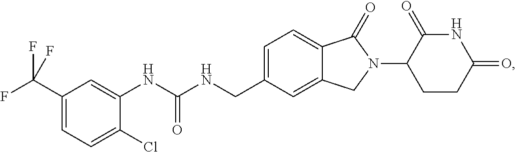 [SMILES: O=C1CCC(N2Cc3cc(CNC(=O)Nc4cc(C(F)(F)F)ccc4Cl)ccc3C2=O)C(=O)N1]

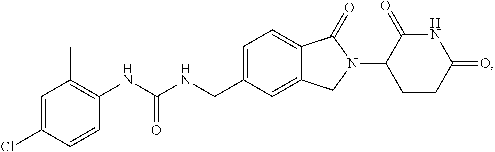 [SMILES: Cc1cc(Cl)ccc1NC(=O)NCc1ccc2c(c1)CN(C1CCC(=O)NC1=O)C2=O]

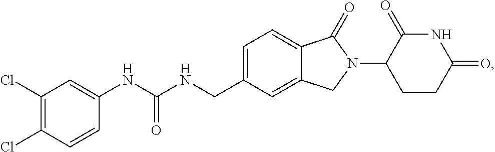 [SMILES: O=C1CCC(N2Cc3cc(CNC(=O)Nc4ccc(Cl)c(Cl)c4)ccc3C2=O)C(=O)N1]

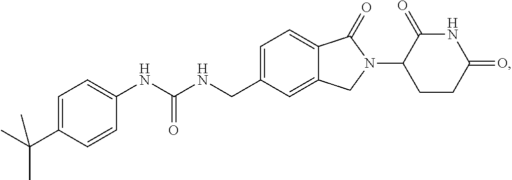 [SMILES: CC(C)(C)c1ccc(NC(=O)NCc2ccc3c(c2)CN(C2CCC(=O)NC2=O)C3=O)cc1]

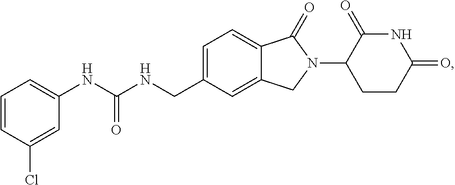 [SMILES: O=C1CCC(N2Cc3cc(CNC(=O)Nc4cccc(Cl)c4)ccc3C2=O)C(=O)N1]

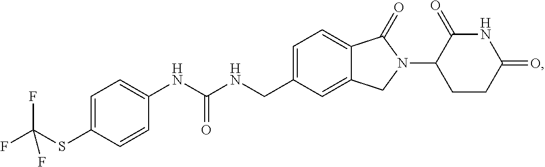 [SMILES: O=C1CCC(N2Cc3cc(CNC(=O)Nc4ccc(SC(F)(F)F)cc4)ccc3C2=O)C(=O)N1]

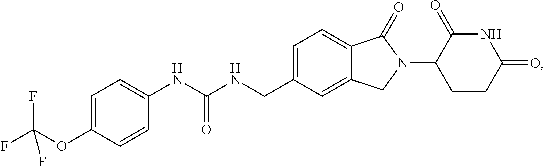 [SMILES: O=C1CCC(N2Cc3cc(CNC(=O)Nc4ccc(OC(F)(F)F)cc4)ccc3C2=O)C(=O)N1]

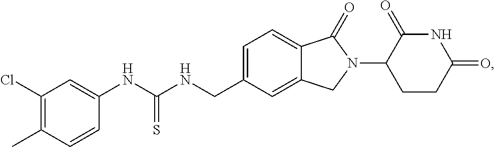 [SMILES: Cc1ccc(NC(=S)NCc2ccc3c(c2)CN(C2CCC(=O)NC2=O)C3=O)cc1Cl]

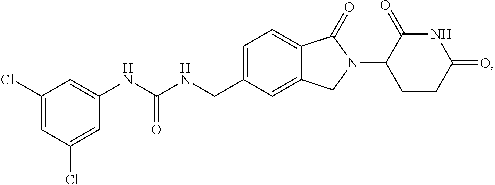 [SMILES: O=C1CCC(N2Cc3cc(CNC(=O)Nc4cc(Cl)cc(Cl)c4)ccc3C2=O)C(=O)N1]

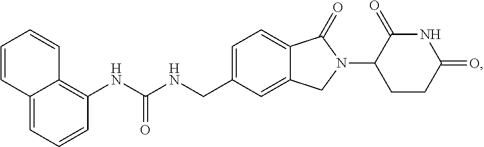 [SMILES: O=C1CCC(N2Cc3cc(CNC(=O)Nc4cccc5ccccc45)ccc3C2=O)C(=O)N1]

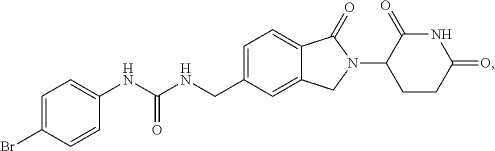 [SMILES: O=C1CCC(N2Cc3cc(CNC(=O)Nc4ccc(Br)cc4)ccc3C2=O)C(=O)N1]

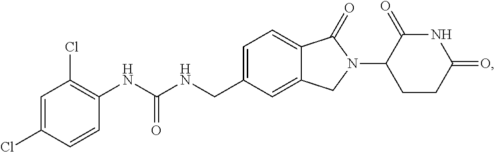 [SMILES: O=C1CCC(N2Cc3cc(CNC(=O)Nc4ccc(Cl)cc4Cl)ccc3C2=O)C(=O)N1]

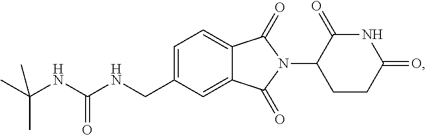 [SMILES: CC(C)(C)NC(=O)NCc1ccc2c(c1)C(=O)N(C1CCC(=O)NC1=O)C2=O]

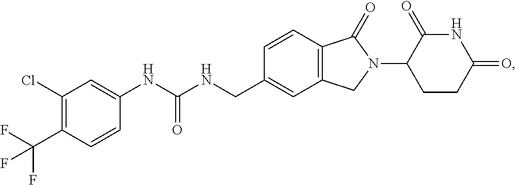 [SMILES: O=C1CCC(N2Cc3cc(CNC(=O)Nc4ccc(C(F)(F)F)c(Cl)c4)ccc3C2=O)C(=O)N1]

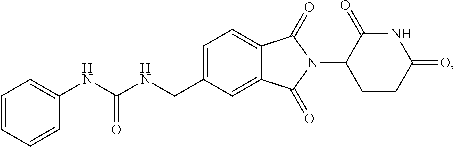 [SMILES: O=C1CCC(N2C(=O)c3ccc(CNC(=O)Nc4ccccc4)cc3C2=O)C(=O)N1]

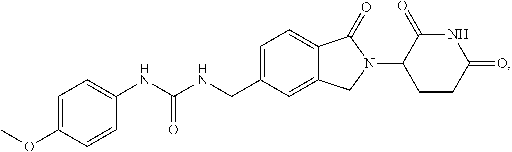 [SMILES: COc1ccc(NC(=O)NCc2ccc3c(c2)CN(C2CCC(=O)NC2=O)C3=O)cc1]

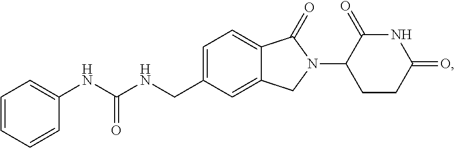 [SMILES: O=C1CCC(N2Cc3cc(CNC(=O)Nc4ccccc4)ccc3C2=O)C(=O)N1]

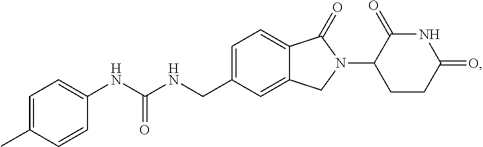 [SMILES: Cc1ccc(NC(=O)NCc2ccc3c(c2)CN(C2CCC(=O)NC2=O)C3=O)cc1]

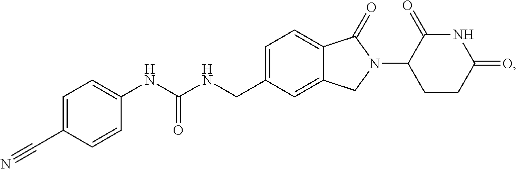 [SMILES: N#Cc1ccc(NC(=O)NCc2ccc3c(c2)CN(C2CCC(=O)NC2=O)C3=O)cc1]

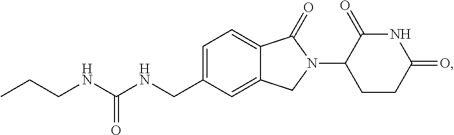 [SMILES: CCCNC(=O)NCc1ccc2c(c1)CN(C1CCC(=O)NC1=O)C2=O]

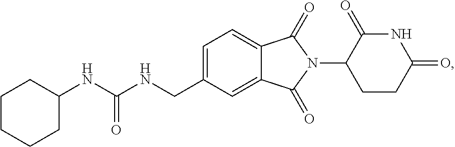 [SMILES: O=C1CCC(N2C(=O)c3ccc(CNC(=O)NC4CCCCC4)cc3C2=O)C(=O)N1]

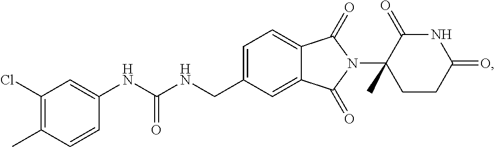 [SMILES: Cc1ccc(NC(=O)NCc2ccc3c(c2)C(=O)N([C@@]2(C)CCC(=O)NC2=O)C3=O)cc1Cl]

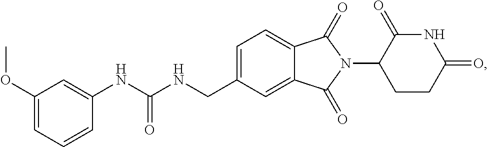 [SMILES: COc1cccc(NC(=O)NCc2ccc3c(c2)C(=O)N(C2CCC(=O)NC2=O)C3=O)c1]

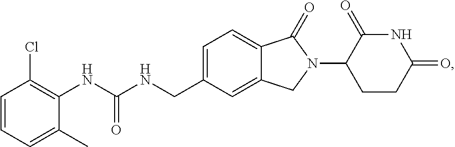 [SMILES: Cc1cccc(Cl)c1NC(=O)NCc1ccc2c(c1)CN(C1CCC(=O)NC1=O)C2=O]

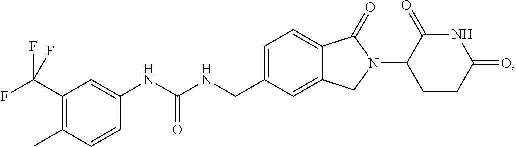 [SMILES: Cc1ccc(NC(=O)NCc2ccc3c(c2)CN(C2CCC(=O)NC2=O)C3=O)cc1C(F)(F)F]

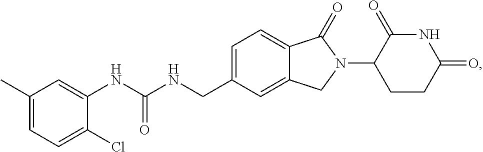 [SMILES: Cc1ccc(Cl)c(NC(=O)NCc2ccc3c(c2)CN(C2CCC(=O)NC2=O)C3=O)c1]

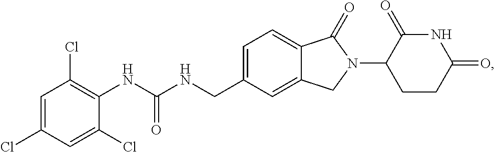 [SMILES: O=C1CCC(N2Cc3cc(CNC(=O)Nc4c(Cl)cc(Cl)cc4Cl)ccc3C2=O)C(=O)N1]

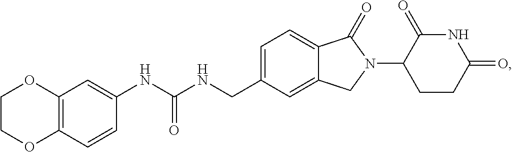 [SMILES: O=C1CCC(N2Cc3cc(CNC(=O)Nc4ccc5c(c4)OCCO5)ccc3C2=O)C(=O)N1]

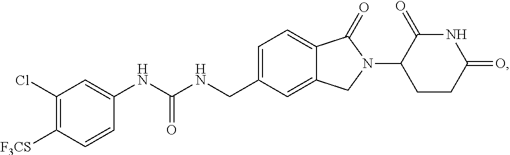

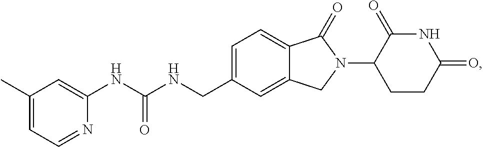 [SMILES: Cc1ccnc(NC(=O)NCc2ccc3c(c2)CN(C2CCC(=O)NC2=O)C3=O)c1]

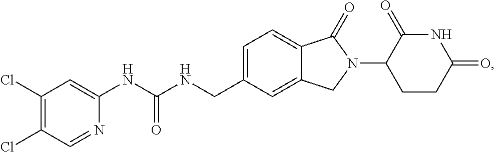 [SMILES: O=C1CCC(N2Cc3cc(CNC(=O)Nc4cc(Cl)c(Cl)cn4)ccc3C2=O)C(=O)N1]

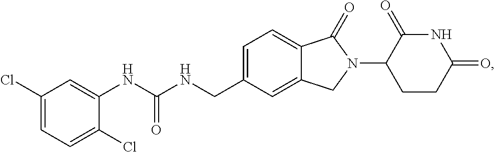 [SMILES: O=C1CCC(N2Cc3cc(CNC(=O)Nc4cc(Cl)ccc4Cl)ccc3C2=O)C(=O)N1]

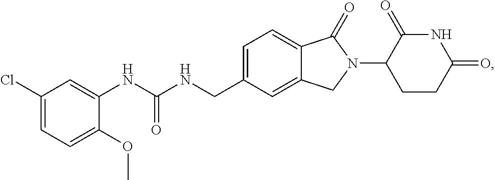 [SMILES: COc1ccc(Cl)cc1NC(=O)NCc1ccc2c(c1)CN(C1CCC(=O)NC1=O)C2=O]

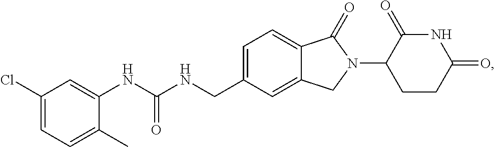 [SMILES: Cc1ccc(Cl)cc1NC(=O)NCc1ccc2c(c1)CN(C1CCC(=O)NC1=O)C2=O]

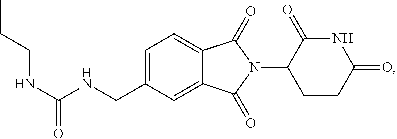 [SMILES: CCCNC(=O)NCc1ccc2c(c1)C(=O)N(C1CCC(=O)NC1=O)C2=O]

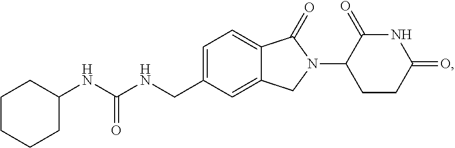 [SMILES: O=C1CCC(N2Cc3cc(CNC(=O)NC4CCCCC4)ccc3C2=O)C(=O)N1]

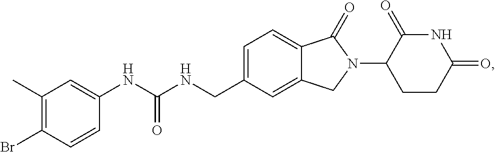 [SMILES: Cc1cc(NC(=O)NCc2ccc3c(c2)CN(C2CCC(=O)NC2=O)C3=O)ccc1Br]

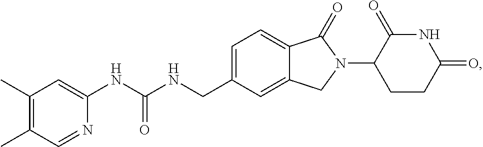 [SMILES: Cc1cnc(NC(=O)NCc2ccc3c(c2)CN(C2CCC(=O)NC2=O)C3=O)cc1C]

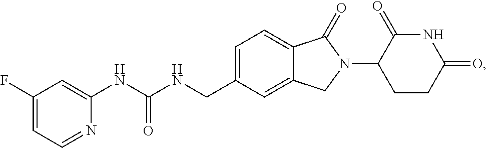 [SMILES: O=C1CCC(N2Cc3cc(CNC(=O)Nc4cc(F)ccn4)ccc3C2=O)C(=O)N1]

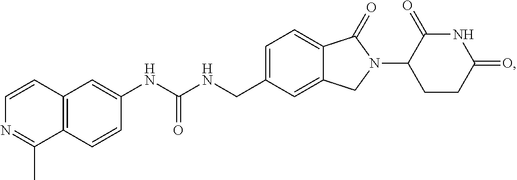 [SMILES: Cc1nccc2cc(NC(=O)NCc3ccc4c(c3)CN(C3CCC(=O)NC3=O)C4=O)ccc12]

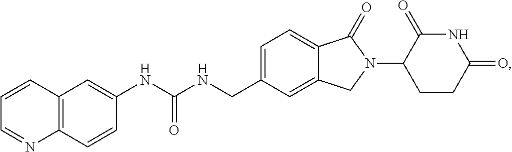 [SMILES: O=C1CCC(N2Cc3cc(CNC(=O)Nc4ccc5ncccc5c4)ccc3C2=O)C(=O)N1]

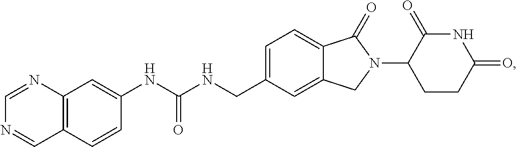 [SMILES: O=C1CCC(N2Cc3cc(CNC(=O)Nc4ccc5cncnc5c4)ccc3C2=O)C(=O)N1]

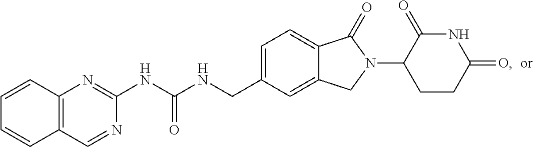 [SMILES: O=C1CCC(N2Cc3cc(CNC(=O)Nc4ncc5ccccc5n4)ccc3C2=O)C(=O)N1]

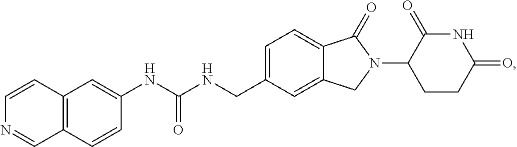 [SMILES: O=C1CCC(N2Cc3cc(CNC(=O)Nc4ccc5cnccc5c4)ccc3C2=O)C(=O)N1]

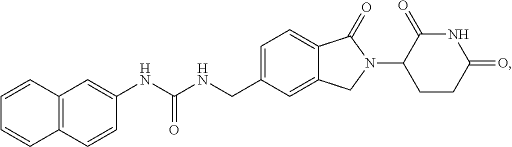 [SMILES: O=C1CCC(N2Cc3cc(CNC(=O)Nc4ccc5ccccc5c4)ccc3C2=O)C(=O)N1]

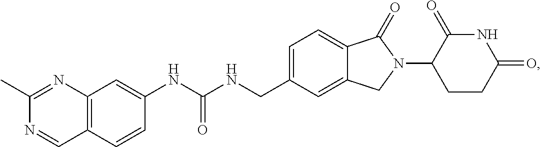 [SMILES: Cc1ncc2ccc(NC(=O)NCc3ccc4c(c3)CN(C3CCC(=O)NC3=O)C4=O)cc2n1]

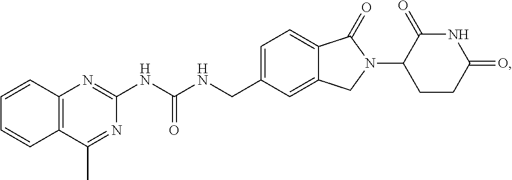 [SMILES: Cc1nc(NC(=O)NCc2ccc3c(c2)CN(C2CCC(=O)NC2=O)C3=O)nc2ccccc12]

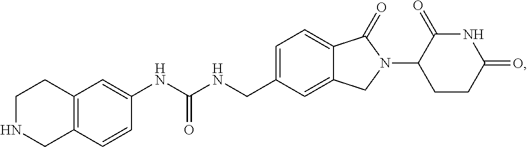 [SMILES: O=C1CCC(N2Cc3cc(CNC(=O)Nc4ccc5c(c4)CCNC5)ccc3C2=O)C(=O)N1]

In one embodiment, the immunomodulatory compound is:

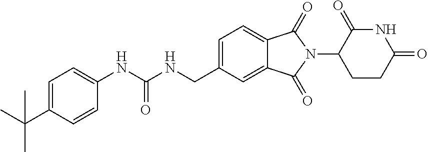 [SMILES: CC(C)(C)c1ccc(NC(=O)NCc2ccc3c(c2)C(=O)N(C2CCC(=O)NC2=O)C3=O)cc1]

or a pharmaceutically acceptable salt, solvate, prodrug, or stereoisomer thereof.

In one embodiment, the immunomodulatory compound is:

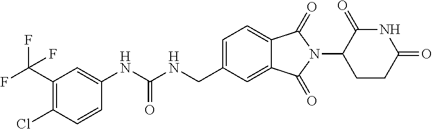 [SMILES: O=C1CCC(N2C(=O)c3ccc(CNC(=O)Nc4ccc(Cl)c(C(F)(F)F)c4)cc3C2=O)C(=O)N1]

or a pharmaceutically acceptable salt, solvate, prodrug, or stereoisomer thereof.

In one embodiment, the immunomodulatory compound is:

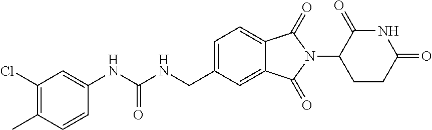 [SMILES: Cc1ccc(NC(=O)NCc2ccc3c(c2)C(=O)N(C2CCC(=O)NC2=O)C3=O)cc1Cl]

or a pharmaceutically acceptable salt, solvate, prodrug, or stereoisomer thereof.

In one embodiment, the immunomodulatory compound is:

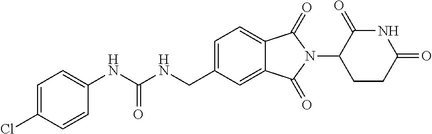 [SMILES: O=C1CCC(N2C(=O)c3ccc(CNC(=O)Nc4ccc(Cl)cc4)cc3C2=O)C(=O)N1]

or a pharmaceutically acceptable salt, solvate, prodrug, or stereoisomer thereof.

In one embodiment, the immunomodulatory compound is:

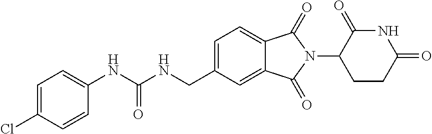 [SMILES: O=C1CCC(N2C(=O)c3ccc(CNC(=O)Nc4ccc(Cl)cc4)cc3C2=O)C(=O)N1]

or a pharmaceutically acceptable salt, solvate, prodrug, or stereoisomer thereof.

Still other specific IMiD® immunomodulatory drugs provided herein belong to a class of 4′-O-substituted isoindoline compounds disclosed in U.S. Pat. No. 8,153,659, the entirety of which is incorporated herein by reference. Representative compounds are of formula XII:

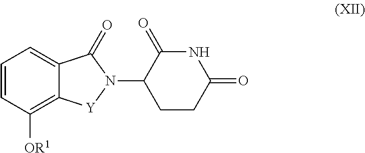

or a pharmaceutically acceptable salt, solvate, prodrug, clathrate, or stereoisomer thereof, wherein Y is C═O or CH₂, and R¹ is hydrogen, alkyl, alkenyl, alkynyl, aryl, aralkyl, cycloalkyl, cycloalkylalkyl, heterocyclyl, heterocyclylalkyl, heteroaryl, heteroarylalkyl, arylaminocarbonyl, alkylcarbonyl, alkylaminocarbonyl, dialkylaminocarbonyl, alkoxycarbonyl, cycloalkylcarbonyl, heteroarylcarbonyl or heterocyclylcarbonyl; where R¹ is optionally substituted with one or more, in certain embodiments, 1, 2, 3 or 4 substituents, one, two or three groups selected from alkoxy, halo, alkyl, carboxy, alkylaminocarbonyl, alkoxycarbonyl, nitro, amine, nitrile, haloalkyl, hydroxy, and alkylsulfonyl.

In one embodiment, Y is C═O. In another embodiment, Y is CH₂.

In certain embodiments, R¹ is alkyl, alkenyl, alkynyl, aryl, aralkyl, cycloalkyl, cycloalkylalkyl, heterocyclyl, heterocyclylalkyl, heteroaryl or heteroarylalkyl, optionally substituted with one or more, in one embodiment, one, two or three groups selected from alkoxy, halo, alkyl and alkylsulfonyl. In one embodiment, R¹ is aryl, aralkyl or heteroarylalkyl. In certain embodiments, the aryl or heteroaryl ring in group R¹ is a 5 or 6 membered monocyclic ring. In certain embodiments, the heteroaryl ring in R¹ group is a 5 or 6 membered monocyclic ring containing 1-3 heteroatoms selected from O, N and S. In certain embodiments, the aryl or heteroaryl ring in group R¹ is a bicyclic ring. In certain embodiments, the heteroaryl ring contains 1-3 heteroatoms selected from O, N and S and is attached to the alkyl group via a hetero atom in the ring. In certain embodiments, the heteroaryl ring is attached to the alkyl group via a carbon atom in the ring.

In one embodiment, R¹ is phenyl, benzyl, naphthylmethyl, quinolylmethyl, benzofurylmethyl, benzothienylmethyl, furylmethyl or thienylmethyl, optionally substituted with one or more, in one embodiment, one, two or three groups selected from alkoxy, halo, alkyl and alkylsulfonyl. In one embodiment, R¹ is optionally substituted with one or two substituents selected from methoxy, chloro, bromo, fluoro, methyl and methylsulfonyl.

In other embodiments, R¹ is 2-methoxyphenyl, benzyl, 3-chlorobenzyl, 4-chlorobenzyl, 3,4-dichlorobenzyl, 3,5-dichlorobenzyl, 3-fluorobenzyl, 3-bromobenzyl, 3-methylbenzyl, 4-methylsulfonylbenzyl, 3-methoxybenzyl, naphthylmethyl, 3-quinolylmethyl, 2-quinolylmethyl, 2-benzofurylmethyl, 2-benzothienylmethyl, 3-chlorothien-2-ylmethyl, 4-fluorobenzothien-2-ylmethyl, 2-furylmethyl, 5-chlorothien-2-ylmethyl or 1-naphth-2-ylethyl.

In one embodiment, R¹ is heterocyclyl. In certain embodiments, the heterocyclyl ring in R¹ group is a 5 or 6 membered monocyclic ring containing 1-3 heteroatoms selected from O, N and S. In certain embodiments, the heterocyclyl ring in group R¹ is piperidinyl or tetrahydropyranyl.

Representative compounds are of formula:

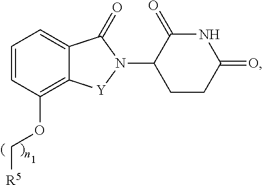

wherein Y is C═O or CH₂, and R⁵ is aryl or heteroaryl, optionally substituted with one, two or three groups selected from alkyl, halo, alkoxy, carboxy, alkylaminocarbonyl, alkoxycarbonyl, nitro, amine, nitrile, haloalkyl, hydroxy, and alkylsulfonyl; n₁ is 0-5, and the other variables are as described elsewhere herein.

In one embodiment, Y is C═O. In another embodiment, Y is CH₂.

In one embodiment, n₁ is 0 or 1. In certain embodiments, R⁵ is selected from phenyl, naphthyl, furyl, thienyl, benzofuryl, benzothienyl and quinolyl, optionally substituted with one or two groups selected from methyl, methoxy, chloro, fluoro, bromo and methylsulfonyl. In other embodiments, R⁵ is phenyl, 3-chlorophenyl, 4-chlorophenyl, 3,4-dichlorophenyl, 3,5-dichlorophenyl, 3-fluorophenyl, 3-bromophenyl, 3-methylphenyl, 4-methylsulfonylphenyl, 3-methoxyphenyl, naphthyl, 3-quinolyl, 2-quinolyl, 2-benzofuryl, 2-benzothienyl, 3-chlorothien-2-yl, 4-fluorobenzothien-2-yl, 2-furyl, 5-chlorothien-2-yl or 1-naphth-2-yl.

In one embodiment, n₁ is 0 or 1. In certain embodiments, R⁵ is selected from phenyl, benzyl, naphthyl, furyl, thienyl, benzofuryl, benzothienyl and quinolyl, optionally substituted with one or two groups selected from methyl, methoxy, chloro, fluoro, bromo and methylsulfonyl.

Other representative compounds are of formula

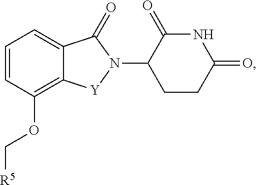

wherein the variables are as described elsewhere herein.

In one embodiment, Y is C═O. In another embodiment, Y is CH₂.

In one embodiment, R⁵ is

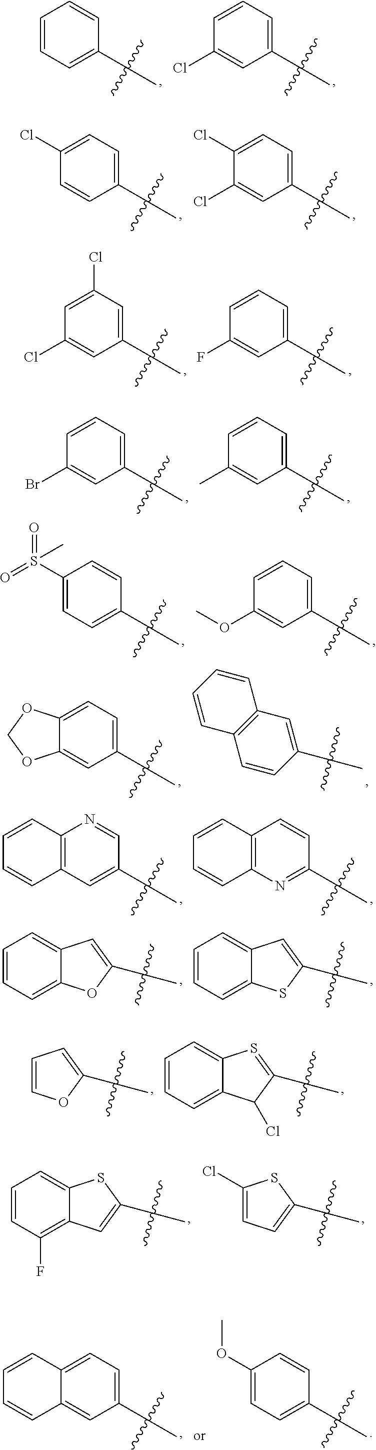

Examples include, but are not limited to, those listed in Table N, below, or a pharmaceutically acceptable salt, solvate (e.g., hydrate), prodrug, clathrate, or stereoisomer thereof:

TABLE N

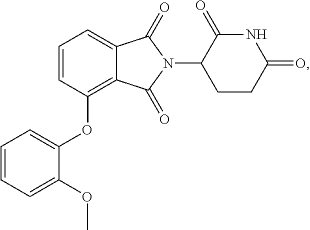 [SMILES: COc1ccccc1Oc1cccc2c1C(=O)N(C1CCC(=O)NC1=O)C2=O]

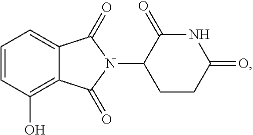 [SMILES: O=C1CCC(N2C(=O)c3cccc(O)c3C2=O)C(=O)N1]

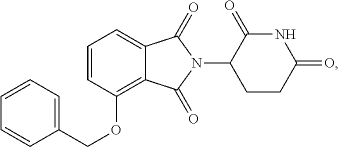 [SMILES: O=C1CCC(N2C(=O)c3cccc(OCc4ccccc4)c3C2=O)C(=O)N1]

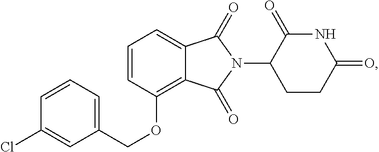 [SMILES: O=C1CCC(N2C(=O)c3cccc(OCc4cccc(Cl)c4)c3C2=O)C(=O)N1]

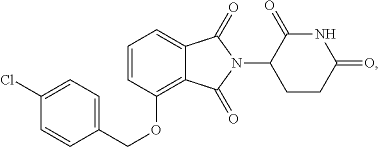 [SMILES: O=C1CCC(N2C(=O)c3cccc(OCc4ccc(Cl)cc4)c3C2=O)C(=O)N1]

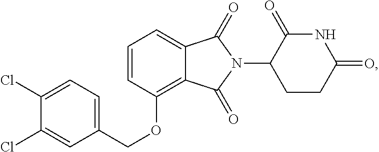 [SMILES: O=C1CCC(N2C(=O)c3cccc(OCc4ccc(Cl)c(Cl)c4)c3C2=O)C(=O)N1]

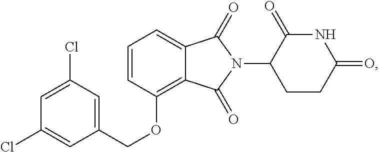 [SMILES: O=C1CCC(N2C(=O)c3cccc(OCc4cc(Cl)cc(Cl)c4)c3C2=O)C(=O)N1]

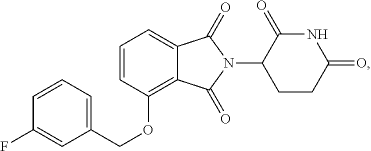 [SMILES: O=C1CCC(N2C(=O)c3cccc(OCc4cccc(F)c4)c3C2=O)C(=O)N1]

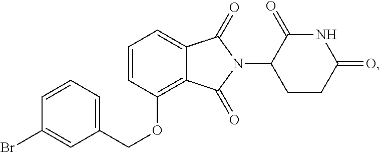 [SMILES: O=C1CCC(N2C(=O)c3cccc(OCc4cccc(Br)c4)c3C2=O)C(=O)N1]

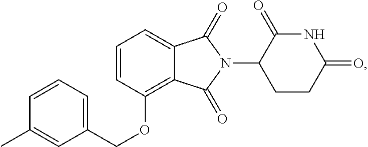 [SMILES: Cc1cccc(COc2cccc3c2C(=O)N(C2CCC(=O)NC2=O)C3=O)c1]

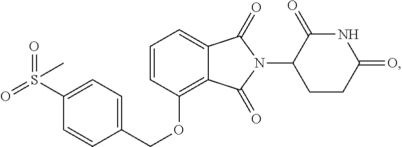 [SMILES: CS(=O)(=O)c1ccc(COc2cccc3c2C(=O)N(C2CCC(=O)NC2=O)C3=O)cc1]

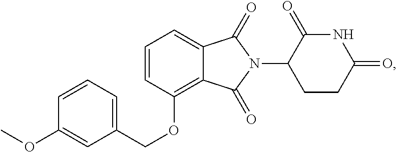 [SMILES: COc1cccc(COc2cccc3c2C(=O)N(C2CCC(=O)NC2=O)C3=O)c1]

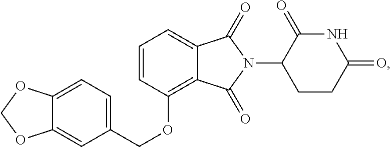 [SMILES: O=C1CCC(N2C(=O)c3cccc(OCc4ccc5c(c4)OCO5)c3C2=O)C(=O)N1]

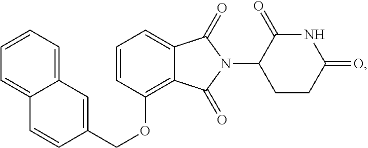 [SMILES: O=C1CCC(N2C(=O)c3cccc(OCc4ccc5ccccc5c4)c3C2=O)C(=O)N1]

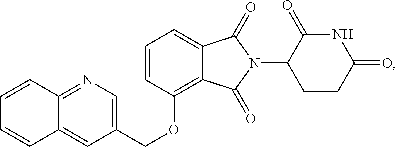 [SMILES: O=C1CCC(N2C(=O)c3cccc(OCc4cnc5ccccc5c4)c3C2=O)C(=O)N1]

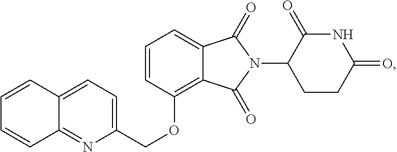 [SMILES: O=C1CCC(N2C(=O)c3cccc(OCc4ccc5ccccc5n4)c3C2=O)C(=O)N1]

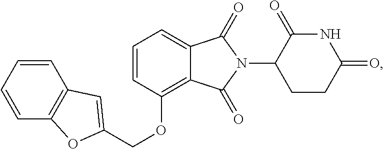 [SMILES: O=C1CCC(N2C(=O)c3cccc(OCc4cc5ccccc5o4)c3C2=O)C(=O)N1]

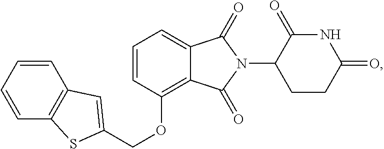 [SMILES: O=C1CCC(N2C(=O)c3cccc(OCc4cc5ccccc5s4)c3C2=O)C(=O)N1]

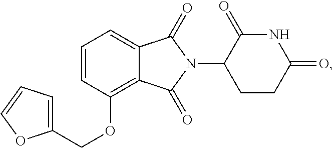 [SMILES: O=C1CCC(N2C(=O)c3cccc(OCc4ccco4)c3C2=O)C(=O)N1]

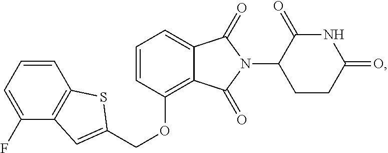 [SMILES: O=C1CCC(N2C(=O)c3cccc(OCc4cc5c(F)cccc5s4)c3C2=O)C(=O)N1]

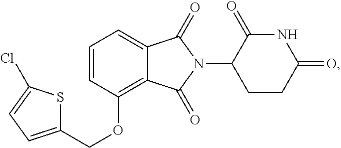 [SMILES: O=C1CCC(N2C(=O)c3cccc(OCc4ccc(Cl)s4)c3C2=O)C(=O)N1]

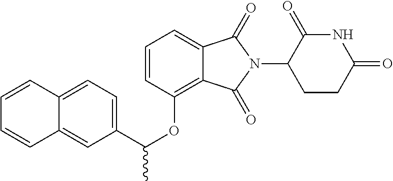 [SMILES: CC(Oc1cccc2c1C(=O)N(C1CCC(=O)NC1=O)C2=O)c1ccc2ccccc2c1]

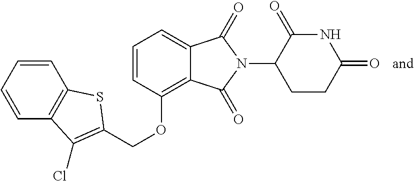 [SMILES: O=C1CCC(N2C(=O)c3cccc(OCc4sc5ccccc5c4Cl)c3C2=O)C(=O)N1]

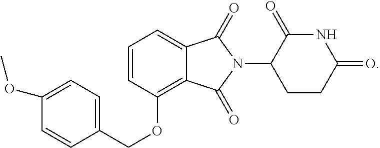 [SMILES: COc1ccc(COc2cccc3c2C(=O)N(C2CCC(=O)NC2=O)C3=O)cc1]

In certain embodiments, the compound is that listed in Table O, below, or a pharmaceutically acceptable salt, solvate (e.g., hydrate), prodrug, clathrate, or stereoisomer thereof:

TABLE O

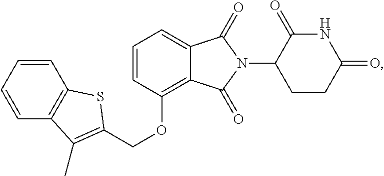 [SMILES: Cc1c(COc2cccc3c2C(=O)N(C2CCC(=O)NC2=O)C3=O)sc2ccccc12]

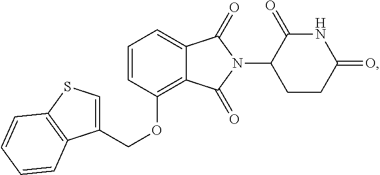 [SMILES: O=C1CCC(N2C(=O)c3cccc(OCc4csc5ccccc45)c3C2=O)C(=O)N1]

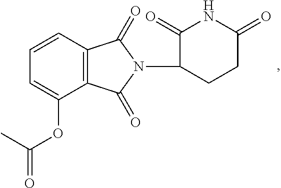 [SMILES: CC(=O)Oc1cccc2c1C(=O)N(C1CCC(=O)NC1=O)C2=O]

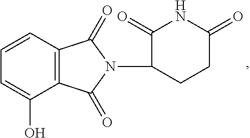 [SMILES: O=C1CCC(N2C(=O)c3cccc(O)c3C2=O)C(=O)N1]

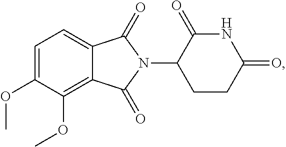 [SMILES: COc1ccc2c(c1OC)C(=O)N(C1CCC(=O)NC1=O)C2=O]

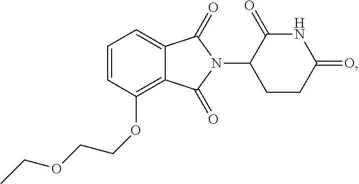 [SMILES: CCOCCOc1cccc2c1C(=O)N(C1CCC(=O)NC1=O)C2=O]

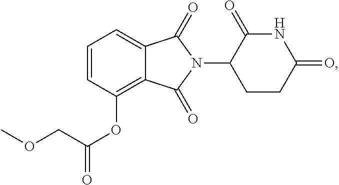 [SMILES: COCC(=O)Oc1cccc2c1C(=O)N(C1CCC(=O)NC1=O)C2=O]

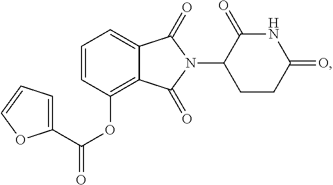 [SMILES: O=C1CCC(N2C(=O)c3cccc(OC(=O)c4ccco4)c3C2=O)C(=O)N1]

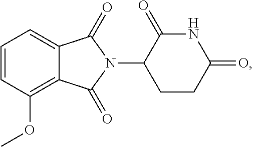 [SMILES: COc1cccc2c1C(=O)N(C1CCC(=O)NC1=O)C2=O]

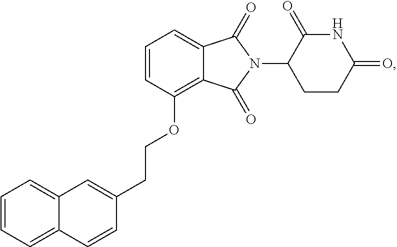 [SMILES: O=C1CCC(N2C(=O)c3cccc(OCCc4ccc5ccccc5c4)c3C2=O)C(=O)N1]

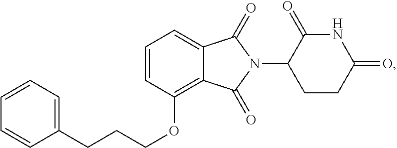 [SMILES: O=C1CCC(N2C(=O)c3cccc(OCCCc4ccccc4)c3C2=O)C(=O)N1]

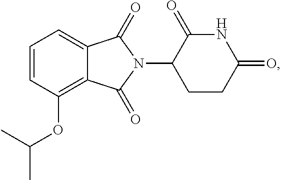 [SMILES: CC(C)Oc1cccc2c1C(=O)N(C1CCC(=O)NC1=O)C2=O]

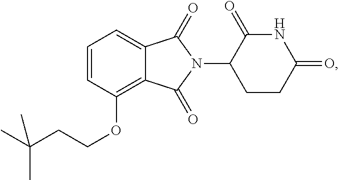 [SMILES: CC(C)(C)CCOc1cccc2c1C(=O)N(C1CCC(=O)NC1=O)C2=O]

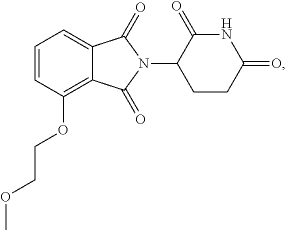 [SMILES: COCCOc1cccc2c1C(=O)N(C1CCC(=O)NC1=O)C2=O]

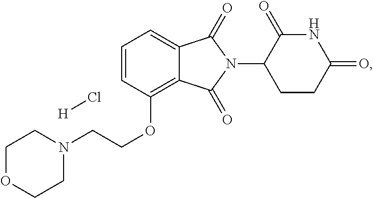

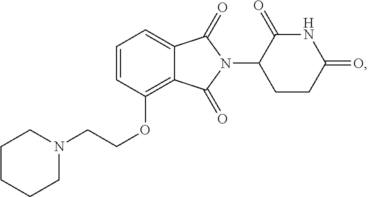 [SMILES: O=C1CCC(N2C(=O)c3cccc(OCCN4CCCCC4)c3C2=O)C(=O)N1]

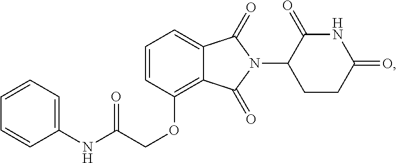 [SMILES: O=C1CCC(N2C(=O)c3cccc(OCC(=O)Nc4ccccc4)c3C2=O)C(=O)N1]

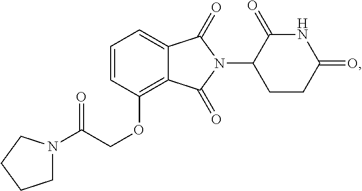 [SMILES: O=C1CCC(N2C(=O)c3cccc(OCC(=O)N4CCCC4)c3C2=O)C(=O)N1]

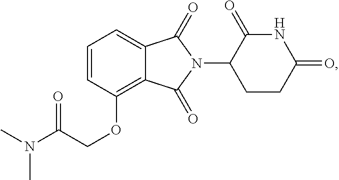 [SMILES: CN(C)C(=O)COc1cccc2c1C(=O)N(C1CCC(=O)NC1=O)C2=O]

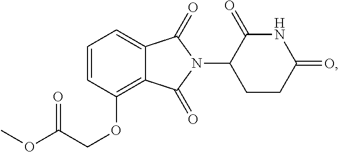 [SMILES: COC(=O)COc1cccc2c1C(=O)N(C1CCC(=O)NC1=O)C2=O]

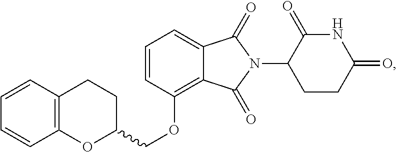 [SMILES: O=C1CCC(N2C(=O)c3cccc(OCC4CCc5ccccc5O4)c3C2=O)C(=O)N1]

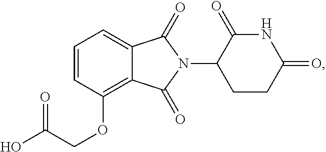 [SMILES: O=C(O)COc1cccc2c1C(=O)N(C1CCC(=O)NC1=O)C2=O]

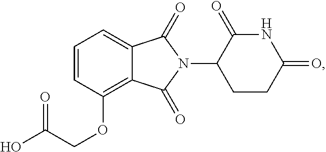 [SMILES: O=C(O)COc1cccc2c1C(=O)N(C1CCC(=O)NC1=O)C2=O]

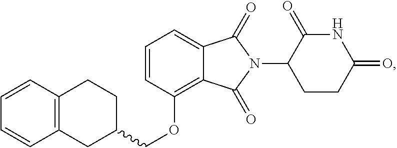 [SMILES: O=C1CCC(N2C(=O)c3cccc(OCC4CCc5ccccc5C4)c3C2=O)C(=O)N1]

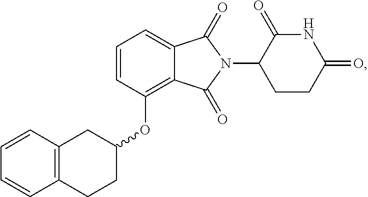 [SMILES: O=C1CCC(N2C(=O)c3cccc(OC4CCc5ccccc5C4)c3C2=O)C(=O)N1]

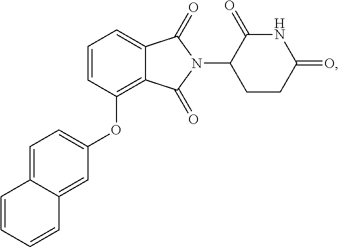 [SMILES: O=C1CCC(N2C(=O)c3cccc(Oc4ccc5ccccc5c4)c3C2=O)C(=O)N1]

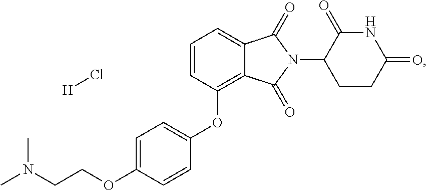 [SMILES: CN(C)CCOc1ccc(Oc2cccc3c2C(=O)N(C2CCC(=O)NC2=O)C3=O)cc1 ; Cl]

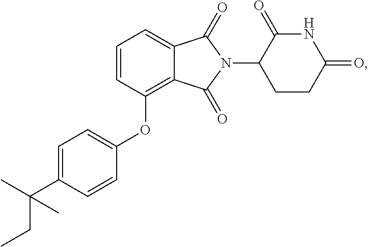 [SMILES: CCC(C)(C)c1ccc(Oc2cccc3c2C(=O)N(C2CCC(=O)NC2=O)C3=O)cc1]

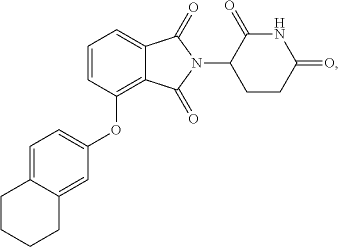 [SMILES: O=C1CCC(N2C(=O)c3cccc(Oc4ccc5c(c4)CCCC5)c3C2=O)C(=O)N1]

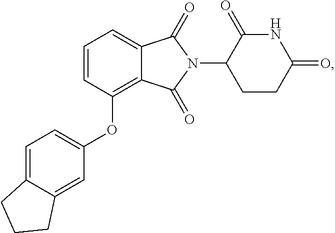 [SMILES: O=C1CCC(N2C(=O)c3cccc(Oc4ccc5c(c4)CCC5)c3C2=O)C(=O)N1]

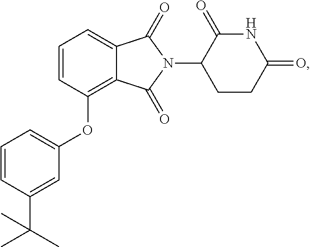 [SMILES: CC(C)(C)c1cccc(Oc2cccc3c2C(=O)N(C2CCC(=O)NC2=O)C3=O)c1]

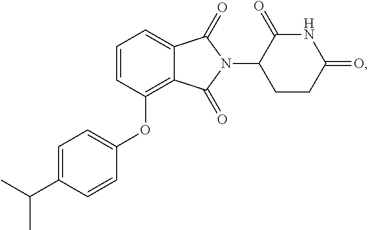 [SMILES: CC(C)c1ccc(Oc2cccc3c2C(=O)N(C2CCC(=O)NC2=O)C3=O)cc1]

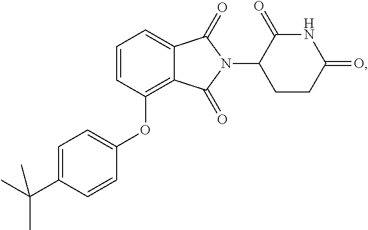 [SMILES: CC(C)(C)c1ccc(Oc2cccc3c2C(=O)N(C2CCC(=O)NC2=O)C3=O)cc1]

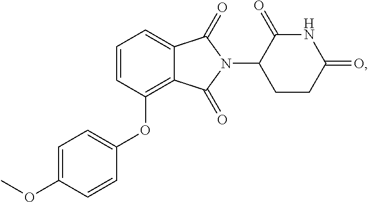 [SMILES: COc1ccc(Oc2cccc3c2C(=O)N(C2CCC(=O)NC2=O)C3=O)cc1]

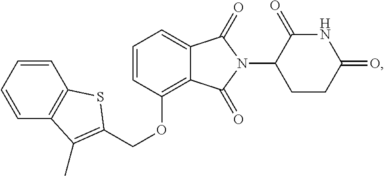 [SMILES: Cc1c(COc2cccc3c2C(=O)N(C2CCC(=O)NC2=O)C3=O)sc2ccccc12]

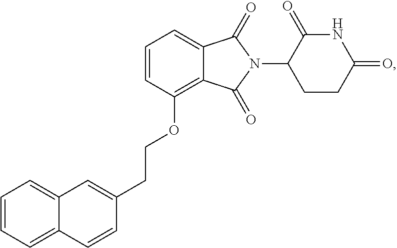 [SMILES: O=C1CCC(N2C(=O)c3cccc(OCCc4ccc5ccccc5c4)c3C2=O)C(=O)N1]

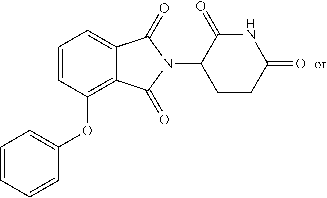 [SMILES: O=C1CCC(N2C(=O)c3cccc(Oc4ccccc4)c3C2=O)C(=O)N1]

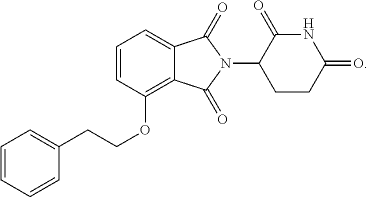 [SMILES: O=C1CCC(N2C(=O)c3cccc(OCCc4ccccc4)c3C2=O)C(=O)N1]

In one embodiment, the compound is selected from those listed in Table P, below, or a pharmaceutically acceptable salt, solvate (e.g., hydrate), prodrug, clathrate, or stereoisomer thereof:

TABLE P

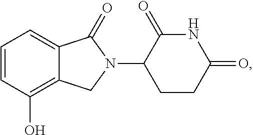 [SMILES: O=C1CCC(N2Cc3c(O)cccc3C2=O)C(=O)N1]

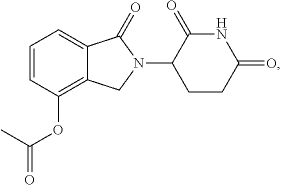 [SMILES: CC(=O)Oc1cccc2c1CN(C1CCC(=O)NC1=O)C2=O]

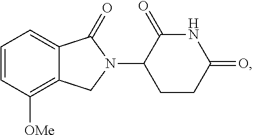 [SMILES: COc1cccc2c1CN(C1CCC(=O)NC1=O)C2=O]

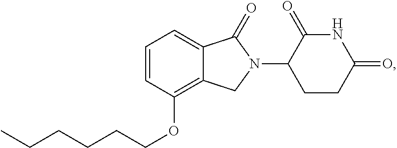 [SMILES: CCCCCCOc1cccc2c1CN(C1CCC(=O)NC1=O)C2=O]

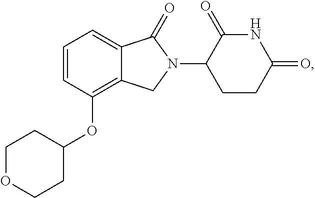 [SMILES: O=C1CCC(N2Cc3c(OC4CCOCC4)cccc3C2=O)C(=O)N1]

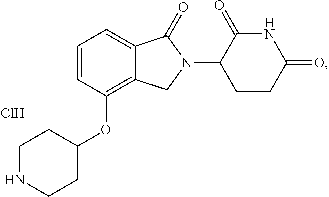 [SMILES: Cl ; O=C1CCC(N2Cc3c(OC4CCNCC4)cccc3C2=O)C(=O)N1]

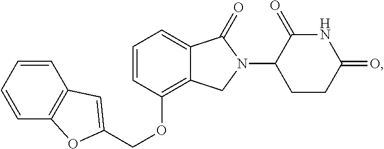 [SMILES: O=C1CCC(N2Cc3c(OCc4cc5ccccc5o4)cccc3C2=O)C(=O)N1]

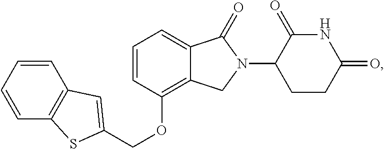 [SMILES: O=C1CCC(N2Cc3c(OCc4cc5ccccc5s4)cccc3C2=O)C(=O)N1]

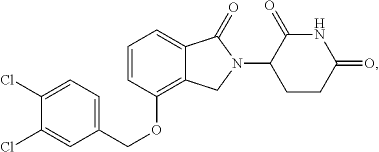 [SMILES: O=C1CCC(N2Cc3c(OCc4ccc(Cl)c(Cl)c4)cccc3C2=O)C(=O)N1]

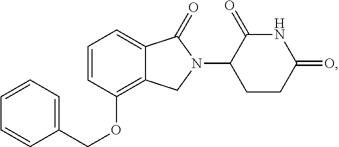 [SMILES: O=C1CCC(N2Cc3c(OCc4ccccc4)cccc3C2=O)C(=O)N1]

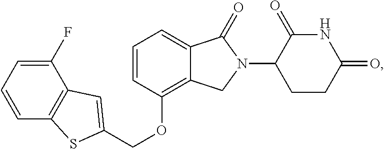 [SMILES: O=C1CCC(N2Cc3c(OCc4cc5c(F)cccc5s4)cccc3C2=O)C(=O)N1]

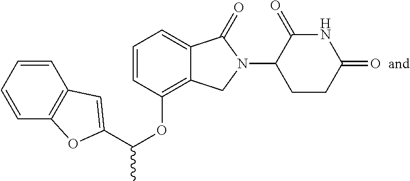 [SMILES: CC(Oc1cccc2c1CN(C1CCC(=O)NC1=O)C2=O)c1cc2ccccc2o1]

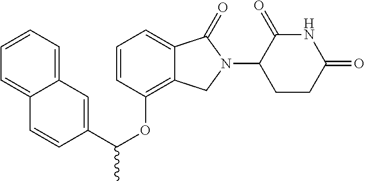 [SMILES: CC(Oc1cccc2c1CN(C1CCC(=O)NC1=O)C2=O)c1ccc2ccccc2c1]

Still other specific IMiD® immunomodulatory drugs provided herein belong to a class of isoindoline compounds disclosed in U.S. Pat. No. 8,129,375, the entirety of which is incorporated herein by reference. Representative compounds are of formula XIII:

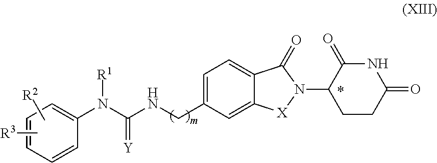

or a pharmaceutically acceptable salt, solvate, prodrug, or stereoisomer thereof, wherein:

X is C(═O) or CH₂;

Y is O, cyanamido (N—≡N), or amido (NH);

m is an integer of 0, 1, 2, or 3;

R¹ is hydrogen or C₁₋₆ alkyl;

R² is hydrogen, —NO₂, C₁₋₁₀ alkyl, C₀₋₆ alkyl- (5 to 10 membered heteroaryl), C₀₋₆ alkyl- (5 to 6 membered heterocyclyl), C₀₋₆ alkyl-OH, C₀₋₄ alkyl-NH₂, —NHCO—C₁₋₆ alkyl, —OR²¹, or —(CH₂—Z)₀₋₂— (5 to 10 membered heteroaryl), where each heteroaryl and heterocyclyl is optionally substituted with one or more C₁₋₆ alkyl;

R³ is hydrogen, halogen, —NO₂, C₀₋₆ alkyl- (5 to 10 membered heteroaryl), C₀₋₆ alkyl- (5 to 6 membered heterocyclyl), C₀₋₆ alkyl-OH, C₀₋₄ alkyl-NH₂, —NHCO—C₁₋₆ alkyl, —OR²¹, or —(CH₂—Z)₀₋₂— (5 to 10 membered heteroaryl), where each heteroaryl and heterocyclyl is optionally substituted with one or more C₁₋₆ alkyl;

R²¹ is C₆₋₁₀ aryl, 5 to 10 membered heteroaryl, 5 to 6 membered heterocyclyl, or —CO(CH₂)₀₋₂R²², wherein the aryl, heteroaryl, and heterocyclyl are each optionally substituted with one or more C₁₋₆ alkyl;

R²² is —NH₂ or 5 to 6 membered heterocyclyl; and

Z is CH₂, NH, or O;

with the proviso that when R¹ is hydrogen, then R² is not hydrogen or C₁₋₁₀ alkyl;

with the proviso that when Y is O, then R³ is not halogen; and

with the proviso that when Y is O and R³ is halogen, then R² is C₀₋₆ alkyl- (5-6 membered heterocyclyl).

In certain embodiments, X is CH₂. In certain embodiments, X is C(═O).

In certain embodiments, Y is O. In certain embodiments, Y is cyanamido. In certain embodiments, Y is amido.

In certain embodiments, Z is CH₂. In certain embodiments, Z is NH. In certain embodiments, Z is O.

In certain embodiments, m is 0. In certain embodiments, m is 1. In certain embodiments, m is 2. In certain embodiments, m is 3.

In certain embodiments, R¹ is hydrogen. In certain embodiments, R¹ is C₁₋₆ alkyl, optionally substituted with one, two, or three substituents Q as described herein. In certain embodiments, R¹ is methyl.

In certain embodiments, R² is hydrogen. In certain embodiments, R² is halogen. In certain embodiments, R² is nitro. In certain embodiments, R² is C₁₋₁₀ alkyl. In certain embodiments, R² is C₀₋₆ alkyl- (5 to 10 membered heteroaryl), where the heteroaryl is optionally substituted with one or more C₁₋₆ alkyl. In certain embodiments, R² is C₀₋₆ alkyl-(5 to 6 membered heterocyclyl), where the heterocyclyl is optionally substituted with one or more C₁₋₆ alkyl. In certain embodiments, R² is C₀₋₆ alkyl-OH. In certain embodiments, R² is C₀₋₄ alkyl-NH₂. In certain embodiments, R² is —NHCO—C₁₋₆ alkyl. In certain embodiments, R² is —OR²¹, wherein R²¹ is as described herein. In certain embodiments, R² is or —(CH₂—Y)₀₋₂— (5 to 10 membered heteroaryl), where the heteroaryl is optionally substituted with one or more C₁₋₆ alkyl. In certain embodiments, R² is hydrogen, amino, acetamido, hydroxy, nitro, aminomethyl, hydroxymethyl, 2-methyl-1H-imidazol-1-yl, 3-methyl-1,2,4-oxadiazol-5-yl, 4-methylpiperazin-1-yl)methyl, 2-methyl-2H-pyrazol-3-yl, 1-methyl-1H-pyrazol-3-yl, 2-methylthiazol-4-yl, 4-methyl-4H-1,2,4-triazol-3-yl, morpholinomethyl, (pyridin-4-yl)methyl, (pyridin-4-yloxy)methyl, phenoxy, pyridin-2-yloxy, piperidin-4-yloxy, 2-aminoacetoxy, or 2-piperazin-1-ylacetoxy.

In certain embodiments, R³ is hydrogen. In certain embodiments, R³ is nitro. In certain embodiments, R³ is C₀₋₆ alkyl- (5 to 10 membered heteroaryl), where the heteroaryl is optionally substituted with one or more C₁₋₆ alkyl. In certain embodiments, R³ is C₀₋₆ alkyl- (5 to 6 membered heterocyclyl), where the heterocyclyl is optionally substituted with one or more C₁₋₆ alkyl. In certain embodiments, R³ is C₀₋₆ alkyl-OH. In certain embodiments, R³ is C₀₋₄ alkyl-NH₂. In certain embodiments, R³ is —NHCO—C₁₋₆ alkyl. In certain embodiments, R³ is —OR²¹, wherein R²¹ is as described herein. In certain embodiments, R³ is or —(CH₂—Y)₀₋₂— (5 to 10 membered heteroaryl), where the heteroaryl is optionally substituted with one or more C₁₋₆ alkyl. In certain embodiments, R³ is hydrogen, amino, acetamido, hydroxy, nitro, methyl, aminomethyl, hydroxymethyl, 2-methyl-1H-imidazol-1-yl, 3-methyl-1,2,4-oxadiazol-5-yl, 4-methylpiperazin-1-yl)methyl, 2-methyl-2H-pyrazol-3-yl, 1-methyl-1H-pyrazol-3-yl, 2-methylthiazol-4-yl, 4-methyl-4H-1,2,4-triazol-3-yl, morpholinomethyl, (pyridin-4-yl)methyl, (pyridin-4-yloxy)methyl, phenoxy, pyridin-2-yloxy, piperidin-4-yloxy, 2-aminoacetoxy, or 2-piperazin-1-ylacetoxy.

In one embodiment, the compound is selected from those listed in Table Q, below:

TABLE Q

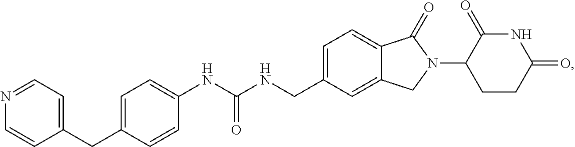 [SMILES: O=C1CCC(N2Cc3cc(CNC(=O)Nc4ccc(Cc5ccncc5)cc4)ccc3C2=O)C(=O)N1]

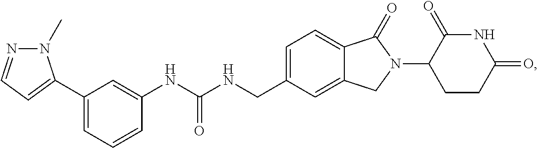 [SMILES: Cn1nccc1-c1cccc(NC(=O)NCc2ccc3c(c2)CN(C2CCC(=O)NC2=O)C3=O)c1]

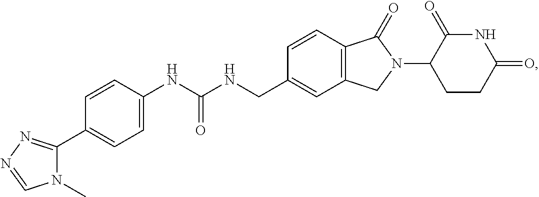 [SMILES: Cn1cnnc1-c1ccc(NC(=O)NCc2ccc3c(c2)CN(C2CCC(=O)NC2=O)C3=O)cc1]

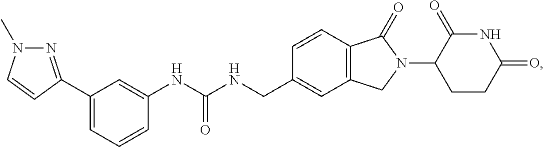 [SMILES: Cn1ccc(-c2cccc(NC(=O)NCc3ccc4c(c3)CN(C3CCC(=O)NC3=O)C4=O)c2)n1]

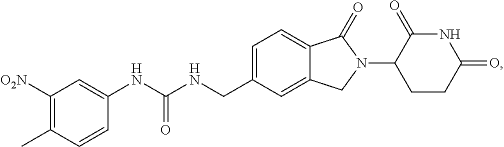 [SMILES: Cc1ccc(NC(=O)NCc2ccc3c(c2)CN(C2CCC(=O)NC2=O)C3=O)cc1[N+](=O)[O-]]

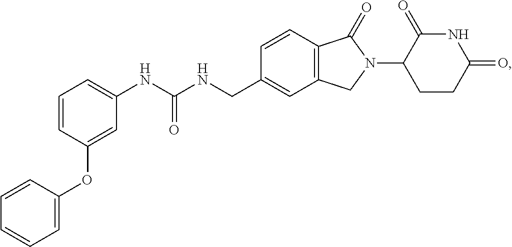 [SMILES: O=C1CCC(N2Cc3cc(CNC(=O)Nc4cccc(Oc5ccccc5)c4)ccc3C2=O)C(=O)N1]

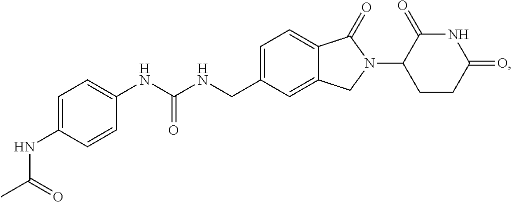 [SMILES: CC(=O)Nc1ccc(NC(=O)NCc2ccc3c(c2)CN(C2CCC(=O)NC2=O)C3=O)cc1]

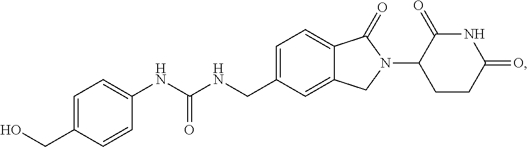 [SMILES: O=C1CCC(N2Cc3cc(CNC(=O)Nc4ccc(CO)cc4)ccc3C2=O)C(=O)N1]

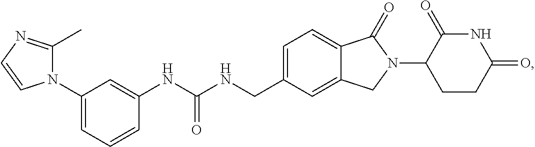 [SMILES: Cc1nccn1-c1cccc(NC(=O)NCc2ccc3c(c2)CN(C2CCC(=O)NC2=O)C3=O)c1]

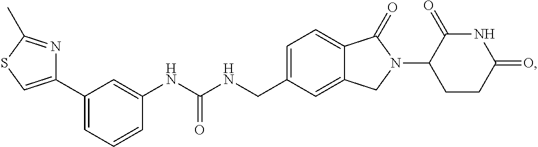 [SMILES: Cc1nc(-c2cccc(NC(=O)NCc3ccc4c(c3)CN(C3CCC(=O)NC3=O)C4=O)c2)cs1]

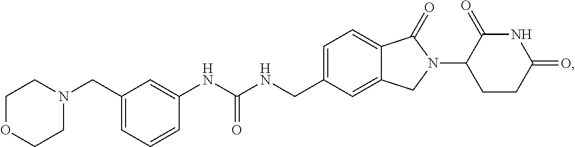 [SMILES: O=C1CCC(N2Cc3cc(CNC(=O)Nc4cccc(CN5CCOCC5)c4)ccc3C2=O)C(=O)N1]

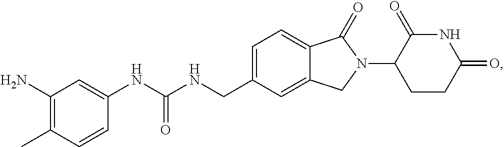 [SMILES: Cc1ccc(NC(=O)NCc2ccc3c(c2)CN(C2CCC(=O)NC2=O)C3=O)cc1N]

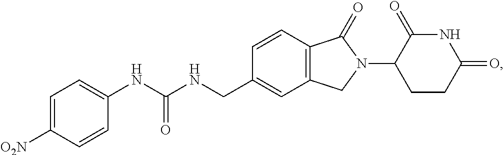 [SMILES: O=C1CCC(N2Cc3cc(CNC(=O)Nc4ccc([N+](=O)[O-])cc4)ccc3C2=O)C(=O)N1]

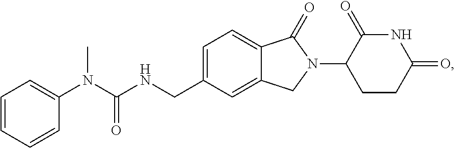 [SMILES: CN(C(=O)NCc1ccc2c(c1)CN(C1CCC(=O)NC1=O)C2=O)c1ccccc1]

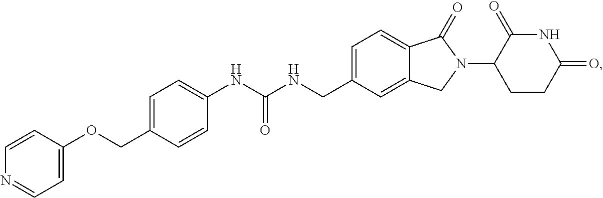 [SMILES: O=C1CCC(N2Cc3cc(CNC(=O)Nc4ccc(COc5ccncc5)cc4)ccc3C2=O)C(=O)N1]

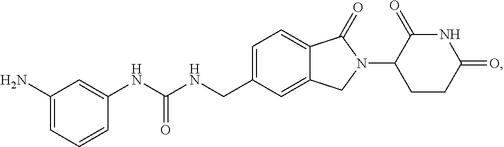 [SMILES: Nc1cccc(NC(=O)NCc2ccc3c(c2)CN(C2CCC(=O)NC2=O)C3=O)c1]

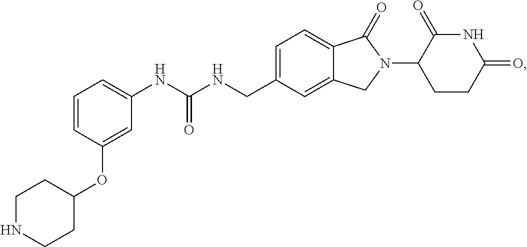 [SMILES: O=C1CCC(N2Cc3cc(CNC(=O)Nc4cccc(OC5CCNCC5)c4)ccc3C2=O)C(=O)N1]

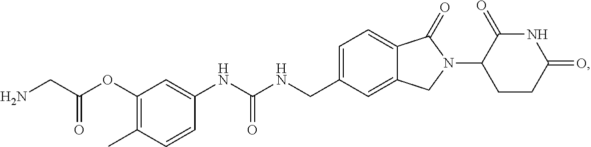 [SMILES: Cc1ccc(NC(=O)NCc2ccc3c(c2)CN(C2CCC(=O)NC2=O)C3=O)cc1OC(=O)CN]

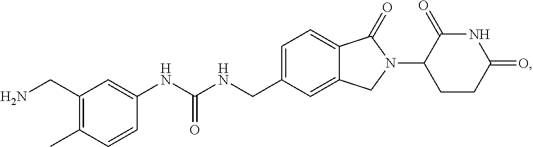 [SMILES: Cc1ccc(NC(=O)NCc2ccc3c(c2)CN(C2CCC(=O)NC2=O)C3=O)cc1CN]

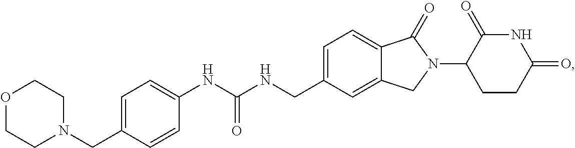 [SMILES: O=C1CCC(N2Cc3cc(CNC(=O)Nc4ccc(CN5CCOCC5)cc4)ccc3C2=O)C(=O)N1]

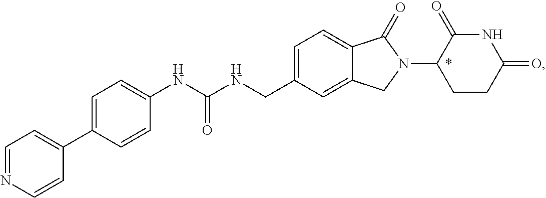

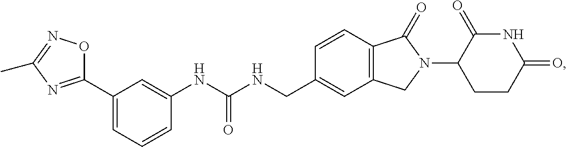 [SMILES: Cc1noc(-c2cccc(NC(=O)NCc3ccc4c(c3)CN(C3CCC(=O)NC3=O)C4=O)c2)n1]

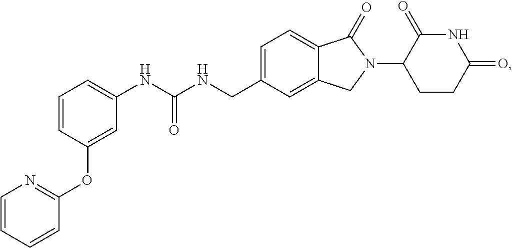 [SMILES: O=C1CCC(N2Cc3cc(CNC(=O)Nc4cccc(Oc5ccccn5)c4)ccc3C2=O)C(=O)N1]

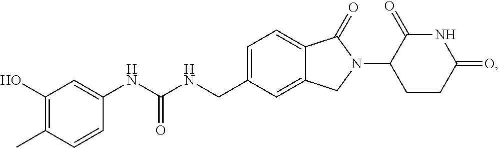 [SMILES: Cc1ccc(NC(=O)NCc2ccc3c(c2)CN(C2CCC(=O)NC2=O)C3=O)cc1O]

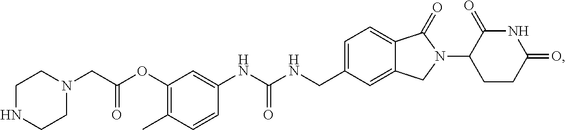 [SMILES: Cc1ccc(NC(=O)NCc2ccc3c(c2)CN(C2CCC(=O)NC2=O)C3=O)cc1OC(=O)CN1CCNCC1]

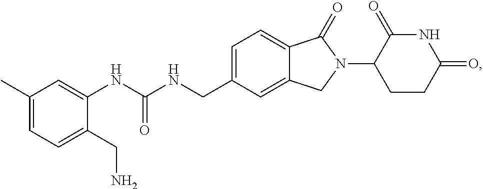 [SMILES: Cc1ccc(CN)c(NC(=O)NCc2ccc3c(c2)CN(C2CCC(=O)NC2=O)C3=O)c1]

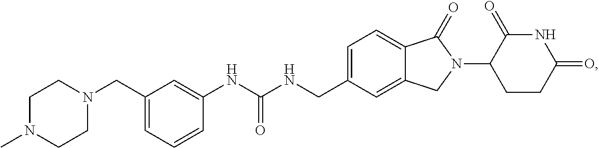 [SMILES: CN1CCN(Cc2cccc(NC(=O)NCc3ccc4c(c3)CN(C3CCC(=O)NC3=O)C4=O)c2)CC1]

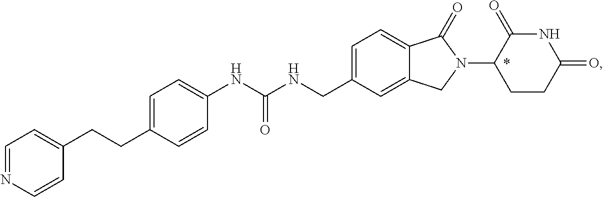

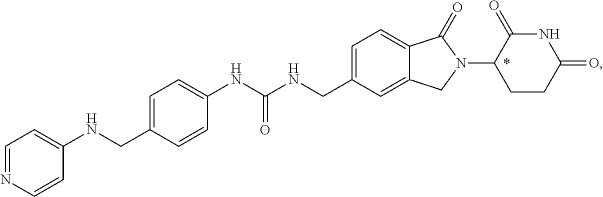

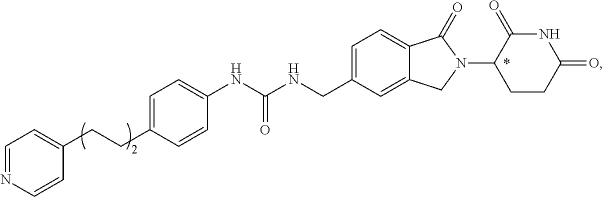

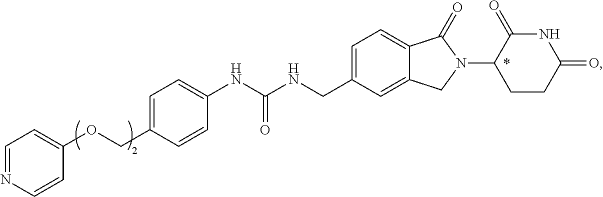

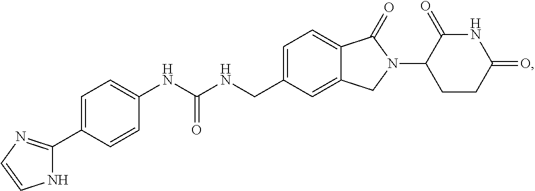 [SMILES: O=C1CCC(N2Cc3cc(CNC(=O)Nc4ccc(-c5ncc[nH]5)cc4)ccc3C2=O)C(=O)N1]

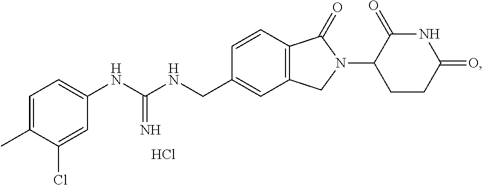 [SMILES: Cc1ccc(NC(=N)NCc2ccc3c(c2)CN(C2CCC(=O)NC2=O)C3=O)cc1Cl ; Cl]

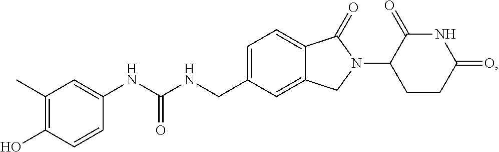 [SMILES: Cc1cc(NC(=O)NCc2ccc3c(c2)CN(C2CCC(=O)NC2=O)C3=O)ccc1O]

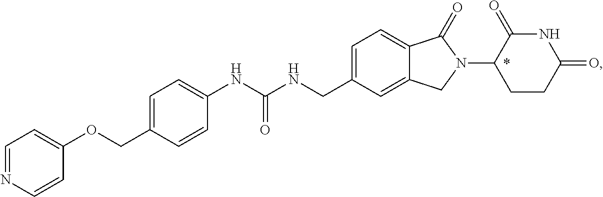

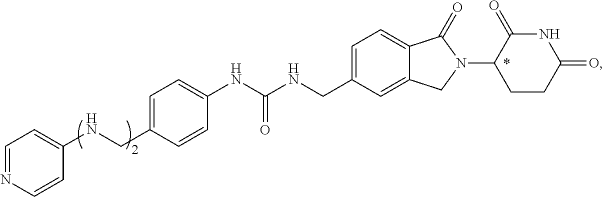

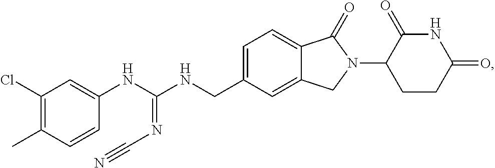 [SMILES: Cc1ccc(N/C(=N\C#N)NCc2ccc3c(c2)CN(C2CCC(=O)NC2=O)C3=O)cc1Cl]

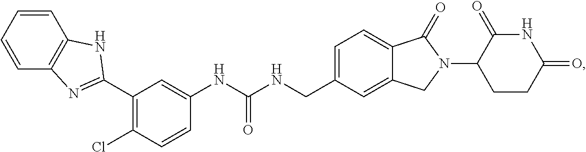 [SMILES: O=C1CCC(N2Cc3cc(CNC(=O)Nc4ccc(Cl)c(-c5nc6ccccc6[nH]5)c4)ccc3C2=O)C(=O)N1]

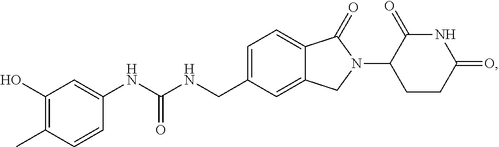 [SMILES: Cc1ccc(NC(=O)NCc2ccc3c(c2)CN(C2CCC(=O)NC2=O)C3=O)cc1O]

or a pharmaceutically acceptable salt, solvate, prodrug, and stereoisomer thereof.

In another embodiment, representative compounds are of Formula XIV:

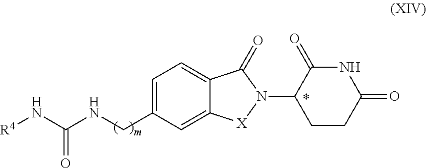

or a pharmaceutically acceptable salt, solvate, prodrug, or stereoisomer thereof, wherein:

X is C(═O) or CH₂;

m is an integer of 0, 1, 2, or 3;

R⁴ is C₃₋₁₀ cycloalkyl, 5 to 10 membered heterocyclyl, 5 to 10 membered heteroaryl, or C₀₋₄ alkyl-NR⁴¹R⁴²; wherein the cycloalkyl, heterocyclyl, and heteroaryl are each optionally substituted with one or more halogen, C₁₋₆ alkyl, —CO—NR⁴³R⁴⁴, —COOR⁴⁵, or C₀₋₄ alkyl-C₆₋₁₀ aryl, wherein the aryl itself may be optionally substituted with one or more halogen; and

R⁴¹, R⁴², R⁴³, R⁴⁴, and R⁴⁵ are each independently hydrogen or C₁₋₆ alkyl.

In certain embodiments, X is CH₂. In certain embodiments, X is C(═O).

In certain embodiments, m is 0. In certain embodiments, m is 1. In certain embodiments, m is 2. In certain embodiments, m is 3.

In certain embodiments, R⁴ is C₃₋₁₀ cycloalkyl, optionally substituted with one or more (C₁₋₆) alkyl or C₀₋₄ alkyl-C₆₋₁₀ aryl. In certain embodiments, R⁴ is 5 to 6 membered heterocyclyl, optionally substituted with one or more (C₁₋₆) alkyl or C₀₋₄ alkyl-C₆₋₁₀ aryl. In certain embodiments, R⁴ is C₀₋₄ alkyl-NR⁴¹R⁴², wherein R⁴¹ and R⁴² are each described herein.

In certain embodiments, R⁴ is 3-(N,N-diethylamino)propyl, 4-acetamidophenyl, 3-(2-aminoacetoxy)-4-methylphenyl, 3-aminomethyl-4-methylphenyl, 2-aminomethyl-5-methylphenyl, 3-aminophenyl, 3-amino-4-methylphenyl, 3-chloro-4-methylphenyl, 4-hydroxymethylphenyl, 3-hydroxy-4-methylphenyl, 3-(2-methyl-1H-imidazol-1-yl)phenyl, 4-methyl-3-nitrophenyl, 3-(3-methyl-1,2,4-oxadiazol-5-yl)phenyl, 4-methyl-3-(2-piperazin-1-ylacetoxy)-phenyl, 3-((4-methylpiperazin-1-yl)methyl)phenyl, 3-(1-methyl-1H-pyrazol-3-yl)phenyl, 3-(2-methyl-2H-pyrazol-3-yl)phenyl, 3-(2-methylthiazol-4-yl)phenyl, 4-(4-methyl-4H-1,2,4-triazol-3-yl)phenyl, 3-(morpholinomethyl)phenyl, 4-(morpholinomethyl)phenyl, 4-nitrophenyl, phenyl, 3-(piperidin-4-yloxy)phenyl, 4-(pyridin-4-yl)methylphenyl, 4-((pyridin-4-yloxy)methyl)phenyl, 3-(pyridin-2-yloxy)phenyl, 3-phenoxyphenyl, 4-tert-butylcyclohexyl, cis-4-tert-butylcyclohexyl, trans-4-tert-butylcyclohexyl, 4-methylcyclohexyl, cis-4-methylcyclohexyl, trans-4-methylcyclohexyl, 1-benzylpiperidin-4-yl, 4-methyltetrahydro-2H-pyran-4-yl, piperidin-4-yl, 4-phenylcyclohexyl, cis-4-phenylcyclohexyl, or trans-4-phenylcyclohexyl.

In one embodiment, the compound is selected from those listed in Table R, below:

TABLE R

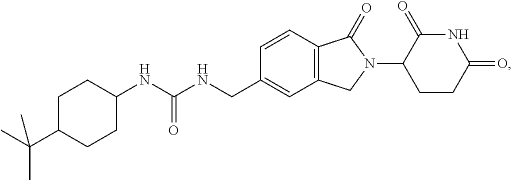 [SMILES: CC(C)(C)C1CCC(NC(=O)NCc2ccc3c(c2)CN(C2CCC(=O)NC2=O)C3=O)CC1]

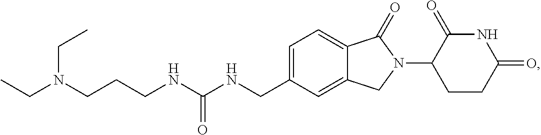 [SMILES: CCN(CC)CCCNC(=O)NCc1ccc2c(c1)CN(C1CCC(=O)NC1=O)C2=O]

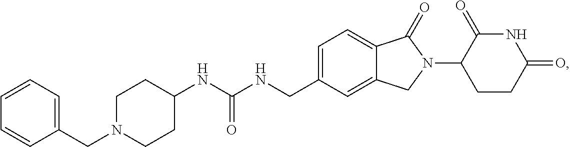 [SMILES: O=C1CCC(N2Cc3cc(CNC(=O)NC4CCN(Cc5ccccc5)CC4)ccc3C2=O)C(=O)N1]

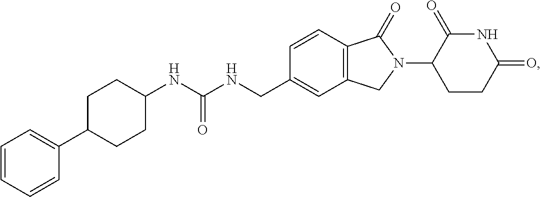 [SMILES: O=C1CCC(N2Cc3cc(CNC(=O)NC4CCC(c5ccccc5)CC4)ccc3C2=O)C(=O)N1]

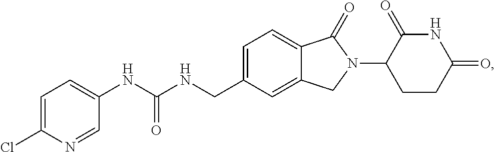 [SMILES: O=C1CCC(N2Cc3cc(CNC(=O)Nc4ccc(Cl)nc4)ccc3C2=O)C(=O)N1]

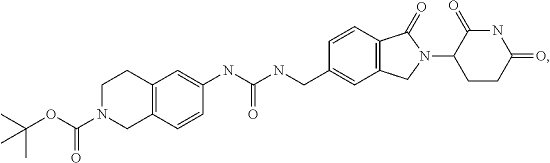 [SMILES: CC(C)(C)OC(=O)N1CCc2cc(NC(=O)NCc3ccc4c(c3)CN(C3CCC(=O)NC3=O)C4=O)ccc2C1]

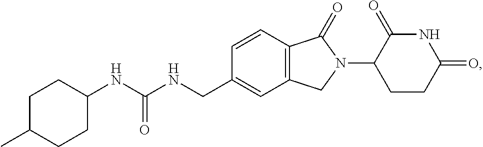 [SMILES: CC1CCC(NC(=O)NCc2ccc3c(c2)CN(C2CCC(=O)NC2=O)C3=O)CC1]

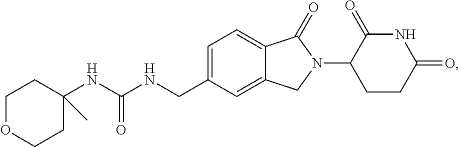 [SMILES: CC1(NC(=O)NCc2ccc3c(c2)CN(C2CCC(=O)NC2=O)C3=O)CCOCC1]

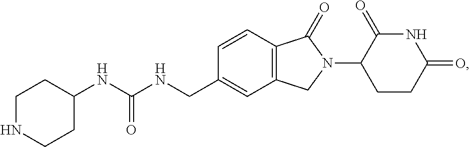 [SMILES: O=C1CCC(N2Cc3cc(CNC(=O)NC4CCNCC4)ccc3C2=O)C(=O)N1]

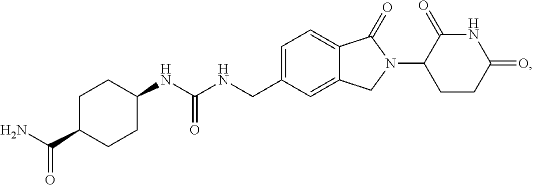 [SMILES: NC(=O)[C@H]1CC[C@@H](NC(=O)NCc2ccc3c(c2)CN(C2CCC(=O)NC2=O)C3=O)CC1]

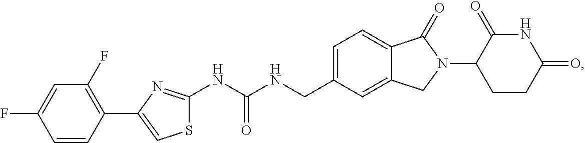 [SMILES: O=C1CCC(N2Cc3cc(CNC(=O)Nc4nc(-c5ccc(F)cc5F)cs4)ccc3C2=O)C(=O)N1]

or a pharmaceutically acceptable salt, solvate, prodrug, or stereoisomer thereof.

In yet another embodiment, representative compounds are of Formula XV:

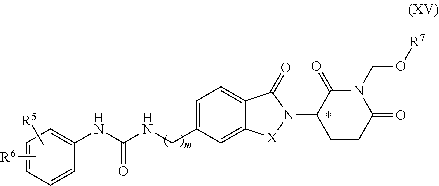

or a pharmaceutically acceptable salt, solvate, prodrug, or stereoisomer thereof, wherein:

X is C(═O) or CH₂;

m is an integer of 0, 1, 2, or 3;

R⁵ and R⁶ are each independently: hydrogen, halo, C₁₋₆ alkyl, oxo, —NO₂, C₁₋₆ alkoxy, —Z—C₁₋₆ alkyl, C₀₋₆ alkyl- (5 to 10 membered heteroaryl), C₀₋₆ alkyl- (5 to 6 membered heterocyclyl), C₀₋₆ alkyl-OH, C₀₋₄ alkyl-NH₂, —NHCO—C₁₋₆ alkyl, —OR²¹, or —(CH₂—Y)₀₋₂— (5 to 10 membered heteroaryl),

wherein Z is S or SO₂;
wherein R²¹ is as defined above;
wherein each heteroaryl and heterocyclyl above is optionally substituted with one or more C₁₋₆ alkyl; and
wherein the alkyl or alkoxy above may be optionally substituted with one or more: halogen; cyano; nitro; amino; C₁₋₆ alkylidenedioxy; C₁₋₆ alkoxy, itself optionally substituted with one or more halogens; or C₁₋₆ alkylthio, itself optionally substituted with one or more halogens;

R⁷ is —COR⁷¹ or —PO(OR⁷²)(OR⁷³);

R⁷¹ is C₁₋₁₀ alkyl, C₆₋₁₀ aryl, or 5 to 6 membered heterocyclyl; wherein the alkyl, aryl, heterocyclyl may be optionally substituted with one or more amino, C₁₋₆ alkylamino, di(C₁₋₆ alkyl)amino, or —COOR⁷⁴; and

R⁷², R⁷³, and R⁷⁴ are each independently hydrogen or C₁₋₁₀ alkyl.

In certain embodiments, X is CH₂. In certain embodiments, X is C(═O).

In certain embodiments, m is 0. In certain embodiments, m is 1. In certain embodiments, m is 2. In certain embodiments, m is 3.

In certain embodiments, R⁵ is hydrogen. In certain embodiments, R⁵ is halo. In certain embodiments, R⁵ is fluoro or chloro.

In certain embodiments, R⁶ is hydrogen. In certain embodiments, R⁶ is halo. In certain embodiments, R⁶ is fluoro or chloro.

In certain embodiments, R⁷ is —COR⁴¹, wherein R⁴¹ is as described herein. In certain embodiments, R⁷ is —PO(OR⁴²))(OR⁴³), wherein R⁴² and R⁴³ are each as described herein.

In one embodiment, the compound is selected from those listed in Table S, below:

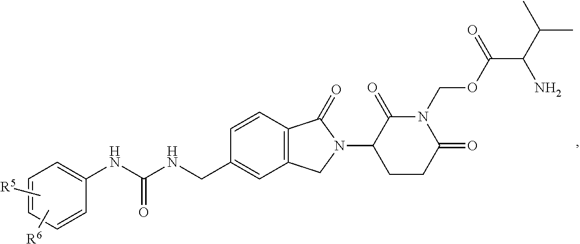

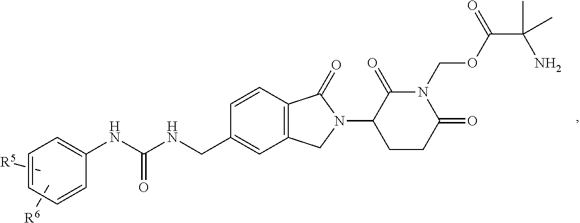

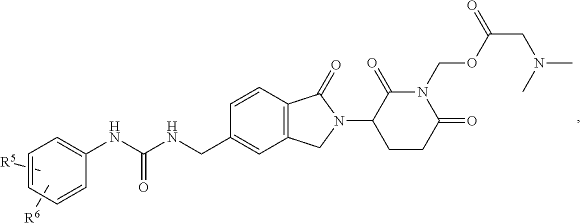

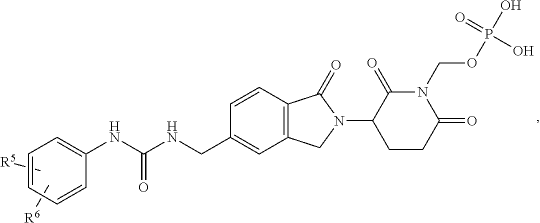

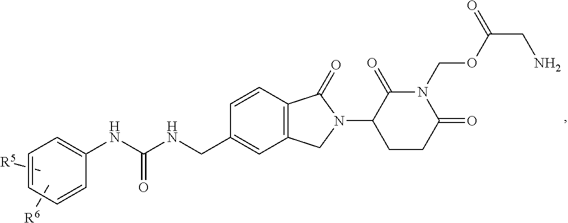

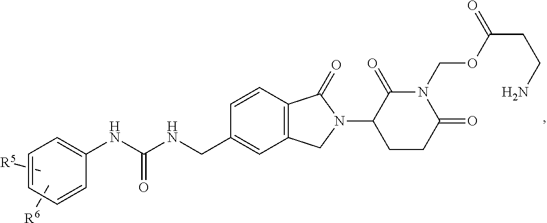

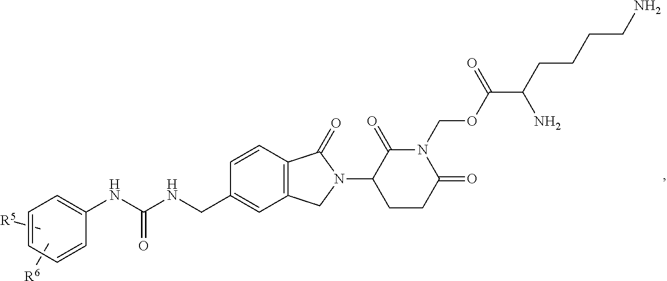

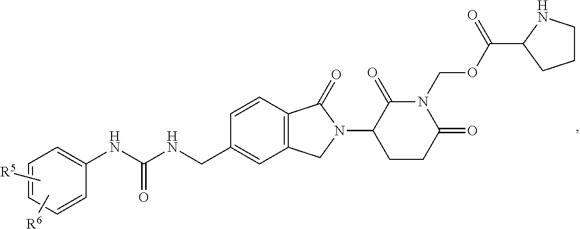

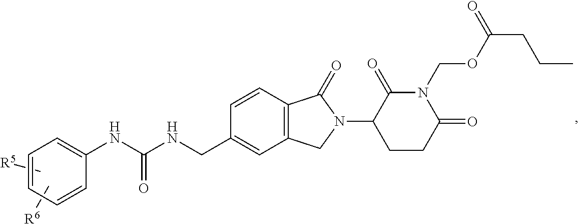

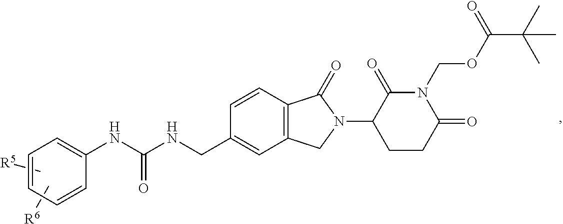

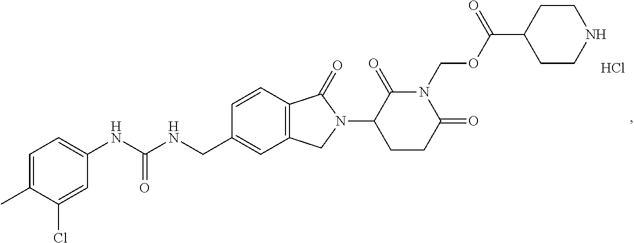 [SMILES: Cc1ccc(NC(=O)NCc2ccc3c(c2)CN(C2CCC(=O)N(COC(=O)C4CCNCC4)C2=O)C3=O)cc1Cl ; Cl ; ","]

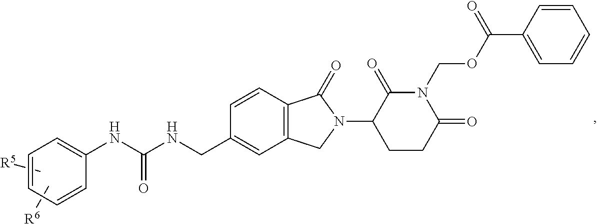

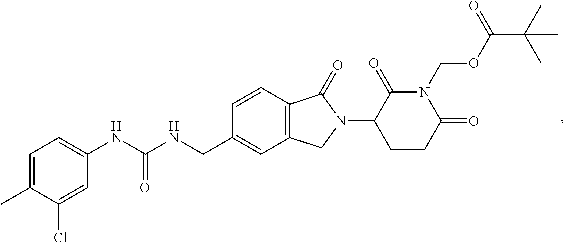 [SMILES: Cc1ccc(NC(=O)NCc2ccc3c(c2)CN(C2CCC(=O)N(COC(=O)C(C)(C)C)C2=O)C3=O)cc1Cl ; ","]

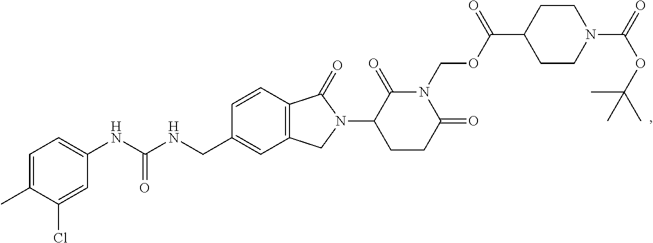 [SMILES: Cc1ccc(NC(=O)NCc2ccc3c(c2)CN(C2CCC(=O)N(COC(=O)C4CCN(C(=O)OC(C)(C)C)CC4)C2=O)C3=O)cc1Cl ; ","]

or a pharmaceutically acceptable salt, solvate, prodrug, or stereoisomer thereof, wherein R⁵ and R⁶ are as defined above.

In yet another embodiment, representative compounds are of Formula XVI:

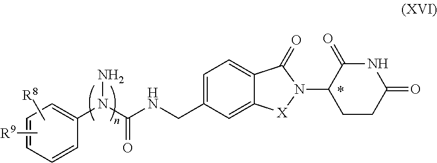

or a pharmaceutically acceptable salt, solvate, prodrug, or stereoisomer thereof, wherein:

X is C(═O) or CH₂;

n is an integer of 0 or 1;

R⁸ is hydrogen or halo; and

R⁹ is hydrogen, amino, or 5 to 10 membered heteroaryl or heterocyclyl;

with the proviso that when m is 0, R⁹ is not hydrogen.

In certain embodiments, X is CH₂. In certain embodiments, X is C(═O).

In certain embodiments, n is 0. In certain embodiments, n is 1.

In certain embodiments, R⁸ is hydrogen. In certain embodiments, R⁸ is halo. In certain embodiments, R⁸ is fluoro or chloro.

In certain embodiments, R⁹ is hydrogen. In certain embodiments, R⁹ is amino. In certain embodiments, R⁹ is 5 to 10 membered heteroaryl. In certain embodiments, R⁹ is 5 to 10 membered heterocyclyl.

In one embodiment, the compound is:

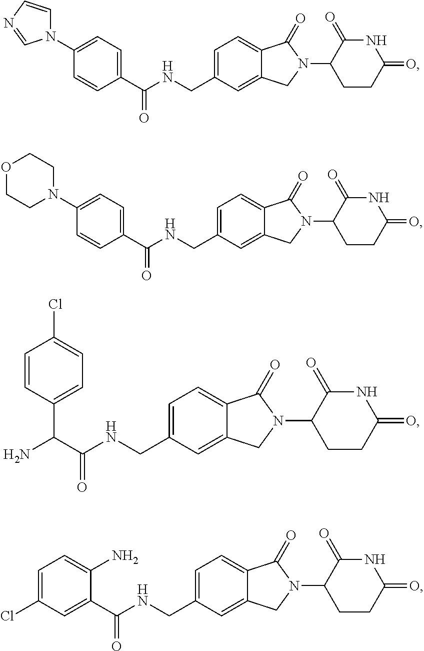 [SMILES: O=C1CCC(N2Cc3cc(CNC(=O)c4ccc(-n5ccnc5)cc4)ccc3C2=O)C(=O)N1 ; O=C1CCC(N2Cc3cc(CNC(=O)c4ccc(N5CCOCC5)cc4)ccc3C2=O)C(=O)N1 ; NC(C(=O)NCc1ccc2c(c1)CN(C1CCC(=O)NC1=O)C2=O)c1ccc(Cl)cc1 ; Nc1ccc(Cl)cc1C(=O)NCc1ccc2c(c1)CN(C1CCC(=O)NC1=O)C2=O]

or a pharmaceutically acceptable salt, solvate, prodrug, or stereoisomer thereof.

In yet another embodiment, representative compounds are of Formula XVII:

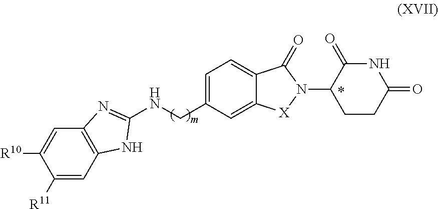

or a pharmaceutically acceptable salt, solvate, prodrug, or stereoisomer thereof, wherein:

X is C(═O) or CH₂;

m is an integer of 0, 1, 2, or 3;

R¹⁰ and R¹¹ are each independently hydrogen, halo, C₁₋₆ alkyl, or C₆₋₁₀ aryloxy, wherein the alkyl and aryl are each optionally substituted with one or more halo.

In certain embodiments, X is CH₂. In certain embodiments, X is C(═O).

In certain embodiments, m is 0. In certain embodiments, m is 1. In certain embodiments, m is 2. In certain embodiments, m is 3.

In certain embodiments, R¹⁰ is hydrogen. In certain embodiments, R¹⁰ is halo. In certain embodiments, R¹⁰ is fluoro or chloro. In certain embodiments, R¹⁰ is C₁₋₆ alkyl, optionally substituted with one or more halo. In certain embodiments, R¹⁰ is C₆₋₁₀ aryloxy, optionally substituted with one or more halo.

In certain embodiments, R¹¹ is hydrogen. In certain embodiments, R¹¹ is halo. In certain embodiments, R¹¹ is fluoro or chloro. In certain embodiments, R¹¹ is C₁₋₆ alkyl, optionally substituted with one or more halo. In certain embodiments, R¹¹ is C₆₋₁₀ aryloxy, optionally substituted with one or more halo.

In one embodiment, the compound is selected from those listed in Table T, below:

TABLE T

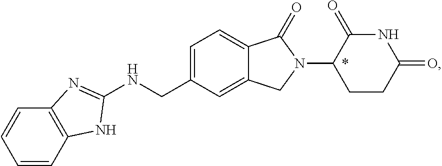

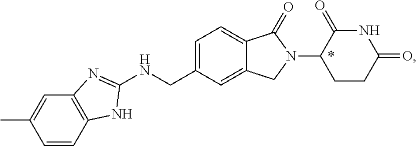

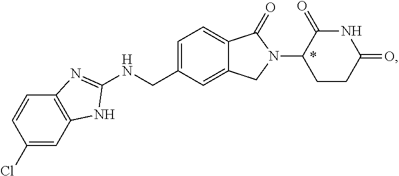

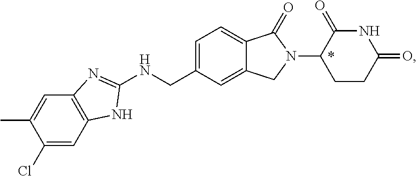

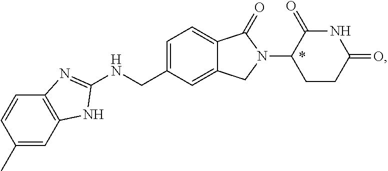

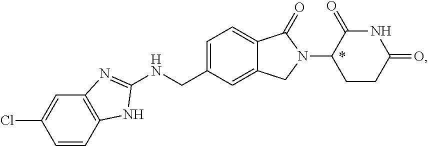

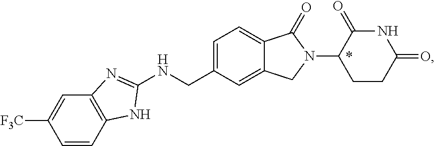

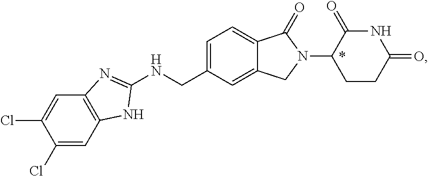

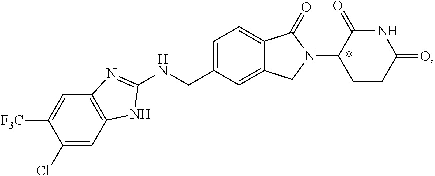

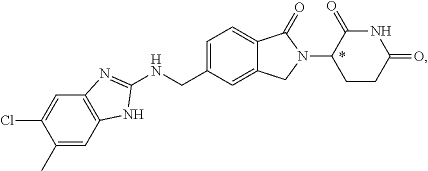

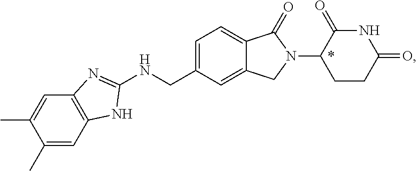

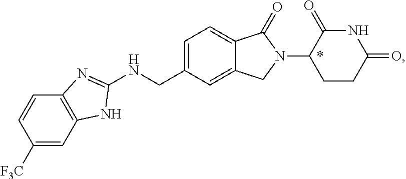

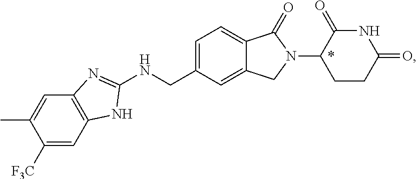

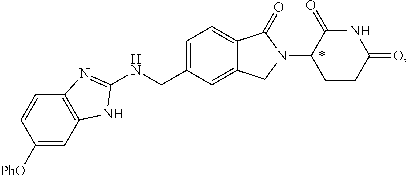

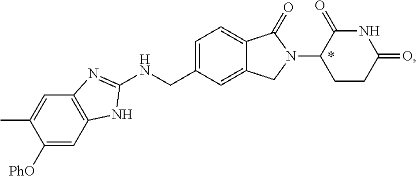

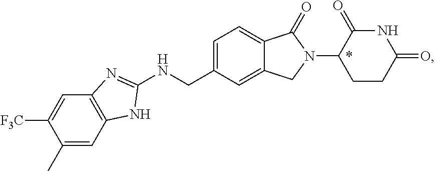

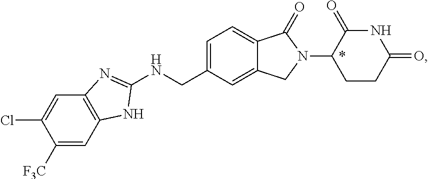

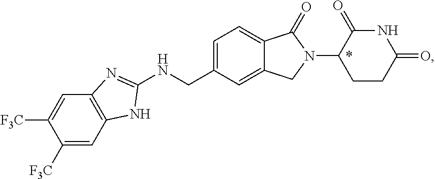

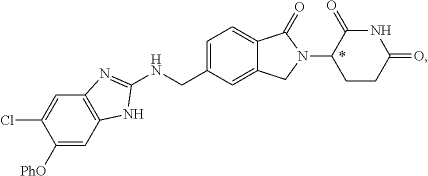

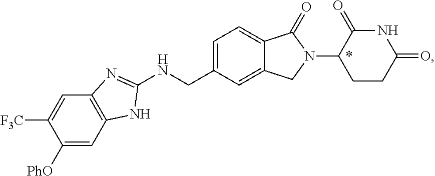

or a pharmaceutically acceptable salt, solvate, prodrug, or stereoisomer thereof.

In yet another embodiment, representative compounds are of Formula XVIII:

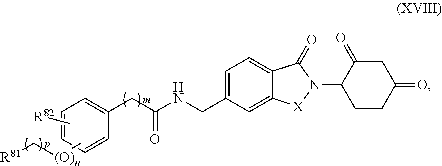

or a pharmaceutically acceptable salt, solvate, prodrug, or stereoisomer thereof, wherein:

### X is CH₂ or C═O

m and n are each independently 0 or 1;
p is 0, 1, 2, or 3;
R⁸¹ is 5 to 6 membered heterocyclyl, optionally substituted with C₁₋₆ alkyl; and
R⁸² is hydrogen or halogen.

In one embodiment, X is CH₂. In another embodiment, X is C═O.

In one embodiment, m is 0. In another embodiment, m is 1. In another embodiment, n is 0. In another embodiment, n is 1.

In one embodiment, p is 0. In another embodiment, p is 1. In another embodiment, p is 2. In another embodiment, p is 3.

In one embodiment, R⁸¹ is 5 membered heterocycle. In another embodiment, the 5 membered heterocycle is substituted with C₁₋₆ alkyl. In another embodiment, R⁸¹ is 6 membered heterocycle. In another embodiment, the 6 membered heterocycle is substituted with C₁₋₆ alkyl.

In one embodiment, R⁸² is hydrogen. In another embodiment, R⁸² is halogen.

In one embodiment, the compound is selected from those listed in Table U, below:

TABLE U

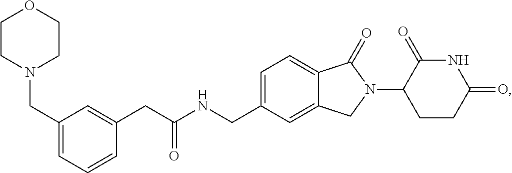 [SMILES: O=C(Cc1cccc(CN2CCOCC2)c1)NCc1ccc2c(c1)CN(C1CCC(=O)NC1=O)C2=O]

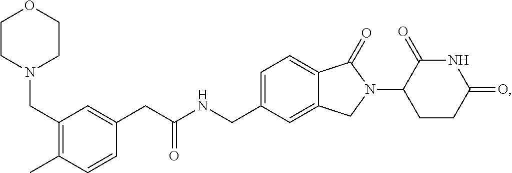 [SMILES: Cc1ccc(CC(=O)NCc2ccc3c(c2)CN(C2CCC(=O)NC2=O)C3=O)cc1CN1CCOCC1]

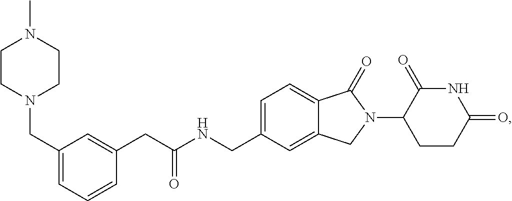 [SMILES: CN1CCN(Cc2cccc(CC(=O)NCc3ccc4c(c3)CN(C3CCC(=O)NC3=O)C4=O)c2)CC1]

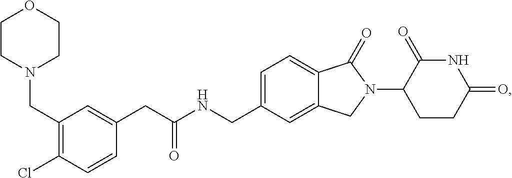 [SMILES: O=C(Cc1ccc(Cl)c(CN2CCOCC2)c1)NCc1ccc2c(c1)CN(C1CCC(=O)NC1=O)C2=O]

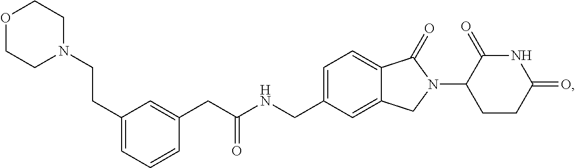 [SMILES: O=C(Cc1cccc(CCN2CCOCC2)c1)NCc1ccc2c(c1)CN(C1CCC(=O)NC1=O)C2=O]

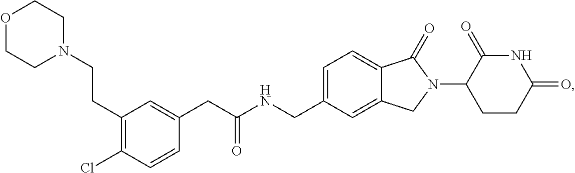 [SMILES: O=C(Cc1ccc(Cl)c(CCN2CCOCC2)c1)NCc1ccc2c(c1)CN(C1CCC(=O)NC1=O)C2=O]

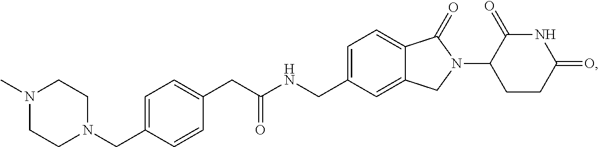 [SMILES: CN1CCN(Cc2ccc(CC(=O)NCc3ccc4c(c3)CN(C3CCC(=O)NC3=O)C4=O)cc2)CC1]

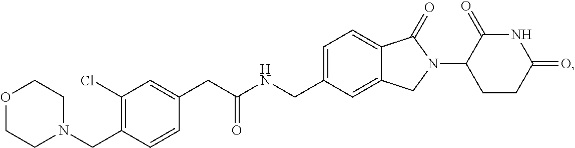 [SMILES: O=C(Cc1ccc(CN2CCOCC2)c(Cl)c1)NCc1ccc2c(c1)CN(C1CCC(=O)NC1=O)C2=O]

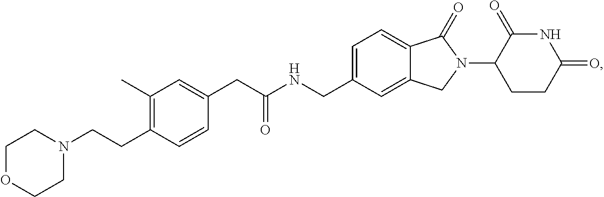 [SMILES: Cc1cc(CC(=O)NCc2ccc3c(c2)CN(C2CCC(=O)NC2=O)C3=O)ccc1CCN1CCOCC1]

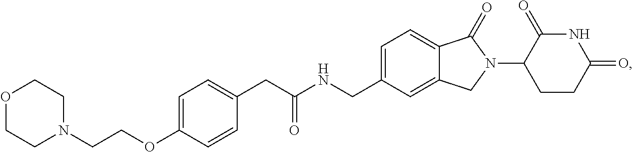 [SMILES: O=C(Cc1ccc(OCCN2CCOCC2)cc1)NCc1ccc2c(c1)CN(C1CCC(=O)NC1=O)C2=O]

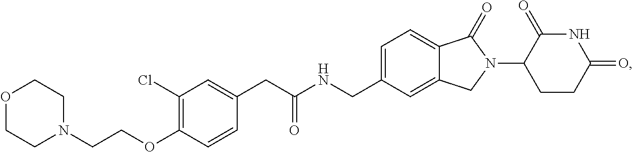 [SMILES: O=C(Cc1ccc(OCCN2CCOCC2)c(Cl)c1)NCc1ccc2c(c1)CN(C1CCC(=O)NC1=O)C2=O]

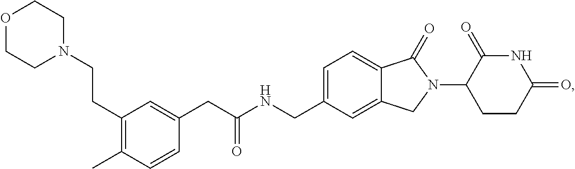 [SMILES: Cc1ccc(CC(=O)NCc2ccc3c(c2)CN(C2CCC(=O)NC2=O)C3=O)cc1CCN1CCOCC1]

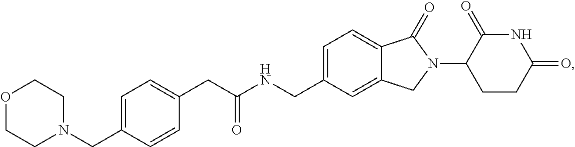 [SMILES: O=C(Cc1ccc(CN2CCOCC2)cc1)NCc1ccc2c(c1)CN(C1CCC(=O)NC1=O)C2=O]

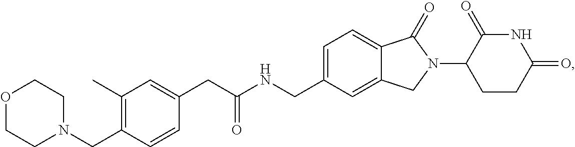 [SMILES: Cc1cc(CC(=O)NCc2ccc3c(c2)CN(C2CCC(=O)NC2=O)C3=O)ccc1CN1CCOCC1]

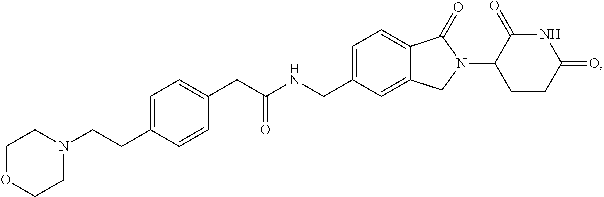 [SMILES: O=C(Cc1ccc(CCN2CCOCC2)cc1)NCc1ccc2c(c1)CN(C1CCC(=O)NC1=O)C2=O]

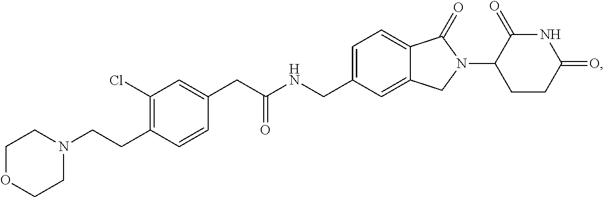 [SMILES: O=C(Cc1ccc(CCN2CCOCC2)c(Cl)c1)NCc1ccc2c(c1)CN(C1CCC(=O)NC1=O)C2=O]

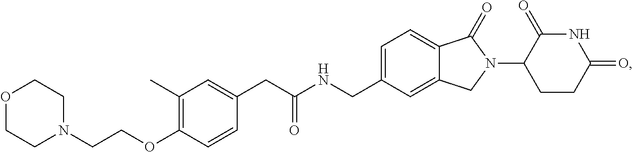 [SMILES: Cc1cc(CC(=O)NCc2ccc3c(c2)CN(C2CCC(=O)NC2=O)C3=O)ccc1OCCN1CCOCC1]

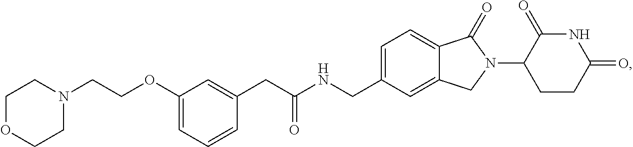 [SMILES: O=C(Cc1cccc(OCCN2CCOCC2)c1)NCc1ccc2c(c1)CN(C1CCC(=O)NC1=O)C2=O]

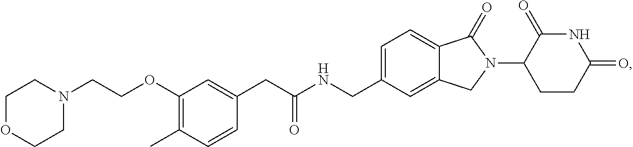 [SMILES: Cc1ccc(CC(=O)NCc2ccc3c(c2)CN(C2CCC(=O)NC2=O)C3=O)cc1OCCN1CCOCC1]

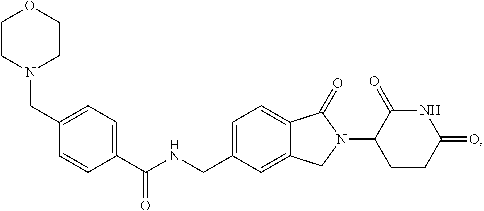 [SMILES: O=C1CCC(N2Cc3cc(CNC(=O)c4ccc(CN5CCOCC5)cc4)ccc3C2=O)C(=O)N1]

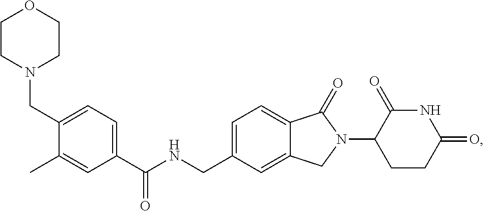 [SMILES: Cc1cc(C(=O)NCc2ccc3c(c2)CN(C2CCC(=O)NC2=O)C3=O)ccc1CN1CCOCC1]

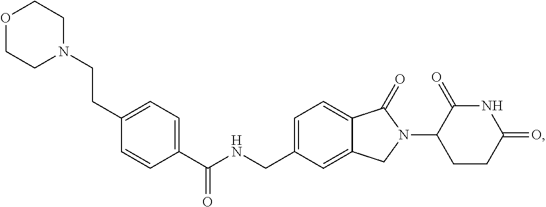 [SMILES: O=C1CCC(N2Cc3cc(CNC(=O)c4ccc(CCN5CCOCC5)cc4)ccc3C2=O)C(=O)N1]

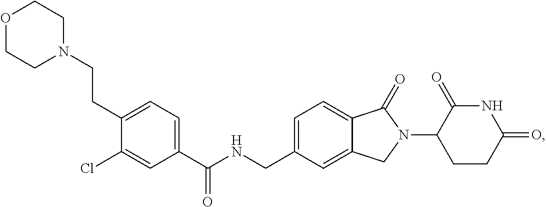 [SMILES: O=C1CCC(N2Cc3cc(CNC(=O)c4ccc(CCN5CCOCC5)c(Cl)c4)ccc3C2=O)C(=O)N1]

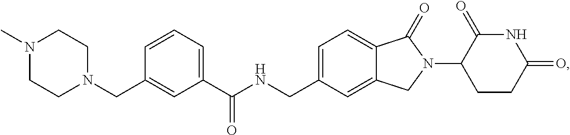 [SMILES: CN1CCN(Cc2cccc(C(=O)NCc3ccc4c(c3)CN(C3CCC(=O)NC3=O)C4=O)c2)CC1]

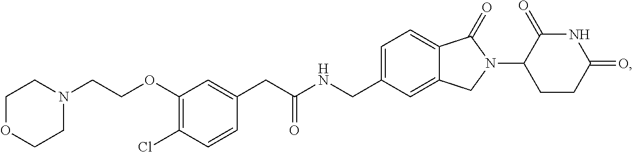 [SMILES: O=C(Cc1ccc(Cl)c(OCCN2CCOCC2)c1)NCc1ccc2c(c1)CN(C1CCC(=O)NC1=O)C2=O]

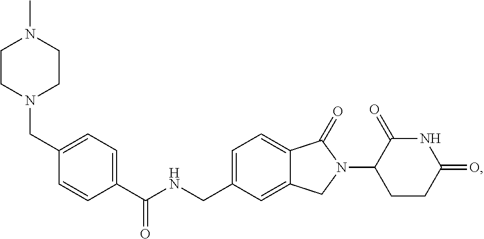 [SMILES: CN1CCN(Cc2ccc(C(=O)NCc3ccc4c(c3)CN(C3CCC(=O)NC3=O)C4=O)cc2)CC1]

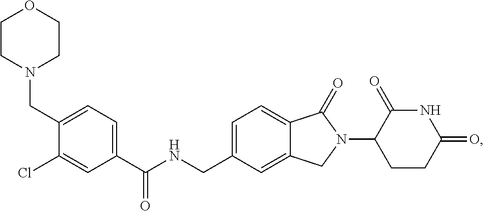 [SMILES: O=C1CCC(N2Cc3cc(CNC(=O)c4ccc(CN5CCOCC5)c(Cl)c4)ccc3C2=O)C(=O)N1]

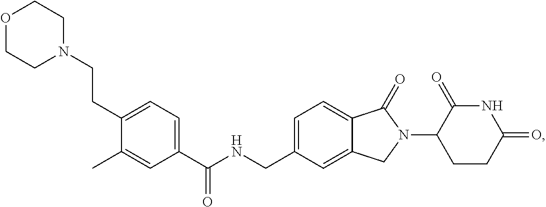 [SMILES: Cc1cc(C(=O)NCc2ccc3c(c2)CN(C2CCC(=O)NC2=O)C3=O)ccc1CCN1CCOCC1]

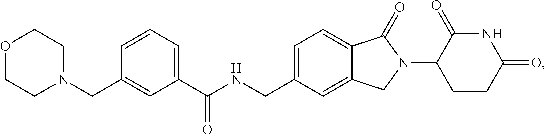 [SMILES: O=C1CCC(N2Cc3cc(CNC(=O)c4cccc(CN5CCOCC5)c4)ccc3C2=O)C(=O)N1]

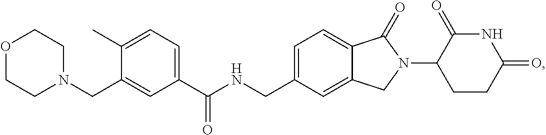 [SMILES: Cc1ccc(C(=O)NCc2ccc3c(c2)CN(C2CCC(=O)NC2=O)C3=O)cc1CN1CCOCC1]

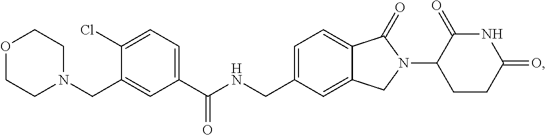 [SMILES: O=C1CCC(N2Cc3cc(CNC(=O)c4ccc(Cl)c(CN5CCOCC5)c4)ccc3C2=O)C(=O)N1]

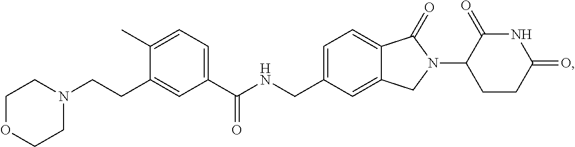 [SMILES: Cc1ccc(C(=O)NCc2ccc3c(c2)CN(C2CCC(=O)NC2=O)C3=O)cc1CCN1CCOCC1]

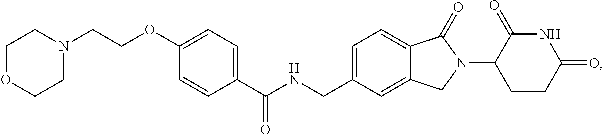 [SMILES: O=C1CCC(N2Cc3cc(CNC(=O)c4ccc(OCCN5CCOCC5)cc4)ccc3C2=O)C(=O)N1]

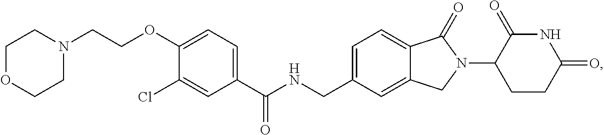 [SMILES: O=C1CCC(N2Cc3cc(CNC(=O)c4ccc(OCCN5CCOCC5)c(Cl)c4)ccc3C2=O)C(=O)N1]

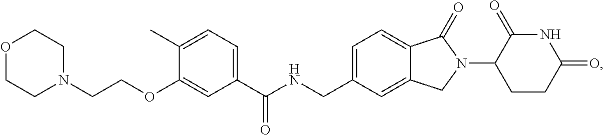 [SMILES: Cc1ccc(C(=O)NCc2ccc3c(c2)CN(C2CCC(=O)NC2=O)C3=O)cc1OCCN1CCOCC1]

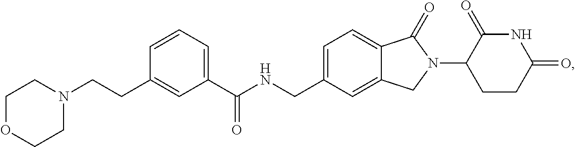 [SMILES: O=C1CCC(N2Cc3cc(CNC(=O)c4cccc(CCN5CCOCC5)c4)ccc3C2=O)C(=O)N1]

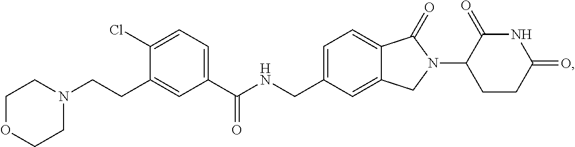 [SMILES: O=C1CCC(N2Cc3cc(CNC(=O)c4ccc(Cl)c(CCN5CCOCC5)c4)ccc3C2=O)C(=O)N1]

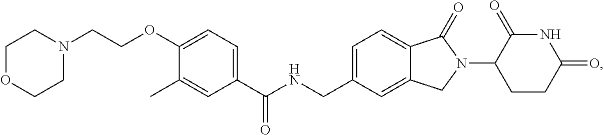 [SMILES: Cc1cc(C(=O)NCc2ccc3c(c2)CN(C2CCC(=O)NC2=O)C3=O)ccc1OCCN1CCOCC1]

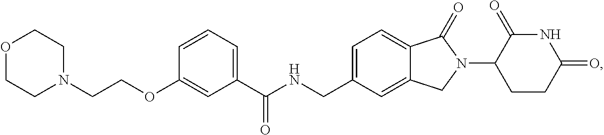 [SMILES: O=C1CCC(N2Cc3cc(CNC(=O)c4cccc(OCCN5CCOCC5)c4)ccc3C2=O)C(=O)N1]

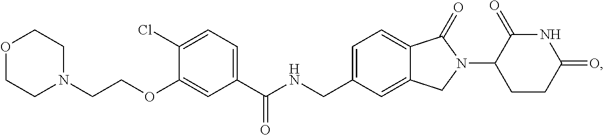 [SMILES: O=C1CCC(N2Cc3cc(CNC(=O)c4ccc(Cl)c(OCCN5CCOCC5)c4)ccc3C2=O)C(=O)N1]

or a pharmaceutically acceptable salt, solvate, prodrug, or stereoisomer thereof.

In yet another embodiment, representative compounds are of the following formula in Table V, below:

TABLE V

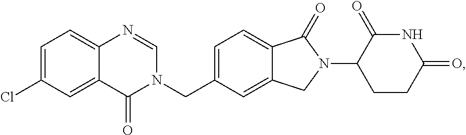 [SMILES: O=C1CCC(N2Cc3cc(Cn4cnc5ccc(Cl)cc5c4=O)ccc3C2=O)C(=O)N1]

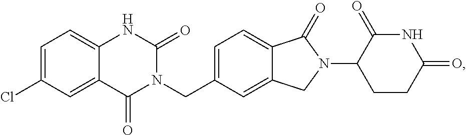 [SMILES: O=C1CCC(N2Cc3cc(Cn4c(=O)[nH]c5ccc(Cl)cc5c4=O)ccc3C2=O)C(=O)N1]

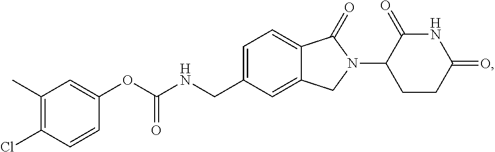 [SMILES: Cc1cc(OC(=O)NCc2ccc3c(c2)CN(C2CCC(=O)NC2=O)C3=O)ccc1Cl]

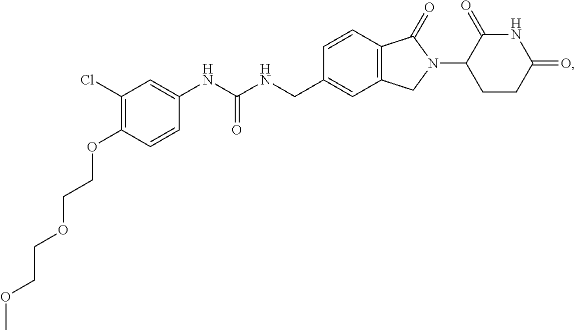 [SMILES: COCCOCCOc1ccc(NC(=O)NCc2ccc3c(c2)CN(C2CCC(=O)NC2=O)C3=O)cc1Cl]

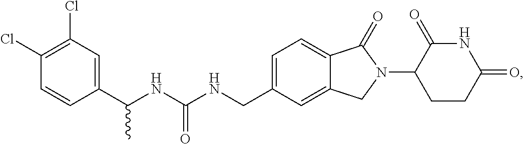 [SMILES: CC(NC(=O)NCc1ccc2c(c1)CN(C1CCC(=O)NC1=O)C2=O)c1ccc(Cl)c(Cl)c1]

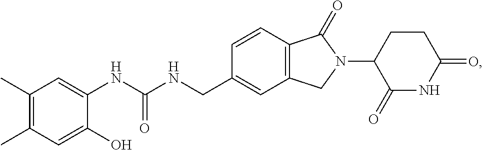 [SMILES: Cc1cc(O)c(NC(=O)NCc2ccc3c(c2)CN(C2CCC(=O)NC2=O)C3=O)cc1C]

or a pharmaceutically acceptable salt, solvate, prodrug, or stereoisomer thereof.

Still other specific IMiD® immunomodulatory drugs provided herein belong to a class of 4′-arylmethoxy isoindoline compounds disclosed in U.S. Patent Application Publication No. US 2011/0196150, the entirety of which is incorporated herein by reference. Representative compounds are of formula XIX:

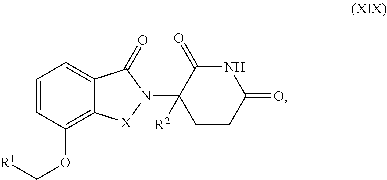

or a pharmaceutically acceptable salt, solvate or stereoisomer thereof, wherein:

### X is C═O or CH₂;

### R¹ is —Y—R³;

R² is H or (C₁-C₆)alkyl;
Y is: 6 to 10 membered aryl, heteroaryl or heterocycle, each of which may be optionally substituted with one or more halogen; or a bond;
R³ is: —(CH₂)ₙ-aryl, —O—(CH₂)ₙ-aryl or —(CH₂)ₙ—O-aryl, wherein the aryl is optionally substituted with one or more: (C₁-C₆)alkyl, itself optionally substituted with one or more halogen; (C₁-C₆)alkoxy, itself substituted with one or more halogen; oxo; amino; carboxyl; cyano; hydroxyl; halogen; deuterium; 6 to 10 membered aryl or heteroaryl, optionally substituted with one or more (C₁-C₆)alkyl, (C₁-C₆)alkoxy or halogen; —CONH₂; or
—COO—(C₁-C₆)alkyl, wherein the alkyl may be optionally substituted with one or more halogen;
—(CH₂)ₙ-heterocycle, —O—(CH₂)ₙ-heterocycle or —(CH₂)ₙ—O-heterocycle, wherein the heterocycle is optionally substituted with one or more: (C₁-C₆)alkyl, itself optionally substituted with one or more halogen; (C₁-C₆)alkoxy, itself substituted with one or more halogen; oxo; amino; carboxyl; cyano; hydroxyl; halogen; deuterium; 6 to 10 membered aryl or heteroaryl, optionally substituted with one or more (C₁-C₆)alkyl, (C₁-C₆)alkoxy or halogen; —CONH₂; or —COO—(C₁-C₆)alkyl, wherein the alkyl may be optionally substituted with one or more halogen; or —(CH₂)ₙ-heteroaryl, —O—(CH₂)ₙ-heteroaryl or —(CH₂)ₙ—O-heteroaryl, wherein the heteroaryl is optionally substituted with one or more: (C₁-C₆)alkyl, itself optionally substituted with one or more halogen; (C₁-C₆)alkoxy, itself substituted with one or more halogen; oxo; amino; carboxyl; cyano; hydroxyl; halogen; deuterium; 6 to 10 membered aryl or heteroaryl, optionally substituted with one or more (C₁-C₆)alkyl, (C₁-C₆)alkoxy or halogen; —CONH₂; or —COO—(C₁-C₆)alkyl, wherein the alkyl may be optionally substituted with one or more halogen; and n is 0, 1, 2 or 3.

In one embodiment, X is C═O. In another embodiment, C is CH₂.

In one embodiment, R² is H. In another embodiment, R² is (C₁-C₆)alkyl.

In one embodiment, Y is aryl. In another embodiment, Y is heteroaryl. In another embodiment, Y is heterocycle. In another embodiment, Y is a bond.

In one embodiment, R³ is unsubstituted —(CH₂)ₙ-aryl. In another embodiment, R³ is —(CH₂)ₙ-aryl substituted with one or more (C₁-C₆)alkyl, itself optionally substituted with one or more halogen. In another embodiment, R³ is —(CH₂)ₙ-aryl substituted with one or more (C₁-C₆)alkoxy, itself substituted with one or more halogen. In another embodiment, R³ is —(CH₂)ₙ-aryl substituted with one or more oxo. In another embodiment, R³ is —(CH₂)ₙ-aryl substituted with one or more amino. In another embodiment, R³ is —(CH₂)ₙ-aryl substituted with one or more carboxyl. In another embodiment, R³ is —(CH₂)ₙ-aryl substituted with one or more cyano. In another embodiment, R³ is —(CH₂)ₙ-aryl substituted with one or more hydroxyl. In another embodiment, R³ is —(CH₂)ₙ-aryl substituted with one or more halogen. In another embodiment, R³ is —(CH₂)ₙ-aryl substituted with one or more deuterium. In another embodiment, R³ is —(CH₂)ₙ-aryl substituted with one or more 6 to 10 membered aryl, optionally substituted with one or more (C₁-C₆)alkyl. In another embodiment, R³ is —(CH₂)ₙ-aryl substituted with one or more 6 to 10 membered heteroaryl, optionally substituted with one or more (C₁-C₆)alkyl, (C₁-C₆)alkoxy or halogen. In another embodiment, R³ is —(CH₂)ₙ-aryl substituted with one or more —CONH₂. In another embodiment, R³ is —(CH₂)ₙ-aryl substituted with one or more —COO—(C₁-C₆)alkyl, wherein the alkyl may be optionally substituted with one or more halogen.

In one embodiment, R³ is unsubstituted —O—(CH₂)ₙ-aryl. In another embodiment, R³ is —O—(CH₂)ₙ-aryl substituted with one or more (C₁-C₆)alkyl, itself optionally substituted with one or more halogen. In another embodiment, R³ is —O—(CH₂)ₙ-aryl substituted with one or more (C₁-C₆)alkoxy, itself substituted with one or more halogen. In another embodiment, R³ is —O—(CH₂)ₙ-aryl substituted with one or more oxo. In another embodiment, R³ is —O—(CH₂)ₙ-aryl substituted with one or more amino. In another embodiment, R³ is —O—(CH₂)ₙ-aryl substituted with one or more carboxyl. In another embodiment, R³ is —O—(CH₂)ₙ-aryl substituted with one or more cyano. In another embodiment, R³ is —O—(CH₂)ₙ-aryl substituted with one or more hydroxyl. In another embodiment, R³ is —O—(CH₂)ₙ-aryl substituted with one or more halogen. In another embodiment, R³ is —O—(CH₂)ₙ-aryl substituted with one or more deuterium. In another embodiment, R³ is —O—(CH₂)ₙ-aryl substituted with one or more 6 to 10 membered aryl, optionally substituted with one or more (C₁-C₆)alkyl. In another embodiment, R³ is —O—(CH₂)ₙ-aryl substituted with one or more 6 to 10 membered heteroaryl, optionally substituted with one or more (C₁-C₆)alkyl, (C₁-C₆)alkoxy or halogen. In another embodiment, R³ is —O—(CH₂)ₙ-aryl substituted with one or more —CONH₂. In another embodiment, R³ is —O—(CH₂)ₙ-aryl substituted with one or more —COO—(C₁-C₆)alkyl, wherein the alkyl may be optionally substituted with one or more halogen.

In one embodiment, R³ is unsubstituted —(CH₂)ₙ—O-aryl. In another embodiment, R³ is —(CH₂)ₙ—O-aryl substituted with one or more (C₁-C₆)alkyl, itself optionally substituted with one or more halogen. In another embodiment, R³ is —(CH₂)ₙ—O-aryl substituted with one or more (C₁-C₆)alkoxy, itself substituted with one or more halogen. In another embodiment, R³ is —(CH₂)ₙ—O-aryl substituted with one or more oxo. In another embodiment, R³ is —(CH₂)ₙ—O-aryl substituted with one or more amino. In another embodiment, R³ is —(CH₂)ₙ—O-aryl substituted with one or more carboxyl. In another embodiment, R³ is —(CH₂)ₙ—O-aryl substituted with one or more cyano. In another embodiment, R³ is —(CH₂)ₙ—O-aryl substituted with one or more hydroxyl. In another embodiment, R³ is —(CH₂)ₙ—O-aryl substituted with one or more halogen. In another embodiment, R³ is —(CH₂)ₙ—O-aryl substituted with one or more deuterium. In another embodiment, R³ is —(CH₂)ₙ—O-aryl substituted with one or more 6 to 10 membered aryl, optionally substituted with one or more (C₁-C₆)alkyl. In another embodiment, R³ is —(CH₂)ₙ—O-aryl substituted with one or more 6 to 10 membered heteroaryl, optionally substituted with one or more (C₁-C₆)alkyl, (C₁-C₆)alkoxy or halogen. In another embodiment, R³ is —(CH₂)ₙ—O-aryl substituted with one or more —CONH₂. In another embodiment, R³ is —(CH₂)ₙ—O-aryl substituted with one or more —COO—(C₁-C₆)alkyl, wherein the alkyl may be optionally substituted with one or more halogen.

In one embodiment, R³ is unsubstituted —(CH₂)ₙ-heterocycle. In another embodiment, R³ is —(CH₂)ₙ-heterocycle substituted with one or more (C₁-C₆)alkyl, itself optionally substituted with one or more halogen. In another embodiment, R³ is —(CH₂)ₙ-heterocycle substituted with one or more (C₁-C₆)alkoxy, itself substituted with one or more halogen. In another embodiment, R³ is —(CH₂)ₙ-heterocycle substituted with one or more oxo. In another embodiment, R³ is —(CH₂)ₙ-heterocycle substituted with one or more amino. In another embodiment, R³ is —(CH₂)ₙ-heterocycle substituted with one or more carboxyl. In another embodiment, R³ is —(CH₂)ₙ-heterocycle substituted with one or more cyano. In another embodiment, R³ is —(CH₂)ₙ-heterocycle substituted with one or more hydroxyl. In another embodiment, R³ is —(CH₂)ₙ-heterocycle substituted with one or more halogen. In another embodiment, R³ is —(CH₂)ₙ-heterocycle substituted with one or more deuterium. In another embodiment, R³ is —(CH₂)ₙ-heterocycle substituted with one or more 6 to 10 membered aryl, optionally substituted with one or more (C₁-C₆)alkyl. In another embodiment, R³ is —(CH₂)ₙ-heterocycle substituted with one or more 6 to 10 membered heteroaryl, optionally substituted with one or more (C₁-C₆)alkyl, (C₁-C₆)alkoxy or halogen. In another embodiment, R³ is —(CH₂)ₙ-heterocycle substituted with one or more —CONH₂. In another embodiment, R³ is —(CH₂)ₙ-heterocycle substituted with one or more —COO—(C₁-C₆)alkyl, wherein the alkyl may be optionally substituted with one or more halogen.

In one embodiment, R³ is unsubstituted —O—(CH₂)ₙ-heterocycle. In another embodiment, R³ is —O—(CH₂)ₙ-heterocycle substituted with one or more (C₁-C₆)alkyl, itself optionally substituted with one or more halogen. In another embodiment, R³ is —O—(CH₂)ₙ-heterocycle substituted with one or more (C₁-C₆)alkoxy, itself substituted with one or more halogen. In another embodiment, R³ is —O—(CH₂)ₙ-heterocycle substituted with one or more oxo. In another embodiment, R³ is —O—(CH₂)ₙ-heterocycle substituted with one or more amino. In another embodiment, R³ is —O—(CH₂)ₙ-heterocycle substituted with one or more carboxyl. In another embodiment, R³ is —O—(CH₂)ₙ-heterocycle substituted with one or more cyano. In another embodiment, R³ is —O—(CH₂)ₙ-heterocycle substituted with one or more hydroxyl. In another embodiment, R³ is —O—(CH₂)ₙ-heterocycle substituted with one or more halogen. In another embodiment, R³ is —O—(CH₂)ₙ-heterocycle substituted with one or more deuterium. In another embodiment, R³ is —O—(CH₂)ₙ-heterocycle substituted with one or more 6 to 10 membered aryl, optionally substituted with one or more (C₁-C₆)alkyl. In another embodiment, R³ is —O—(CH₂)ₙ-heterocycle substituted with one or more 6 to 10 membered heteroaryl, optionally substituted with one or more (C₁-C₆)alkyl, (C₁-C₆)alkoxy or halogen. In another embodiment, R³ is —O—(CH₂)ₙ-heterocycle substituted with one or more —CONH₂. In another embodiment, R³ is —O—(CH₂)ₙ-heterocycle substituted with one or more —COO—(C₁-C₆)alkyl, wherein the alkyl may be optionally substituted with one or more halogen.

In one embodiment, R³ is unsubstituted —(CH₂)ₙ—O-heterocycle. In another embodiment, R³ is —(CH₂)ₙ—O-heterocycle substituted with one or more (C₁-C₆)alkyl, itself optionally substituted with one or more halogen. In another embodiment, R³ is —(CH₂)ₙ—O-heterocycle substituted with one or more (C₁-C₆)alkoxy, itself substituted with one or more halogen. In another embodiment, R³ is —(CH₂)ₙ—O-heterocycle substituted with one or more oxo. In another embodiment, R³ is —(CH₂)ₙ—O-heterocycle substituted with one or more amino. In another embodiment, R³ is —(CH₂)ₙ—O-heterocycle substituted with one or more carboxyl. In another embodiment, R³ is —(CH₂)ₙ—O-heterocycle substituted with one or more cyano. In another embodiment, R³ is —(CH₂)ₙ—O-heterocycle substituted with one or more hydroxyl. In another embodiment, R³ is —(CH₂)ₙ—O-heterocycle substituted with one or more halogen. In another embodiment, R³ is —(CH₂)ₙ—O-heterocycle substituted with one or more deuterium. In another embodiment, R³ is —(CH₂)ₙ—O-heterocycle substituted with one or more 6 to 10 membered aryl, optionally substituted with one or more (C₁-C₆)alkyl. In another embodiment, R³ is —(CH₂)ₙ—O-heterocycle substituted with one or more 6 to 10 membered heteroaryl, optionally substituted with one or more (C₁-C₆)alkyl, (C₁-C₆)alkoxy or halogen. In another embodiment, R³ is —(CH₂)ₙ—O-heterocycle substituted with one or more —CONH₂. In another embodiment, R³ is —(CH₂)ₙ—O-heterocycle substituted with one or more —COO—(C₁-C₆)alkyl, wherein the alkyl may be optionally substituted with one or more halogen.

In one embodiment, R³ is unsubstituted —(CH₂)ₙ-heteroaryl. In another embodiment, R³ is —(CH₂)ₙ-heteroaryl substituted with one or more (C₁-C₆)alkyl, itself optionally substituted with one or more halogen. In another embodiment, R³ is —(CH₂)ₙ-heteroaryl substituted with one or more (C₁-C₆)alkoxy, itself substituted with one or more halogen. In another embodiment, R³ is —(CH₂)ₙ-heteroaryl substituted with one or more oxo. In another embodiment, R³ is —(CH₂)ₙ-heteroaryl substituted with one or more amino. In another embodiment, R³ is —(CH₂)ₙ-heteroaryl substituted with one or more carboxyl. In another embodiment, R³ is —(CH₂)ₙ-heteroaryl substituted with one or more cyano. In another embodiment, R³ is —(CH₂)ₙ-heteroaryl substituted with one or more hydroxyl. In another embodiment, R³ is —(CH₂)ₙ-heteroaryl substituted with one or more halogen. In another embodiment, R³ is —(CH₂)ₙ-heteroaryl substituted with one or more deuterium. In another embodiment, R³ is —(CH₂)ₙ-heteroaryl substituted with one or more 6 to 10 membered aryl, optionally substituted with one or more (C₁-C₆)alkyl. In another embodiment, R³ is —(CH₂)ₙ-heteroaryl substituted with one or more 6 to 10 membered heteroaryl, optionally substituted with one or more (C₁-C₆)alkyl, (C₁-C₆)alkoxy or halogen. In another embodiment, R³ is —(CH₂)ₙ-heteroaryl substituted with one or more —CONH₂. In another embodiment, R³ is —(CH₂)ₙ-heteroaryl substituted with one or more —COO—(C₁-C₆)alkyl, wherein the alkyl may be optionally substituted with one or more halogen.

In one embodiment, R³ is unsubstituted —O—(CH₂)ₙ-heteroaryl. In another embodiment, R³ is —O—(CH₂)ₙ-heteroaryl substituted with one or more (C₁-C₆)alkyl, itself optionally substituted with one or more halogen. In another embodiment, R³ is —O—(CH₂)ₙ-heteroaryl substituted with one or more (C₁-C₆)alkoxy, itself substituted with one or more halogen. In another embodiment, R³ is —O—(CH₂)ₙ-heteroaryl substituted with one or more oxo. In another embodiment, R³ is —O—(CH₂)ₙ-heteroaryl substituted with one or more amino. In another embodiment, R³ is —O—(CH₂)ₙ-heteroaryl substituted with one or more carboxyl. In another embodiment, R³ is —O—(CH₂)ₙ-heteroaryl substituted with one or more cyano. In another embodiment, R³ is —O—(CH₂)ₙ-heteroaryl substituted with one or more hydroxyl. In another embodiment, R³ is —O—(CH₂)ₙ-heteroaryl substituted with one or more halogen. In another embodiment, R³ is —O—(CH₂)ₙ-heteroaryl substituted with one or more deuterium. In another embodiment, R³ is —O—(CH₂)ₙ-heteroaryl substituted with one or more 6 to 10 membered aryl, optionally substituted with one or more (C₁-C₆)alkyl. In another embodiment, R³ is —O—(CH₂)ₙ-heteroaryl substituted with one or more 6 to 10 membered heteroaryl, optionally substituted with one or more (C₁-C₆)alkyl, (C₁-C₆)alkoxy or halogen. In another embodiment, R³ is —O—(CH₂)ₙ-heteroaryl substituted with one or more —CONH₂. In another embodiment, R³ is —O—(CH₂)ₙ-heteroaryl substituted with one or more —COO—(C₁-C₆)alkyl, wherein the alkyl may be optionally substituted with one or more halogen.

In one embodiment, R³ is unsubstituted —(CH₂)ₙ—O-heteroaryl. In another embodiment, R³ is —(CH₂)ₙ—O-heteroaryl substituted with one or more (C₁-C₆)alkyl, itself optionally substituted with one or more halogen. In another embodiment, R³ is —(CH₂)ₙ—O-heteroaryl substituted with one or more (C₁-C₆)alkoxy, itself substituted with one or more halogen. In another embodiment, R³ is —(CH₂)ₙ—O-heteroaryl substituted with one or more oxo. In another embodiment, R³ is —(CH₂)ₙ—O-heteroaryl substituted with one or more amino. In another embodiment, R³ is —(CH₂)ₙ—O-heteroaryl substituted with one or more carboxyl. In another embodiment, R³ is —(CH₂)ₙ—O-heteroaryl substituted with one or more cyano. In another embodiment, R³ is —(CH₂)ₙ—O-heteroaryl substituted with one or more hydroxyl. In another embodiment, R³ is —(CH₂)ₙ—O-heteroaryl substituted with one or more halogen. In another embodiment, R³ is —(CH₂)ₙ—O-heteroaryl substituted with one or more deuterium. In another embodiment, R³ is —(CH₂)ₙ—O-heteroaryl substituted with one or more 6 to 10 membered aryl, optionally substituted with one or more (C₁-C₆)alkyl. In another embodiment, R³ is —(CH₂)ₙ—O-heteroaryl substituted with one or more 6 to 10 membered heteroaryl, optionally substituted with one or more (C₁-C₆)alkyl, (C₁-C₆)alkoxy or halogen. In another embodiment, R³ is —(CH₂)ₙ—O-heteroaryl substituted with one or more —CONH₂. In another embodiment, R³ is —(CH₂)ₙ—O-heteroaryl substituted with one or more —COO—(C₁-C₆)alkyl, wherein the alkyl may be optionally substituted with one or more halogen.

In one embodiment, n is 0. In another embodiment, n is 1. In another embodiment, n is 2.

All of the specific combinations that can result from the definition provided herein for X, R¹, R², Y, R³ and n are encompassed.

In one embodiment, X is CH₂.

In one embodiment, Y is aryl. In another embodiment, Y is phenyl.

In another embodiment wherein Y is phenyl, R³ is —(CH₂)ₙ-heterocycle. In one embodiment, the heterocycle is morpholinyl, piperidinyl or pyrrolidinyl.

In one embodiment, Y is a heteroaryl. In another embodiment, Y is a 10 membered hetero aryl. In another embodiment, Y is benzo[d]thiazole. In another embodiment, Y is benzofuran. In another embodiment, Y is quinoline.

In another embodiment where Y is heteroaryl, R³ is —(CH₂)ₙ-heterocycle. In one embodiment, the heterocycle is morpholinyl, piperidinyl or pyrrolidinyl.

In one embodiment, Y is a bond. In another embodiment where Y is a bond, R³ is —(CH₂)ₙ-heterocycle or —(CH₂)ₙ-heteroaryl.

In one embodiment, examples include, but are not limited to those listed in Table W, below:

TABLE W

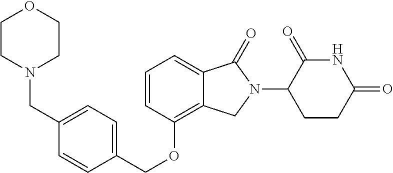 [SMILES: O=C1CCC(N2Cc3c(OCc4ccc(CN5CCOCC5)cc4)cccc3C2=O)C(=O)N1]

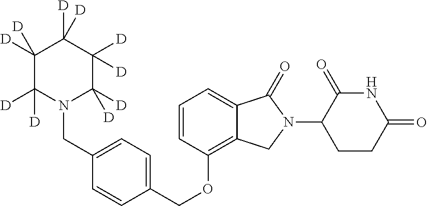 [SMILES: [2H]C1([2H])N(Cc2ccc(COc3cccc4c3CN(C3CCC(=O)NC3=O)C4=O)cc2)C([2H])([2H])C([2H])([2H])C([2H])([2H])C1([2H])[2H]]

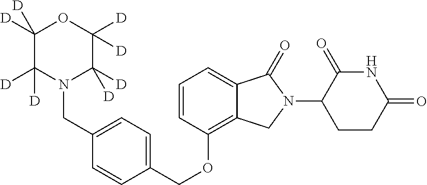 [SMILES: [2H]C1([2H])OC([2H])([2H])C([2H])([2H])N(Cc2ccc(COc3cccc4c3CN(C3CCC(=O)NC3=O)C4=O)cc2)C1([2H])[2H]]

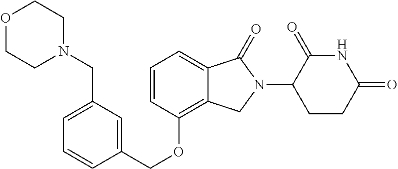 [SMILES: O=C1CCC(N2Cc3c(OCc4cccc(CN5CCOCC5)c4)cccc3C2=O)C(=O)N1]

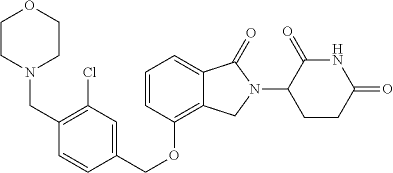 [SMILES: O=C1CCC(N2Cc3c(OCc4ccc(CN5CCOCC5)c(Cl)c4)cccc3C2=O)C(=O)N1]

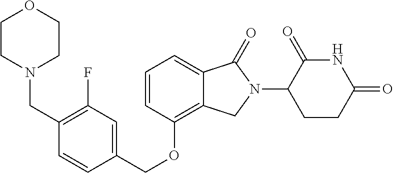 [SMILES: O=C1CCC(N2Cc3c(OCc4ccc(CN5CCOCC5)c(F)c4)cccc3C2=O)C(=O)N1]

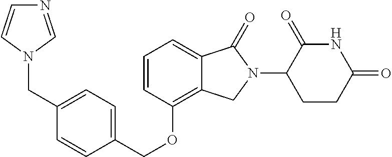 [SMILES: O=C1CCC(N2Cc3c(OCc4ccc(Cn5ccnc5)cc4)cccc3C2=O)C(=O)N1]

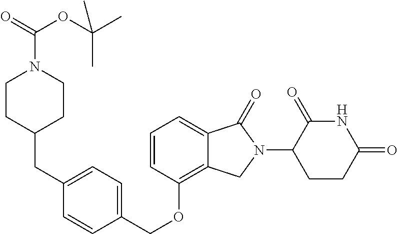 [SMILES: CC(C)(C)OC(=O)N1CCC(Cc2ccc(COc3cccc4c3CN(C3CCC(=O)NC3=O)C4=O)cc2)CC1]

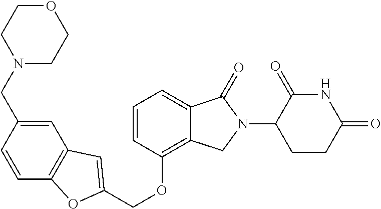 [SMILES: O=C1CCC(N2Cc3c(OCc4cc5cc(CN6CCOCC6)ccc5o4)cccc3C2=O)C(=O)N1]

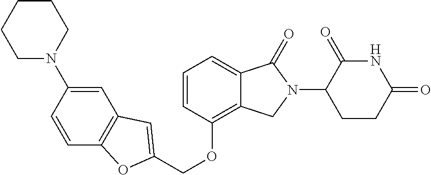 [SMILES: O=C1CCC(N2Cc3c(OCc4cc5cc(N6CCCCC6)ccc5o4)cccc3C2=O)C(=O)N1]

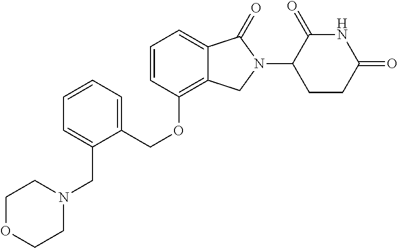 [SMILES: O=C1CCC(N2Cc3c(OCc4ccccc4CN4CCOCC4)cccc3C2=O)C(=O)N1]

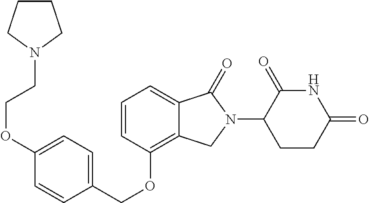 [SMILES: O=C1CCC(N2Cc3c(OCc4ccc(OCCN5CCCC5)cc4)cccc3C2=O)C(=O)N1]

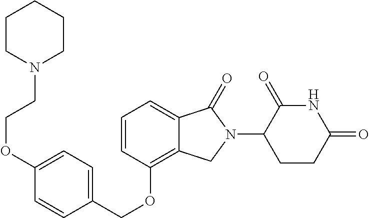 [SMILES: O=C1CCC(N2Cc3c(OCc4ccc(OCCN5CCCCC5)cc4)cccc3C2=O)C(=O)N1]

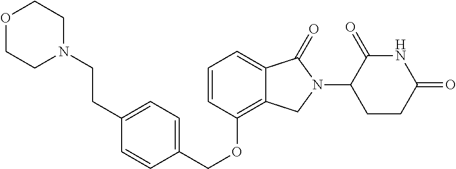 [SMILES: O=C1CCC(N2Cc3c(OCc4ccc(CCN5CCOCC5)cc4)cccc3C2=O)C(=O)N1]

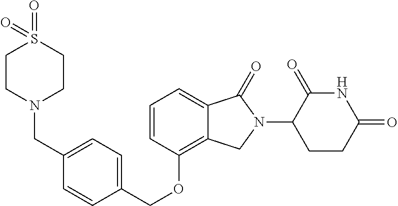 [SMILES: O=C1CCC(N2Cc3c(OCc4ccc(CN5CCS(=O)(=O)CC5)cc4)cccc3C2=O)C(=O)N1]

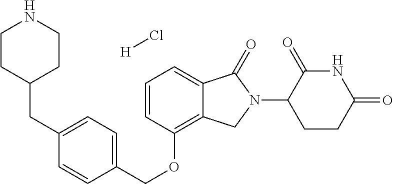

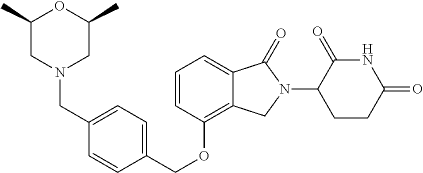 [SMILES: C[C@@H]1CN(Cc2ccc(COc3cccc4c3CN(C3CCC(=O)NC3=O)C4=O)cc2)C[C@H](C)O1]

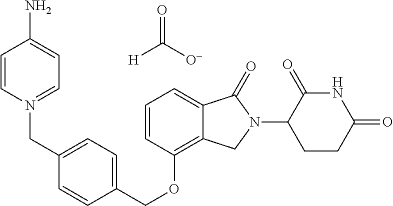

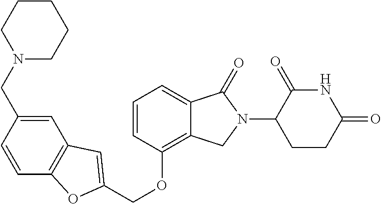 [SMILES: O=C1CCC(N2Cc3c(OCc4cc5cc(CN6CCCCC6)ccc5o4)cccc3C2=O)C(=O)N1]

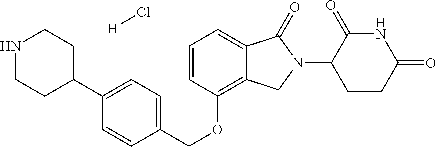

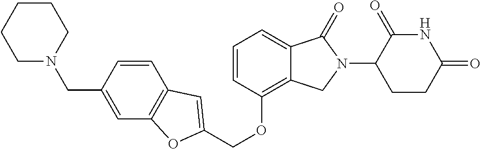 [SMILES: O=C1CCC(N2Cc3c(OCc4cc5ccc(CN6CCCCC6)cc5o4)cccc3C2=O)C(=O)N1]

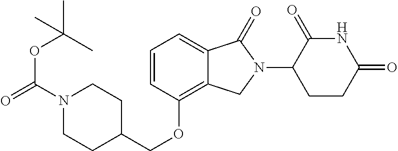 [SMILES: CC(C)(C)OC(=O)N1CCC(COc2cccc3c2CN(C2CCC(=O)NC2=O)C3=O)CC1]

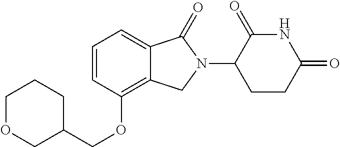 [SMILES: O=C1CCC(N2Cc3c(OCC4CCCOC4)cccc3C2=O)C(=O)N1]

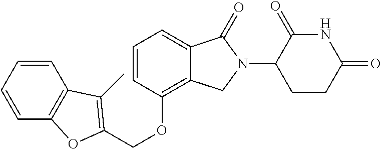 [SMILES: Cc1c(COc2cccc3c2CN(C2CCC(=O)NC2=O)C3=O)oc2ccccc12]

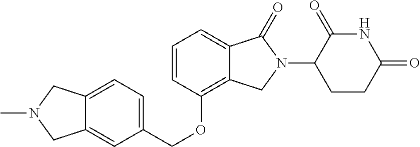 [SMILES: CN1Cc2ccc(COc3cccc4c3CN(C3CCC(=O)NC3=O)C4=O)cc2C1]

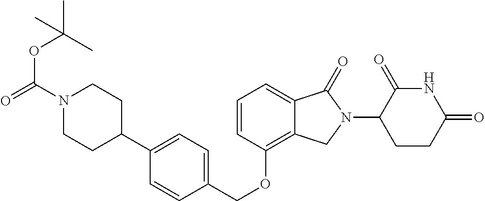 [SMILES: CC(C)(C)OC(=O)N1CCC(c2ccc(COc3cccc4c3CN(C3CCC(=O)NC3=O)C4=O)cc2)CC1]

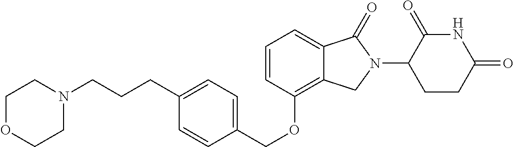 [SMILES: O=C1CCC(N2Cc3c(OCc4ccc(CCCN5CCOCC5)cc4)cccc3C2=O)C(=O)N1]

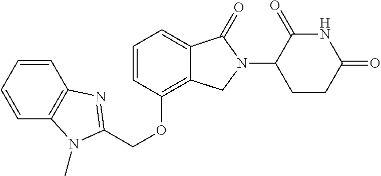 [SMILES: Cn1c(COc2cccc3c2CN(C2CCC(=O)NC2=O)C3=O)nc2ccccc21]

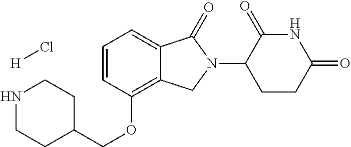

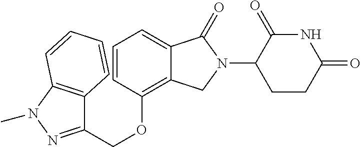 [SMILES: Cn1nc(COc2cccc3c2CN(C2CCC(=O)NC2=O)C3=O)c2ccccc21]

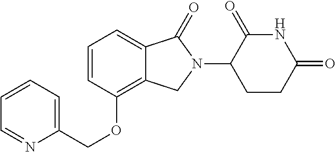 [SMILES: O=C1CCC(N2Cc3c(OCc4ccccn4)cccc3C2=O)C(=O)N1]

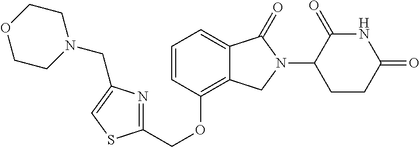 [SMILES: O=C1CCC(N2Cc3c(OCc4nc(CN5CCOCC5)cs4)cccc3C2=O)C(=O)N1]

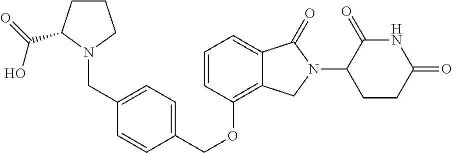 [SMILES: O=C1CCC(N2Cc3c(OCc4ccc(CN5CCC[C@H]5C(=O)O)cc4)cccc3C2=O)C(=O)N1]

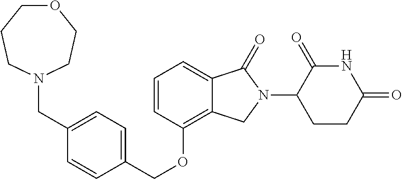 [SMILES: O=C1CCC(N2Cc3c(OCc4ccc(CN5CCCOCC5)cc4)cccc3C2=O)C(=O)N1]

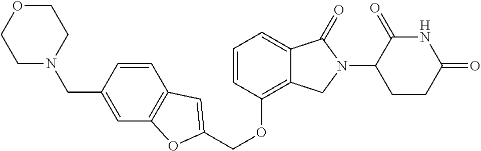 [SMILES: O=C1CCC(N2Cc3c(OCc4cc5ccc(CN6CCOCC6)cc5o4)cccc3C2=O)C(=O)N1]

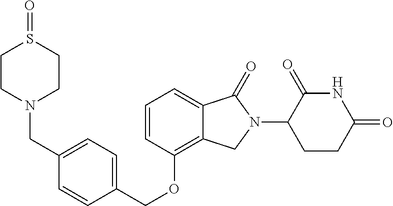 [SMILES: O=C1CCC(N2Cc3c(OCc4ccc(CN5CCS(=O)CC5)cc4)cccc3C2=O)C(=O)N1]

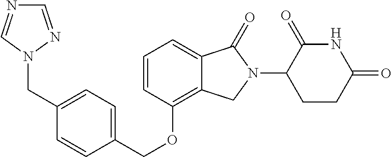 [SMILES: O=C1CCC(N2Cc3c(OCc4ccc(Cn5cncn5)cc4)cccc3C2=O)C(=O)N1]

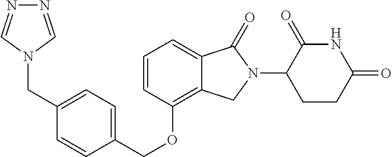 [SMILES: O=C1CCC(N2Cc3c(OCc4ccc(Cn5cnnc5)cc4)cccc3C2=O)C(=O)N1]

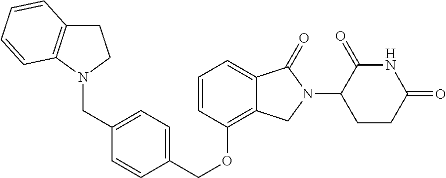 [SMILES: O=C1CCC(N2Cc3c(OCc4ccc(CN5CCc6ccccc65)cc4)cccc3C2=O)C(=O)N1]

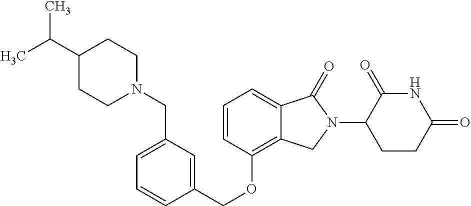 [SMILES: CC(C)C1CCN(Cc2cccc(COc3cccc4c3CN(C3CCC(=O)NC3=O)C4=O)c2)CC1]

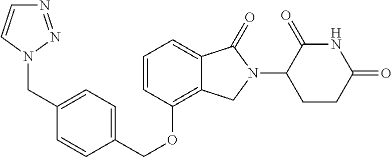 [SMILES: O=C1CCC(N2Cc3c(OCc4ccc(Cn5ccnn5)cc4)cccc3C2=O)C(=O)N1]

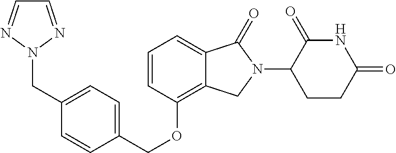 [SMILES: O=C1CCC(N2Cc3c(OCc4ccc(Cn5nccn5)cc4)cccc3C2=O)C(=O)N1]

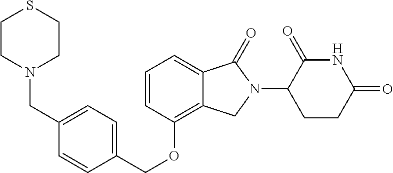 [SMILES: O=C1CCC(N2Cc3c(OCc4ccc(CN5CCSCC5)cc4)cccc3C2=O)C(=O)N1]

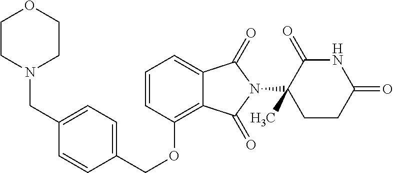

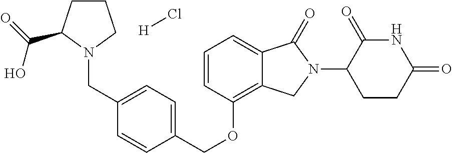

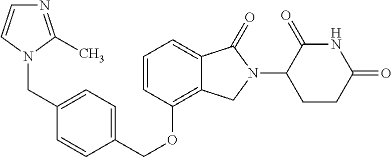 [SMILES: Cc1nccn1Cc1ccc(COc2cccc3c2CN(C2CCC(=O)NC2=O)C3=O)cc1]

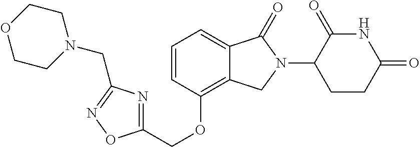 [SMILES: O=C1CCC(N2Cc3c(OCc4nc(CN5CCOCC5)no4)cccc3C2=O)C(=O)N1]

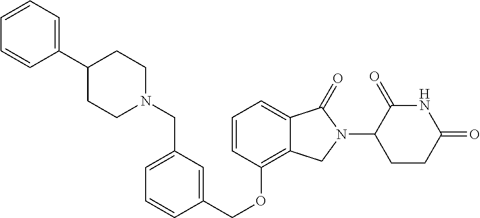 [SMILES: O=C1CCC(N2Cc3c(OCc4cccc(CN5CCC(c6ccccc6)CC5)c4)cccc3C2=O)C(=O)N1]

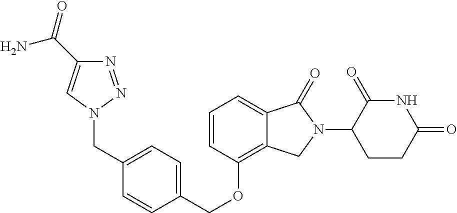 [SMILES: NC(=O)c1cn(Cc2ccc(COc3cccc4c3CN(C3CCC(=O)NC3=O)C4=O)cc2)nn1]

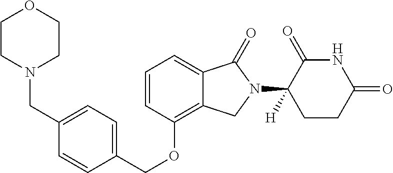 [SMILES: O=C1CC[C@@H](N2Cc3c(OCc4ccc(CN5CCOCC5)cc4)cccc3C2=O)C(=O)N1]

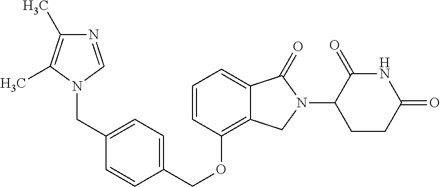 [SMILES: Cc1ncn(Cc2ccc(COc3cccc4c3CN(C3CCC(=O)NC3=O)C4=O)cc2)c1C]

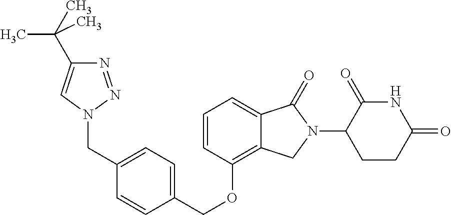 [SMILES: CC(C)(C)c1cn(Cc2ccc(COc3cccc4c3CN(C3CCC(=O)NC3=O)C4=O)cc2)nn1]

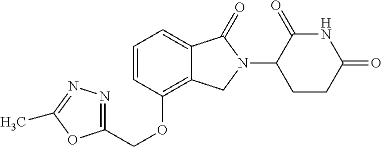 [SMILES: Cc1nnc(COc2cccc3c2CN(C2CCC(=O)NC2=O)C3=O)o1]

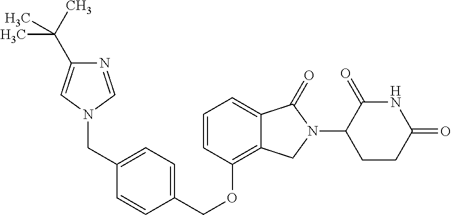 [SMILES: CC(C)(C)c1cn(Cc2ccc(COc3cccc4c3CN(C3CCC(=O)NC3=O)C4=O)cc2)cn1]

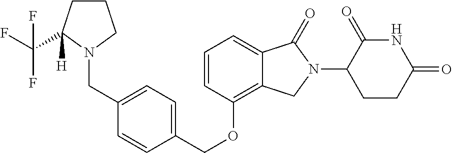

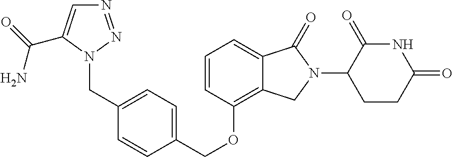 [SMILES: NC(=O)c1cnnn1Cc1ccc(COc2cccc3c2CN(C2CCC(=O)NC2=O)C3=O)cc1]

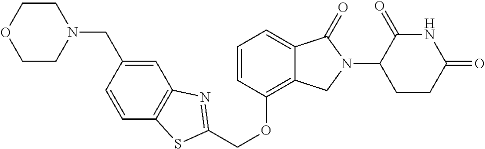 [SMILES: O=C1CCC(N2Cc3c(OCc4nc5cc(CN6CCOCC6)ccc5s4)cccc3C2=O)C(=O)N1]

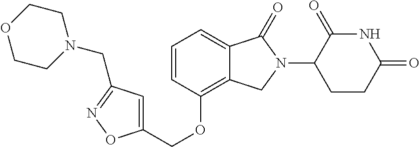 [SMILES: O=C1CCC(N2Cc3c(OCc4cc(CN5CCOCC5)no4)cccc3C2=O)C(=O)N1]

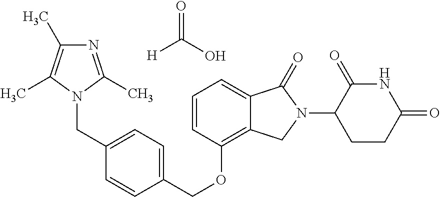

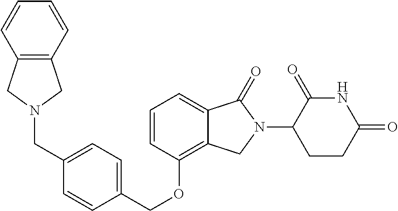 [SMILES: O=C1CCC(N2Cc3c(OCc4ccc(CN5Cc6ccccc6C5)cc4)cccc3C2=O)C(=O)N1]

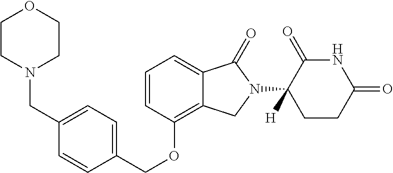 [SMILES: O=C1CC[C@H](N2Cc3c(OCc4ccc(CN5CCOCC5)cc4)cccc3C2=O)C(=O)N1]

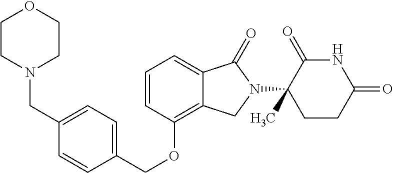 [SMILES: C[C@]1(N2Cc3c(OCc4ccc(CN5CCOCC5)cc4)cccc3C2=O)CCC(=O)NC1=O]

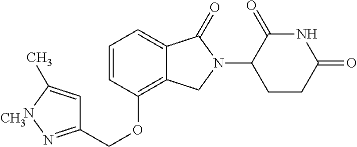

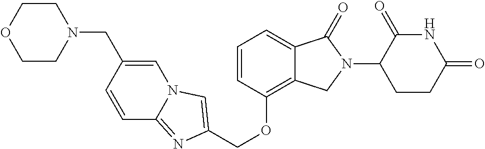 [SMILES: O=C1CCC(N2Cc3c(OCc4cn5cc(CN6CCOCC6)ccc5n4)cccc3C2=O)C(=O)N1]

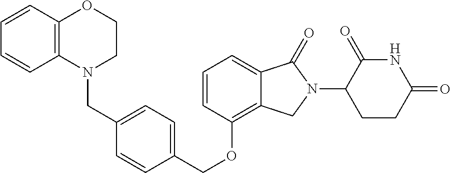 [SMILES: O=C1CCC(N2Cc3c(OCc4ccc(CN5CCOc6ccccc65)cc4)cccc3C2=O)C(=O)N1]

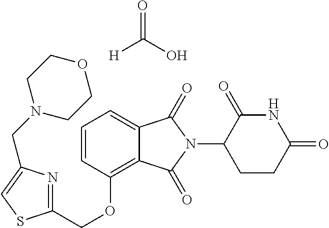

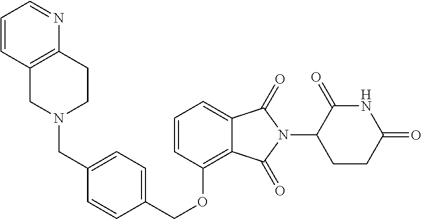 [SMILES: O=C1CCC(N2C(=O)c3cccc(OCc4ccc(CN5CCc6ncccc6C5)cc4)c3C2=O)C(=O)N1]

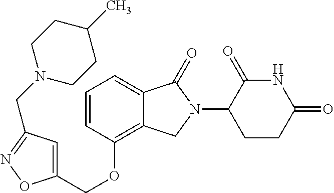 [SMILES: CC1CCN(Cc2cc(COc3cccc4c3CN(C3CCC(=O)NC3=O)C4=O)on2)CC1]

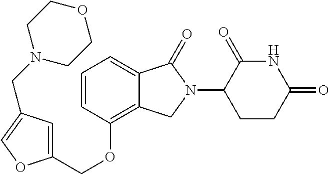 [SMILES: O=C1CCC(N2Cc3c(OCc4cc(CN5CCOCC5)co4)cccc3C2=O)C(=O)N1]

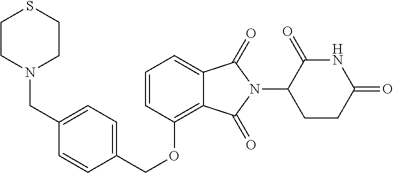 [SMILES: O=C1CCC(N2C(=O)c3cccc(OCc4ccc(CN5CCSCC5)cc4)c3C2=O)C(=O)N1]

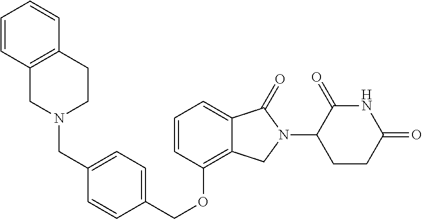 [SMILES: O=C1CCC(N2Cc3c(OCc4ccc(CN5CCc6ccccc6C5)cc4)cccc3C2=O)C(=O)N1]

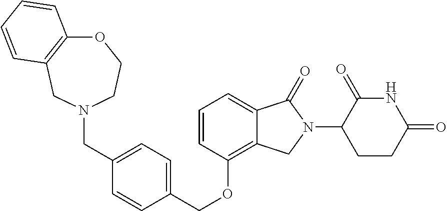 [SMILES: O=C1CCC(N2Cc3c(OCc4ccc(CN5CCOc6ccccc6C5)cc4)cccc3C2=O)C(=O)N1]

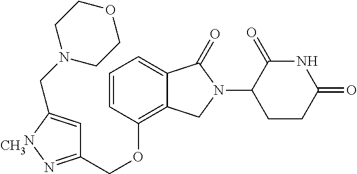

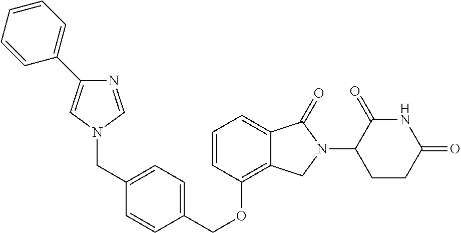 [SMILES: O=C1CCC(N2Cc3c(OCc4ccc(Cn5cnc(-c6ccccc6)c5)cc4)cccc3C2=O)C(=O)N1]

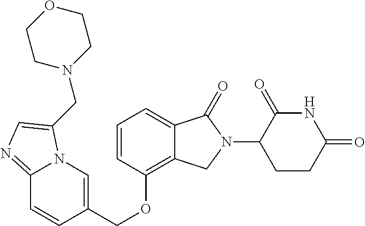 [SMILES: O=C1CCC(N2Cc3c(OCc4ccc5ncc(CN6CCOCC6)n5c4)cccc3C2=O)C(=O)N1]

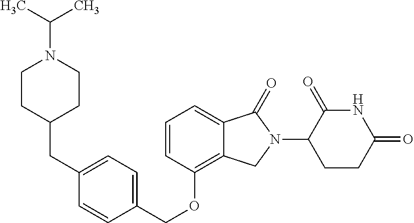 [SMILES: CC(C)N1CCC(Cc2ccc(COc3cccc4c3CN(C3CCC(=O)NC3=O)C4=O)cc2)CC1]

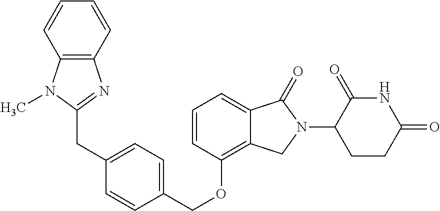 [SMILES: Cn1c(Cc2ccc(COc3cccc4c3CN(C3CCC(=O)NC3=O)C4=O)cc2)nc2ccccc21]

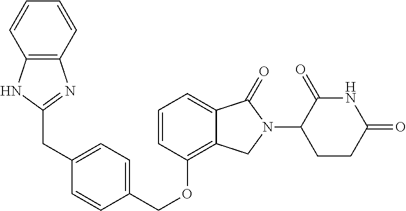 [SMILES: O=C1CCC(N2Cc3c(OCc4ccc(Cc5nc6ccccc6[nH]5)cc4)cccc3C2=O)C(=O)N1]

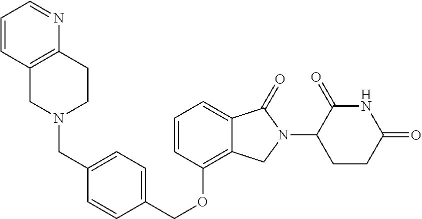 [SMILES: O=C1CCC(N2Cc3c(OCc4ccc(CN5CCc6ncccc6C5)cc4)cccc3C2=O)C(=O)N1]

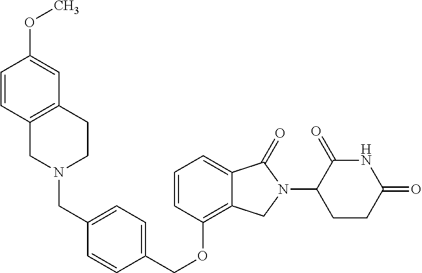 [SMILES: COc1ccc2c(c1)CCN(Cc1ccc(COc3cccc4c3CN(C3CCC(=O)NC3=O)C4=O)cc1)C2]

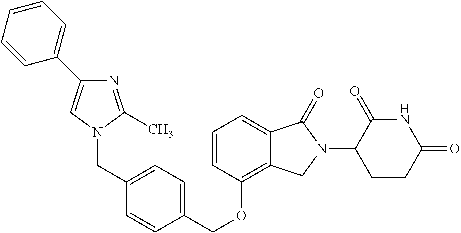 [SMILES: Cc1nc(-c2ccccc2)cn1Cc1ccc(COc2cccc3c2CN(C2CCC(=O)NC2=O)C3=O)cc1]

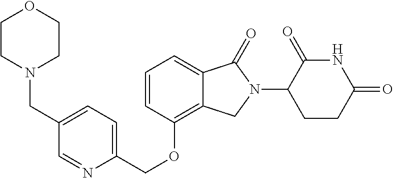 [SMILES: O=C1CCC(N2Cc3c(OCc4ccc(CN5CCOCC5)cn4)cccc3C2=O)C(=O)N1]

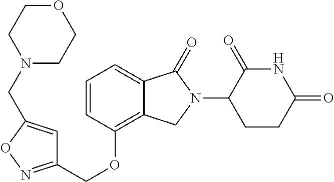 [SMILES: O=C1CCC(N2Cc3c(OCc4cc(CN5CCOCC5)on4)cccc3C2=O)C(=O)N1]

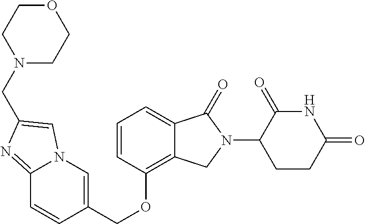 [SMILES: O=C1CCC(N2Cc3c(OCc4ccc5nc(CN6CCOCC6)cn5c4)cccc3C2=O)C(=O)N1]

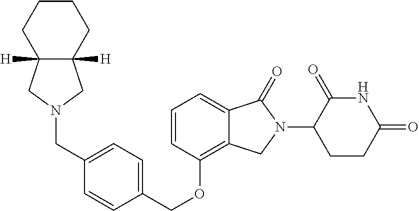 [SMILES: O=C1CCC(N2Cc3c(OCc4ccc(CN5C[C@H]6CCCC[C@H]6C5)cc4)cccc3C2=O)C(=O)N1]

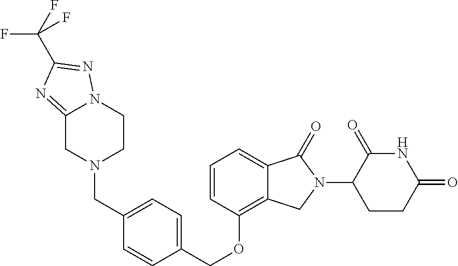 [SMILES: O=C1CCC(N2Cc3c(OCc4ccc(CN5CCn6nc(C(F)(F)F)nc6C5)cc4)cccc3C2=O)C(=O)N1]

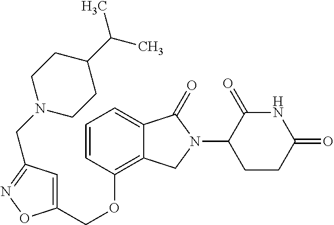 [SMILES: CC(C)C1CCN(Cc2cc(COc3cccc4c3CN(C3CCC(=O)NC3=O)C4=O)on2)CC1]

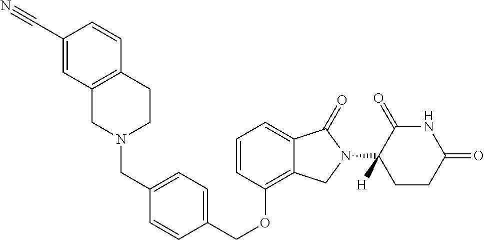

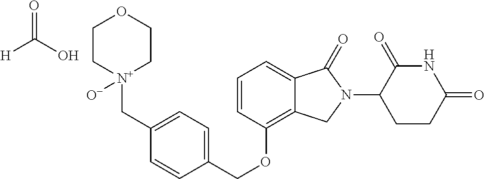

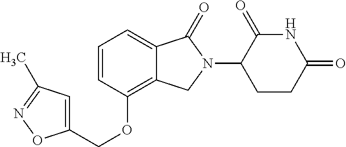 [SMILES: Cc1cc(COc2cccc3c2CN(C2CCC(=O)NC2=O)C3=O)on1]

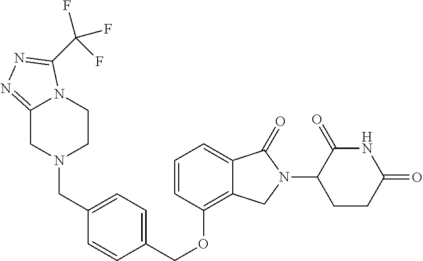 [SMILES: O=C1CCC(N2Cc3c(OCc4ccc(CN5CCn6c(nnc6C(F)(F)F)C5)cc4)cccc3C2=O)C(=O)N1]

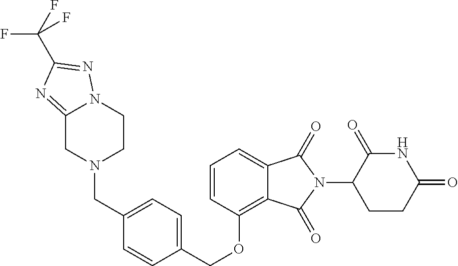 [SMILES: O=C1CCC(N2C(=O)c3cccc(OCc4ccc(CN5CCn6nc(C(F)(F)F)nc6C5)cc4)c3C2=O)C(=O)N1]

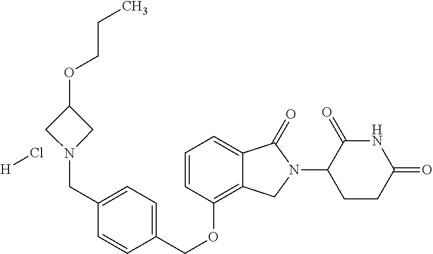

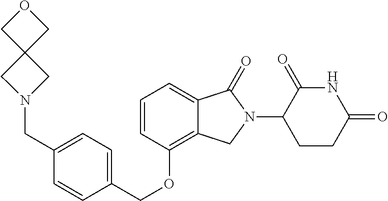 [SMILES: O=C1CCC(N2Cc3c(OCc4ccc(CN5CC6(COC6)C5)cc4)cccc3C2=O)C(=O)N1]

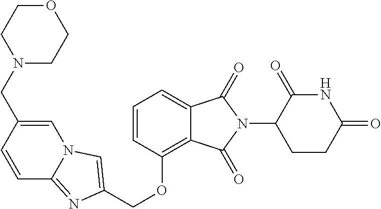 [SMILES: O=C1CCC(N2C(=O)c3cccc(OCc4cn5cc(CN6CCOCC6)ccc5n4)c3C2=O)C(=O)N1]

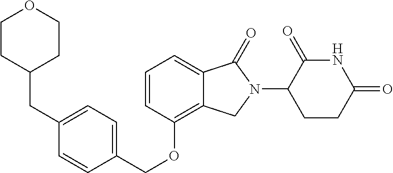 [SMILES: O=C1CCC(N2Cc3c(OCc4ccc(CC5CCOCC5)cc4)cccc3C2=O)C(=O)N1]

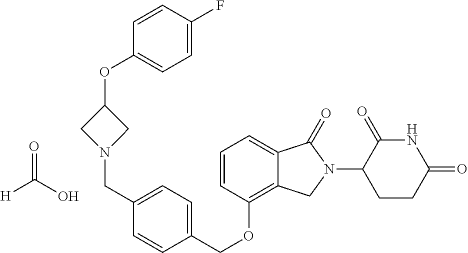

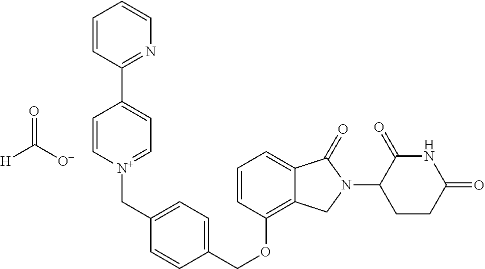

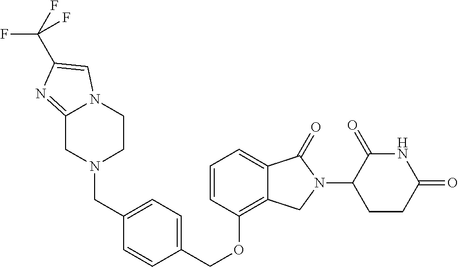 [SMILES: O=C1CCC(N2Cc3c(OCc4ccc(CN5CCn6cc(C(F)(F)F)nc6C5)cc4)cccc3C2=O)C(=O)N1]

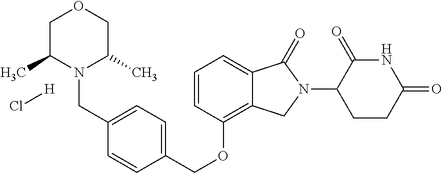

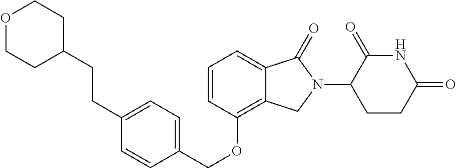 [SMILES: O=C1CCC(N2Cc3c(OCc4ccc(CCC5CCOCC5)cc4)cccc3C2=O)C(=O)N1]

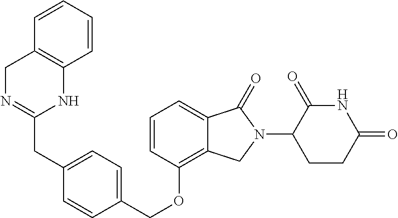 [SMILES: O=C1CCC(N2Cc3c(OCc4ccc(CC5=NCc6ccccc6N5)cc4)cccc3C2=O)C(=O)N1]

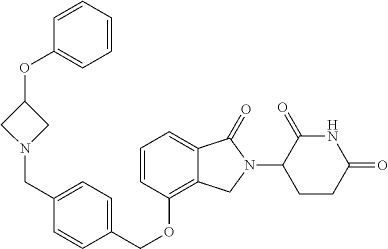 [SMILES: O=C1CCC(N2Cc3c(OCc4ccc(CN5CC(Oc6ccccc6)C5)cc4)cccc3C2=O)C(=O)N1]

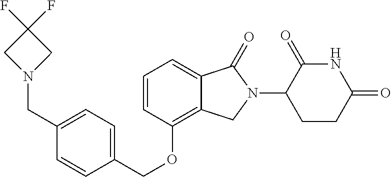 [SMILES: O=C1CCC(N2Cc3c(OCc4ccc(CN5CC(F)(F)C5)cc4)cccc3C2=O)C(=O)N1]

or a pharmaceutically acceptable salt, solvate or stereoisomer thereof.

In another embodiment, representative compounds are of formula (XX):

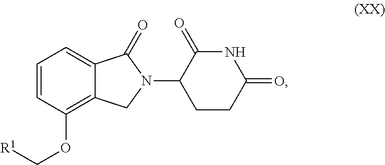

or a pharmaceutically acceptable salt, solvate or stereoisomer thereof, wherein:
R⁴ is unsubstituted 9 to 10 membered bicyclic ring is benzothiazole, quinoline, isoquinoline, naphthalene, 2,3-dihydro-1H-indene, benzo[d][1,2,3]triazole, imidazo[1,2-a]pyridine, benzofuran, 2,3-dihydrobenzofuran, benzothiophene, benzo[d]oxazole isoindoline or chroman; with the proviso that if the bicyclic ring is benzofuran or benzothiophene, then the ring is not connected to the isoindole ring through the 2-position.

In one embodiment, R⁴ is benzothiazole. In another embodiment, R⁴ is quinoline. In another embodiment, R⁴ is isoquinoline. In another embodiment, R⁴ is naphthalene. In another embodiment, R⁴ is 2,3-dihydro-1H-indene. In another embodiment, R⁴ is benzo[d][1,2,3]triazole. In another embodiment, R⁴ is imidazo[1,2-a]pyridine. In another embodiment, R⁴ is benzofuran. In another embodiment, R⁴ is 2,3-dihydrobenzofuran. In another embodiment, R⁴ is benzothiophene. In another embodiment, R⁴ is benzo[d]oxazole isoindoline. In another embodiment, R⁴ is chroman.

In one embodiment, specific examples include, but are not limited to those listed in Table X, below:

TABLE X

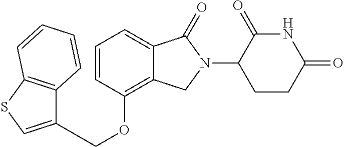 [SMILES: O=C1CCC(N2Cc3c(OCc4csc5ccccc45)cccc3C2=O)C(=O)N1]

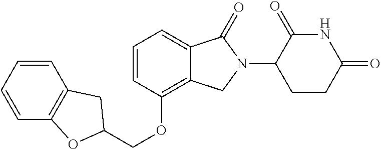 [SMILES: O=C1CCC(N2Cc3c(OCC4Cc5ccccc5O4)cccc3C2=O)C(=O)N1]

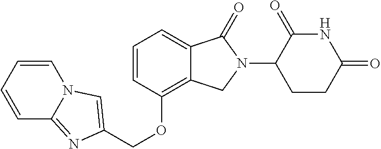 [SMILES: O=C1CCC(N2Cc3c(OCc4cn5ccccc5n4)cccc3C2=O)C(=O)N1]

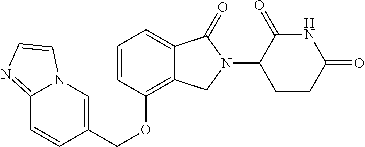 [SMILES: O=C1CCC(N2Cc3c(OCc4ccc5nccn5c4)cccc3C2=O)C(=O)N1]

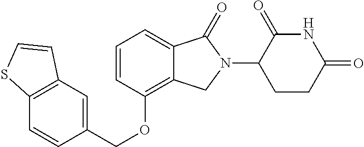 [SMILES: O=C1CCC(N2Cc3c(OCc4ccc5sccc5c4)cccc3C2=O)C(=O)N1]

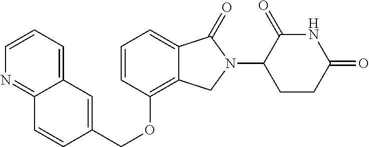 [SMILES: O=C1CCC(N2Cc3c(OCc4ccc5ncccc5c4)cccc3C2=O)C(=O)N1]

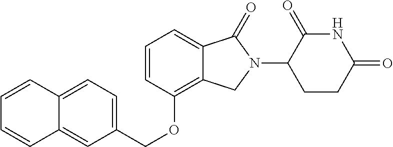 [SMILES: O=C1CCC(N2Cc3c(OCc4ccc5ccccc5c4)cccc3C2=O)C(=O)N1]

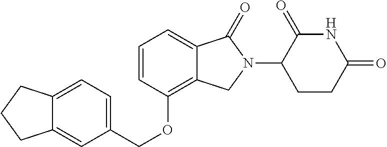 [SMILES: O=C1CCC(N2Cc3c(OCc4ccc5c(c4)CCC5)cccc3C2=O)C(=O)N1]

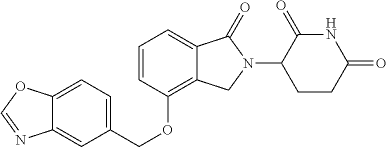 [SMILES: O=C1CCC(N2Cc3c(OCc4ccc5ocnc5c4)cccc3C2=O)C(=O)N1]

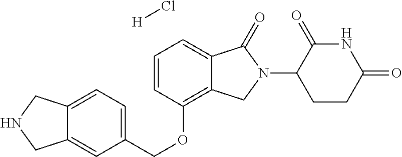

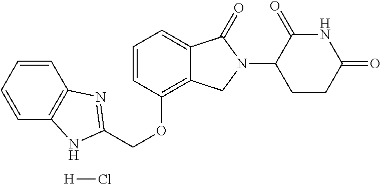

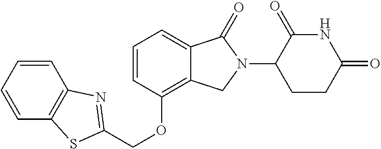 [SMILES: O=C1CCC(N2Cc3c(OCc4nc5ccccc5s4)cccc3C2=O)C(=O)N1]

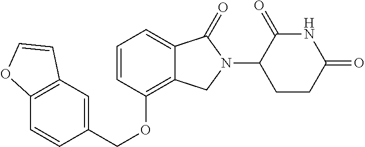 [SMILES: O=C1CCC(N2Cc3c(OCc4ccc5occc5c4)cccc3C2=O)C(=O)N1]

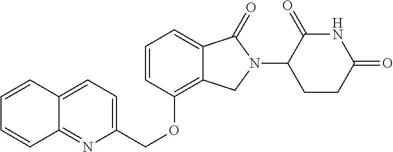 [SMILES: O=C1CCC(N2Cc3c(OCc4ccc5ccccc5n4)cccc3C2=O)C(=O)N1]

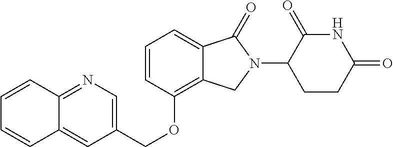 [SMILES: O=C1CCC(N2Cc3c(OCc4cnc5ccccc5c4)cccc3C2=O)C(=O)N1]

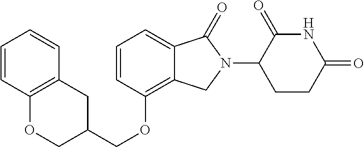 [SMILES: O=C1CCC(N2Cc3c(OCC4COc5ccccc5C4)cccc3C2=O)C(=O)N1]

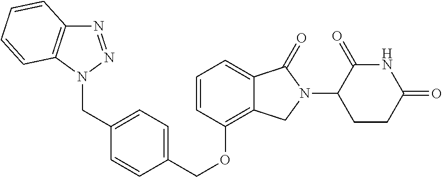 [SMILES: O=C1CCC(N2Cc3c(OCc4ccc(Cn5nnc6ccccc65)cc4)cccc3C2=O)C(=O)N1]

or a pharmaceutically acceptable salt, solvate or stereoisomer thereof.

In another embodiment, representative compounds are of formula (XXI):

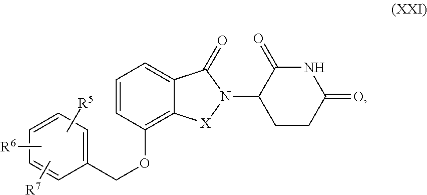

or a pharmaceutically acceptable salt, solvate or stereoisomer thereof, wherein:

### X is CH₂ or C═O;

R⁵, R⁶ and R⁷ are each independently hydrogen, halogen, nitro, carbamoyl, amino, —SO₂R⁸, —CONR⁹R¹⁰, —(C₁-C₆)alkyl or —(C₁-C₆)alkoxy, said alkyl or alkoxy may be optionally substituted with one or more halogen, amino, hydroxyl, or NR⁹R¹⁰;
R⁸ is: (C₁-C₆)alkyl, optionally substituted with (C₁-C₆)alkyl or (C₆-C₁₀)aryl; amino, optionally substituted with (C₁-C₆)alkyl or (C₆-C₁₀)aryl; or 6 to 10 membered heterocycle, optionally substituted with (C₁-C₆)alkyl or (C₆-C₁₀)aryl;
R⁹ and R¹⁰ are each independently hydrogen, 6 to 10 membered aryl, —COO—(C₁-C₆)alkyl, —(C₀-C₆)alkyl-CHO, —(C₀-C₆)alkyl-COOH, —(C₀-C₆)alkyl-NR^{9′}R^{10′}, —(C₀-C₆)alkyl- (5 to 10 membered heterocycle), —(C₁-C₆)alkyl-OH, —(C₁-C₆)alkyl-O—(C₁-C₆)alkyl, (C₁-C₆)alkyl, or (C₃-C₆)cycloalkyl; or
R⁹ and R¹⁰ together may form an optionally substituted 5 to 6 membered ring containing one or more heteroatoms; and
R^{9′} and R^{10′} are each independently hydrogen or (C₁-C₆)alkyl;
with the proviso that all of R⁵-R⁷ cannot be hydrogen; and
with the proviso that if one of R⁵-R⁷ is hydrogen and the remaining two of R⁵-R⁷ are both chloride, then the two chloride atoms cannot be on 3 and 4 position of the phenyl ring.

In one embodiment, R⁵ is hydrogen. In another embodiment, R⁵ is halogen. In another embodiment, R⁵ is nitro. In another embodiment, R⁵ is carbamoyl. In another embodiment, R⁵ is amino. In another embodiment, R⁵ is —SO₂R⁸. In another embodiment, R⁵ is —CONR⁹R¹⁰. In another embodiment, R⁵ is —(C₁-C₆)alkyl, optionally substituted with one or more halogen, amino, hydroxyl, or NR⁹R¹⁰. In another embodiment, R⁵ is —(C₁-C₆)alkoxy, optionally substituted with one or more halogen, amino, hydroxyl or NR⁹R¹⁰.

In one embodiment, R⁶ is hydrogen. In another embodiment, R⁶ is halogen. In another embodiment, R⁶ is nitro. In another embodiment, R⁶ is carbamoyl. In another embodiment, R⁶ is amino. In another embodiment, R⁶ is —SO₂R⁸. In another embodiment, R⁶ is —CONR⁹R¹⁰. In another embodiment, R⁶ is —(C₁-C₆)alkyl, optionally substituted with one or more halogen, amino, hydroxyl, or NR⁹R¹⁰. In another embodiment, R⁶ is —(C₁-C₆)alkoxy, optionally substituted with one or more halogen, amino, hydroxyl or NR⁹R¹⁰.

In one embodiment, R⁷ is hydrogen. In another embodiment, R⁷ is halogen. In another embodiment, R⁷ is nitro. In another embodiment, R⁷ is carbamoyl. In another embodiment, R⁷ is amino. In another embodiment, R⁷ is —SO₂R⁸. In another embodiment, R⁷ is —CONR⁹R¹⁰. In another embodiment, R⁷ is —(C₁-C₆)alkyl, optionally substituted with one or more halogen, amino, hydroxyl, or NR⁹R¹⁰. In another embodiment, R⁷ is —(C₁-C₆)alkoxy, optionally substituted with one or more halogen, amino, hydroxyl or NR⁹R¹⁰.

In one embodiment, R⁸ is (C₁-C₆)alkyl, optionally substituted with (C₁-C₆)alkyl or (C₆-C₁₀)aryl. In another embodiment, R⁸ is amino, optionally substituted with (C₁-C₆)alkyl or (C₆-C₁₀)aryl. In another embodiment, R⁸ is 6 to 10 membered heterocycle, optionally substituted with (C₁-C₆)alkyl or (C₆-C₁₀)aryl.

In one embodiment, R⁹ is hydrogen. In another embodiment, R⁹ is 6 to 10 membered aryl. In another embodiment, R⁹ is —COO—(C₁-C₆)alkyl. In another embodiment, R⁹ is —(C₀-C₆)alkyl-CHO. In another embodiment, R⁹ is —(C₀-C₆)alkyl-COOH. In another embodiment, R⁹ is —(C₀-C₆)alkyl-NR^{9′}R^{10′}. In another embodiment, R⁹ is —(C₀-C₆)alkyl- (5 to 10 membered heterocycle). In another embodiment, R⁹ is —(C₁-C₆)alkyl-OH. In another embodiment, R⁹ is —(C₁-C₆)alkyl-O—(C₁-C₆)alkyl. In another embodiment, R⁹ is (C₁-C₆)alkyl. In another embodiment, R⁹ is (C₃-C₆)cycloalkyl.

In one embodiment, R¹⁰ is hydrogen. In another embodiment, R¹⁰ is 6 to 10 membered aryl. In another embodiment, R¹⁰ is —COO—(C₁-C₆)alkyl. In another embodiment, R¹⁰ is —(C₀-C₆)alkyl-CHO. In another embodiment, R¹⁰ is —(C₀-C₆)alkyl-COOH. In another embodiment, R¹⁰ is —(C₀-C₆)alkyl-NR^{9′}R^{10′}. In another embodiment, R¹⁰ is —(C₀-C₆)alkyl- (5 to 10 membered heterocycle). In another embodiment, R¹⁰ is —(C₁-C₆)alkyl-OH. In another embodiment, R¹⁰ is —(C₁-C₆)alkyl-O—(C₁-C₆)alkyl. In another embodiment, R¹⁰ is (C₁-C₆)alkyl. In another embodiment, R¹⁰ is (C₃-C₆)cycloalkyl.

In one embodiment, R⁹ and R¹⁰ together form a 5 to 6 membered ring. In one embodiment, the ring contains one or more heteroatoms. In one embodiment, the heteroatoms are selected from the group consisting of N, S and O.

In one embodiment, R^{9′} is hydrogen. In another embodiment, R^{9′} is (C₁-C₆)alkyl.

In one embodiment, R^{10′} is hydrogen. In another embodiment, R^{10′} is (C₁-C₆)alkyl.

In certain embodiments, provided herein are compounds that result from any combination of R⁵-R¹⁰ and R^{9′}-R^{10′}.

In one embodiment, one of R⁵-R⁷ is hydrogen and the remaining two of R⁵-R⁷ are halogen. In one embodiment, one of R⁵-R⁷ is hydrogen and the remaining two of R⁵-R⁷ are (C₁-C₆)alkoxy. In one embodiment, one of R⁵-R⁷ is hydrogen and the remaining two of R⁵-R⁷ are (C₁-C₆)alkyl. In one embodiment, R⁵ is hydrogen, R⁶ is halogen, and R⁷ is (C₁-C₆)alkoxy.

In one embodiment, two of R⁵-R⁷ are hydrogen and the remaining one of R⁵-R⁷ is halogen. In one embodiment, two of R⁵-R⁷ are hydrogen and the remaining one of R⁵-R⁷ is (C₁-C₆)alkoxy. In one embodiment, two of R⁵-R⁷ are hydrogen and the remaining one of R⁵-R⁷ is (C₁-C₆)alkyl.

In one embodiment, specific examples include, but are not limited to those listed in Table Y, below:

TABLE Y

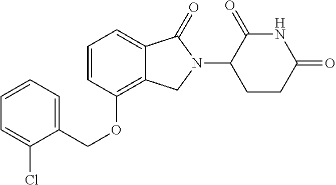 [SMILES: O=C1CCC(N2Cc3c(OCc4ccccc4Cl)cccc3C2=O)C(=O)N1]

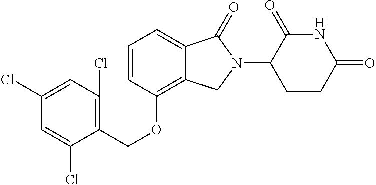 [SMILES: O=C1CCC(N2Cc3c(OCc4c(Cl)cc(Cl)cc4Cl)cccc3C2=O)C(=O)N1]

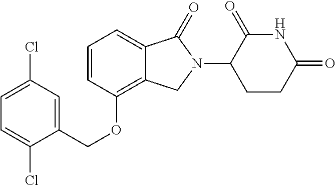 [SMILES: O=C1CCC(N2Cc3c(OCc4cc(Cl)ccc4Cl)cccc3C2=O)C(=O)N1]

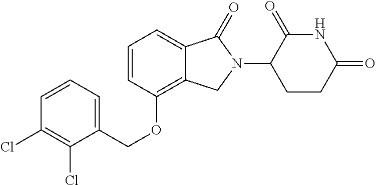 [SMILES: O=C1CCC(N2Cc3c(OCc4cccc(Cl)c4Cl)cccc3C2=O)C(=O)N1]

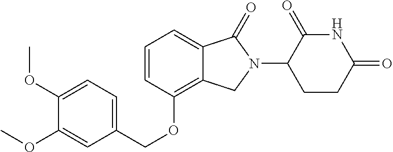 [SMILES: COc1ccc(COc2cccc3c2CN(C2CCC(=O)NC2=O)C3=O)cc1OC]

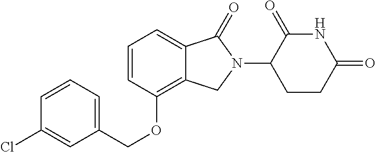 [SMILES: O=C1CCC(N2Cc3c(OCc4cccc(Cl)c4)cccc3C2=O)C(=O)N1]

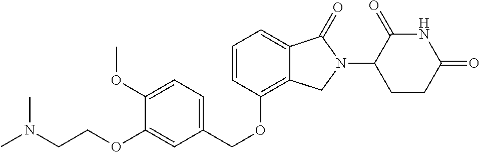 [SMILES: COc1ccc(COc2cccc3c2CN(C2CCC(=O)NC2=O)C3=O)cc1OCCN(C)C]

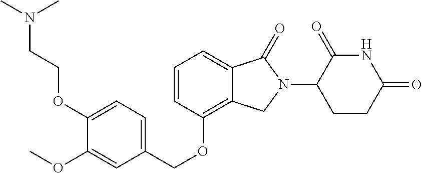 [SMILES: COc1cc(COc2cccc3c2CN(C2CCC(=O)NC2=O)C3=O)ccc1OCCN(C)C]

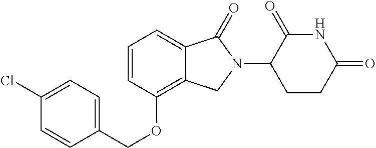 [SMILES: O=C1CCC(N2Cc3c(OCc4ccc(Cl)cc4)cccc3C2=O)C(=O)N1]

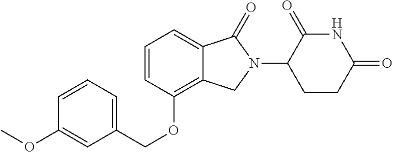 [SMILES: COc1cccc(COc2cccc3c2CN(C2CCC(=O)NC2=O)C3=O)c1]

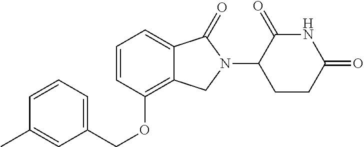 [SMILES: Cc1cccc(COc2cccc3c2CN(C2CCC(=O)NC2=O)C3=O)c1]

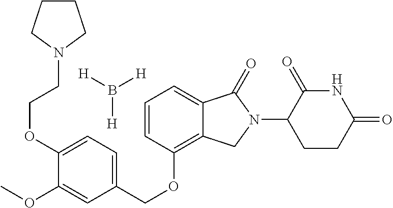

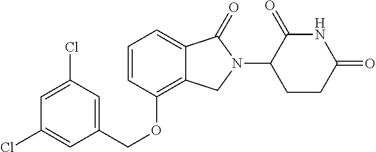 [SMILES: O=C1CCC(N2Cc3c(OCc4cc(Cl)cc(Cl)c4)cccc3C2=O)C(=O)N1]

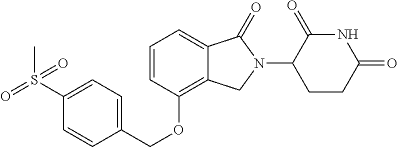 [SMILES: CS(=O)(=O)c1ccc(COc2cccc3c2CN(C2CCC(=O)NC2=O)C3=O)cc1]

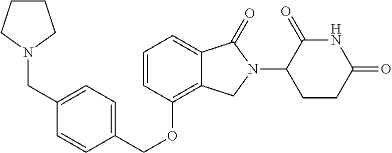 [SMILES: O=C1CCC(N2Cc3c(OCc4ccc(CN5CCCC5)cc4)cccc3C2=O)C(=O)N1]

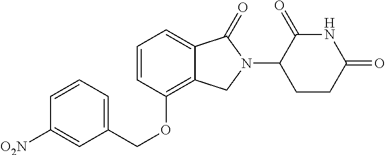 [SMILES: O=C1CCC(N2Cc3c(OCc4cccc([N+](=O)[O-])c4)cccc3C2=O)C(=O)N1]

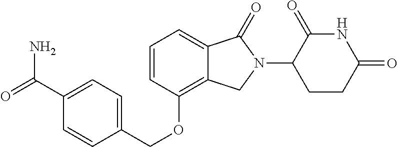 [SMILES: NC(=O)c1ccc(COc2cccc3c2CN(C2CCC(=O)NC2=O)C3=O)cc1]

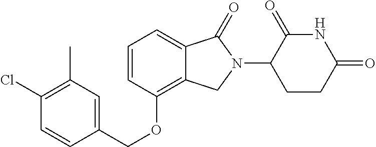 [SMILES: Cc1cc(COc2cccc3c2CN(C2CCC(=O)NC2=O)C3=O)ccc1Cl]

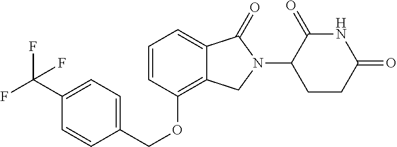 [SMILES: O=C1CCC(N2Cc3c(OCc4ccc(C(F)(F)F)cc4)cccc3C2=O)C(=O)N1]

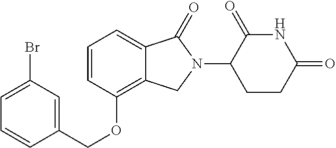 [SMILES: O=C1CCC(N2Cc3c(OCc4cccc(Br)c4)cccc3C2=O)C(=O)N1]

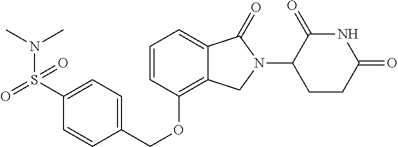 [SMILES: CN(C)S(=O)(=O)c1ccc(COc2cccc3c2CN(C2CCC(=O)NC2=O)C3=O)cc1]

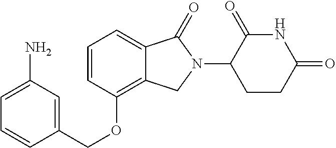 [SMILES: Nc1cccc(COc2cccc3c2CN(C2CCC(=O)NC2=O)C3=O)c1]

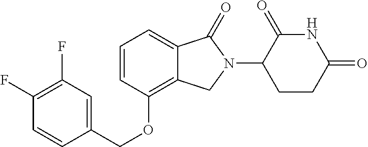 [SMILES: O=C1CCC(N2Cc3c(OCc4ccc(F)c(F)c4)cccc3C2=O)C(=O)N1]

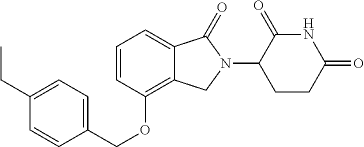 [SMILES: CCc1ccc(COc2cccc3c2CN(C2CCC(=O)NC2=O)C3=O)cc1]

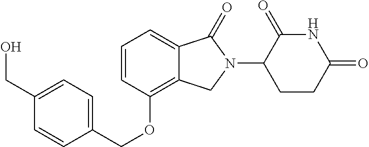 [SMILES: O=C1CCC(N2Cc3c(OCc4ccc(CO)cc4)cccc3C2=O)C(=O)N1]

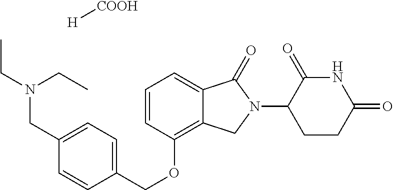 [SMILES: O=CO ; CCN(CC)Cc1ccc(COc2cccc3c2CN(C2CCC(=O)NC2=O)C3=O)cc1]

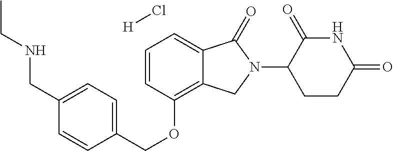

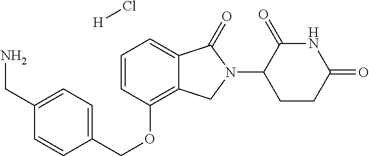

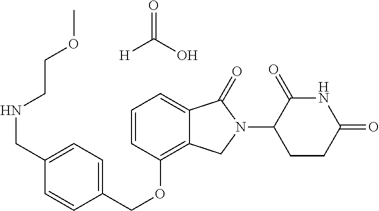

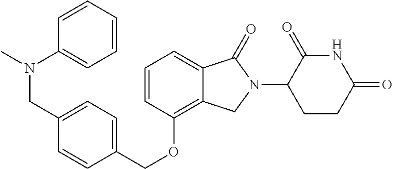 [SMILES: CN(Cc1ccc(COc2cccc3c2CN(C2CCC(=O)NC2=O)C3=O)cc1)c1ccccc1]

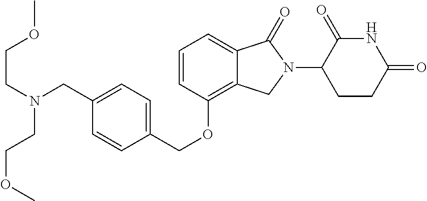 [SMILES: COCCN(CCOC)Cc1ccc(COc2cccc3c2CN(C2CCC(=O)NC2=O)C3=O)cc1]

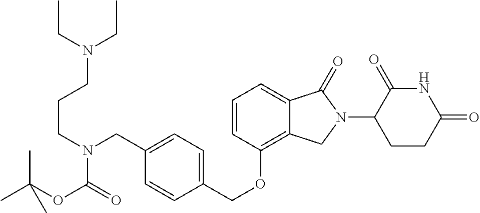 [SMILES: CCN(CC)CCCN(Cc1ccc(COc2cccc3c2CN(C2CCC(=O)NC2=O)C3=O)cc1)C(=O)OC(C)(C)C]

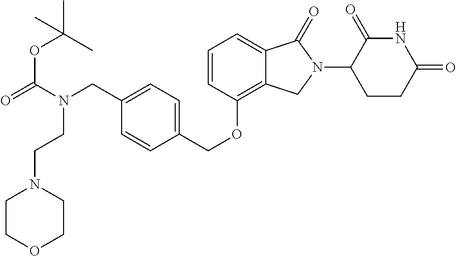 [SMILES: CC(C)(C)OC(=O)N(CCN1CCOCC1)Cc1ccc(COc2cccc3c2CN(C2CCC(=O)NC2=O)C3=O)cc1]

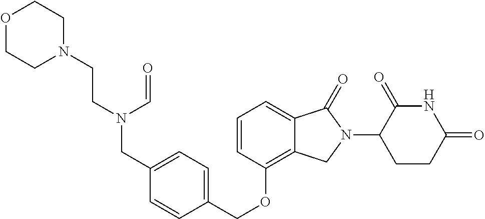 [SMILES: O=CN(CCN1CCOCC1)Cc1ccc(COc2cccc3c2CN(C2CCC(=O)NC2=O)C3=O)cc1]

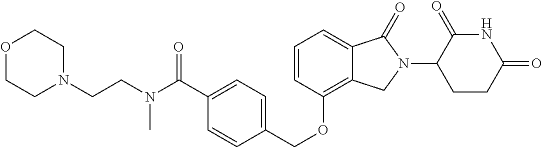 [SMILES: CN(CCN1CCOCC1)C(=O)c1ccc(COc2cccc3c2CN(C2CCC(=O)NC2=O)C3=O)cc1]

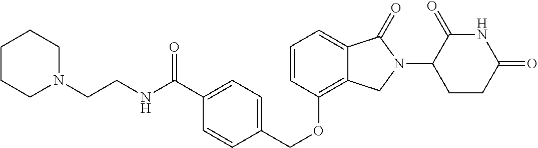 [SMILES: O=C1CCC(N2Cc3c(OCc4ccc(C(=O)NCCN5CCCCC5)cc4)cccc3C2=O)C(=O)N1]

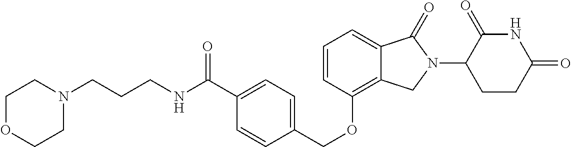 [SMILES: O=C1CCC(N2Cc3c(OCc4ccc(C(=O)NCCCN5CCOCC5)cc4)cccc3C2=O)C(=O)N1]

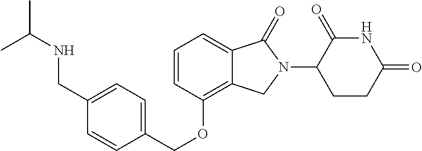 [SMILES: CC(C)NCc1ccc(COc2cccc3c2CN(C2CCC(=O)NC2=O)C3=O)cc1]

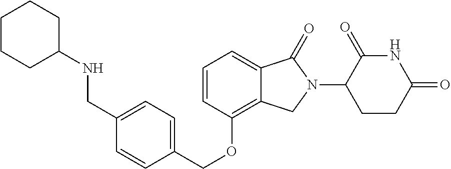 [SMILES: O=C1CCC(N2Cc3c(OCc4ccc(CNC5CCCCC5)cc4)cccc3C2=O)C(=O)N1]

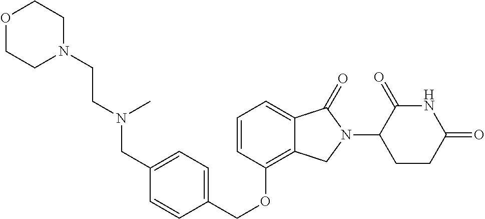 [SMILES: CN(CCN1CCOCC1)Cc1ccc(COc2cccc3c2CN(C2CCC(=O)NC2=O)C3=O)cc1]

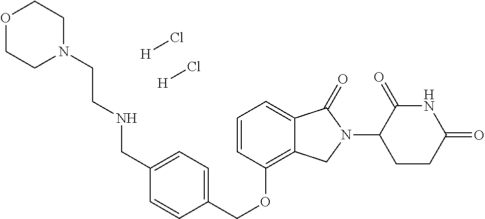

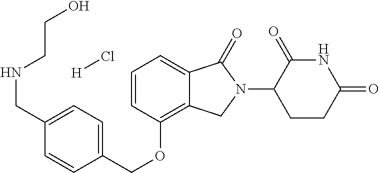

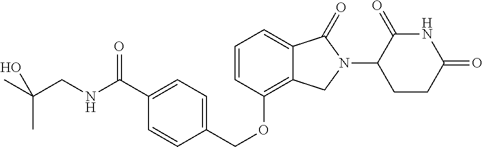 [SMILES: CC(C)(O)CNC(=O)c1ccc(COc2cccc3c2CN(C2CCC(=O)NC2=O)C3=O)cc1]

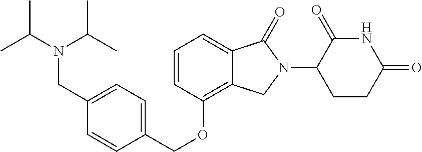 [SMILES: CC(C)N(Cc1ccc(COc2cccc3c2CN(C2CCC(=O)NC2=O)C3=O)cc1)C(C)C]

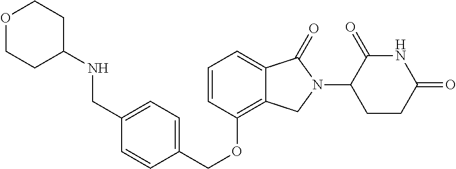 [SMILES: O=C1CCC(N2Cc3c(OCc4ccc(CNC5CCOCC5)cc4)cccc3C2=O)C(=O)N1]

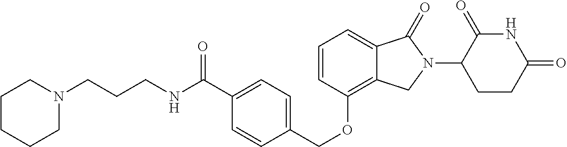 [SMILES: O=C1CCC(N2Cc3c(OCc4ccc(C(=O)NCCCN5CCCCC5)cc4)cccc3C2=O)C(=O)N1]

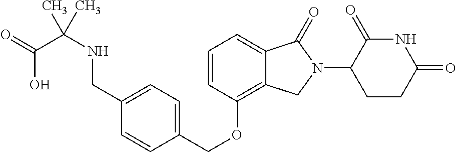 [SMILES: CC(C)(NCc1ccc(COc2cccc3c2CN(C2CCC(=O)NC2=O)C3=O)cc1)C(=O)O]

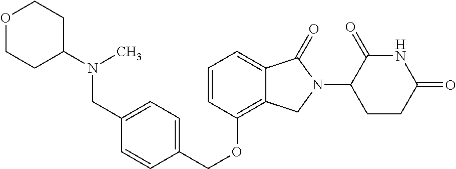 [SMILES: CN(Cc1ccc(COc2cccc3c2CN(C2CCC(=O)NC2=O)C3=O)cc1)C1CCOCC1]

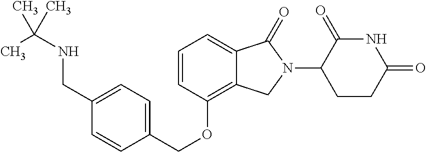 [SMILES: CC(C)(C)NCc1ccc(COc2cccc3c2CN(C2CCC(=O)NC2=O)C3=O)cc1]

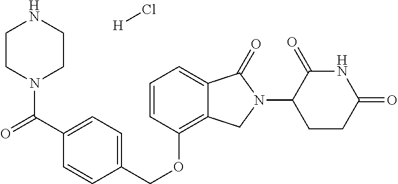 [SMILES: O=C1CCC(N2Cc3c(OCc4ccc(C(=O)N5CCNCC5)cc4)cccc3C2=O)C(=O)N1 ; Cl]

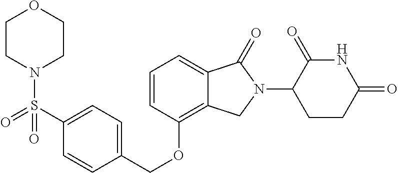 [SMILES: O=C1CCC(N2Cc3c(OCc4ccc(S(=O)(=O)N5CCOCC5)cc4)cccc3C2=O)C(=O)N1]

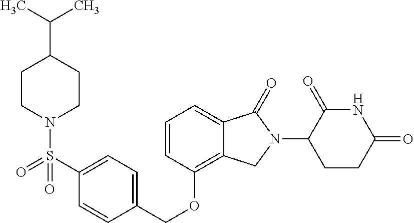 [SMILES: CC(C)C1CCN(S(=O)(=O)c2ccc(COc3cccc4c3CN(C3CCC(=O)NC3=O)C4=O)cc2)CC1]

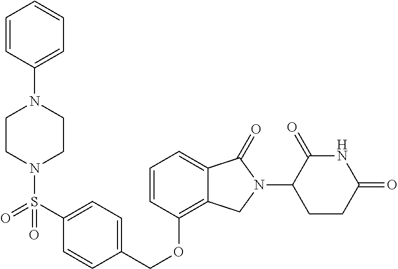 [SMILES: O=C1CCC(N2Cc3c(OCc4ccc(S(=O)(=O)N5CCN(c6ccccc6)CC5)cc4)cccc3C2=O)C(=O)N1]

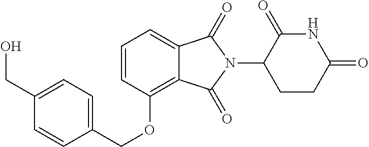 [SMILES: O=C1CCC(N2C(=O)c3cccc(OCc4ccc(CO)cc4)c3C2=O)C(=O)N1]

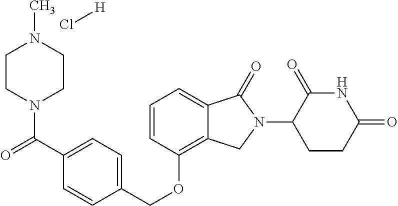

or a pharmaceutically acceptable salt, solvate or stereoisomer thereof.

In another embodiment, representative compounds are of formula (XXII):

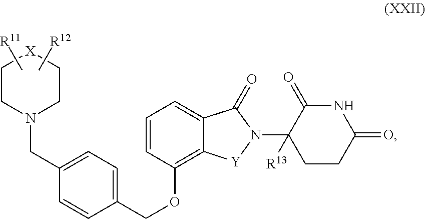

or a pharmaceutically acceptable salt, solvate or stereoisomer thereof, wherein:
X is N or C;
Y is CH₂ or C═O;
R¹¹ and R¹² are each independently hydrogen, —(C₁-C₆)alkyl, —(C₁-C₆)alkyl-(C₃-C₆)cycloalkyl, —(C₁-C₆)alkoxy, —(C₆-C₁₀)aryl, —CO(C₁-C₆)alkyl, —CO(C₃-C₆)cycloalkyl, —CO(C₆-C₁₀)aryl, —COO(C₁-C₆)alkyl, halogen, hydroxyl, oxo, 3 to 10 membered heterocycle, 6 to 10 membered heteroaryl, —NHCO(C₁-C₆)alkyl, —(CH₂)ₙ-phenyl, —SO₂(C₁-C₆)alkyl, —SO₂(C₃-C₆)cycloalkyl, —SO₂(C₆-C₁₀)aryl or —NR¹⁴R¹⁵, wherein the alkyl, aryl or heteroaryl portion of each of the groups may be optionally substituted with one or more halogen, hydroxyl or —(C₁-C₆)alkoxy;
R¹³ is hydrogen or —(C₁-C₆)alkyl;
R¹⁴ and R¹⁵ are each independently hydrogen or —(C₁-C₆)alkyl; and
n is 0, 1, 2 or 3.

In one embodiment, X is N. In another embodiment, X is C.

In one embodiment, Y is CH₂. In another embodiment, Y is C═O.

In one embodiment, R¹¹ is hydrogen. In another embodiment, R¹¹ is —(C₁-C₆)alkyl. In another embodiment, R¹¹ is —(C₁-C₆)alkyl-(C₃-C₆)cycloalkyl. In another embodiment, R¹¹ is —(C₁-C₆)alkoxy. In another embodiment, R¹¹ is —(C₆-C₁₀)aryl. In another embodiment, R¹¹ is —CO(C₁-C₆)alkyl. In another embodiment, R¹¹ is —CO(C₃-C₆)cycloalkyl. In another embodiment, R¹¹ is —CO(C₆-C₁₀)aryl. In another embodiment, R¹¹ is —COO(C₁-C₆)alkyl. In another embodiment, R¹¹ is halogen. In another embodiment, R¹¹ is hydroxyl. In another embodiment, R¹¹ is oxo. In another embodiment, R¹¹ is 3 to 10 membered heterocycle. In another embodiment, R¹¹ is 6 to 10 membered heteroaryl. In another embodiment, R¹¹ is —NHCO(C₁-C₆)alkyl. In another embodiment, R¹¹ is —(CH₂)ₙ-phenyl. In another embodiment, R¹¹ is —SO²(C₁-C₆)alkyl. In another embodiment, R¹¹ is —SO²(C₃-C₆)cycloalkyl. In another embodiment, R¹¹ is —SO₂(C₆-C₁₀)aryl. In another embodiment, R¹¹ is —NR¹⁴R¹⁵. In another embodiment, is the alkyl, aryl or heteroaryl portion of R¹¹ is substituted with one or more halogen, hydroxyl and/or —(C₁-C₆)alkoxy.

In one embodiment, R¹² is hydrogen. In another embodiment, R¹² is —(C₁-C₆)alkyl. In another embodiment, R¹² is —(C₁-C₆)alkyl-(C₃-C₆)cycloalkyl. In another embodiment, R¹² is —(C₁-C₆)alkoxy. In another embodiment, R¹² is —(C₆-C₁₀)aryl. In another embodiment, R¹² is —CO(C₁-C₆)alkyl. In another embodiment, R¹² is —CO(C₃-C₆)cycloalkyl. In another embodiment, R¹² is —CO(C₆-C₁₀)aryl. In another embodiment, R¹² is —COO(C₁-C₆)alkyl. In another embodiment, R¹² is halogen. In another embodiment, R¹² is hydroxyl. In another embodiment, R¹² is oxo. In another embodiment, R¹² is 3 to 10 membered heterocycle. In another embodiment, R¹² is 6 to 10 membered heteroaryl. In another embodiment, R¹² is —NHCO(C₁-C₆)alkyl. In another embodiment, R¹² is —(CH₂)ₙ-phenyl. In another embodiment, R¹² is —SO₂(C₁-C₆)alkyl. In another embodiment, R¹² is —SO₂(C₃-C₆)cycloalkyl. In another embodiment, R¹² is —SO₂(C₆-C₁₀)aryl. In another embodiment, R¹² is —NR¹⁴R¹⁵. In another embodiment, is the alkyl, aryl or heteroaryl portion of R¹² is substituted with one or more halogen, hydroxyl and/or —(C₁-C₆)alkoxy.

In one embodiment, R¹³ is hydrogen. In another embodiment, R¹³ is —(C₁-C₆)alkyl.

In one embodiment, R¹⁴ is hydrogen. In another embodiment, R¹⁴ is —(C₁-C₆)alkyl.

In one embodiment, R¹⁵ is hydrogen. In another embodiment, R¹⁵ is —(C₁-C₆)alkyl.

In one embodiment, n is 0. In another embodiment, n is 1. In another embodiment, n is 2. In another embodiment, n is 3.

In one embodiment, provided herein are compounds that result from any combination of X, Y, R¹¹-R¹⁵ and n as defined above.

In one embodiment, specific examples include, but are not limited to those listed in Table Z, below:

TABLE Z

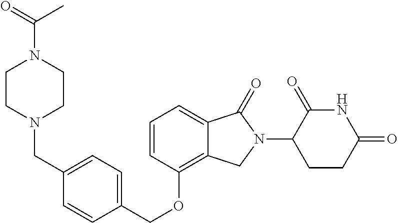 [SMILES: CC(=O)N1CCN(Cc2ccc(COc3cccc4c3CN(C3CCC(=O)NC3=O)C4=O)cc2)CC1]

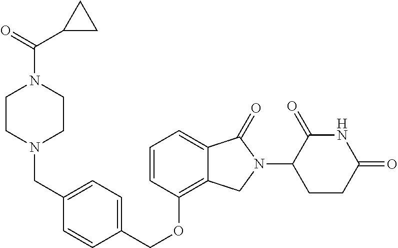 [SMILES: O=C1CCC(N2Cc3c(OCc4ccc(CN5CCN(C(=O)C6CC6)CC5)cc4)cccc3C2=O)C(=O)N1]

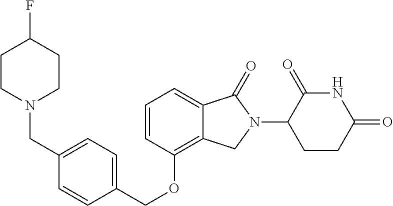 [SMILES: O=C1CCC(N2Cc3c(OCc4ccc(CN5CCC(F)CC5)cc4)cccc3C2=O)C(=O)N1]

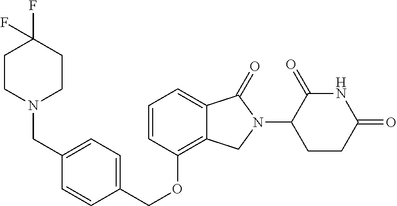 [SMILES: O=C1CCC(N2Cc3c(OCc4ccc(CN5CCC(F)(F)CC5)cc4)cccc3C2=O)C(=O)N1]

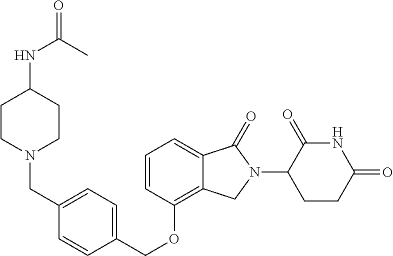 [SMILES: CC(=O)NC1CCN(Cc2ccc(COc3cccc4c3CN(C3CCC(=O)NC3=O)C4=O)cc2)CC1]

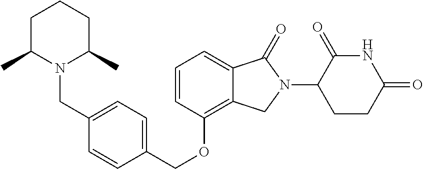 [SMILES: C[C@@H]1CCC[C@H](C)N1Cc1ccc(COc2cccc3c2CN(C2CCC(=O)NC2=O)C3=O)cc1]

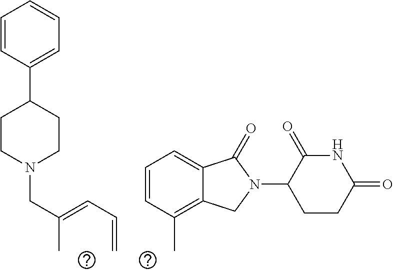

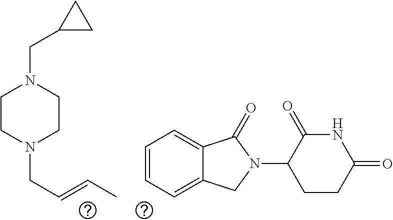

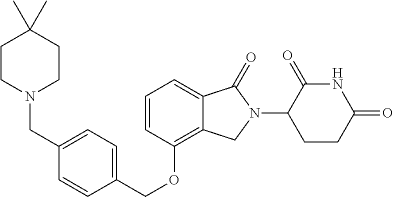 [SMILES: CC1(C)CCN(Cc2ccc(COc3cccc4c3CN(C3CCC(=O)NC3=O)C4=O)cc2)CC1]

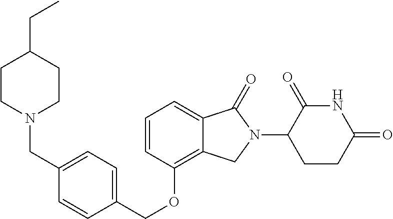 [SMILES: CCC1CCN(Cc2ccc(COc3cccc4c3CN(C3CCC(=O)NC3=O)C4=O)cc2)CC1]

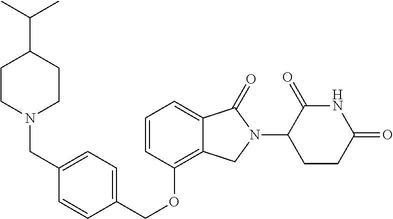 [SMILES: CC(C)C1CCN(Cc2ccc(COc3cccc4c3CN(C3CCC(=O)NC3=O)C4=O)cc2)CC1]

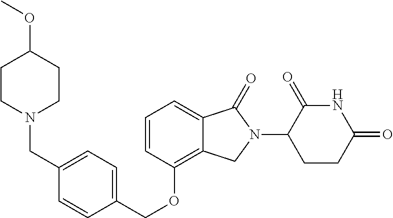 [SMILES: COC1CCN(Cc2ccc(COc3cccc4c3CN(C3CCC(=O)NC3=O)C4=O)cc2)CC1]

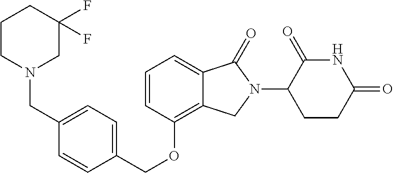 [SMILES: O=C1CCC(N2Cc3c(OCc4ccc(CN5CCCC(F)(F)C5)cc4)cccc3C2=O)C(=O)N1]

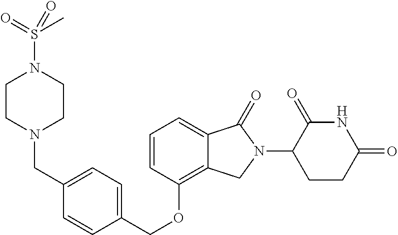 [SMILES: CS(=O)(=O)N1CCN(Cc2ccc(COc3cccc4c3CN(C3CCC(=O)NC3=O)C4=O)cc2)CC1]

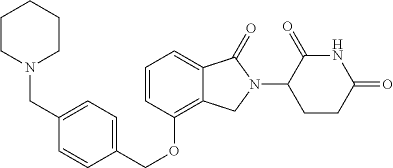 [SMILES: O=C1CCC(N2Cc3c(OCc4ccc(CN5CCCCC5)cc4)cccc3C2=O)C(=O)N1]

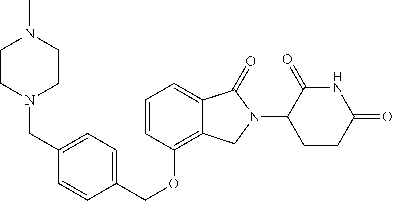 [SMILES: CN1CCN(Cc2ccc(COc3cccc4c3CN(C3CCC(=O)NC3=O)C4=O)cc2)CC1]

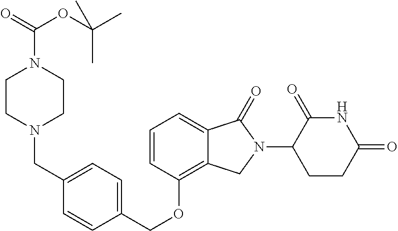 [SMILES: CC(C)(C)OC(=O)N1CCN(Cc2ccc(COc3cccc4c3CN(C3CCC(=O)NC3=O)C4=O)cc2)CC1]

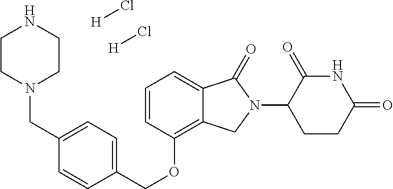

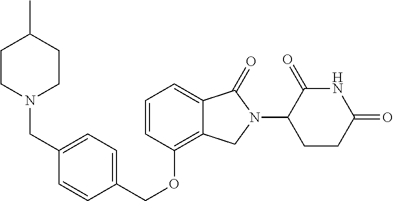 [SMILES: CC1CCN(Cc2ccc(COc3cccc4c3CN(C3CCC(=O)NC3=O)C4=O)cc2)CC1]

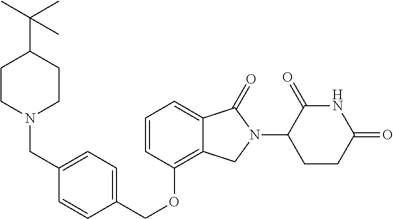 [SMILES: CC(C)(C)C1CCN(Cc2ccc(COc3cccc4c3CN(C3CCC(=O)NC3=O)C4=O)cc2)CC1]

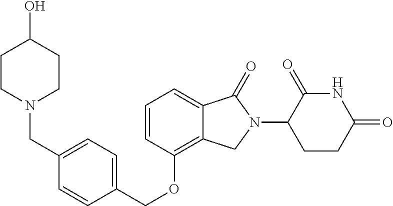 [SMILES: O=C1CCC(N2Cc3c(OCc4ccc(CN5CCC(O)CC5)cc4)cccc3C2=O)C(=O)N1]

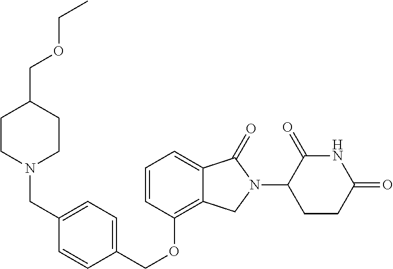 [SMILES: CCOCC1CCN(Cc2ccc(COc3cccc4c3CN(C3CCC(=O)NC3=O)C4=O)cc2)CC1]

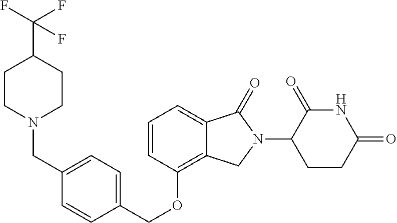 [SMILES: O=C1CCC(N2Cc3c(OCc4ccc(CN5CCC(C(F)(F)F)CC5)cc4)cccc3C2=O)C(=O)N1]

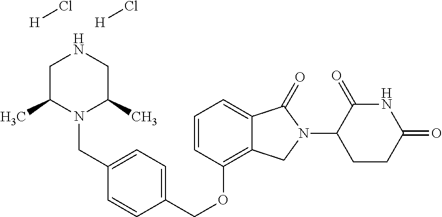

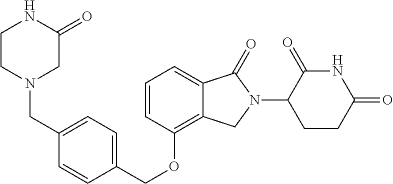 [SMILES: O=C1CN(Cc2ccc(COc3cccc4c3CN(C3CCC(=O)NC3=O)C4=O)cc2)CCN1]

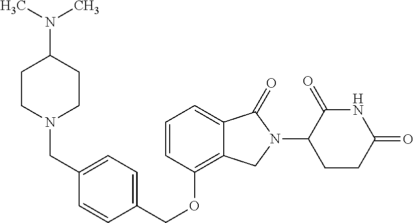 [SMILES: CN(C)C1CCN(Cc2ccc(COc3cccc4c3CN(C3CCC(=O)NC3=O)C4=O)cc2)CC1]

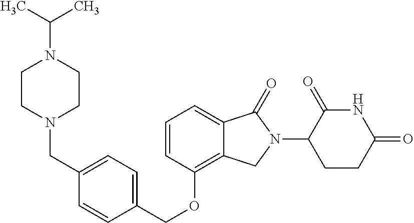 [SMILES: CC(C)N1CCN(Cc2ccc(COc3cccc4c3CN(C3CCC(=O)NC3=O)C4=O)cc2)CC1]

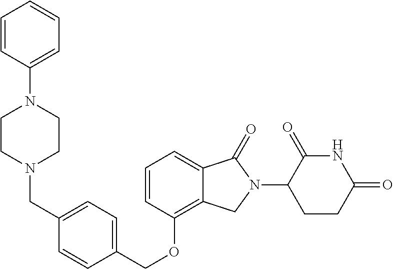 [SMILES: O=C1CCC(N2Cc3c(OCc4ccc(CN5CCN(c6ccccc6)CC5)cc4)cccc3C2=O)C(=O)N1]

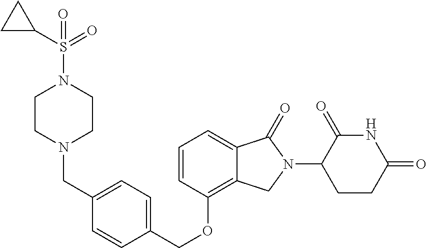 [SMILES: O=C1CCC(N2Cc3c(OCc4ccc(CN5CCN(S(=O)(=O)C6CC6)CC5)cc4)cccc3C2=O)C(=O)N1]

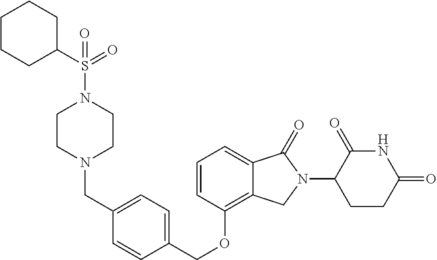 [SMILES: O=C1CCC(N2Cc3c(OCc4ccc(CN5CCN(S(=O)(=O)C6CCCCC6)CC5)cc4)cccc3C2=O)C(=O)N1]

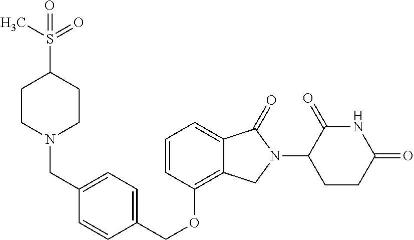 [SMILES: CS(=O)(=O)C1CCN(Cc2ccc(COc3cccc4c3CN(C3CCC(=O)NC3=O)C4=O)cc2)CC1]

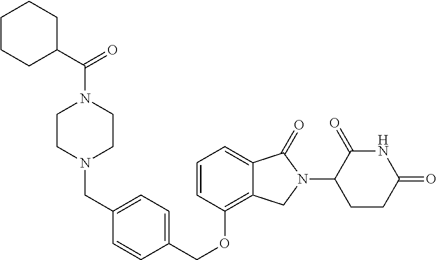 [SMILES: O=C1CCC(N2Cc3c(OCc4ccc(CN5CCN(C(=O)C6CCCCC6)CC5)cc4)cccc3C2=O)C(=O)N1]

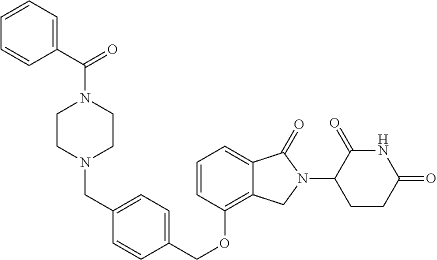 [SMILES: O=C1CCC(N2Cc3c(OCc4ccc(CN5CCN(C(=O)c6ccccc6)CC5)cc4)cccc3C2=O)C(=O)N1]

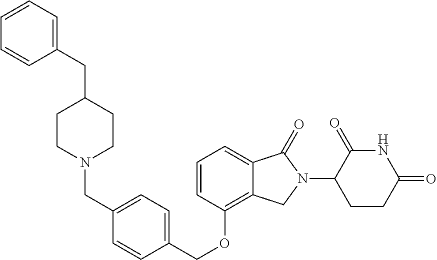 [SMILES: O=C1CCC(N2Cc3c(OCc4ccc(CN5CCC(Cc6ccccc6)CC5)cc4)cccc3C2=O)C(=O)N1]

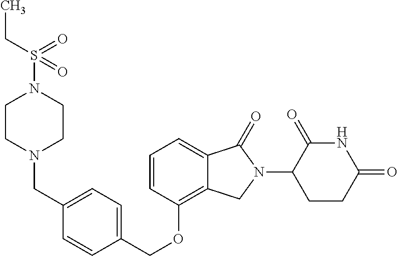 [SMILES: CCS(=O)(=O)N1CCN(Cc2ccc(COc3cccc4c3CN(C3CCC(=O)NC3=O)C4=O)cc2)CC1]

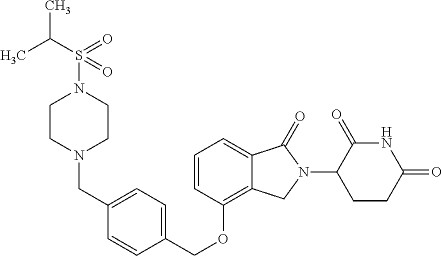 [SMILES: CC(C)S(=O)(=O)N1CCN(Cc2ccc(COc3cccc4c3CN(C3CCC(=O)NC3=O)C4=O)cc2)CC1]

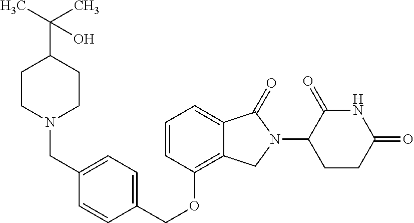 [SMILES: CC(C)(O)C1CCN(Cc2ccc(COc3cccc4c3CN(C3CCC(=O)NC3=O)C4=O)cc2)CC1]

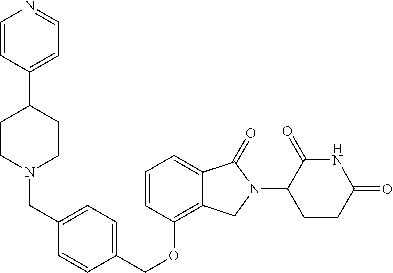 [SMILES: O=C1CCC(N2Cc3c(OCc4ccc(CN5CCC(c6ccncc6)CC5)cc4)cccc3C2=O)C(=O)N1]

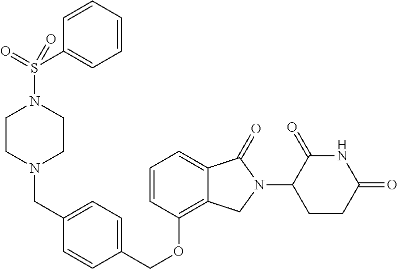 [SMILES: O=C1CCC(N2Cc3c(OCc4ccc(CN5CCN(S(=O)(=O)c6ccccc6)CC5)cc4)cccc3C2=O)C(=O)N1]

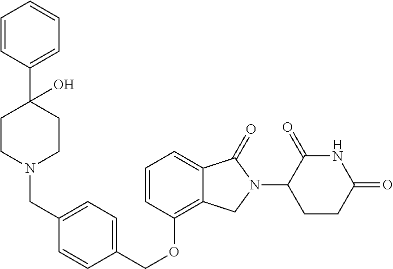 [SMILES: O=C1CCC(N2Cc3c(OCc4ccc(CN5CCC(O)(c6ccccc6)CC5)cc4)cccc3C2=O)C(=O)N1]

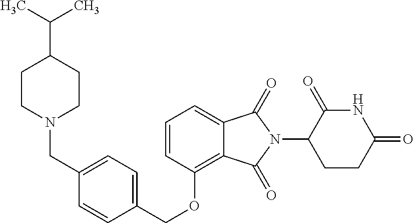 [SMILES: CC(C)C1CCN(Cc2ccc(COc3cccc4c3C(=O)N(C3CCC(=O)NC3=O)C4=O)cc2)CC1]

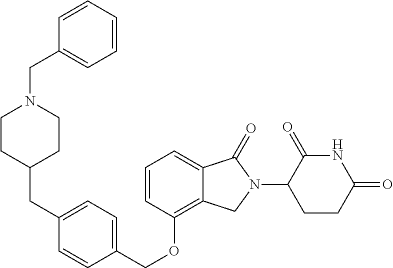 [SMILES: O=C1CCC(N2Cc3c(OCc4ccc(CC5CCN(Cc6ccccc6)CC5)cc4)cccc3C2=O)C(=O)N1]

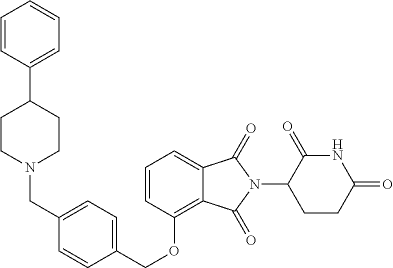 [SMILES: O=C1CCC(N2C(=O)c3cccc(OCc4ccc(CN5CCC(c6ccccc6)CC5)cc4)c3C2=O)C(=O)N1]

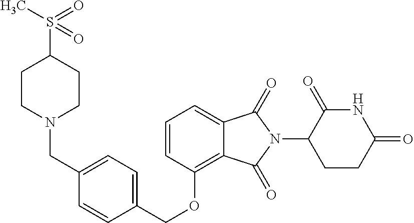 [SMILES: CS(=O)(=O)C1CCN(Cc2ccc(COc3cccc4c3C(=O)N(C3CCC(=O)NC3=O)C4=O)cc2)CC1]

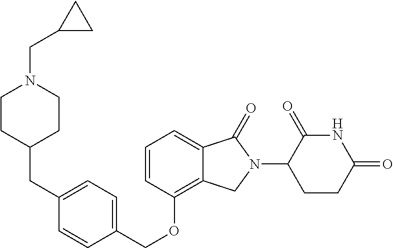 [SMILES: O=C1CCC(N2Cc3c(OCc4ccc(CC5CCN(CC6CC6)CC5)cc4)cccc3C2=O)C(=O)N1]

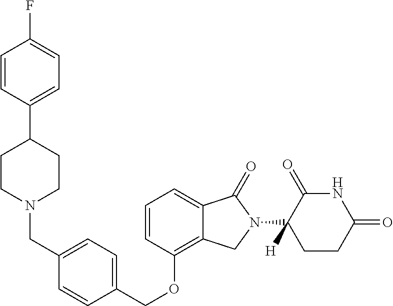

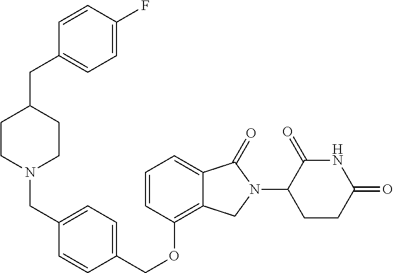 [SMILES: O=C1CCC(N2Cc3c(OCc4ccc(CN5CCC(Cc6ccc(F)cc6)CC5)cc4)cccc3C2=O)C(=O)N1]

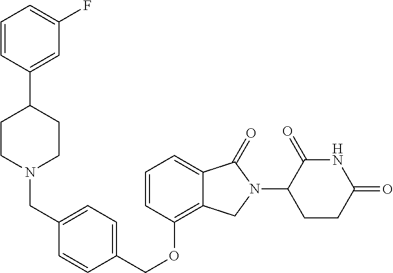 [SMILES: O=C1CCC(N2Cc3c(OCc4ccc(CN5CCC(c6cccc(F)c6)CC5)cc4)cccc3C2=O)C(=O)N1]

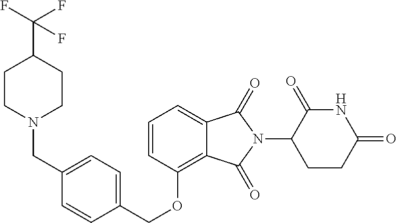 [SMILES: O=C1CCC(N2C(=O)c3cccc(OCc4ccc(CN5CCC(C(F)(F)F)CC5)cc4)c3C2=O)C(=O)N1]

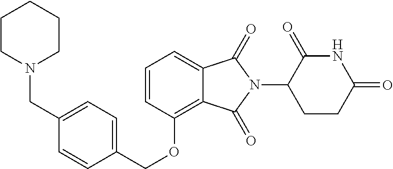 [SMILES: O=C1CCC(N2C(=O)c3cccc(OCc4ccc(CN5CCCCC5)cc4)c3C2=O)C(=O)N1]

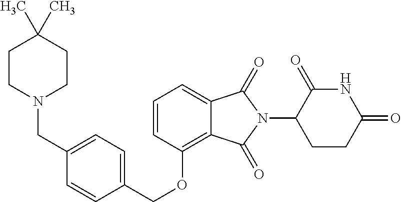 [SMILES: CC1(C)CCN(Cc2ccc(COc3cccc4c3C(=O)N(C3CCC(=O)NC3=O)C4=O)cc2)CC1]

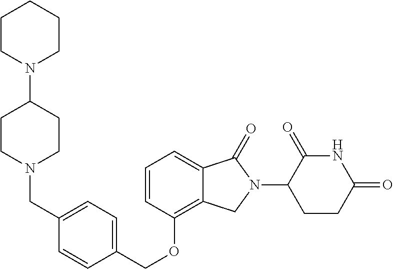 [SMILES: O=C1CCC(N2Cc3c(OCc4ccc(CN5CCC(N6CCCCC6)CC5)cc4)cccc3C2=O)C(=O)N1]

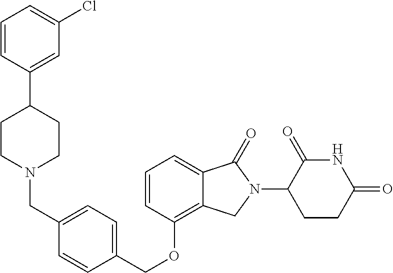 [SMILES: O=C1CCC(N2Cc3c(OCc4ccc(CN5CCC(c6cccc(Cl)c6)CC5)cc4)cccc3C2=O)C(=O)N1]

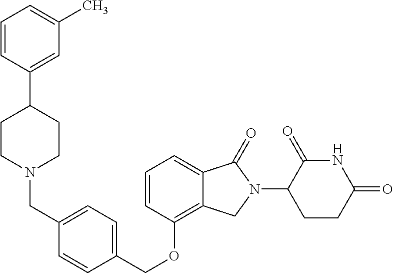 [SMILES: Cc1cccc(C2CCN(Cc3ccc(COc4cccc5c4CN(C4CCC(=O)NC4=O)C5=O)cc3)CC2)c1]

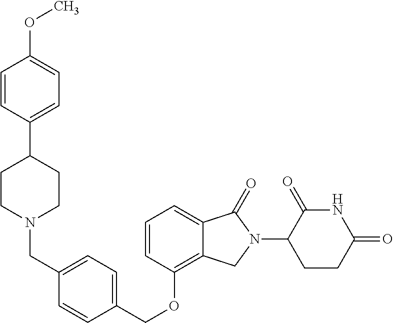 [SMILES: COc1ccc(C2CCN(Cc3ccc(COc4cccc5c4CN(C4CCC(=O)NC4=O)C5=O)cc3)CC2)cc1]

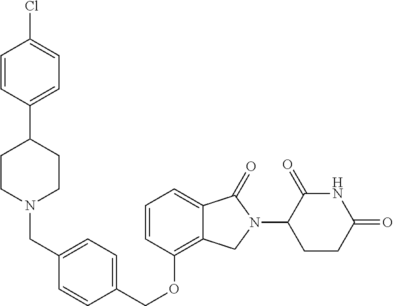 [SMILES: O=C1CCC(N2Cc3c(OCc4ccc(CN5CCC(c6ccc(Cl)cc6)CC5)cc4)cccc3C2=O)C(=O)N1]

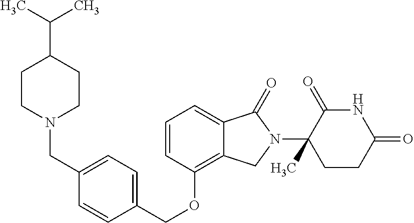 [SMILES: CC(C)C1CCN(Cc2ccc(COc3cccc4c3CN([C@@]3(C)CCC(=O)NC3=O)C4=O)cc2)CC1]

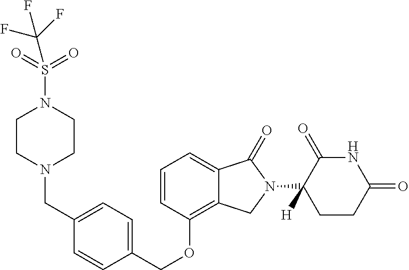

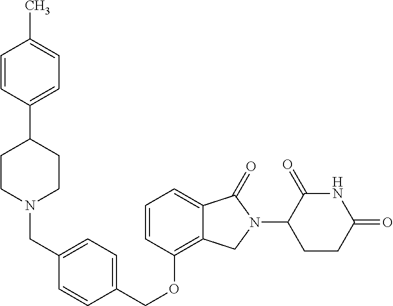 [SMILES: Cc1ccc(C2CCN(Cc3ccc(COc4cccc5c4CN(C4CCC(=O)NC4=O)C5=O)cc3)CC2)cc1]

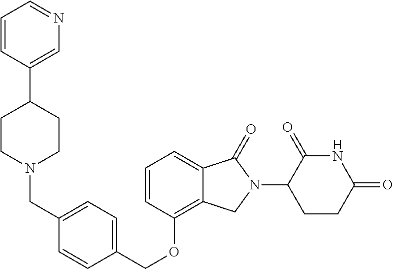 [SMILES: O=C1CCC(N2Cc3c(OCc4ccc(CN5CCC(c6cccnc6)CC5)cc4)cccc3C2=O)C(=O)N1]

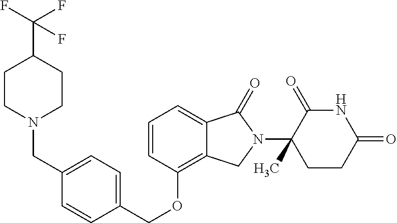 [SMILES: C[C@]1(N2Cc3c(OCc4ccc(CN5CCC(C(F)(F)F)CC5)cc4)cccc3C2=O)CCC(=O)NC1=O]

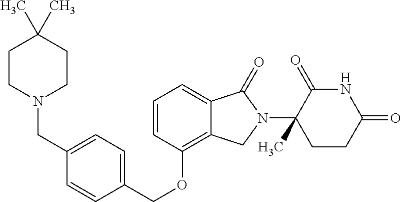 [SMILES: CC1(C)CCN(Cc2ccc(COc3cccc4c3CN([C@@]3(C)CCC(=O)NC3=O)C4=O)cc2)CC1]

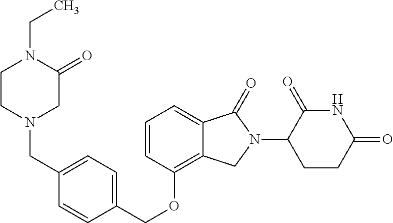 [SMILES: CCN1CCN(Cc2ccc(COc3cccc4c3CN(C3CCC(=O)NC3=O)C4=O)cc2)CC1=O]

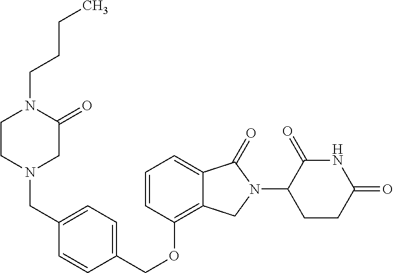 [SMILES: CCCCN1CCN(Cc2ccc(COc3cccc4c3CN(C3CCC(=O)NC3=O)C4=O)cc2)CC1=O]

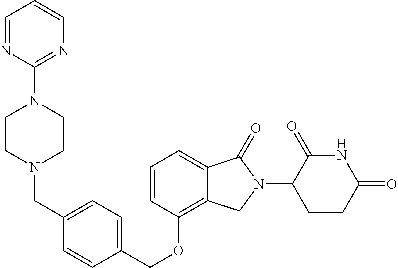 [SMILES: O=C1CCC(N2Cc3c(OCc4ccc(CN5CCN(c6ncccn6)CC5)cc4)cccc3C2=O)C(=O)N1]

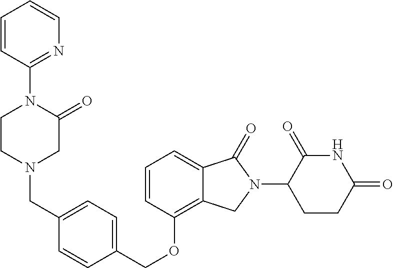 [SMILES: O=C1CCC(N2Cc3c(OCc4ccc(CN5CCN(c6ccccn6)C(=O)C5)cc4)cccc3C2=O)C(=O)N1]

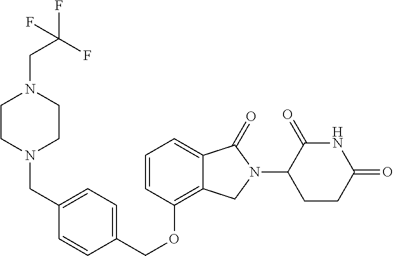 [SMILES: O=C1CCC(N2Cc3c(OCc4ccc(CN5CCN(CC(F)(F)F)CC5)cc4)cccc3C2=O)C(=O)N1]

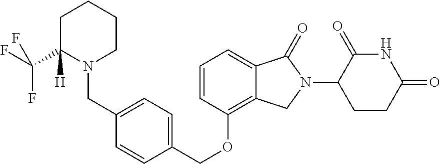

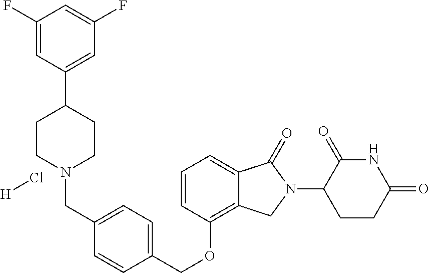

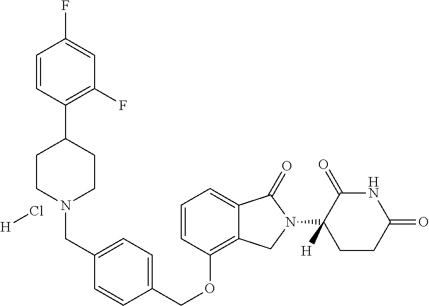

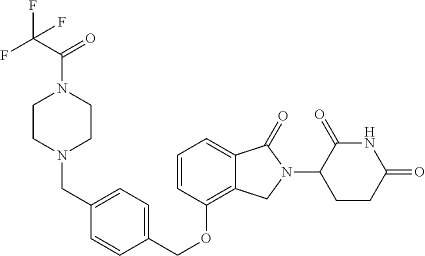 [SMILES: O=C1CCC(N2Cc3c(OCc4ccc(CN5CCN(C(=O)C(F)(F)F)CC5)cc4)cccc3C2=O)C(=O)N1]

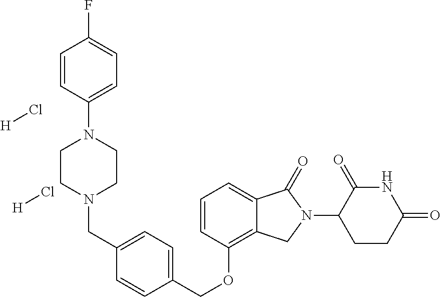

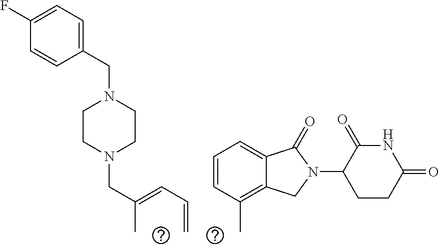

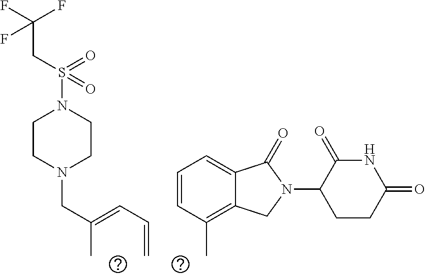

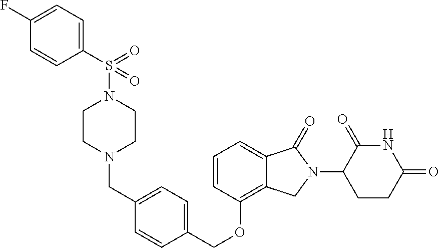 [SMILES: O=C1CCC(N2Cc3c(OCc4ccc(CN5CCN(S(=O)(=O)c6ccc(F)cc6)CC5)cc4)cccc3C2=O)C(=O)N1]

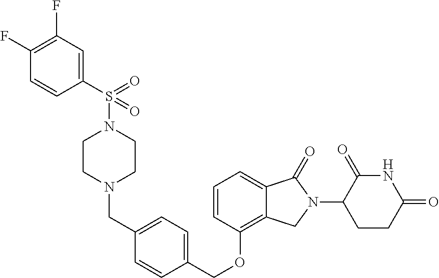 [SMILES: O=C1CCC(N2Cc3c(OCc4ccc(CN5CCN(S(=O)(=O)c6ccc(F)c(F)c6)CC5)cc4)cccc3C2=O)C(=O)N1]

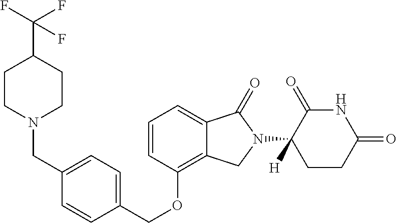 [SMILES: O=C1CC[C@H](N2Cc3c(OCc4ccc(CN5CCC(C(F)(F)F)CC5)cc4)cccc3C2=O)C(=O)N1]

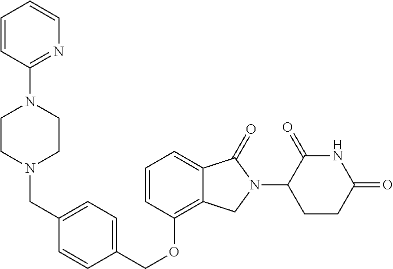 [SMILES: O=C1CCC(N2Cc3c(OCc4ccc(CN5CCN(c6ccccn6)CC5)cc4)cccc3C2=O)C(=O)N1]

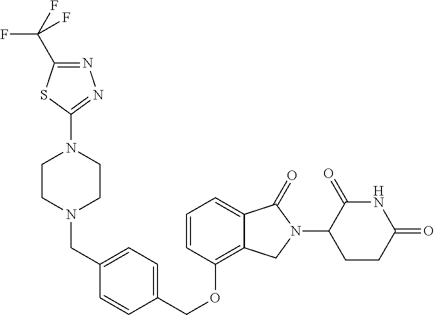 [SMILES: O=C1CCC(N2Cc3c(OCc4ccc(CN5CCN(c6nnc(C(F)(F)F)s6)CC5)cc4)cccc3C2=O)C(=O)N1]

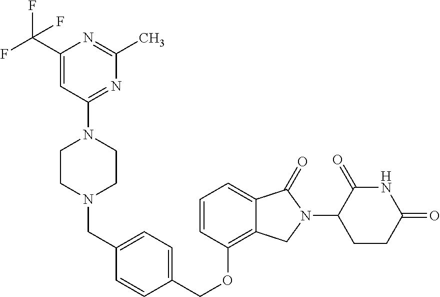 [SMILES: Cc1nc(N2CCN(Cc3ccc(COc4cccc5c4CN(C4CCC(=O)NC4=O)C5=O)cc3)CC2)cc(C(F)(F)F)n1]

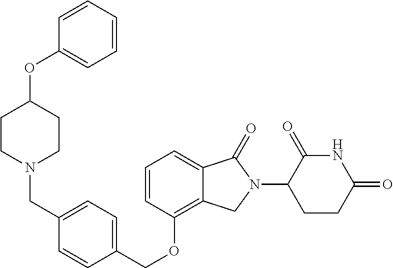 [SMILES: O=C1CCC(N2Cc3c(OCc4ccc(CN5CCC(Oc6ccccc6)CC5)cc4)cccc3C2=O)C(=O)N1]

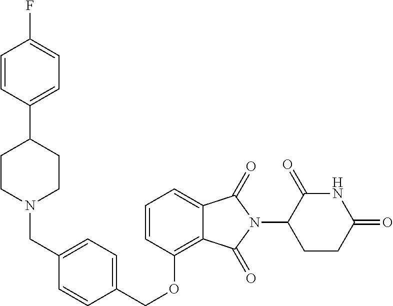 [SMILES: O=C1CCC(N2C(=O)c3cccc(OCc4ccc(CN5CCC(c6ccc(F)cc6)CC5)cc4)c3C2=O)C(=O)N1]

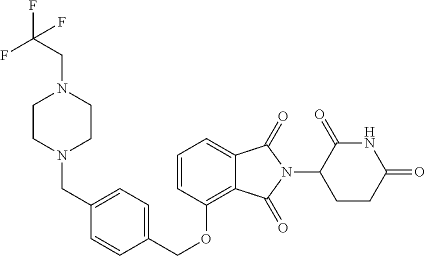 [SMILES: O=C1CCC(N2C(=O)c3cccc(OCc4ccc(CN5CCN(CC(F)(F)F)CC5)cc4)c3C2=O)C(=O)N1]

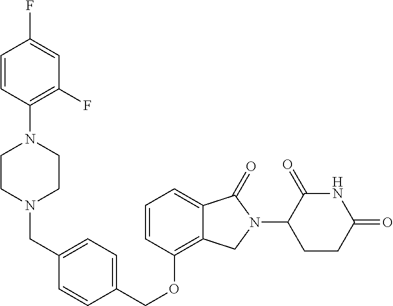 [SMILES: O=C1CCC(N2Cc3c(OCc4ccc(CN5CCN(c6ccc(F)cc6F)CC5)cc4)cccc3C2=O)C(=O)N1]

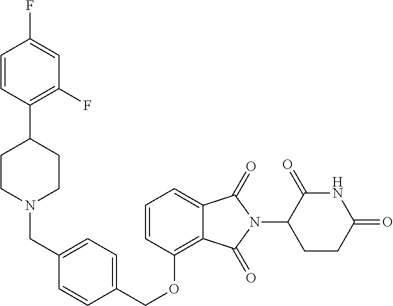 [SMILES: O=C1CCC(N2C(=O)c3cccc(OCc4ccc(CN5CCC(c6ccc(F)cc6F)CC5)cc4)c3C2=O)C(=O)N1]

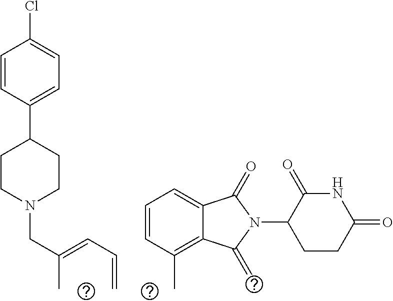

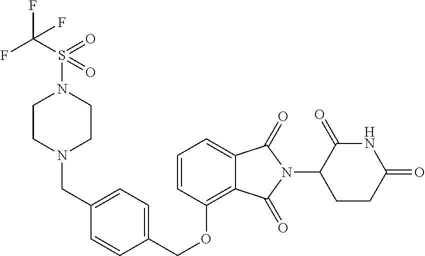 [SMILES: O=C1CCC(N2C(=O)c3cccc(OCc4ccc(CN5CCN(S(=O)(=O)C(F)(F)F)CC5)cc4)c3C2=O)C(=O)N1]

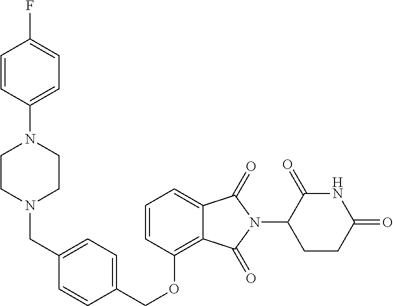 [SMILES: O=C1CCC(N2C(=O)c3cccc(OCc4ccc(CN5CCN(c6ccc(F)cc6)CC5)cc4)c3C2=O)C(=O)N1]

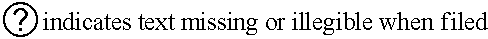

or a pharmaceutically acceptable salt, solvate or stereoisomer thereof.

In another embodiment, representative compounds are those listed in Table AA, below:

TABLE AA

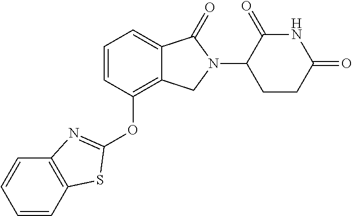 [SMILES: O=C1CCC(N2Cc3c(Oc4nc5ccccc5s4)cccc3C2=O)C(=O)N1]

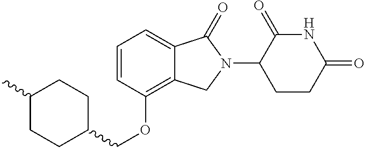 [SMILES: CC1CCC(COc2cccc3c2CN(C2CCC(=O)NC2=O)C3=O)CC1]

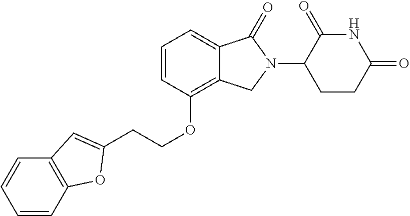 [SMILES: O=C1CCC(N2Cc3c(OCCc4cc5ccccc5o4)cccc3C2=O)C(=O)N1]

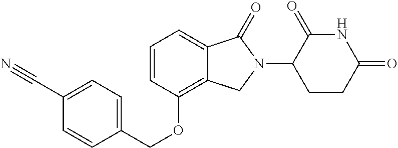 [SMILES: N#Cc1ccc(COc2cccc3c2CN(C2CCC(=O)NC2=O)C3=O)cc1]

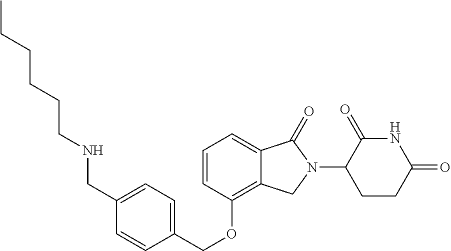 [SMILES: CCCCCCNCc1ccc(COc2cccc3c2CN(C2CCC(=O)NC2=O)C3=O)cc1]

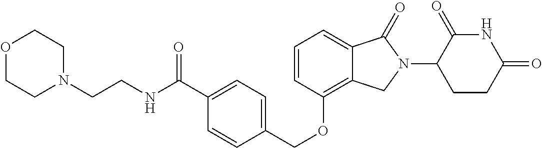 [SMILES: O=C1CCC(N2Cc3c(OCc4ccc(C(=O)NCCN5CCOCC5)cc4)cccc3C2=O)C(=O)N1]

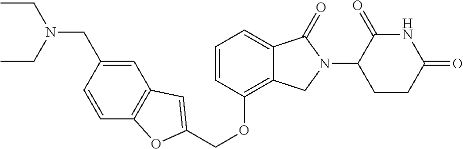 [SMILES: CCN(CC)Cc1ccc2oc(COc3cccc4c3CN(C3CCC(=O)NC3=O)C4=O)cc2c1]

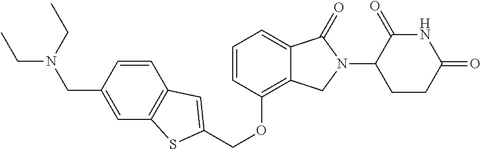 [SMILES: CCN(CC)Cc1ccc2cc(COc3cccc4c3CN(C3CCC(=O)NC3=O)C4=O)sc2c1]

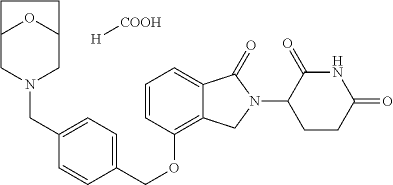

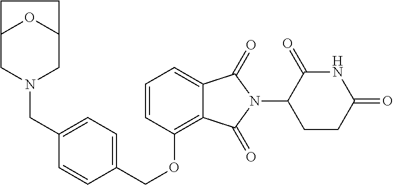 [SMILES: O=C1CCC(N2C(=O)c3cccc(OCc4ccc(CN5CC6CCC(C5)O6)cc4)c3C2=O)C(=O)N1]

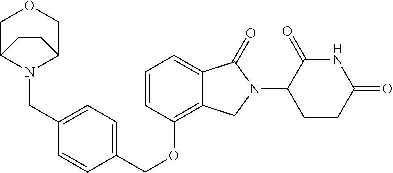 [SMILES: O=C1CCC(N2Cc3c(OCc4ccc(CN5C6CCC5COC6)cc4)cccc3C2=O)C(=O)N1]

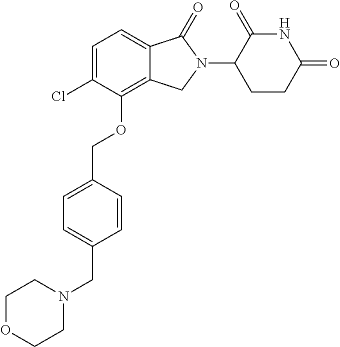 [SMILES: O=C1CCC(N2Cc3c(ccc(Cl)c3OCc3ccc(CN4CCOCC4)cc3)C2=O)C(=O)N1]

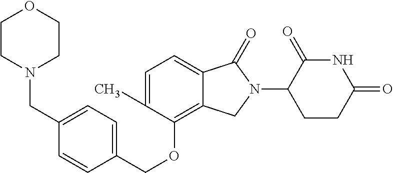 [SMILES: Cc1ccc2c(c1OCc1ccc(CN3CCOCC3)cc1)CN(C1CCC(=O)NC1=O)C2=O]

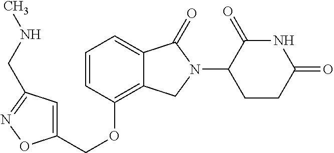 [SMILES: CNCc1cc(COc2cccc3c2CN(C2CCC(=O)NC2=O)C3=O)on1]

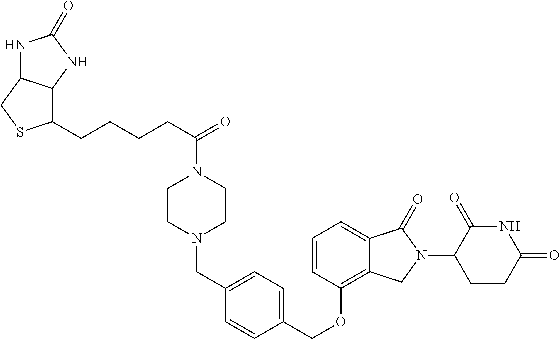 [SMILES: O=C1CCC(N2Cc3c(OCc4ccc(CN5CCN(C(=O)CCCCC6SCC7NC(=O)NC76)CC5)cc4)cccc3C2=O)C(=O)N1]

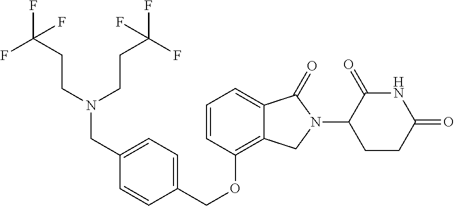 [SMILES: O=C1CCC(N2Cc3c(OCc4ccc(CN(CCC(F)(F)F)CCC(F)(F)F)cc4)cccc3C2=O)C(=O)N1]

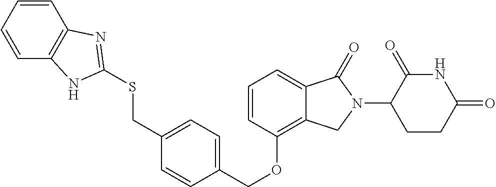 [SMILES: O=C1CCC(N2Cc3c(OCc4ccc(CSc5nc6ccccc6[nH]5)cc4)cccc3C2=O)C(=O)N1]

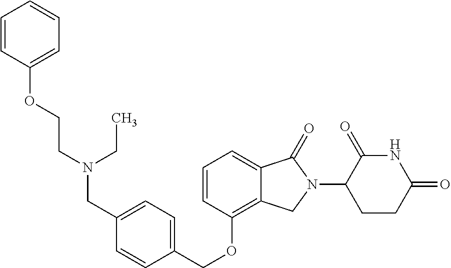 [SMILES: CCN(CCOc1ccccc1)Cc1ccc(COc2cccc3c2CN(C2CCC(=O)NC2=O)C3=O)cc1]

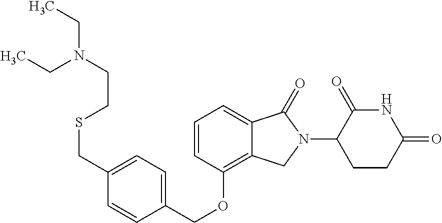 [SMILES: CCN(CC)CCSCc1ccc(COc2cccc3c2CN(C2CCC(=O)NC2=O)C3=O)cc1]

or a pharmaceutically acceptable salt, solvate or stereoisomer thereof.

In one embodiment, the immunomodulatory compound is:

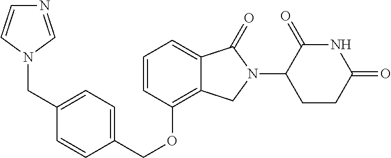 [SMILES: O=C1CCC(N2Cc3c(OCc4ccc(Cn5ccnc5)cc4)cccc3C2=O)C(=O)N1]

or a pharmaceutically acceptable salt, solvate, prodrug, or stereoisomer thereof.

In one embodiment, the immunomodulatory compound is:

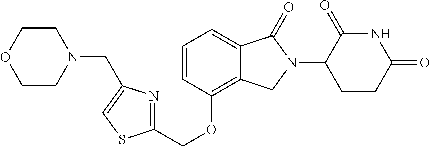 [SMILES: O=C1CCC(N2Cc3c(OCc4nc(CN5CCOCC5)cs4)cccc3C2=O)C(=O)N1]

or a pharmaceutically acceptable salt, solvate, prodrug, or stereoisomer thereof.

In one embodiment, the immunomodulatory compound is:

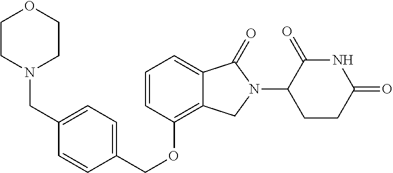 [SMILES: O=C1CCC(N2Cc3c(OCc4ccc(CN5CCOCC5)cc4)cccc3C2=O)C(=O)N1]

or a pharmaceutically acceptable salt, solvate, prodrug, or stereoisomer thereof.

All of the compounds described can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

Illustrative IMiD® immunomodulatory drugs include, but are not limited to, lenalidomide (REVLIMID®) pomalidomide (Actimid™; POMALYST®), (S)-3-(4-(4-(morpholinomethyl)benzyloxy)-1-oxoisoindolin-2-yl)piperidine-2,6-dione, N-[2-(2,6-Dioxo-piperidin-3-yl)-1-oxo-2,3-dihydro-1H-isoindol-4-ylmethyl]-2-phenyl-acetamide, 2-(2,6-Dioxopiperidin-3-yl)-4-phenylaminoisoindole-1,3-dione, 2-[2-(2,6-Dioxopiperidin-3-yl)-1,3-dioxo-2,3-dihydro-1H-isoindol-4-ylamino]-N-methylacetamide, 1-[2-(2,6-Dioxo-piperidin-3-yl)-1,3-dioxo-2,3-dihydro-1H-isoindol-4-ylmethyl]-3-p-tolyl-urea, or N-[2-(2,6-Dioxo-piperidin-3-yl)-1,3-dioxo-2,3-dihydro-1H-isoindol-4-ylmethyl]-2-pyridin-4-yl-acetamide.

### 5.5 Anti-CD20 Antibodies

CD20, the first B-cell specific antigen defined by the monoclonal antibody tositumomab, plays a critical role in B-cell development. Human CD20 is a 297 amino acid (30- to 35-kDa) phosphoprotein with four transmembrane domains encoded by the gene MS4A1 located on chromosome 11q12.2. CD20 plays a critical role in B-cell development and is a biomarker for immunotherapies targeting B-cell derived diseases. CD20 is an integral membrane protein expressed by B lymphocytes in early stages of differentiation and by most B cell lymphomas, but not by differentiated plasma cells. CD20 remains on the membrane of B cells without dissociation or internalization upon antibody binding. CD20 functions though binding to the Src family of tyrosine kinases, such as Lyn, Fyn and Lck, and believed to be involved as a result in the phosphorylation cascade of intracellular proteins. Anti-CD20 antibodies are broadly classified into type I and type II antibodies. Both types of anti-CD 20 antibodies exhibit equal ability in activating Fc-FcγR interactions such as antibody-dependent cellular cytotoxicity (ADCC) and phagocytosis. Type I anti-CD20 antibodies redistribute CD20 into membrane lipid rafts and potently activate complement-dependent cytotoxicity (CDC). Type II anti-CD20 antibodies weakly activate CDC but more potently induce direct programmed cell death.

A person of ordinary skill in the art can readily identify and select additional anti-CD20 antibodies that are useful in the present invention. For example, in some embodiments, such antibodies are described, for example, in U.S. Pat. Nos. 8,153,125, 8,147,832, 8,101,179, 8,084,582, 8,057,793 and 7,879,984, and U.S. Patent Publication Nos. 2011/0129412, 2012/0183545, 2012/0134990 and 2012/0034185.

In some embodiments, an anti-CD20 antibody for use in the present invention is a type I antibody. In some embodiments, an anti-CD20 for use in the present invention is a type II antibody.

In some embodiments, an anti-CD20 antibody is an antibody that binds to a CD20 epitope selected from 170ANPS173 and 182YCYSI185.

In some embodiments, an anti-CD20 antibody has a binding affinity (Kd) for an epitope of CD20 of less than 12 nM, less than 11 nM, less than 10 nM, less than 9 nM, less than 8 nM, less than 7 nM, less than 6 nM, less than 5 nM, less than 4 nM, less than 3 nM, less than 2 nM or less than 1 nM.

Rituximab is but one example of an anti-CD20 antibody. In some embodiments, an anti-CD20 antibody for use in the present invention includes, for example, rituximab (Rituxan® or MabThera®), Gazyva® (i.e., obinutuzumab) and Arzerra® (ofatumumab). For ease of reference, provided methods and regimens detailed herein refer to an exemplary anti-CD20 antibody (i.e., rituximab); however, such reference is not intended to limit the present invention to a single anti-CD20 antibody. Indeed, all references to rituximab, or a biosimilar thereof, are to be read by a person skilled in the art to encompass the class of anti-CD20 antibodies. For example, it will be appreciated that the anti-CD20 antibodies ofatumumab (Arzerra®) or obinutuzumab (Gazyva®) can instead be administered in each instance where reference is made to a CD20 antibody or rituximab. In some such embodiments, ofatumumab is administered in 12 doses according to the following schedule: 300 mg initial dose, followed 1 week later by 2000 mg dose weekly for 7 doses, followed 4 weeks later by 2000 mg every 4 weeks for 4 doses. In some such embodiments, obinutuzumab is administered for six 28-day cycles as follows: 100 mg on day 1, cycle 1; 900 mg on day 2 cycle 1; 1000 mg on days 8 and 15 of cycle 1; and 1000 mg on day 1 of cycles 2-6. Accordingly, in some embodiments, the term “rituximab” encompasses all corresponding anti-CD20 antibodies that fulfill the requirements necessary for obtaining a marketing authorization as an identical or biosimilar product in a country or territory selected from the group of countries consisting of the USA, Europe and Japan.

In some embodiments, an anti-CD20 antibody has the same or similar activity as rituximab, or a biosimilar thereof. In some embodiments, an anti-CD20 antibody binds to the same or similar region or epitope as rituximab or a fragment thereof. In some embodiments, an anti-CD20 antibody competes with the binding of rituximab or a fragment thereof to CD20. In some embodiments, an anti-CD20 antibody is bioequivalent to rituximab or a fragment thereof. In some embodiments, an anti-CD20 antibody is a biosimilar of rituximab or a fragment thereof. In some embodiments, an anti-CD20 antibody is a variant or derivative of rituximab, including functional fragments, derivatives, or antibody conjugates.

Rituximab (Rituxan® or MabThera®) is a genetically engineered cytolytic, chimeric murine/human monoclonal IgG1 kappa antibody directed against the CD20 cell-surface molecule present in normal B lymphocytes and B-cell CLL and in most forms of non-Hodgkin's B-cell lymphomas. Rituximab has a binding affinity for the CD20 antigen of approximately 8.0 nM. Rituximab can induce complement-dependent cellular cytotoxicity (CDC) and antibody-dependent cellular cytotoxicity (ADCC), leading to its clinical activity against lymphoma cells. Rituximab can also lead to apoptosis of B cells upon binding to CD20, thereby leading to direct inhibition of cellular growth.

Rituximab is produced by mammalian cell (Chinese Hamster Ovary) suspension culture in a nutrient medium containing the antibiotic gentamicin. Gentamicin is not detectable in the final product. Rituximab is a sterile, clear, colorless, preservative-free liquid concentrate for intravenous administration. Rituximab is supplied at a concentration of 10 mg/mL in either 100 mg/10 mL or 500 mg/50 mL single-use vials. Rituximab is formulated in polysorbate 80 (0.7 mg/mL), sodium citrate dihydrate (7.35 mg/mL), sodium chloride (9 mg/mL) and water for injection. The pH of Rituxan® (or MabThera®) is 6.5.

Rituximab has been investigated in clinical studies and approved for treatment of patients with CLL in combination with fludarabine and cyclophosphamide, as well as patients with rheumatoid arthritis in combination with methotrexate. Rituximab is also approved for treatment of non-Hodgkin's lymphoma, Wegener's Granulomatosis and Microscopic Polyangiitis.

### 5.6 Methods of Use

Provided herein are methods for treating or preventing a cancer, comprising administering an effective amount of a TOR kinase inhibitor and an effective amount of an IMiD® immunomodulatory drug to a patient having a cancer. In some embodiments, the cancer is resistant to IMiD® immunomodulatory drug treatment.

Further provided herein are methods for treating or preventing a cancer resistant to IMiD® immunomodulatory drug treatment, comprising administering an effective amount of a TOR kinase inhibitor (e.g., alone or in the absence of an IMiD® immunomodulatory drug) to a patient having a cancer resistant to IMiD® immunomodulatory drug treatment.

Further provided herein are methods for preventing resistance to treatment of a cancer, the methods comprising administering an effective amount of a TOR kinase inhibitor and an effective amount of an IMiD® immunomodulatory drug to a patient having a cancer. In one embodiment, the resistance is resistance to IMiD® immunomodulatory drug treatment. In another, the resistance is resistance to TOR kinase inhibitor treatment.

Provided herein are methods for treating or preventing a cancer, comprising administering an effective amount of a TOR kinase inhibitor and an effective amount of dexamethasone to a patient having a cancer.

In certain embodiments, the cancer is a bloodborne tumor.

In certain embodiments, the cancer is a lymphoma, a leukemia or a multiple myeloma.

In certain embodiments, the cancer is non-Hodgkin's lymphoma. In certain embodiments, the non-Hodgkin's lymphoma is diffuse large B-cell lymphoma (DLBCL), follicular lymphoma (FL), acute myeloid leukemia (AML), mantle cell lymphoma (MCL), or ALK⁺ anaplastic large cell lymphoma. In one embodiment, the non-Hodgkin's lymphoma is advanced solid non-Hodgkin's lymphoma. In one embodiment, the non-Hodgkin's lymphoma is diffuse large B-cell lymphoma (DLBCL).

In certain embodiments, the cancer is diffuse large B-cell lymphoma (DLBCL).

In certain embodiments, the cancer is a B-cell lymphoma.

In certain embodiments, the B-cell lymphoma is a B-cell non-Hodgkin's lymphoma selected from diffuse large B-cell lymphoma, Burkitt's lymphoma/leukemia, mantle cell lymphoma, mediastinal (thymic) large B-cell lymphoma, follicular lymphoma, marginal zone lymphoma (including extranodal marginal zone B-cell lymphoma and nodal marginal zone B-cell lymphoma), lymphoplamacytic lymphoma/Waldenstrom macroglobulinemia. In some embodiments, the B-cell lymphoma is chronic lymphocytic leukemia/small lymphocytic lymphoma (CLL/SLL). In one embodiment, the B-cell lymphoma is Waldenstrom macroglobulinemia.

In one embodiment, the B-cell non-Hodgkin's lymphoma is refractory B-cell non-Hodgkin's lymphoma. In one embodiment, the B-cell non-Hodgkin's lymphoma is relapsed B-cell non-Hodgkin's lymphoma.

In certain embodiments, the cancer is a T-cell lymphoma.

The B-cell disorders chronic lymphocytic leukemia/small lymphocytic lymphoma (CLL/SLL) represent 2 ends of a spectrum of the same disease process differing in the degree of blood/marrow involvement (CLL) versus lymph node involvement (SLL).

In other embodiments, the cancer is a multiple myeloma.

In certain embodiments, the cancer is a cancer of the head, neck, eye, mouth, throat, esophagus, bronchus, larynx, pharynx, chest, bone, lung, colon, rectum, stomach, prostate, urinary bladder, uterine, cervix, breast, ovaries, testicles or other reproductive organs, skin, thyroid, blood, lymph nodes, kidney, liver, pancreas, and brain or central nervous system.

In other embodiments, the cancer is a solid tumor. In certain embodiments, the solid tumor is a relapsed or refractory solid tumor.

In one embodiment, the solid tumor is a neuroendocrine tumor. In certain embodiments, the neuroendocrine tumor is a neuroendocrine tumor of gut origin. In certain embodiments, the neuroendocrine tumor is of non-pancreatic origin. In certain embodiments, the neuroendocrine tumor is non-pancreatic of gut origin. In certain embodiments, the neuroendocrine tumor is of unknown primary origin. In certain embodiments, the neuroendocrine tumor is a symptomatic endocrine producing tumor or a nonfunctional tumor. In certain embodiments, the neuroendocrine tumor is locally unresectable, metastatic moderate, well differentiated, low (grade 1) or intermediate (grade 2).

In one embodiment, the solid tumor is non-small cell lung cancer (NSCLC).

In another embodiment, the solid tumor is glioblastoma multiforme (GBM).

In another embodiment, the solid tumor is hepatocellular carcinoma (HCC).

In another embodiment, the solid tumor is breast cancer. In one embodiment, the breast cancer is hormone receptor positive. In one embodiment, the breast cancer is estrogen receptor positive (ER+, ER+/Her2 or ER+/Her2+). In one embodiment, the breast cancer is estrogen receptor negative (ER−/Her2+). In one embodiment, the breast cancer is triple negative (TN) (breast cancer that does not express the genes and/or protein corresponding to the estrogen receptor (ER), progesterone receptor (PR), and that does not overexpress the Her2/neu protein).

In another embodiment, the solid tumor is colorectal cancer (CRC).

In another embodiment, the solid tumor is salivary cancer.

In another embodiment, the solid tumor is pancreatic cancer.

In another embodiment, the solid tumor is adenocystic cancer.

In another embodiment, the solid tumor is adrenal cancer.

In another embodiment, the solid tumor is esophageal cancer, renal cancer, leiomyosarcoma, or paraganglioma.

In one embodiment, the solid tumor is an advanced solid tumor.

In another embodiment, the cancer is head and neck squamous cell carcinoma.

In another embodiment, the cancer is E-twenty six (ETS) overexpressing castration-resistant prostate cancer.

In another embodiment, the cancer is E-twenty six (ETS) overexpressing Ewings sarcoma.

In other embodiments, the cancer is an advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant giolma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karyotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrespectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In a specific embodiment, the cancer is metastatic. In another embodiment, the cancer is refractory or resistant to chemotherapy or radiation; in particular, refractory to thalidomide.

In other embodiments, the cancer is a cancer associated with the pathways involving mTOR, PI3K, or Akt kinases and mutants or isoforms thereof. Other cancers within the scope of the methods provided herein include those associated with the pathways of the following kinases: PI3Kα, PI3Kβ, PI3Kδ, KDR, GSK3α, GSK3β, ATM, ATX, ATR, cFMS, and/or DNA-PK kinases and mutants or isoforms thereof. In some embodiments, the cancers associated with mTOR/PI3K/Akt pathways include solid and blood-borne tumors, for example, multiple myeloma, mantle cell lymphoma, diffused large B-cell lymphoma, acute myeloid lymphoma, follicular lymphoma, chronic lymphocytic leukemia; and solid tumors, for example, breast, lung, endometrial, ovarian, gastric, cervical, and prostate cancer; glioblastoma; renal carcinoma; hepatocellular carcinoma; colon carcinoma; neuroendocrine tumors; head and neck tumors; and sarcomas, such as Ewing's sarcoma.

In certain embodiments, provided herein are methods for achieving an International Workshop on Chronic Lymphocytic Leukemia (IWCLL) response definition of a complete response, partial response or stable disease in a patient having chronic lymphocytic leukemia, comprising administering an effective amount of a TOR kinase inhibitor in combination with an IMiD® immunomodulatory drug to said patient. In certain embodiments, provided herein are methods for achieving a Response Evaluation Criteria in Solid Tumors (for example, RECIST 1.1) of complete response, partial response or stable disease in a patient having a solid tumor, comprising administering an effective amount of a TOR kinase inhibitor in combination with an IMiD® immunomodulatory drug to said patient. In certain embodiments, provided herein are methods for achieving a National Cancer Institute-Sponsored Working Group on Chronic Lymphocytic Leukemia (NCI-WG CLL) response definition of complete response, partial response or stable disease in a patient having leukemia, comprising administering an effective amount of a TOR kinase inhibitor in combination with an IMiD® immunomodulatory drug to said patient. In certain embodiments, provided herein are methods for achieving a Prostate Cancer Working Group 2 (PCWG2) Criteria of complete response, partial response or stable disease in a patient having prostate cancer, comprising administering an effective amount of a TOR kinase inhibitor in combination with an IMiD® immunomodulatory drug to said patient. In certain embodiments, provided herein are methods for achieving an International Workshop Criteria (IWC) for non-Hodgkin's lymphoma of complete response, partial response or stable disease in a patient having non-Hodgkin's lymphoma, comprising administering an effective amount of a TOR kinase inhibitor in combination with an IMiD® immunomodulatory drug to said patient. In certain embodiments, provided herein are methods for achieving an International Uniform Response Criteria (IURC) for multiple myeloma of complete response, partial response or stable disease in a patient having multiple myeloma, comprising administering an effective amount of a TOR kinase inhibitor in combination with an IMiD® immunomodulatory drug to said patient. In certain embodiments, provided herein are methods for achieving a Responses Assessment for Neuro-Oncology (RANO) Working Group for glioblastoma multiforme of complete response, partial response or stable disease in a patient having glioblastoma multiforme, comprising administering an effective amount of a TOR kinase inhibitor in combination with an IMiD® immunomodulatory drug to said patient.

In certain embodiments, provided herein are methods for increasing survival without tumor progression of a patient having a cancer, comprising administering an effective amount of a TOR kinase inhibitor in combination with an effective amount of an IMiD® immunomodulatory drug to said patient.

In one embodiment, provided herein are methods for preventing or delaying a Response Evaluation Criteria in Solid Tumors (for example, RECIST 1.1) of progressive disease in a patient, comprising administering an effective amount of a TOR kinase inhibitor in combination with an effective amount of an IMiD® immunomodulatory drug to a patient having a cancer. In one embodiment the prevention or delaying of progressive disease is characterized or achieved by a change in overall size of the target lesions, of for example, between −30% and +20% compared to pre-treatment. In another embodiment, the change in size of the target lesions is a reduction in overall size of more than 30%, for example, more than 50% reduction in target lesion size compared to pre-treatment. In another, the prevention is characterized or achieved by a reduction in size or a delay in progression of non-target lesions compared to pre-treatment. In one embodiment, the prevention is achieved or characterized by a reduction in the number of target lesions compared to pre-treatment. In another, the prevention is achieved or characterized by a reduction in the number or quality of non-target lesions compared to pre-treatment. In one embodiment, the prevention is achieved or characterized by the absence or the disappearance of target lesions compared to pre-treatment. In another, the prevention is achieved or characterized by the absence or the disappearance of non-target lesions compared to pre-treatment. In another embodiment, the prevention is achieved or characterized by the prevention of new lesions compared to pre-treatment. In yet another embodiment, the prevention is achieved or characterized by the prevention of clinical signs or symptoms of disease progression compared to pre-treatment, such as cancer-related cachexia or increased pain.

In certain embodiments, provided herein are methods for decreasing the size of target lesions in a patient compared to pre-treatment, comprising administering an effective amount of a TOR kinase inhibitor in combination with an effective amount of an IMiD® immunomodulatory drug to a patient having a cancer.

In certain embodiments, provided herein are methods for decreasing the size of a non-target lesion in a patient compared to pre-treatment, comprising administering an effective amount of a TOR kinase inhibitor in combination with an effective amount of an IMiD® immunomodulatory drug to a patient having a cancer.

In certain embodiments, provided herein are methods for achieving a reduction in the number of target lesions in a patient compared to pre-treatment, comprising administering an effective amount of a TOR kinase inhibitor in combination with an effective amount of an IMiD® immunomodulatory drug to a patient having a cancer.

In certain embodiments, provided herein are methods for achieving a reduction in the number of non-target lesions in a patient compared to pre-treatment, comprising administering an effective amount of a TOR kinase inhibitor in combination with an effective amount of an IMiD® immunomodulatory drug to a patient having a cancer.

In certain embodiments, provided herein are methods for achieving an absence of all target lesions in a patient, comprising administering an effective amount of a TOR kinase inhibitor in combination with an effective amount of an IMiD® immunomodulatory drug to a patient having a cancer.

In certain embodiments, provided herein are methods for achieving an absence of all non-target lesions in a patient, comprising administering an effective amount of a TOR kinase inhibitor in combination with an effective amount of an IMiD® immunomodulatory drug to a patient having a cancer.

In certain embodiments, provided herein are methods for treating a cancer, the methods comprising administering an effective amount of a TOR kinase inhibitor in combination with an effective amount of an IMiD® immunomodulatory drug to a patient having a cancer, wherein the treatment results in a complete response, partial response or stable disease, as determined by Response Evaluation Criteria in Solid Tumors (for example, RECIST 1.1).

In certain embodiments, provided herein are methods for treating a cancer, the methods comprising administering an effective amount of a TOR kinase inhibitor in combination with an effective amount of an IMiD® immunomodulatory drug to a patient having a cancer, wherein the treatment results in a reduction in target lesion size, a reduction in non-target lesion size and/or the absence of new target and/or non-target lesions, compared to pre-treatment.

In certain embodiments, provided herein are methods for treating a cancer, the methods comprising administering an effective amount of a TOR kinase inhibitor in combination with an effective amount of an IMiD® immunomodulatory drug to a patient having a cancer, wherein the treatment results in prevention or retarding of clinical progression, such as cancer-related cachexia or increased pain.

In some embodiments, provided herein are methods for treating a cancer, the methods comprising administering an effective amount of a TOR kinase inhibitor in combination with an effective amount of an IMiD® immunomodulatory drug to a patient having a cancer, wherein the treatment results in one or more of inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), and/or increased Overall Survival (OS), among others.

Provided herein are methods for the treatment or management of cancer using Ikaros, Aiolos, as a predictive or prognostic factor for the combination of a TOR kinase inhibitor and an IMiD® immunomodulatory drug. In certain embodiments, provided herein are methods for screening or identifying cancer patients as described herein (e.g., multiple myeloma, DLBCL, mantle cell lymphoma, follicular lymphoma, acute myeloblastic leukemia, chronic lymphocytic leukemia, and/or MDS patients), for treatment with a combination of a TOR kinase inhibitor and an IMiD® immunomodulatory drug, using Ikaros, Aiolos, as a predictive or prognostic factor. In one embodiment, provided herein is a method of predicting patient response to treatment of cancer with a combination provided herein, the method comprising obtaining biological material from the patient, and measuring the presence or absence of Ikaros, or Aiolos. In one embodiment, the mRNA or protein is purified from the tumor and the presence or absence of a biomarker is measured by gene or protein expression analysis. In certain embodiments, the presence or absence of a biomarker is measured by quantitative real-time PCR (QRT-PCR), microarray, flow cytometry or immunofluorescence. In other embodiments, the presence or absence of a biomarker is measured by enzyme-linked immunosorbent assay-based methodologies (ELISA) or other similar methods known in the art. Biomarkers associated with non-Hodgkin's lymphomas are described, for example, in U.S. Patent Publication No. 2011/0223157, the entirety of which is incorporated by reference in its entirety. In certain embodiments, the biomarker is Aiolos. In another embodiment, the biomarker is Ikaros. In certain embodiments, the biomarker is both Ikaros and Aiolos. In certain embodiments, the biomarker is a combination of biomarkers provided herein. In certain embodiments, the biomarker(s) further comprises CRBN. In specific embodiments, the cancer is DLBCL.

In another embodiment, provided herein is a method of predicting patient response to treatment in a cancer patient, the method comprising obtaining cancer cells from the patient, culturing the cells in the presence or absence of the combination of a TOR kinase inhibitor and an IMiD® immunomodulatory drug, purifying protein or RNA from the cultured cells, and measuring the presence or absence of a biomarker by, e.g., protein or gene expression analysis. The expression monitored may be, for example, mRNA expression or protein expression. In one embodiment, the cancer patient is a lymphoma, leukemia, multiple myeloma, solid tumor, non-Hodgkin's lymphoma, DLBCL, mantle cell lymphoma, follicular lymphoma, acute myeloblastic leukemia, chronic lymphocytic leukemia, MDS or melanoma patient. In certain embodiments, the biomarker is Aiolos. In another embodiment, the biomarker is Ikaros. In certain embodiments, the biomarker is both Ikaros and Aiolos. In certain embodiments, the biomarker(s) further comprises CRBN. In specific embodiments, the cancer is DLBCL.

In another embodiment, provided herein is a method of monitoring tumor response to the combination of a TOR kinase inhibitor and an IMiD® immunomodulatory drug treatment in a cancer patient. The method comprises obtaining a biological sample from the patient, measuring the expression of a biomarker in the biological sample, administering the combination of a TOR kinase inhibitor and an IMiD® immunomodulatory drug to the patient, thereafter obtaining a second biological sample from the patient, measuring biomarker expression in the second biological sample, and comparing the levels of expression, where an increased level of biomarker expression after treatment indicates the likelihood of an effective tumor response. In certain embodiments, the biomarker is Aiolos. In another embodiment, the biomarker is Ikaros. In certain embodiments, the biomarker is both Ikaros and Aiolos. In certain embodiments, the biomarker(s) further comprises CRBN. In specific embodiments, the cancer is DLBCL.

In certain embodiments, CRBN protein levels are not down-regulated or decreased, whereas Ikaros protein levels and/or Aiolos protein levels are down-regulated or decreased. In some embodiments, such a phenotype indicates the patient has, or may be developing, an acquired resistance to the compound. In certain embodiments, the biomarker is c-Myc. In certain embodiments, c-Myc levels are decreased. In other embodiments, the biomarker is CD44. In certain embodiments, CD44 levels are increased. In some embodiments, such a phenotype indicates the patient has, or may be developing, an acquired resistance to the compound. In other embodiments, a decrease in the level of Ikaros and/or Aiolos protein levels indicates an effective treatment with the compound.

In one embodiment, a decreased level of biomarker expression after treatment indicates the likelihood of effective tumor response. The biomarker expression monitored can be, for example, mRNA expression or protein expression. In certain embodiments, the biomarker is Aiolos. In another embodiment, the biomarker is Ikaros. In certain embodiments, the biomarker is both Ikaros and Aiolos. In specific embodiments, the tumor is DLBCL.

In one embodiment, an increased level of biomarker expression after treatment indicates the likelihood of effective tumor response. The biomarker expression monitored can be, for example, mRNA expression or protein expression. In specific embodiments, the tumor is DLBCL.

In another aspect, provided herein are methods of assessing the efficacy of a combination of a TOR kinase inhibitor and an IMiD® immunomodulatory drug in treating cancer, comprising: (a) administering the combination to a patient having cancer; (b) obtaining a first sample from the patient; (c) determining the level of a CRBN-associated protein in the first sample; and (d) comparing the level of the CRBN-associated protein from step (c) to the level of the same protein obtained from a reference sample, wherein a change in the level as compared to the reference is indicative of the efficacy of the combination in treating the cancer. In certain embodiments, the CRBN-associated protein is Ikaros. In other embodiments, the CRBN-associated protein is Aiolos. In some embodiments, the CRBN-associated protein is Ikaros and Aiolos. In some embodiments, provided herein are methods of assessing the efficacy of a combination of a TOR kinase inhibitor and an IMiD® immunomodulatory drug in treating cancer, comprising: (a) administering the combination to a patient having cancer; (b) obtaining a first sample from the patient; (c) determining the level of a Ikaros and/or Aiolos protein in the first sample; and (d) comparing the level of the Ikaros and/or Aiolos from step (c) to the level of the same protein obtained from a reference sample, wherein a decrease in the Ikaros and/or Aiolos protein level as compared to the reference is indicative of the efficacy of combination in treating the cancer.

In some embodiments, the sample is obtained from a tumor biopsy, node biopsy, or a biopsy from bone marrow, spleen, liver, brain or breast.

In certain embodiments, step (c) comprises: (i) contacting the proteins within the first sample from step (b) with a first antibody that immunospecifically binds to a CRBN-associated protein; (ii) contacting the proteins bound to the first antibody with a second antibody with a detectable label, wherein the second antibody immunospecifically binds to the CRBN-associated protein, and wherein the second antibody immunospecifically binds to a different epitope on the CRBN-associated protein than the first antibody; (iii) detecting the presence of second antibody bound to the proteins; and (iv) determining the amount of the CRBN-associated protein based on the amount of detectable label in the second antibody.

In certain embodiments, step (c) comprises: (i) contacting the RNA within the first sample with a primer comprising a sequence specifically binding to the RNA to generate a first DNA molecule having a sequence complementary to the RNA; (ii) amplifying the DNA corresponding to a segment of a gene encoding the CRBN-associated protein; and (iii) determining the RNA level of the CRBN-associated protein based on the amount of the amplified DNA.

In certain embodiments, the combination is likely efficacious in treating the cancer if the level (e.g., protein or RNA level) of the CRBN-associated protein as compared to the reference decreases. In certain embodiments, the combination is likely efficacious in treating the cancer if the level (e.g., protein or RNA level) of the CRBN-associated protein as compared to the reference increases. In one embodiment, the reference is prepared by using a second sample obtained from the patient prior to administration of the combination to the subject; wherein the second sample is from the same source as the first sample. In another embodiment, the reference is prepared by using a second sample obtained from a healthy subject not having a cancer; wherein the second sample is from the same source as the first sample. In certain embodiments, the CRBN-associated protein is Ikaros, and the level of Ikaros protein decreases as compared to the reference. In other embodiments, the CRBN-associated protein is Aiolos, and the level of Aiolos protein decreases as compared to the reference. In some embodiments, the CRBN-associated protein is Ikaros and Aiolos, and the levels of both the Ikaros protein and Aiolos protein decrease as compared to the reference.

In one embodiment of the methods provided herein, the CRBN-associated protein is IKZF3 (Aiolos) having a molecular weight of 58 kDa. In another embodiment of the methods provided herein, the CRBN-associated protein is IKZF3 (Aiolos) having a molecular weight of 42 kDa. In another embodiment, the combination of a TOR kinase inhibitor and an IMiD® immunomodulatory drug down-regulate Aiolos expression (e.g., protein or gene expression). In specific embodiments, the Aiolos protein levels decrease.

In various embodiments of the methods provided herein, the combination of a TOR kinase inhibitor and an IMiD® immunomodulatory drug down-regulate Ikaros expression (e.g., protein or gene expression). In certain embodiments, the combination of a TOR kinase inhibitor and an IMiD® immunomodulatory drug decrease Ikaros protein levels. In some embodiments, the Aiolos protein levels decrease, and the Ikaros protein levels decrease.

CRBN or a CRBN-associated protein (e.g., Ikaros, Aiolos, or a combination thereof) can be utilized as a biomarker(s) to indicate the effectiveness or progress of a disease treatment with a the combination of a TOR kinase inhibitor and an IMiD® immunomodulatory drug. Thus, in certain embodiments, the methods provided herein are useful for characterizing a disease or disorder (e.g., cancer, for example, DLBCL) in a subject, prior to, during or after the subject receiving a treatment with a TOR kinase inhibitor and a 5-Substituted Quinazolinone.

In certain embodiments, the sensitivity of a DLBCL or a patient having DLBCL, to therapy with the combination of a TOR kinase inhibitor and an IMiD® immunomodulatory drug is related to Aiolos and/or Ikaros levels.

In various embodiments of the methods provided herein, the CRBN-associated protein is Ikaros, Aiolos, or a combination thereof. In some embodiments, these CRBN-associated proteins are evaluated in combination with other CRBN-associated proteins provided herein, such as Ikaros, Aiolos, In certain embodiments, Ikaros and Aiolos are evaluated. In other embodiments, Ikaros, Aiolos and CRBN are evaluated, or any combination thereof.

Aiolos (IKZF3) is a member of the Ikaros family of zinc-finger proteins. IKZF3 is a hematopoietic-specific transcription factor involved in the regulation of lymphocyte development (e.g., B lymphocyte proliferation and differentiation). The DNA-binding domain of IKZF3 recognizes the core motif of GGGA. IKZF3 was shown to participates in chromatin remodeling, regulates Bcl family members, binds to HDACs, mSin3, Mi-2 in T cells and acts as a transcriptional repressor. Aiolos-Foxp3 interaction has been shown to silence IL-2 expression in human T cells.

In some embodiments, the TOR kinase inhibitor is a compound as described herein. In one embodiment, the TOR kinase inhibitor is a compound of formula (I). In one embodiment, the TOR kinase inhibitor is a compound from Table A. In one embodiment, the TOR kinase inhibitor is Compound 1 (a TOR kinase inhibitor set forth herein having molecular formula C₂₁H₂₇N₅O₃). In one embodiment, the TOR kinase inhibitor is Compound 2 (a TOR kinase inhibitor set forth herein having molecular formula C₁₆H₁₆N₈O). In one embodiment, the TOR kinase inhibitor is Compound 3 (a TOR kinase inhibitor set forth herein having molecular formula C₂₀H₂₅N₅O₃). In one embodiment, Compound 1 is 7-(6-(2-hydroxypropan-2-yl)pyridin-3-yl)-1-((1r,4r)-4-methoxycyclohexyl)-3,4-dihydropyrazino-[2,3-b]pyrazin-2(1H)-one, alternatively named 7-(6-(2-hydroxypropan-2-yl)pyridin-3-yl)-1-((trans)-4-methoxycyclohexyl)-3,4-dihydropyrazino[2,3-b]pyrazin-2(1H)-one, or 7-(6-(2-hydroxypropan-2-yl)pyridin-3-yl)-1-((1R*,4R*)-4-methoxycyclohexyl)-3,4-dihydropyrazino[2,3-b]pyrazin-2(1H)-one. In another embodiment, Compound 2 is 1-ethyl-7-(2-methyl-6-(1H-1,2,4-triazol-3-yl)pyridin-3-yl)-3,4-dihydropyrazino[2,3-b]pyrazin-2(1H)-one, or a tautomer thereof, for example, 1-ethyl-7-(2-methyl-6-(4H-1,2,4-triazol-3-yl)pyridin-3-yl)-3,4-dihydropyrazino[2,3-b]pyrazin-2(1H)-one, or 1-ethyl-7-(2-methyl-6-(1H-1,2,4-triazol-5-yl)pyridin-3-yl)-3,4-dihydropyrazino[2,3-b]pyrazin-2(1H)-one. In another embodiment, Compound 3 is 1-((trans)-4-hydroxycyclohexyl)-7-(6-(2-hydroxypropan-2-yl)pyridin-3-yl)-3,4-dihydropyrazino[2,3-b]pyrazin-2(1H)-one, alternatively named 1-((1r,4r)-4-hydroxycyclohexyl)-7-(6-(2-hydroxypropan-2-yl)pyridin-3-yl)-3,4-dihydropyrazino[2,3-b]pyrazin-2(1H)-one. In one embodiment, Compound 3 is a metabolite of Compound 1.

In some embodiments, the IMiD® immunomodulatory drug is a compound as described herein. In one embodiment, the IMiD® immunomodulatory drug is lenalidomide. In another, the IMiD® immunomodulatory drug is pomalidomide. In yet another embodiment, the IMiD® immunomodulatory drug is (S)-3-(4-(4-(morpholinomethyl)benzyloxy)-1-oxoisoindolin-2-yl)piperidine-2,6-dione, N-[2-(2,6-Dioxo-piperidin-3-yl)-1-oxo-2,3-dihydro-1H-isoindol-4-ylmethyl]-2-phenyl-acetamide, 2-(2,6-Dioxopiperidin-3-yl)-4-phenylaminoisoindole-1,3-dione, 2-[2-(2,6-Dioxopiperidin-3-yl)-1,3-dioxo-2,3-dihydro-1H-isoindol-4-ylamino]-N-methylacetamide, 1-[2-(2,6-Dioxo-piperidin-3-yl)-1,3-dioxo-2,3-dihydro-1H-isoindol-4-ylmethyl]-3-p-tolyl-urea, or N-[2-(2,6-Dioxo-piperidin-3-yl)-1,3-dioxo-2,3-dihydro-1H-isoindol-4-ylmethyl]-2-pyridin-4-yl-acetamide.

A TOR kinase inhibitor administered in combination with an IMiD® immunomodulatory drug can be further combined with radiation therapy or surgery. In certain embodiments, a TOR kinase inhibitor is administered in combination with an IMiD® immunomodulatory drug to patient who is undergoing radiation therapy, has previously undergone radiation therapy or will be undergoing radiation therapy. In certain embodiments, a TOR kinase inhibitor is administered in combination with an IMiD® immunomodulatory drug to a patient who has undergone surgery, such as tumor removal surgery.

Further provided herein are methods for treating patients who have been previously treated for a cancer, as well as those who have not previously been treated. Further provided herein are methods for treating patients who have undergone surgery in an attempt to treat a cancer, as well as those who have not. Because patients with a cancer have heterogenous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with a cancer.

In one embodiment, a TOR kinase inhibitor is administered in combination with an IMiD® immunomodulatory drug and an anti-CD20 antibody, for example, rituximab (Rituxan® or MabThera®). Accordingly, provided herein are methods for treating or preventing a cancer, comprising administering an effective amount of a TOR kinase inhibitor, an effective amount of an IMiD® immunomodulatory drug and an effective amount of an anti-CD20 antibody, for example, rituximab (Rituxan® or MabThera®), to a patient having a cancer. In a specific embodiment, Compound 1 is administered in combination with an IMiD® immunomodulatory drug and an anti-CD20 antibody, for example, rituximab (Rituxan® or MabThera®). In a particular embodiment, the cancer treated or prevented with a combination of a TOR kinase inhibitor, an IMiD® immunomodulatory drug and an anti-CD20 antibody, for example, rituximab (Rituxan® or MabThera®), is diffuse large B-cell lymphomas (DLBCL).

In certain embodiments, a TOR kinase inhibitor is administered in combination with an IMiD® immunomodulatory drug to a patient in cycles. Cycling therapy involves the administration of an active agent(s) for a period of time, followed by a rest for a period of time, and repeating this sequential administration. Cycling therapy can reduce the development of resistance, avoid or reduce the side effects, and/or improves the efficacy of the treatment. The administration of a TOR kinase inhibitor, an IMiD® immunomodulatory drug and an anti-CD20 antibody, for example, rituximab (Rituxan® or MabThera®), in combination can also be carried out in such cycles.

In some embodiments, a TOR kinase inhibitor is administered once daily, or QD, an IMiD® immunomodulatory drug is administered twice daily, or BID, and an anti-CD20 antibody, for example, rituximab (Rituxan® or MabThera®), is administered once monthly or once every 4 weeks. Alternatively and/or additionally, in one or more 28-day cycles, a TOR kinase inhibitor may be administered once daily, an IMiD® immunomodulatory drug may be administered once or twice daily and an anti-CD20 antibody, for example, rituximab (Rituxan® or MabThera®), may be administered once.

In one embodiment, a TOR kinase inhibitor is administered in combination with an IMiD® immunomodulatory drug daily in single or divided doses for about 3 days, about 5 days, about one week, about two weeks, about three weeks, about four weeks (e.g., 28 days), about five weeks, about six weeks, about seven weeks, about eight weeks, about ten weeks, about fifteen weeks, or about twenty weeks, followed by a rest period of about 1 day to about ten weeks. In one embodiment, the methods provided herein contemplate cycling treatments of about one week, about two weeks, about three weeks, about four weeks, about five weeks, about six weeks, about eight weeks, about ten weeks, about fifteen weeks, or about twenty weeks. In some embodiments, a TOR kinase inhibitor is administered in combination with a an IMiD® immunomodulatory drug in single or divided doses for about 3 days, about 5 days, about one week, about two weeks, about three weeks, about four weeks (e.g., 28 days), about five weeks, or about six weeks with a rest period of about 1, 2, 3, 4, 5, 6, 7, 8, 9, 10, 12, 14, 16, 18, 20, 22, 24, 26, 28, 29, or 30 days. In some embodiments, the rest period is 1 day. In some embodiments, the rest period is 3 days. In some embodiments, the rest period is 7 days. In some embodiments, the rest period is 14 days. In some embodiments, the rest period is 28 days. The frequency, number and length of dosing cycles can be increased or decreased.

In one embodiment, the methods provided herein comprise: i) administering to the subject a first daily dose of a TOR kinase inhibitor in combination with an IMiD® immunomodulatory drug; ii) optionally resting for a period of at least one day where an IMiD® immunomodulatory drug is not administered to the subject; iii) administering a second dose of a TOR kinase inhibitor in combination with an IMiD® immunomodulatory drug to the subject; and iv) repeating steps ii) to iii) a plurality of times.

In one embodiment, the methods provided herein comprise administering to the subject a dose of an IMiD® immunomodulatory drug on day 1, followed by administering a TOR kinase inhibitor in combination with an IMiD® immunomodulatory drug to the subject on day 2 and subsequent days.

In certain embodiments, a TOR kinase inhibitor in combination with an IMiD® immunomodulatory drug is administered continuously for between about 1 and about 52 weeks. In certain embodiments, a TOR kinase inhibitor in combination with an IMiD® immunomodulatory drug is administered continuously for about 0.5, 1, 2, 3, 4, 5, 6, 7, 8, 9, 10, 11, or 12 months. In certain embodiments, a TOR kinase inhibitor in combination with an IMiD® immunomodulatory drug is administered continuously for about 7, about 14, about 21, about 28, about 35, about 42, about 84, or about 112 days.

In certain embodiments, when a TOR kinase inhibitor is administered in combination with an IMiD® immunomodulatory drug, the TOR kinase inhibitor is administered continuously for 28 days, while an IMiD® immunomodulatory drug is administered continuously for 21 days followed by 7 days without administration of an IMiD® immunomodulatory drug. In certain embodiments, when a TOR kinase inhibitor is administered in combination with an IMiD® immunomodulatory drug, the TOR kinase inhibitor is administered on one or more days for 28 days, while an IMiD® immunomodulatory drug is administered continuously for 21 days followed by 7 days without administration of an IMiD® immunomodulatory drug. In one embodiment, in a 28 day cycle, an IMiD® immunomodulatory drug is administered alone on Day 1, an IMiD® immunomodulatory drug and the TOR kinase inhibitor are administered in combination on Days 2-21 and the TOR kinase inhibitor is administered alone on Days 22-28. In some such embodiments, starting with Cycle 2 both an IMiD® immunomodulatory drug and the TOR kinase inhibitor are administered on Day 1, an IMiD® immunomodulatory drug is continued through Day 21, while the TOR kinase inhibitor is continued through Day 28. The 28 day cycles, as described above, can be continued for as long needed, such as for 1, 2, 3, 4, 5, 6, 7, 8, 9, 10, 11 or 12 months or longer.

In certain embodiments, when a TOR kinase inhibitor is administered in combination with an IMiD® immunomodulatory drug, in a 28 day cycle, an IMiD® immunomodulatory drug is administered alone on Days 1-7 and the TOR kinase inhibitor is administered alone on Days 8-28. Such 28 day cycles can be continued for as long needed, such as for 1, 2, 3, 4, 5, 6, 7, 8, 9, 10, 11 or 12 months or longer.

In certain embodiments, when a TOR kinase inhibitor is administered in combination with an IMiD® immunomodulatory drug, the TOR kinase inhibitor is administered at an amount of about 2.5 mg to about 50 mg per day (such as about 2.5 mg, about 10 mg, about 15 mg, about 16 mg, about 20 mg, about 30 mg or about 45 mg per day) and an IMiD® immunomodulatory drug is administered at an amount of about 0.10 mg to about 150 mg/day (such as about 4 mg, about 5 mg, about 10 mg, about 15 mg, about 20 mg, about 25 mg, about 30 mg, about 35 mg, about 40 mg, about 45 mg or about 50 mg per day). In certain embodiments, about 2.5 mg per day of a TOR kinase inhibitor is administered in combination with about 4 mg, about 5 mg, about 10 mg, about 15 mg, about 20 mg, about 25 mg, about 30 mg, about 35 mg, about 40 mg, about 45 mg or about 50 mg per day of an IMiD® immunomodulatory drug. In certain embodiments, about 10 mg per day of a TOR kinase inhibitor is administered in combination with about 4 mg, about 5 mg, about 10 mg, about 15 mg, about 20 mg, about 25 mg, about 30 mg, about 35 mg, about 40 mg, about 45 mg or about 50 mg per day of an IMiD® immunomodulatory drug. In certain embodiments, about 15 mg per day of a TOR kinase inhibitor is administered in combination with about 4 mg, about 5 mg, about 10 mg, about 15 mg, about 20 mg, about 25 mg, about 30 mg, about 35 mg, about 40 mg, about 45 mg or about 50 mg per day of an IMiD® immunomodulatory drug. In certain embodiments, about 16 mg per day of a TOR kinase inhibitor is administered in combination with about 4 mg, about 5 mg, about 10 mg, about 15 mg, about 20 mg, about 25 mg, about 30 mg, about 35 mg, about 40 mg, about 45 mg or about 50 mg per day of an IMiD® immunomodulatory drug. In certain embodiments, about 20 mg per day of a TOR kinase inhibitor is administered in combination with about 4 mg, about 5 mg, about 10 mg, about 15 mg, about 20 mg, about 25 mg, about 30 mg, about 35 mg, about 40 mg, about 45 mg or about 50 mg per day of an IMiD® immunomodulatory drug. In certain embodiments, about 30 mg per day of a TOR kinase inhibitor is administered in combination with about 4 mg, about 5 mg, about 10 mg, about 15 mg, about 20 mg, about 25 mg, about 30 mg, about 35 mg, about 40 mg, about 45 mg or about 50 mg per day of an IMiD® immunomodulatory drug. In certain embodiments, about 45 mg per day of a TOR kinase inhibitor is administered in combination with about 4 mg, about 5 mg, about 10 mg, about 15 mg, about 20 mg, about 25 mg, about 30 mg, about 35 mg, about 40 mg, about 45 mg or about 50 mg per day of an IMiD® immunomodulatory drug. A TOR kinase inhibitor and an IMiD® immunomodulatory drug can each be independently administered once (QD), twice (BD) or three times (TID) per day.

In certain embodiments, when a TOR kinase inhibitor is administered in combination with an IMiD® immunomodulatory drug, the TOR kinase inhibitor:IMiD® immunomodulatory drug ratio is from about 1:1 to about 1:10. In certain embodiments, when a TOR kinase inhibitor is administered in combination with an IMiD® immunomodulatory drug, the TOR kinase inhibitor:IMiD® immunomodulatory drug ratio is less than about 1:1, less than about 1:3 or less than about 1:10. In certain embodiments, when a TOR kinase inhibitor is administered in combination with an IMiD® immunomodulatory drug, the TOR kinase inhibitor:IMiD® immunomodulatory drug ratio is about 1:1, about 1:3 or about 1:10.

The following embodiments relate to the amount of lenalidomide administered, when lenalidomide is administered in combination with a TOR kinase inhibitor (and optionally dexamethasone, prednisone or an anti-CD20 antibody, for example, rituximab (Rituxan® or MabThera®)). In certain embodiments, when lenalidomide is administered in combination with a TOR kinase inhibitor, about 1 mg to about 50 mg per day or about 5 mg to about 25 mg per day of lenalidomide is administered. In certain embodiments, when a TOR kinase inhibitor is administered in combination with lenalidomide in a 28 day cycle, about 2.5 mg to about 25 mg (e.g., about 25 mg) per day of lenalidomide is administered in combination with the TOR kinase inhibitor on Days 1-21. In certain embodiments, when a TOR kinase inhibitor is administered in combination with lenalidomide in a 28 day cycle, about 2.5 mg to about 25 mg (e.g., about 20 mg) per day of lenalidomide is administered in combination with the TOR kinase inhibitor on Days 2-22. In certain embodiments, when a TOR kinase inhibitor is administered in combination with lenalidomide in a 28 day cycle, about 5 mg to about 25 mg per day of lenalidomide is administered in combination with the TOR kinase inhibitor on Days 1-21, wherein the starting dose of lenalidomide is about 5 mg per day which can be escalated to about 25 mg per day during Days 1-21. In certain embodiments, when a TOR kinase inhibitor is administered in combination with lenalidomide and dexamethasone in a 28 day cycle, about 5 mg to about 25 mg (e.g., about 25 mg) per day of lenalidomide is administered in combination with the TOR kinase inhibitor on Days 1-21 along with about 40 mg per day of dexamethasone on Days 1-4, 9-12 and 17-20 (or after the fourth 28 day cycle, about 40 mg per day of dexamethasone is administered on Days 1-4). In certain embodiments, when a TOR kinase inhibitor is administered in combination with lenalidomide, about 5 mg to about 25 mg every 3 days, every 2 days or every 24 hours of lenalidomide is administered, wherein the starting dose of lenalidomide is about 5 mg every 3 days, every 2 days or every 24 hours, which can be escalated to about 10 mg per day. When a TOR kinase inhibitor is administered in combination with lenalidomide in a 28 day cycle, the TOR kinase inhibitor can be administered on one or more days of 28 day cycle. In a specific embodiment, the TOR kinase inhibitor is administered on every day of the 28 day cycle.

The following embodiments relate to the amount of pomalidomide administered, when pomalidomide is administered in combination with a TOR kinase inhibitor (and optionally dexamethasone, prednisone or an anti-CD20 antibody, for example, rituximab (Rituxan® or MabThera®)). In certain embodiments, when pomalidomide is administered in combination with a TOR kinase inhibitor, about 0.5 mg to about 5 mg per day (e.g., about 1 mg, about 2 mg, about 2.5 mg, about 3 mg or about 4 mg per day) of pomalidomide is administered. In certain embodiments, when a TOR kinase inhibitor is administered in combination with pomalidomide in a 28 day cycle, about 4 mg of pomalidomide is administered PO in combination with the TOR kinase inhibitor on Days 1-21, wherein in the event of toxicities, the amount of pomalidomide administered can be reduced to about 1 mg per day PO, wherein administration of pomalidomide can be discontinued if toxicities continue. In certain embodiments, when a TOR kinase inhibitor is administered in combination with pomalidomide and dexamethasone in a 28 day cycle, about 0.5 mg to about 5 mg (e.g., about 1 mg, about 2 mg, about 2.5 mg, about 3 mg or about 4 mg per day) per day of pomalidomide is administered in combination with the TOR kinase inhibitor on Days 1-21 along with about 40 mg per day of dexamethasone on Days 1-4, 9-12 and 17-20 (or after the fourth 28 day cycle, about 40 mg per day of dexamethasone is administered on Days 1-4). In certain embodiments, when a TOR kinase inhibitor is administered in combination with pomalidomide and dexamethasone in a 28 day cycle, about 0.5 mg to about 5 mg (e.g., about 1 mg, about 2 mg, about 2.5 mg, about 3 mg or about 4 mg per day) per day of pomalidomide is administered in combination with the TOR kinase inhibitor on Days 1-21 along with about 40 mg per day of dexamethasone once per week (or 20 mg per week of dexamethasone for patients greater than 70 years old). When a TOR kinase inhibitor is administered in combination with pomalidomide in a 28 day cycle, the TOR kinase inhibitor can be administered on one or more days of the 28 day cycle. In a specific embodiment, the TOR kinase inhibitor is administered on every day of the 28 day cycle.

The following embodiments relate to the amount of other IMiD® immunomodulatory drugs administered, when administered in combination with a TOR kinase inhibitor (and optionally dexamethasone, prednisone or an anti-CD20 antibody, for example, rituximab (Rituxan® or MabThera®)). In certain embodiments, when an IMiD® immunomodulatory drug is administered in combination with a TOR kinase inhibitor, about 0.03 mg to about 25 mg per day (e.g., about 0.3 mg, about 1 mg, about 2 mg, about 3 mg, about 4 mg, about 5 mg or about 6 mg per day) of an IMiD® immunomodulatory drug is administered. In certain embodiments, when a TOR kinase inhibitor is administered in combination with an IMiD® immunomodulatory drug in a 28 day cycle, about 0.03 mg to about 25 mg per day (e.g., about 0.3 mg, about 1 mg, about 2 mg, about 3 mg, about 4 mg, about 5 mg or about 6 mg per day) of an IMiD® immunomodulatory drug is administered in combination with the TOR kinase inhibitor on Days 1-21. In certain embodiments, when a TOR kinase inhibitor is administered in combination with an IMiD® immunomodulatory drug in a 28 day cycle, about 0.03 mg to about 25 mg per day (e.g., about 0.3 mg, about 1 mg, about 2 mg, about 3 mg, about 4 mg, about 5 mg or about 6 mg per day) of an IMiD® immunomodulatory drug is administered once per day, once every 3 days or once per week. In certain embodiments, the IMiD® immunomodulatory drug is (S)-3-(4-(4-(morpholinomethyl)benzyloxy)-1-oxoisoindolin-2-yl)piperidine-2,6-dione, N-[2-(2,6-Dioxo-piperidin-3-yl)-1-oxo-2,3-dihydro-1H-isoindol-4-ylmethyl]-2-phenyl-acetamide, 2-(2,6-Dioxopiperidin-3-yl)-4-phenylaminoisoindole-1,3-dione, 2-[2-(2,6-Dioxopiperidin-3-yl)-1,3-dioxo-2,3-dihydro-1H-isoindol-4-ylamino]-N-methylacetamide, 1-[2-(2,6-Dioxo-piperidin-3-yl)-1,3-dioxo-2,3-dihydro-1H-isoindol-4-ylmethyl]-3-p-tolyl-urea, or N-[2-(2,6-Dioxo-piperidin-3-yl)-1,3-dioxo-2,3-dihydro-1H-isoindol-4-ylmethyl]-2-pyridin-4-yl-acetamide. When a TOR kinase inhibitor is administered in combination with an IMiD® immunomodulatory drug in a 28 day cycle, the TOR kinase inhibitor can be administered on one or more days of 28 day cycle. In a specific embodiment, the TOR kinase inhibitor is administered on every day of the 28 day cycle.

In certain embodiments, the methods provided herein further comprise the administration of an anti-CD20 antibody, for example, rituximab (Rituxan® or MabThera®), in combination with a TOR kinase inhibitor and an IMiD® immunomodulatory drug, wherein the amount of an anti-CD20 antibody, for example, rituximab (Rituxan® or MabThera®), administered is about 250 mg/m² to about 500 mg/m² once per 28 days, the amount of a TOR kinase inhibitor administered is about 10 mg to about 40 mg daily and the amount of an IMiD® immunomodulatory drug administered is about 0.5 mg to about 5 mg daily. In a particular embodiment, the methods provided herein further comprise the administration of an anti-CD20 antibody, for example, rituximab (Rituxan® or MabThera®), in combination with a TOR kinase inhibitor and an IMiD® immunomodulatory drug, wherein the amount of an anti-CD20 antibody, for example, rituximab (Rituxan® or MabThera®), administered is about 375 mg/m² or about 500 mg/m² once per 28 days, the amount of a TOR kinase inhibitor administered is about 20 mg or about 30 mg daily and the amount of an IMiD® immunomodulatory drug administered is about 2 mg or about 3 mg daily. In some such embodiments, the IMiD® immunomodulatory drug is lenalidomide. In others, the IMiD® immunomodulatory drug is pomalidomide. In yet others, the IMiD® immunomodulatory drug is (S)-3-(4-(4-(morpholinomethyl)benzyloxy)-1-oxoisoindolin-2-yl)piperidine-2,6-dione, N-[2-(2,6-Dioxo-piperidin-3-yl)-1-oxo-2,3-dihydro-1H-isoindol-4-ylmethyl]-2-phenyl-acetamide, 2-(2,6-Dioxopiperidin-3-yl)-4-phenylaminoisoindole-1,3-dione, 2-[2-(2,6-Dioxopiperidin-3-yl)-1,3-dioxo-2,3-dihydro-1H-isoindol-4-ylamino]-N-methylacetamide, 1-[2-(2,6-Dioxo-piperidin-3-yl)-1,3-dioxo-2,3-dihydro-1H-isoindol-4-ylmethyl]-3-p-tolyl-urea, or N-[2-(2,6-Dioxo-piperidin-3-yl)-1,3-dioxo-2,3-dihydro-1H-isoindol-4-ylmethyl]-2-pyridin-4-yl-acetamide.

In some embodiments of the methods provided herein, the methods comprise administering to a patient in need thereof a pharmaceutical composition comprising rituximab, wherein rituximab is administered as an infusion at a rate of 50 mg/hr. In some embodiments, the infusion rate of rituximab is increased by 50 mg/hr every 30 minutes, to a maximum of 400 mg/hr. In some embodiments, the infusion rate of rituximab is increased by 100 mg/hr every 30 minutes, to a maximum of 400 mg/hr. Accordingly, in some embodiments, the infusion rate of rituximab is 100 mg/hr. In some embodiments, the infusion rate of rituximab is 150 mg/hr. In some embodiments, the infusion rate of rituximab is 200 mg/hr. In some embodiments, the infusion rate of rituximab is 250 mg/hr. In some embodiments, the infusion rate of rituximab is 300 mg/hr. In some embodiments, the infusion rate of rituximab is 350 mg/hr. In some embodiments, the infusion rate of rituximab is 400 mg/hr.

In some embodiments, 375 mg/m² rituximab is administered on cycle 1 day 2, and 500 mg/m² rituximab is administered on cycle 2 day 1. In some embodiments, 375 mg/m² rituximab is administered on cycle 1 day 2, and 500 mg/m² rituximab is administered on each of cycle 2 day 1 and cycle 3 day 1. In some embodiments, 375 mg/m² rituximab is administered on cycle 1 day 2, and 500 mg/m² rituximab is administered on each of cycle 2 day 1, cycle 3 day 1 and cycle 4 day 1. In some embodiments, 375 mg/m² rituximab is administered on cycle 1 day 2, and 500 mg/m² rituximab is administered on each of cycle 2 day 1, cycle 3 day 1, cycle 4 day 1 and cycle 5 day 1. In some embodiments, 375 mg/m² rituximab is administered on cycle 1 day 2, and 500 mg/m² rituximab is administered on each of cycle 2 day 1, cycle 3 day 1, cycle 4 day 1, cycle 5 day 1 and cycle 6 day 1.

In certain embodiments, each of the methods provided herein further comprise the administration of an effective amount of dexamethasone in combination with a TOR kinase inhibitor and an IMiD® immunomodulatory drug. In some such embodiments, dexamethasone is administered in a dose between about 10 mg to about 50 mg, for example about 40 mg.

In certain embodiments, each of the methods provided herein further comprise the administration of an effective amount of prednisone in combination with a TOR kinase inhibitor and an IMiD immunomodulatory drug. In some such embodiments, prednisone is administered in a dose between about 10 mg to about 50 mg, for example about 30 mg.

### 5.7 Pharmaceutical Compositions and Routes of Administration

Provided herein are compositions comprising an effective amount of a TOR kinase inhibitor and an effective amount of an IMiD® immunomodulatory drug and compositions, comprising an effective amount of a TOR kinase inhibitor and an IMiD® immunomodulatory drug and a pharmaceutically acceptable carrier or vehicle.

In some embodiments, the pharmaceutical compositions described herein are suitable for oral, parenteral, mucosal, transdermal or topical administration.

The compositions can be administered to a patient orally or parenterally in the conventional form of preparations, such as capsules, microcapsules, tablets, granules, powder, troches, pills, suppositories, injections, suspensions and syrups. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the TOR kinase inhibitor in the pharmaceutical composition may be at a level that will exercise the desired effect; for example, about 0.005 mg/kg of a patient's body weight to about 10 mg/kg of a patient's body weight in unit dosage for both oral and parenteral administration.

The dose of a TOR kinase inhibitor and the dose of an IMiD® immunomodulatory drug to be administered to a patient is rather widely variable and can be subject to the judgment of a health-care practitioner. In general, the TOR kinase inhibitors and an IMiD® immunomodulatory drug can be administered one to four times a day in a dose of about 0.005 mg/kg of a patient's body weight to about 10 mg/kg of a patient's body weight in a patient, but the above dosage may be properly varied depending on the age, body weight and medical condition of the patient and the type of administration. In one embodiment, the dose is about 0.01 mg/kg of a patient's body weight to about 5 mg/kg of a patient's body weight, about 0.05 mg/kg of a patient's body weight to about 1 mg/kg of a patient's body weight, about 0.1 mg/kg of a patient's body weight to about 0.75 mg/kg of a patient's body weight or about 0.25 mg/kg of a patient's body weight to about 0.5 mg/kg of a patient's body weight. In one embodiment, one dose is given per day. In any given case, the amount of the TOR kinase inhibitor administered will depend on such factors as the solubility of the active component, the formulation used and the route of administration.

In another embodiment, provided herein are unit dosage formulations that comprise between about 1 mg and about 2000 mg, about 1 mg and about 200 mg, about 35 mg and about 1400 mg, about 125 mg and about 1000 mg, about 250 mg and about 1000 mg, about 500 mg and about 1000 mg, about 1 mg to about 30 mg, about 1 mg to about 25 mg or about 2.5 mg to about 20 mg of a TOR kinase inhibitor alone or in combination with an IMiD® immunomodulatory drug. In another embodiment, provided herein are unit dosage formulations that comprise 1 mg, 2.5 mg, 5 mg, 8 mg, 10 mg, 15 mg, 20 mg, 30 mg, 35 mg, 45 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a TOR kinase inhibitor alone or in combination with an IMiD® immunomodulatory drug. In another embodiment, provided herein are unit dosage formulations that comprise about 2.5 mg, about 8 mg, about 10 mg, about 15 mg, about 20 mg, about 30 mg or about 45 mg of a TOR kinase inhibitor alone or in combination with an IMiD® immunomodulatory drug.

In a particular embodiment, provided herein are unit dosage formulations comprising about 10 mg, about 15 mg, about 30 mg, about 45 mg, about 50 mg, about 75 mg, about 100 mg or about 400 mg of a TOR kinase inhibitor in combination with an IMiD® immunomodulatory drug. In a particular embodiment, provided herein are unit dosage formulations comprising about 5 mg, about 7.5 mg or about 10 mg of a TOR kinase inhibitor in combination with an IMiD® immunomodulatory drug.

In a particular embodiment, provided herein are unit dosage formulations comprising about 0.10 mg to about 200 mg (such as about 0.1 mg, about 1 mg, about 2 mg, about 3 mg, about 4 mg, about 5 mg, about 7.5 mg, about 10 mg, about 12.5 mg, about 15 mg, about 17.5 mg, about 20 mg, about 25 mg, about 50 mg, about 100 mg, about 150 mg or about 200 mg) of an IMiD® immunomodulatory drug in combination with a TOR kinase inhibitor.

In certain embodiments, provided herein are unit dosage formulations wherein the TOR kinase inhibitor:IMiD® immunomodulatory drug ratio is from about 1:1 to about 1:10. In certain embodiments, provided herein are unit dosage formulations wherein the TOR kinase inhibitor:IMiD® immunomodulatory drug ratio is less than about 1:1, less than about 1:3 or less than about 1:10. In certain embodiments, provided herein are unit dosage formulations wherein the TOR kinase inhibitor:IMiD® immunomodulatory drug ratio is about 1:1, about 1:3 or about 1:10.

A TOR kinase inhibitor can be administered in combination with an IMiD® immunomodulatory drug once, twice, three, four or more times daily.

A TOR kinase inhibitor can be administered in combination with an IMiD® immunomodulatory drug orally for reasons of convenience. In one embodiment, when administered orally, a TOR kinase inhibitor in combination with an IMiD® immunomodulatory drug is administered with a meal and water. In another embodiment, the TOR kinase inhibitor in combination with an IMiD® immunomodulatory drug is dispersed in water or juice (e.g., apple juice or orange juice) and administered orally as a suspension. In another embodiment, when administered orally, a TOR kinase inhibitor in combination with an IMiD® immunomodulatory drug is administered in a fasted state.

The TOR kinase inhibitor can also be administered in combination with an IMiD® immunomodulatory drug intravenously, such as intravenous infusion, or subcutaneously, such as subcutaneous injection. The mode of administration is left to the discretion of the health-care practitioner, and can depend in-part upon the site of the medical condition.

In one embodiment, provided herein are capsules containing a TOR kinase inhibitor in combination with an IMiD® immunomodulatory drug without an additional carrier, excipient or vehicle.

In another embodiment, provided herein are compositions comprising an effective amount of a TOR kinase inhibitor, an effective amount of an IMiD® immunomodulatory drug, and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a TOR kinase inhibitor with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. In one embodiment, the pharmaceutical composition is lactose-free. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders. Illustrative tablet formulations comprising Compound 1 are provided herein.

A lubricant might be necessary in a tablet formulation to prevent the tablet and punches from sticking in the die. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, aligns and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a TOR kinase inhibitor in combination with an IMiD® immunomodulatory drug as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the TOR kinase inhibitor in combination with an IMiD® immunomodulatory drug can be delayed or prolonged by proper formulation. For example, a slowly soluble pellet of the TOR kinase inhibitor in combination with an IMiD® immunomodulatory drug can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the TOR kinase inhibitor in combination with an IMiD® immunomodulatory drug in oily or emulsified vehicles that allow it to disperse slowly in the serum.

In certain embodiments, Compound 1 is administered in a formulation set forth in U.S. Patent Application Publication No. 2013-0142873, published Jun. 6, 2013, which is incorporated herein in its entirety (see particularly paragraph [0323] to paragraph [0424], and paragraph [0636] to paragraph [0655]). In other embodiments, Compound 1 is administered in a formulation set forth in U.S. Provisional Patent Application No. 61/828,506, filed May 29, 2013, which is incorporated herein in its entirety (see particularly paragraph [0246] to paragraph [0403], and paragraph [0571] to paragraph [0586]).

In certain embodiments, the Compound 2 is administered in a formulation set forth in U.S. Provisional Application No. 61/813,064, filed Apr. 17, 2013, which is incorporated herein in its entirety (see particularly paragraph [0168] to paragraph [0189] and paragraph [0262] to paragraph [0294]). In other embodiments, Compound 2 is administered in a formulation set forth in U.S. Provisional Patent Application No. 61/911,201, filed Dec. 3, 2013, which is incorporated herein in its entirety (see particularly paragraph [0170] to paragraph [0190], and paragraph [0264] to paragraph [0296]).

### 5.8 Kits

In certain embodiments, provided herein are kits comprising a TOR kinase inhibitor and an IMiD® immunomodulatory drug.

In certain embodiments, provided herein are kits comprising one or more unit dosage forms of a TOR kinase inhibitor, such as those described herein, and one or more unit dosage forms of an IMiD® immunomodulatory drug, such as those described herein.

In some embodiments, the kits described herein additionally comprise an anti-CD-20 antibody, for example, rituximab (Rituxan® or MabThera®). In other embodiments, the kits additionally comprise dexamethasone or prednisone.

In certain embodiments, the kits provided herein further comprise instructions for use, such as for administering a TOR kinase inhibitor and an IMiD® immunomodulatory drug.

### 6. EXAMPLES

### 6.1 Biochemical Assays

mTOR HTR-FRET Assay.

The following is an example of an assay that can be used to determine the TOR kinase inhibitory activity of a test compound. TOR kinase inhibitors were dissolved in DMSO and prepared as 10 mM stocks and diluted appropriately for the experiments. Reagents were prepared as follows:

“Simple TOR buffer” (used to dilute high glycerol TOR fraction): 10 mM Tris pH 7.4, 100 mM NaCl, 0.1% Tween-20, 1 mM DTT. Invitrogen mTOR (cat#PV4753) was diluted in this buffer to an assay concentration of 0.200 μg/mL.

ATP/Substrate solution: 0.075 mM ATP, 12.5 mM MnCl₂, 50 mM Hepes, pH 7.4, 50 mM β-GOP, 250 nM Microcystin LR, 0.25 mM EDTA, 5 mM DTT, and 3.5 μg/mL GST-p70S6.

Detection reagent solution: 50 mM HEPES, pH 7.4, 0.01% Triton X-100, 0.01% BSA, 0.1 mM EDTA, 12.7 μg/mL Cy5-αGST Amersham (Cat#PA92002V), 9 ng/mL α-phospho p70S6 (Thr389) (Cell Signaling Mouse Monoclonal #9206L), 627 ng/mL α-mouse Lance Eu (Perkin Elmer Cat#AD0077).

To 20 μL of the Simple TOR buffer is added 0.5 μL of test compound in DMSO. To initiate the reaction 5 μL of ATP/Substrate solution was added to 20 μL of the Simple TOR buffer solution (control) and to the compound solution prepared above. The assay was stopped after 60 min by adding 5 μL of a 60 mM EDTA solution; 10 μL of detection reagent solution was then added and the mixture was allowed to sit for at least 2 hours before reading on a Perkin-Elmer Envision Microplate Reader set to detect LANCE Eu TR-FRET (excitation at 320 nm and emission at 495/520 nm).

TOR kinase inhibitors were tested in the TOR HTR-FRET assay and were found to have activity therein, with certain compounds having an IC₅₀ below 10 μM in the assay, with some compounds having an IC₅₀ between and 0.005 nM and 250 nM, others having an IC₅₀ between and 250 nM and 500 nM, others having an IC₅₀ between 500 nM and 1 μM, and others having an IC₅₀ between 1 μM and 10 μM.

DNA-PK Assay.

DNA-PK assay is performed using the procedures supplied in the Promega DNA-PK assay kit (catalog #V7870). DNA-PK enzyme can be purchased from Promega (Promega cat#V5811).

Selected TOR kinase inhibitors as described herein have, or are expected to have, an IC₅₀ below 10 μM in this assay, with some TOR kinase inhibitors as described herein having an IC₅₀ below 1 μM, and others having an IC₅₀ below 0.10 μM.

### 6.2 Cell Based Assays

6.2.1 TNFα Inhibition Assay in hPMBC

Human peripheral blood mononuclear cells (hPBMC) from normal donors are obtained by Ficoll Hypaque (Pharmacia, Piscataway, N.J., USA) density centrifugation. Cells are cultured in RPMI 1640 (Life Technologies, Grand Island, N.Y., USA) supplemented with 10% AB+ human serum (Gemini Bio-products, Woodland, Calif., USA), 2 mM L-glutamine, 100 U/mL penicillin, and 100 μg/mL streptomycin (Life Technologies).

PBMC (2.10⁵ cells) are plated in 96-well flat-bottom Costar tissue culture plates (Corning, N.Y., USA) in triplicate. Cells are stimulated with LPS (from Salmonella abortus equi, Sigma cat. no. L-1887, St. Louis, Mo., USA) at 1 ng/mL final concentration, in the absence or presence of compounds. Compounds provided herein are dissolved in DMSO (Sigma) and further dilutions are done in culture medium immediately before use. The final DMSO concentration in all assays can be about 0.25%. Compounds are added to cells 1 hour before LPS stimulation. Cells are then incubated for 18-20 hours at 37° C. in 5% CO₂, and supernatants are then collected, diluted with culture medium and assayed for TNFα levels by ELISA (Endogen, Boston, Mass., USA). IC₅₀s are calculated using non-linear regression, sigmoidal dose-response, constraining the top to 100% and bottom to 0%, allowing variable slope (GraphPad Prism v3.02).

6.2.2 Tumor Cell Assays.

Materials and Methods.

Cell lines and cell culture: Cell lines were purchased from American Type Culture Collection (ATCC) and maintained in culture medium recommended by ATCC. Ovarian cancer cell lines that were used or can be used include the following: Ovcar-3, Ovcar-4, Ovcar-5, Oncar-8 and Caov-3. Multiple myeloma (MM) cell lines that were used or can be used include the following: NCI-H929, LP-1, MM1.s, U266B1, DF-15 and RPMI-8226 human MM-derived cell lines. The REVLIMID® resistant cell lines H929/R1, H929/R2, H929/R3 and H929/R4 were established by continuous exposure of H929 parental cells (H929) to increasing concentrations of REVLIMID® for a minimum of 5 months. The control cell line H929/D was established by continuous exposure of H929 parental cells to 0.1% DMSO. The established H929/R1, H929/R2, H929/R3 and H929/R4 were pulsed once every 3 days with 10 μM REVLIMID, whereas H929/D was pulsed once every 3 days with 0.1% DMSO. Hepatocellular cancer, breast cancer, lung cancer and melanoma cell lines were purchased from commercial sources (ATCC, DSMZ, HSRRB) and routinely maintained in RPMI1640 or DMEM containing 10% fetal bovine serum at 37° C. with 5% CO₂. Hepatocellular carcinoma (HCC) cell lines that were used or can be used include the following: Hep3B, HepG2, HuH-7, PLC-PRF-5, SK-HEP-1, SNU-182, SNU-387, SNU-398, SNU-423, SNU-449, and SNU-387.

Measurement of Synergism of Cell Proliferation Inhibition Using a TOR Kinase Inhibitor in Combination with a Second Active Agent.

The cell viability assay was first performed with the TOR kinase inhibitor and the individual second active agents, to determine the dose range for subsequent combination studies. To maintain similar potency for the TOR kinase inhibitor and the second active agent, the highest combination dose started at the approximate IC₅₀ for each compound, with a constant ratio of 1:1 or 1:10 during dilutions. The TOR kinase inhibitor and the second active agent were each added to one well containing a final concentration of 0.2% DMSO (in triplicate). In the same plate in triplicate, the cells were treated with the TOR kinase inhibitor and each second active agent either simultaneously or sequentially (containing 0.2% DMSO). The number of cells affected by compound treatment was normalized to the DMSO control (100% viability) and the data was imported into the CalcuSyn software (V2.1, Biosoft). Synergism was quantitated by the combination index (CI) using CalcuSyn according to Chou-Talalay's CI method with mathematical modeling and simulations. The CI value indicates strong synergism if the value is between 0.1-0.3, synergism between 0.3-0.7, moderate synergism 0.7-0.85, slight synergism 0.85-0.90 and nearly additive 0.90-1.10 (Trends Pharmacol. Sci. 4, 450-454, 1983). ED₅₀ is the median effect dose at which a 50% growth inhibition is achieved.

Alternate Cell Viability Assay for MM Cell Lines.

Cell density and viability were monitored using the Vi-cell XR cell viability analyzer (Beckman Coulter). Once cell viability was >90% and cell density was ˜5×10⁵ cells/mL (log phase), the cells were incubated at the indicated concentrations of a TOR kinase inhibitor and/or second active agent at a final concentration of 0.1% vehicle (DMSO). For combination studies, the TOR kinase inhibitor and the second active agent were simultaneously added to cells in triplicate. Cell proliferation was determined after 5 days of treatment by flow cytometry on unfixed cells and using 7-aminoactinomycin D (7AAD) (Molecular Probes, Carlsbad, Calif., USA) exclusion (0.25% final dye concentration) for viability assessment. Flow cytometry was utilized to gate on the target cells and to measure 7AAD negative and 7AAD positive cells. Stained cells were analyzed on a FACS Array flow cytometer with standard BD FACS Array System software (BDBiosciences, Palo Alto, Calif.). The percentage of surviving cells (7AAD negative) was calculated relative to cells treated with vehicle (DMSO) control. For single compound treatments (TOR kinase inhibitor and second active agents separately), the average values from triplicates were plotted to obtain IC₅₀ values using software XLfit from IDBS. The formula used for determining IC₅₀ in XLfit was model number 205, which utilizes a 4 Parameter Logistic Model or Sigmoidal Dose-Response Model to calculate the IC₅₀ values. Results are set forth in Tables 2, 3, 4, 5 and 6.

TABLE 1
Human MM cell lines used

| Cell line | Sensitivity | Classification |
|---|---|---|
| LP-1 | Resistant to dex | cMyc, MMSET, p53mut, p18mut |
| DF15 | Sensitive | cMAF/MAB |
| U266 | Sensitive | CD-1, cMyc, p53mut, RBdel |
| RPMI8266 | Resistant to | cMyc, cMAP/MAB, K-RAS, p53Mut, |
| | lenalidomide | CD-2 |
| H929 | Sensitive | cMyc, MMSET, N-RAS, p18mut |
| H929/D | Sensitive | cMyc, MMSET, N-RAS, p18mut |
| H929/R1 | Resistant to | cMyc, MMSET, N-RAS, p18mut |
| | lenalidomide | |
| H929/R2 | Resistant to | cMyc, MMSET, N-RAS, p18mut |
| | lenalidomide | |
| H929/R3 | Resistant to | cMyc, MMSET, N-RAS, p18mut |
| | lenalidomide | |
| H929/R4 | Resistant to | cMyc, MMSET, N-RAS, p18mut |
| | lenalidomide | |
| MM1.s | Sensitive | cMAF/MAB |

TABLE 2
Combination study of Compound 1 and dexamethasone in
selected MM cell lines

| | Combo | | |
|---|---|---|---|
| | (1:1) | | |
| Cell line | IC50 (μM) | CI @ ED50 | Synergism |
| LP-1 | 0.38 | 0.6 | Synergism |
| DF15 | 0.0073 | 0.52 | Synergism |
| U266 | 0.083 | 0.52 | Synergism |
| RPMI8226 | 0.0003 | 0.053 | Very Strong |
| H929 | 0.044 | 0.29 | Strong Synergism |
| H929D | 0.0986 | 0.50 | Synergism |
| H929/R1 | 0.2 | 0.47 | Synergism |
| H929/R4 | 0.066 | 0.25 | Strong Synergism |
| MM1S | 0.00017 | 0.069 | Very Strong |

TABLE 3
Combination study of Compound 1 and lenalidomide in
selected MM cell lines

| | Combo | | |
|---|---|---|---|
| | (1:1) | | |
| Cell line | IC50 (μM) | CI @ ED50 | Synergism |
| RPMI8226 | 0.144 | 0.69 | Moderate Syn |
| H929 | 0.148 | 0.54 | Synergism |
| MM1S | 0.094 | 0.83 | Moderate Syn |
| LP-1 | 0.410 | 0.56 | Synergism |
| DF15 | 0.074 | 0.68 | Synergism |
| U266 | 0.210 | 0.72 | Modest Syn |
| H929/D1 | 0.130 | 0.58 | Synergism |
| H929/R1 | 0.420 | 1.19 | Slight antagonism |
| H929/R4 | 0.430 | 0.45 | Synergism |

TABLE 4
Combination study of Compound 1 and pomalidomide in
selected MM cell lines.

| | Combo | | |
|---|---|---|---|
| | (1:1) | | |
| Cell Lines | IC50 (μM) | CI @ ED50 | Synergism |
| H929 | 0.04 | 0.74 | Synergism |
| H929/D1 | 0.04 | 0.49 | Synergism |
| H929/R1 | 0.14 | 0.52 | Synergism |
| H929/R2 | 0.00 | 0.65 | Synergism |
| H929/R3 | 0.14 | 0.35 | Synergism |
| H929/R4 | 0.17 | 0.42 | Synergism |

TABLE 5
Combination study of Compound 2 and lenalidomide in
selected MM cell lines.

| | Combo | | |
|---|---|---|---|
| | (1:1) | | |
| Cell Lines | IC50 (μM) | CI @ ED50 | Synergism |
| H929 | 0.06 | 0.58 | Synergism |
| H929/D1 | 0.08 | 0.59 | Synergism |
| H929/R1 | 0.12 | 0.83 | Synergism |
| H929/R2 | 0.17 | 0.77 | Synergism |
| H929/R3 | 0.23 | N/A | No synergism |
| H929/R4 | 0.22 | N/A | No synergism |

N/A = not applicable,
CI not calculated as proliferation curve of lenalidomide had negative slope.

TABLE 6
Combination study of Compound 2 and pomalidomide in
selected MM cell lines.

| | Combo | | |
|---|---|---|---|
| | (1:1) | | |
| Cell Lines | IC50 (μM) | CI @ ED50 | Synergism |
| H929 | 0.02 | 0.30 | Synergism |
| H929/D1 | 0.03 | 0.35 | Synergism |
| H929/R1 | 0.11 | 0.60 | Synergism |
| H929/R2 | 0.12 | 0.71 | Synergism |
| H929/R3 | 0.94 | 0.53 | Synergism |
| H929/R4 | 0.18 | 0.64 | Synergism |

Effect of Compound 1 and Lenalidomide Treatment on Acquisition of Resistance in Multiple Myeloma Cells.

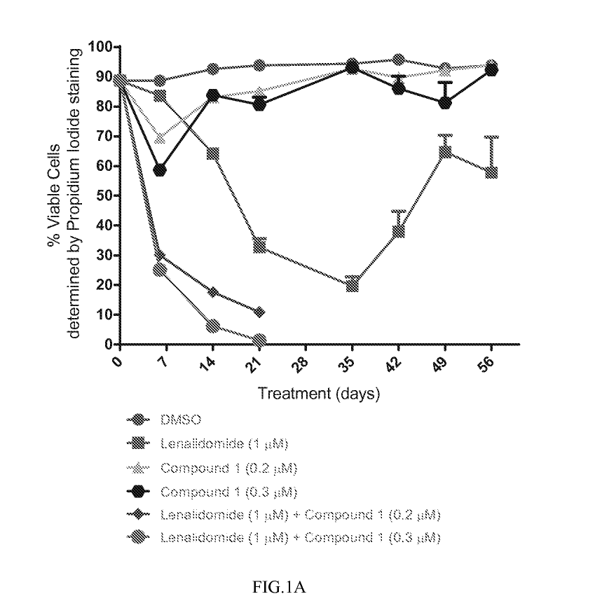

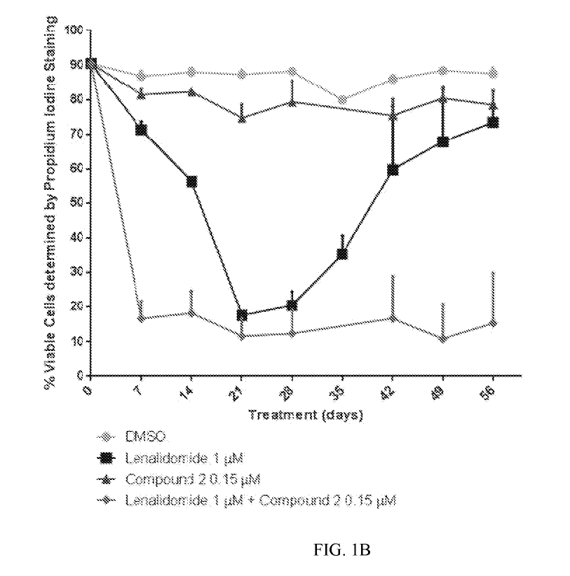

Continuous lenalidomide treatment of responsive myeloma cell lines results in the generation of lenalidomide-resistant myeloma cell lines (see Lopez-Girona A et al. Leukemia 26(11):2326-2335, 2012). Here, the effect of Compound 1 in combination with lenalidomide on the acquisition of resistance was evaluated in vitro. H929 cells were plated in triplicate at a density of 300,000 cells per mL flask in 10 mL of full medium. Lenalidomide, Compound 1 or a combination of lenalidomide with Compound 1 were added at the indicated concentrations (See FIG. 1A) to the culture medium. Every 3-4 days, cells were counted, viability was assessed by propidium iodide staining and flow cytometry, the old medium was removed, cells were washed twice with media, and then plated again at densities of 300,000 cells per mL flask in new full media containing same fresh drug treatment. Co-treatment of Compound 1 with lenalidomide effectively blocked the emergence of resistant H929 cells to either agent, compared to the single agent treatment (FIG. 1A).

Lenalidomide-resistant H929 cell lines (H929 R10-1 through 4) were generated, which have ˜50% reduction in cereblon protein (see Lopez-Girona A et al. Leukemia 26(11):2326-2335, 2012). Single agent Compound 1 showed potent anti proliferative effects on these resistant cell lines independent of cereblon levels. Furthermore, in combination with lenalidomide, dexamethasone or pomalidomide, Compound 1 showed synergistic effects in both lenalidomide-sensitive and resistant myeloma cell lines (Table 5-6). This indicates that Compound 2 activity in multiple myeloma cell lines in vitro, is independent of cereblon protein levels.

Effect of Compound 2 and Lenolidomide Treatment on Acquisition of Resistance in Multiple Myeloma Cells.

Continuous lenalidomide treatment causes emergence of acquired resistance in responsive myeloma cell lines. The effect of Compound 2 on acquisition of resistance was evaluated in vitro. H929 cells were plated in duplicate at a density of 300,000 cells per mL flask in 10 mL of full medium. Lenalidomide, Compound 2 or a combination of lenalidomide with Compound 2 were added at indicated concentrations (See FIG. 1B) to the culture medium. Every 3-4 days, cells were counted, viability assessed by propidium iodide staining and flow cytometry and old medium removed, cells washed twice with media and then plated again at densities of 300,000 cells per mL flask in new full media containing same fresh drug treatment. Co-treatment of compound 2 with lenalidomide effectively blocked the emergence of resistance to either agent (FIG. 1B).

Lenalidomide-resistant H929 cell lines (H929 R10-1 through 4) were generated, which have ˜50% reduction in cereblon protein (see Lopez-Girona A et al. Leukemia 26(11):2326-2335, 2012). Single agent Compound 2 showed potent anti proliferative effects on these resistant cell lines independent of cereblon levels. Furthermore, in combination with lenalidomide or pomalidomide, Compound 2 showed synergistic effects in both lenalidomide-sensitive and resistant myeloma cell lines (Table 2-4). This indicates that Compound 1 activity in multiple myeloma cell lines in vitro, is independent of cereblon protein levels.

Cell Viability Assay for Hepatocellular Cell Lines.

The TOR kinase inhibitor and second agent were added to an empty 384-well flat, clear bottom, black polystyrene, TC-Treated plate (Cat#3712, Corning, Mass.) via an acoustic dispenser (EDC Biosystems). The TOR kinase inhibitor was serially diluted 3-fold across the plate for nine concentrations and the second agent was serially diluted 3-fold down the plate for seven concentrations. An orthogonal titration of the two agents was performed to create 63 different combinations of the compounds. Both compounds were also added alone to determine their affects as single agents. DMSO (no compound) was used as control for 100% viability and background (no cells). Final assay DMSO concentration was 0.2% (v/v). Cells were added directly on top of the compounds at an optimized density to ensure that the cell growth was within the linear detection range of the assay after four days in culture. At its endpoint, cell viability was determined using Promega's CellTiter-Glo Luminescent Cell Viability Assay (Cat#G7573, Promega, Wis.) using the manufacturer's standard operating procedures. Background subtracted luminescence counts were converted to percentages of cell viability with respect to DMSO treated control cells. Dose response curves were generated using XLFit4 (IDBS, UK) by fitting the percentage of control data at each concentration using a 4 Parameter Logistic Model/Sigmoidal Dose-Response Model [y=(A+((B−A)/(1+((C/x)̂D))))]. To evaluate the combinatory effect of the two agents on a cell line, data was analyzed by comparing its combinatory response against the theoretical additive response of the two agents alone. The expected additive effect of two agents (A and B) was calculated using the fractional product method (Webb 1961): (fu)A,B=(fu)A×(fu)B where fu=fraction unaffected by treatment. Synergism of a combination is determined when the observed fraction unaffected in combination is less than (fu)A,B, while an additive effect is determined when the observed fraction unaffected in combination=(fu)A,B. Results are set forth in Table 7.

TABLE 7
Combination of a TOR kinase inhibitor and second active
agents in selected HCC cell lines With Lenalidomide

| HCC cell line | Combination | Synergism |
|---|---|---|
| HepG2 | Compound 1 + Lenalidomide | Weak |
| | | Synergy |

Compound 1 Combinatorial Effects with Lenalidomide in the Human Hepatocellular Carcinoma Anchorage Independent Growth Assay.

Summary.

The effect of Compound 1 on anchorage-independent growth (AIG) was assessed by colony formation assay in 2 Human Hepatocellular Carcinoma cell lines, HepG2 and SK-Hep-1. Compound 1 showed dose-dependent and significant anti-colony forming activity at concentrations of 0.1 to 100 μM in both cell lines. Compound 1 synergistically inhibited colony formation in both cell lines with lenalidomide.

Study Objectives.

The objective of this study was to evaluate the direct effects of Compound 1 and combinations of Compound 1 with lenalidomide on tumor cell anchorage-independent growth in 2 Human Hepatocellular Carcinoma cell lines. This evaluation was performed in colony formation assays.

Materials and Methods.

Cell Lines/Cells. Human cell lines HepG2 and SK-Hep-1 cells were obtained from American Type Culture Collection (ATCC; Manassas, Va.). Cells were cultured in DMEM (Dulbecco's Modified Eagle's Medium) (Mediatech; Mannasas, Va.) with 10% Premium FBS (Lonza, Walkersville, Md.).

Experimental Procedures.

(1) Single Agent Colony Formation Assay. Nobel Agar (1.2 grams; BD; Franklin Lakes, N.J.) was placed in a 100-mL sterile bottle. Sterile water (100 mL) was added and microwaved until the agar boiled. Equal volumes of agar and 2×RPMI medium (ECE Scientific; Doylestown, Pa.) were mixed and 300 μL were transferred to each well in a 24-well flat bottom plate (BD; Franklin Lakes, N.J.). Plates were kept at 4° C. until the agar solidified. Cultures of HepG2 and SK-Hep-1 cells were harvested and resuspended in culture medium at 3.6×10³ cells/mL. Equal volumes of agar, 2×RPMI, and cell suspension (1:1:1) were mixed in a sterile tube and 500 μL/well were immediately transferred into the 24-well plates. Plates were kept at 4° C. until the agar solidified. Culture medium (500 μL) containing compound or DMSO was added to each well (final DMSO concentration for each treatment was 0.2%). Compound 1 was tested at final concentrations of 0.1, 0.3, 1, 3, 10 and 30 μM. Cell treatments were set up in triplicate. Cells were incubated for 8-10 days at 37° C. in a 5% CO₂ atmosphere. Photographs (2× magnification) of each well were taken using a Nikon DXM1200 Digital Camera and Nikon ACT1 software and saved as a TIFF file. ImageQuant TL (GE Healthcare; Piscataway, N.J.) Colony Count Software was used to count colonies. (2) Combination Study Colony Formation Assay. Nobel Agar (1.2 grams; BD; Franklin Lakes, N.J.) was placed in a 100-mL sterile bottle. Sterile water (100 mL) was added and microwaved until the agar boiled. Equal volumes of agar and 2×RPMI medium (ECE Scientific; Doylestown, Pa.) were mixed and 300 μL were transferred to each well in a 24-well flat bottom plate (BD; Franklin Lakes, N.J.). Plates were kept at 4° C. until the agar solidified. Cultures of HepG2 and SK-Hep-1 cells were harvested and resuspended in culture medium at 3.6×10³ cells/mL. Equal volumes of agar, 2×RPMI, and cell suspension (1:1:1) were mixed in a sterile tube and 500 μL/well were immediately transferred into the 24-well plates. Plates were kept at 4° C. until the agar solidified. Culture medium (500 μL) containing compound or DMSO was added to each well (final DMSO concentration for each treatment was 0.2%). Cells were treated with single treatment as follows: Compound 1 was tested at final concentrations of 0.1 and 0.3 μM. Cell treatments were set up in triplicate. Cells were incubated for 8-10 days at 37° C. in a 5% CO₂ atmosphere. Photographs (2× magnification) of each well were taken using a Nikon DXM1200 Digital Camera and Nikon ACT1 software and saved as a TIFF file. ImageQuant TL (GE Healthcare; Piscataway, N.J.) Colony Count Software was used to count colonies.

Data Analysis.

The percentage inhibition of colony formation was calculated by normalizing to DMSO controls (100% control). Significance versus the DMSO control was calculated using One Way ANOVA and Dunnett's Post test or unpaired t tests using GraphPad Prism v5.01. To evaluate the combinatory effect, data from the three independent experiments were analyzed by comparing the combinatory response against the theoretical additive response of the two agents. The expected additive effect of two agents (A and B) was calculated using the fractional product method [Webb]: (fu)A,B=(fu)A×(fu)B; where fu=fraction unaffected by treatment. A synergism of a combination is determined when the observed fraction unaffected in combination is significantly less than (fu)A,B, whereas an additive effect is determined when the observed fraction unaffected in combination equals (fu)A,B. A partially additive effect occurs when the observed fraction unaffected is significantly greater than (fu)A,B.

Results.

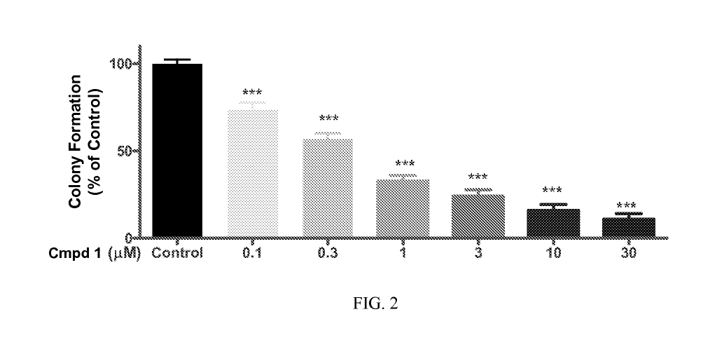

Results from colony formation assays with single agent treatments in HepG2 cells are presented in FIG. 2. HepG2 cells treated with 0.1, 0.3, 1, 3, 10, and 30 μM Compound 1 showed significant inhibition of colony formation at 74, 57, 33, 24, 16 and 11% of control, respectively (p value <0.001).

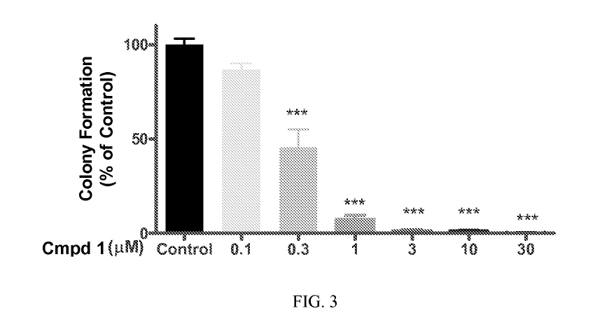

Results from colony formation assays with single agent treatments in SK-Hep-1 cells are presented in FIG. 3. Significant inhibition of colony formation (0-45% of control) was observed in SK-Hep-1 cells after treatment with 0.3-30 μM Compound 1 (p value <0.001).

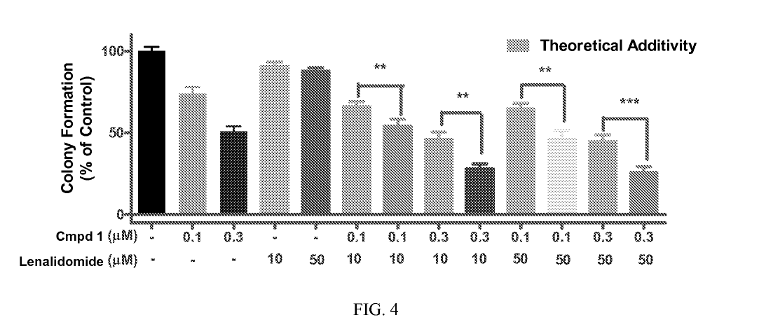

Results from the Compound 1 combination colony formation assays in HepG2 cells are presented in FIG. 4 and Table 8. FIG. 4 shows that there was synergy in all combinations of Compound 1 with lenalidomide (p value 0.01-0.001).

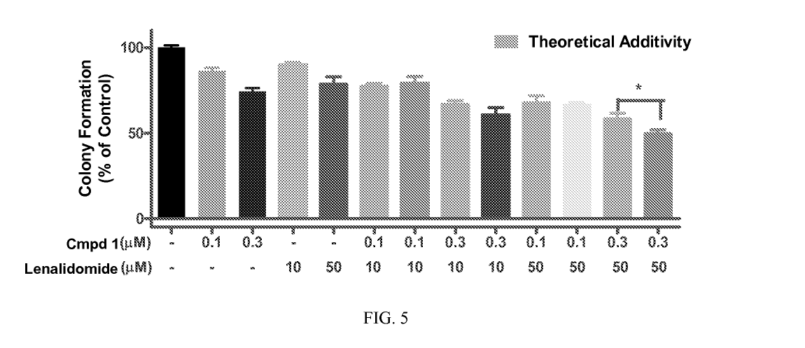

Results from the Compound 1 combination colony formation assays in SK-Hep-1 cells are presented in FIG. 5 and Table 9. FIG. 5 shows 0.1 μM Compound 1 in combination with 10 μM lenalidomide was partially additive (not significant). When 50 μM lenalidomide was combined with 0.1 μM Compound 1 there was an additive effect. The combination of 0.3 μM Compound 1 with 10 μM lenalidomide was additive but 0.3 μM CC- with 50 μM lenalidomide synergistically reduced colony formation (p value <0.05).

Conclusions.

The effect of Compound 1 in combination with lenalidomide on anchorage-independent growth was assessed by colony formation assay in HepG2 and SK-Hep-1 cells. Compound 1 exhibited dose-dependent and significant anti-colony forming in both cell lines at concentrations of 0.1 to 100 μM.

In HepG2 cells, Compound 1 in combination with lenalidomide had synergistic effects.

In SK-HEP-1 cells, Compound 1 in combination with lenalidomide had partially-additive to synergistic effects.

TABLE 8
Results of the Compound 1 HepG2 Colony Formation Assay

| | Colony | | p value of Actual |
|---|---|---|---|
| | Formation | Combination | vs Theoretical % |
| Compound | (% of Control) | Effect | Control |
| 0.1 μM Compound 1 + | 46 | synergism | ** |
| 10 μM lenalidomide | | | |
| 0.1 μM Compound 1 + | 53 | synergism | ** |
| 50 μM lenalidomide | | | |
| 0.3 μM Compound 1 + | 72 | synergism | ** |
| 10 μM lenalidomide | | | |
| 0.3 μM Compound 1 + | 74 | synergism | *** |
| 50 μM lenalidomide | | | |

HepG2 cells were plated in agar and incubated with compound for 8 days before colonies were counted.
Data were calculated as the percentage of inhibition relative to the cells treated with DMSO only = 0% inhibition.
Results represents the mean of n = 3 experiments in triplicate.
Fractional product method was used to calculate combination effects of compound combinations.
***p < 0.001,
**p < 0.01 vs theoretical additivity by unpaired t test.
ns = not significant.

TABLE 9
Results of the Compound 1 SK-Hep-1 Colony Formation Assay

| | Colony | | p value of Actual |
|---|---|---|---|
| | Formation | Combination | vs Theoretical % |
| Compound | (% of Control) | Effect | Control |
| 0.1 μM Compound 1 + | 21 | partially | ns |
| 10 μM lenalidomide | | additive | |
| 0.1 μM Compound 1 + | 34 | additive | ns |
| 50 μM lenalidomide | | | |
| 0.3 μM Compound 1 + | 39 | additive | ns |
| 10 μM lenalidomide | | | |
| 0.3 μM Compound 1 + | 50 | synergism | * |
| 50 μM lenalidomide | | | |

SK-Hep-1 cells were plated in agar and incubated with compound for 8 days before colonies were counted.
Data were calculated as the percentage of inhibition relative to the cells treated with DMSO only = 0% inhibition.
Results represents the mean of n = 3 experiments in triplicate.
Fractional product method was used to calculate combination effects of compound combinations.
*p < 0.05 vs theoretical additivity by unpaired t test.
ns = not significant.

Activity of TOR Kinase Inhibitor and Second Active Agents.

Other examples of second active agents that can be tested in the cell viability assays, using for example an ovarian cancer cell line, in combination with a TOR kinase inhibitor are, for example, other IMiD® immunomodulatory drugs.

Other examples of second active agents that can be tested in the cell viability assays, using for example a multiple myeloma cell line, in combination with a TOR kinase inhibitor are, for example, one or more of dexamethasone and IMiD® immunomodulatory drugs.

Other examples of second active agents that were tested or can be tested in the cell viability assays, using for example a hepatocellular carcinoma cell line, in combination with a TOR kinase inhibitor are, for example, other IMiD® immunomodulatory drugs.

In some examples, a third active agent was or can be tested in the cell viability assays described above, for example, an anti-CD-20 antibody, for example, Rituximab.

### 6.3 In Vivo Assays

DLBCL Xenograft Model.

Human DLBCL (WSU-DLCL2) cancer cell lines are injected into SCID (severe combined immunodeficiency) mice. Cancer cell lines are propagated in culture in vitro. Tumor bearing animals are generated by injecting 1×10⁶ cells into mice. Following inoculation of animals, the tumors are allowed to grow to a certain size prior to randomization. The mice bearing xenograft tumors ranging between 100 and 400 mm³ are pooled together and randomized into various treatment groups. A TOR kinase inhibitor and an IMiD® immunomodulatory drug (and optionally an anti-CD20 antibody, for example, rituximab (Rituxan® or MabThera®)) are administered at various dose levels to tumor-bearing mice. Additionally, reference chemotherapeutic agents such as CHOP therapy (combination of cyclophosphamide, doxorubicin, vincristine and prednisone) and negative controls are included in the study. Routes of administration can include subcutaneous (SC), intraperitoneal (IP), intravenous (IV), intramuscular (IM) and oral (PO). Tumor measurements and body weights are taken over the course of the study and morbidity and mortality are recorded. Tumors are measured twice a week using calipers and tumor volumes calculated using the formula of W²×L/2.

OCI-Ly10 DLBCL Xenograft Model.

OCI-Ly10 cells are derived from a diffuse-large B-cell lymphoma, a type of non-Hodgkins lymphoma. In brief, female CB.17 SCID mice are inoculated with 5×10⁶ OCI-Ly10 cells subcutaneously, and tumor are allowed to grow to approximately 50-300 mm³. The mice bearing xenograft with similarly sized tumors are pooled together and randomized into various treatment groups. A typical efficacy study design involves administering one or more compounds at various dose levels and schedules, based on prior single agent studies, to tumor-bearing mice. Tumor volume is measured biweekly for approximately 28 days of treatment using calipers, and tumor volume is calculated using standard methods, for example, using the formula of W²×L/2. Tumor volume can optionally be measured further post-treatment. Statistical analysis will be performed using standard statistical methods.

### 6.4 DLBCL Clinical Protocol A

A Phase 1B, Multi-Center, Open-Label Study of Novel Combinations and Rituximab in Diffuse Large B Cell Lymphoma.

This study is a Phase 1B, multi-center, open-label study of the TOR kinase inhibitor Compound 1, Compound A (3-(5-Amino-2-methyl-4-oxoquinazolin-3(4H)-yl)-piperidine-2,6-dione), and Compound AA (N-(3-(5-fluoro-2-(4-(2-methoxyethoxy)phenylamino)pyrimidin-4-ylamino)phenyl)acrylamide), when administered in combination and in combination with rituximab, in subjects having Diffuse Large B Cell Lymphoma (DLBCL).

The primary objective of the study is to determine the safety and tolerability of Compound A, Compound 1 and Compound AA, when administered orally as doublets and in combination with rituximab, and to define the non-tolerated dose (NTD) and the maximum tolerated dose (MTD) of each combination. The secondary objectives of the study are to provide information on the preliminary efficacy of each drug combination and to characterize the pharmacokinetics (PK) of Compound A, Compound 1 (and the M1 metabolite) and Compound AA following oral administration as single agents and after combination treatment to assess drug-drug interactions.

Study Design.

This study is a phase 1B dose escalation clinical study of Compound A, Compound 1 and Compound AA administered orally as doublets, and as triplets in combination with rituximab, in subjects with relapsed/refractory DLBCL who have failed at least one line of standard therapy. The study will explore two drug doses for each novel agent using a standard 3+3 dose escalation design with higher dose cohorts including the addition of a fixed dose of rituximab. Treatment arms include: Compound A+rituximab (Arm A), Compound A+Compound 1+/−rituximab (Arm B), Compound A+Compound AA+/−rituximab (Arm C) and Compound AA+Compound 1+/−rituximab (Arm D).

All treatments will be administered in 28-day cycles. Compound A, Compound 1 and Compound AA, are administered orally on continuous dosing schedules either once daily (QD) or twice daily (BID) on days 1-28 of each 28-day cycle. Rituximab, when included in the regimen, will employ a standard fixed dose (375 mg/m²) administered intravenously (IV) on Day 1 of each 28-day cycle only. All three compounds will be explored at two dose levels including: Compound A (2.0 and 3.0 mg QD), Compound 1 (20 and 30 mg QD), and Compound AA (375 and 500 mg BID). The highest two doublet dose levels for Arms B, C, and D will explore the doublets with and without rituximab.

A standard “3+3” dose escalation design will be used to identify initial toxicity of each combination. Subjects will be assigned to study treatment arms based on Investigator choice and open slots. Cohorts of 3 subjects will take study drugs in defined dose increments and, in the event of dose-limiting toxicity (DLT) in 1 of 3 evaluable subjects, cohorts will be expanded to 6 subjects.

An evaluable subject for DLT is defined as one that received at least 80% of the planned doses of Compound A, Compound 1 or Compound AA during Cycle 1; received at least 80% of the planned dose of rituximab during Cycle 1 (in rituximab containing cohorts only); and experienced study drug-related DLT after receiving at least one dose of any study drug. Non-evaluable subjects not due to DLT will be replaced. Additional subjects within any dose cohort may be enrolled at the discretion of the Safety Review Committee (SRC).

A dose will be considered the non-tolerated dose (NTD) when 2 of 6 evaluable subjects in a cohort experience drug-related DLT in Cycle 1. The maximum tolerated dose (MTD) is defined as the last dose level below the NTD with 0 or 1 out of 6 evaluable subjects experiencing DLT during Cycle 1. If 2 of 6 DLT are observed at the first dose level with either combination, a lower dose combination may be explored at the discretion of the SRC. An intermediate dose of Compound 1 (one between the NTD and the last dose level before the NTD) may be evaluated to accurately determine the MTD of the combination.

Following completion of dose escalation, selected combination treatment arms may be expanded up to approximately 20 subjects per arm. Expansion may occur at the MTD established in the dose escalation phase, or at an alternative tolerable combination dose level, based on review of study data.

Paired tumor biopsies for analysis of genetic abnormalities, gene expression and biomarkers of treatment activity are optional in the dose escalation phase but mandatory during the dose expansion phase.

The study population will consist of men and women, 18 years or older, with relapsed or refractory DLBCL, with disease progression following at least one standard first-line treatment regimen. Prior autologous stem cell transplant (greater than 3 months prior to enrollment) is allowed.

Enrollment is expected to take approximately 24 months (18 months for dose escalation, 6 months for expansion). Completion of active treatment and post-treatment follow-up is expected to take 6-12 additional months. The entire study is expected to last approximately 3 years.

Dose levels to be explored in this Phase 1b study are shown below:

| | | | | | | Arm C | | | | | |
|---|---|---|---|---|---|---|---|---|---|---|---|
| | Arm A | | Arm B | | | | Compound | | Arm D | | |
| | Cmpd A | Ritux | Cmpd | Cmpd 1 | Ritux | Cmpd A | AA | Ritux | Cmpd 1 | Cmpd AA | Ritux |
| Dose | (mg/ | (mg/m² | A (mg | (mg | (mg/m² | (mg/bid | (mg | (mg/m² | (mg | (mg bid | (mg/m² |
| Level | daily) | D1q28) | daily) | daily) | D1q28) | daily) | daily) | D1q28) | daily) | daily) | D1q28) |
| 1 | 2 | 375 | 2 | 20 | | 2 | 375 | | 20 | 375 | |
| 2a | | | 2 | 30 | | 2 | 500 | | 20 | 500 | |
| 2b | | | 2 | 30 | 375 | 2 | 500 | 375 | 20 | 500 | 375 |
| 3a | | | 3 | 30 | | 3 | 500 | | 30 | 500 | |
| 3b | 3 | 375 | 3 | 30 | 375 | 3 | 500 | 375 | 30 | 500 | 375 |

If unacceptable toxicity occurs at dose level 1, one starting dose reduction for Compound A (1 mg QD) and Compound 1 (15 mg QD) is allowed. No starting dose reductions for Compound AA are planned.

For Arms A and C, the Compound A dose will be reduced; for Arm D, the Compound 1 dose will be reduced. For Arm B, the safety review committee (SRC) will determine which of the two drugs in the doublet to dose reduce.

In Arm A (Compound A+rituximab), dose escalation will proceed from dose level 1 to 3b, since only Compound A is escalated. In Arms B, C and D dose levels 2b (doublet+rituximab) and 3a (dose escalation of doublet without rituximab) may be enrolled concurrently once dose level 2a (doublet) has been cleared. Both dose levels 2b and 3a must be cleared to move to dose level 3b.

Compound A, Compound 1 and Compound AA will be dosed daily and rituximab will be dosed on Day 1 of each 28-day cycle. For both the dose escalation and expansion phases, slight modifications to the dosing schedule will occur during Cycle 1 in order to facilitate PK and PD evaluation of each drug alone and in combination. Starting with Cycle 2 and thereafter, all oral drugs will start on Day 1 and continue through Day 28 and rituximab will be administered on Day 1.

Administration of study drugs during Cycle 1 is described below:

In Arm B: Compound 1 will be initiated on Cycle 1 Day 1 followed by PK and PD sampling and continue through Day 28. Compound A will be initiated on Cycle 1 Day 2 and continue through Day 28. Rituximab will be administered on Cycle 1 Day 8.

In Arm C: Compound A will be initiated on Cycle 1 Day 1 followed by PK and PD sampling and continue through Day 28. Compound AA will be initiated on Cycle 1 Day 2 and continue through Day 28. Rituximab will be administered on Cycle 1 Day 8.

In Arm D: Compound 1 will be initiated on Cycle 1 Day 1 followed by PK and PD sampling and continue through Day 28. Compound AA will be initiated on Cycle 1 Day 2 and continue through Day 28. Rituximab will be administered on Cycle 1 Day 8.

After the first dose is administered on Day 1 in any cohort, subjects will be observed for at least 28 days before the next higher protocol-specified dose cohort can begin. Intra-subject dose escalation of study drugs is not permitted during Cycle 1 but may be permitted in cycles beyond Cycle 1 if approved by the SRC. Dose reduction and temporary interruption of one or both drugs due to toxicity is allowed, but dose reduction during Cycle 1 will constitute DLT.

Study treatment may be discontinued if there is evidence of disease progression, unacceptable toxicity or subject/physician decision to withdraw. Subjects may continue to receive study drugs beyond disease progression at the discretion of the Investigator.

The estimated total number of subjects to be enrolled during dose escalation is approximately 50 to 100, depending on cohort size. Approximately 30 to 60 additional subjects (10-20 per selected regimen) will be evaluated for safety, PK, PD, and preliminary antitumor effects during the expansion phase.

Subjects will be evaluated for efficacy after every 2 cycles through Cycle 6, every 3 cycles through Cycle 12 and every 6 months thereafter. All treated subjects will be included in the efficacy analyses. The primary efficacy variable is tumor response rate. Tumor response will be determined by the Investigator, based on International Workshop Criteria (IWC) for NHL/DLBCL.

The safety variables for this study include adverse events (AEs), safety clinical laboratory variables, 12-lead electrocardiograms (ECGs), left ventricular ejection fraction (LVEF) assessments, physical examinations, vital signs, exposure to study treatment, assessment of concomitant medications, and pregnancy testing for females of child bearing potentials (FCBP).

During dose escalation, the decision to either evaluate a higher dose level or declare an MTD will be determined by the SRC, based on their review of all available clinical and laboratory safety data for a given dose cohort.

The SRC will also select the dose and schedule of treatment regimens of interest for cohort expansion. One or more regimens may be selected for cohort expansion. The SRC will continue to review safety data regularly throughout the study and make recommendations about study continuation and dose modification, as appropriate.

The concentration-time profiles of Compound A, Compound 1 and Compound AA will be determined from serial blood samples collected after administration of study drugs as single agents and after combination treatment.

The effect of Compound A and Compound AA on Compound 1 and M1 PK will be assessed, as will the effect of Compound AA on Compound A PK. Systemic exposure of Compound A, Compound 1 and the M1 metabolite, and Compound AA will be correlated with safety, PD and activity outcomes.

### 6.5 Clinical Protocol B

A Phase 1B, Multi-Center, Open-Label Study of Novel Combinations and Rituximab in Diffuse Large B Cell Lymphoma.

This study is a Phase 1B, multi-center, open-label study of the TOR kinase inhibitor Compound 1, Compound A (3-(5-Amino-2-methyl-4-oxoquinazolin-3(4H)-yl)-piperidine-2,6-dione), and Compound AA (N-(3-(5-fluoro-2-(4-(2-methoxyethoxy)phenylamino)pyrimidin-4-ylamino)phenyl)acrylamide), when administered in combination and in combination with rituximab, in subjects having Diffuse Large B Cell Lymphoma (DLBCL).

The primary objective of the study is to determine the safety and tolerability of Compound A, Compound 1 and Compound AA, when administered orally as doublets and as triplets in combination with rituximab, determine the safety and tolerability of Compound A when administered in combination with rituximab, and to define the non-tolerated dose (NTD) and the maximum tolerated dose (MTD) and/or the recommended phase 2 dose (RP2D) of each combination. The secondary objectives of the study are to provide information on the preliminary efficacy of each drug combination and to characterize the steady state pharmacokinetics (PK) of Compound A, Compound 1 and Compound AA following combination oral administration as single agents.

Study Design.

This study is a phase 1b dose escalation and expansion clinical study of Compound A, Compound 1 and Compound AA administered orally as doublets, and as triplets in combination with rituximab, as well as a Compound A plus rituximab doublet, in subjects with relapsed/refractory DLBCL who have failed at least one line of standard therapy. The dose escalation phase of the study will explore one or more drug doses for each novel agent using a standard 3+3 dose escalation design with higher dose cohorts including the addition of a fixed dose of rituximab, followed by expansion of selected cohorts of interest. The addition of rituximab can also be evaluated at the doublet MTD if the higher dose levels are not reached. Treatment arms include: Compound A+Compound 1+/−rituximab (Arm A), Compound A+Compound AA+/−rituximab (Arm B), Compound AA+Compound 1+/−rituximab (Arm C), and Compound A+rituximab (Arm D).

All treatments will initially be administered in 28-day cycles. Compound A, Compound 1 and Compound AA, will initially be administered orally on continuous dosing schedules either once daily (QD) or twice daily (BID) on days 1 to 28 of each 28-day cycle. Rituximab, when included in the regimen, will be administered only once in each cycle as a standard fixed intravenous (IV) dose of 375 mg/m² on Day 8 of Cycle 1, and Day 1 of each subsequent cycle. All three compounds will be explored at one or two dose levels including: Compound A (2.0 and 3.0 mg QD), Compound 1 (20 and 30 mg QD), and Compound AA (500 mg BID). The highest two doublet dose levels (or the MTD if at a lower dose level) will explore the combinations with rituximab.

A standard “3+3” dose escalation design will be used to identify initial toxicity of each combination. Subjects will be assigned to study treatment arms based on investigator choice and open slots. Cohorts of 3 subjects will take study drugs in defined dose increments and, in the event of dose-limiting toxicity (DLT) in 1 of 3 evaluable subjects, cohorts will be expanded to 6 subjects.

An evaluable subject for DLT is defined as one that received at least 80% of the planned doses of Compound A, Compound 1 or Compound AA during Cycle 1 without experiencing a DLT, and received at least 80% of the planned dose of rituximab during Cycle 1 (in rituximab containing cohorts only); without experiencing a DLT, or experienced a DLT after receiving at least one dose of any study drug. Non-evaluable subjects will be replaced. Additional subjects within any dose cohort may be enrolled at the discretion of the Safety Review Committee (SRC).

A dose will be considered the NTD when 2 of 6 evaluable subjects in a cohort experience a drug-related DLT in Cycle 1. The MTD is defined as the last dose level(s) below the NTD with 0 or 1 out of 6 evaluable subjects experiencing a DLT during Cycle 1. If 2 of 6 DLTs are observed at the first dose level with either combination, a lower dose combination may be explored at the discretion of the SRC. An intermediate dose of study drugs (one between the NTD and the last dose level before the NTD) may be evaluated to accurately determine the MTD of the combination. Alternative schedules reducing the total exposure of study drug during a cycle may also be evaluated for tolerability.

Following completion of dose escalation, selected combination treatment arms may be expanded up to approximately 20 subjects per arm. Expansion may occur at the MTD established in the dose escalation phase, or at an alternative tolerable combination dose level, based on review of study data.

Paired tumor biopsies for analysis of genetic abnormalities, RNA and protein expression, and biomarkers of treatment activity are optional in the dose escalation phase but mandatory during the dose expansion phase.

The study population will consist of men and women, 18 years or older, with relapsed or refractory DLBCL, with disease progression following at least two prior standard treatment regimens and autologous stem cell transplant (ASCT) in chemotherapy sensitive patients are eligible. Enrollment will also include selected high-risk subjects prior to ASCT and subjects not otherwise eligible for ASCT.

Inclusion Criteria:

Subjects must satisfy all of the following criteria to be enrolled in the study: (1) Understand and voluntarily sign an informed consent document prior to conducting any study related assessments or procedures; (2) Consent to retrieve archival tumor tissue for analysis (in the event that archival tissue is not available an exception may be granted by the Sponsor); (3) Consent to undergo paired tumor biopsies (Screening and on treatment) for genetic analysis and biomarker evaluation (expansion cohorts only) (waiver to this requirement may be given under exceptional circumstances); (4) Men and women, 18 years or older, with histologically or cytologically-confirmed, relapsed or refractory DLBCL (including transformed low grade lymphoma) following at least two prior standard treatment regimens (eg, R-CHOP or similar first-line regimen and at least one second-line salvage regimen) and ASCT in chemotherapy sensitive patients, with the following exceptions: (i) Subjects in the pre-ASCT setting with poor prognosis, defined as primary refractory disease, relapse within 12 months following first-line treatment, “double-hit” lymphomas with Bcl-2/Myc gene rearrangements or overexpression, or high IPI score (2,3) at relapse; (ii) Subjects age >65 refusing, or not otherwise appropriate, per the Investigator's judgment, for ASCT; (5) At least one site of measurable disease (>1.5 cm in the long axis or >1.0 cm in both the long and short axis); (6) ECOG PS of 0 or 1; (7) Subjects must have the following laboratory values: (i) Absolute Neutrophil Count (ANC)≧1.5×10⁹/L without growth factor support for 7 days; (ii) Hemoglobin (Hgb)≧8 g/dL; (iii) Platelets (plt)≧50×10⁹/L without transfusion for 7 days (14 days if received pegfilgrastim); (iv) Potassium within normal limits or correctable with supplements; (v) AST/SGOT and ALT/SGPT≦2.5×Upper Limit of Normal (ULN) or ≦5.0×ULN if liver tumor is present; (vi) Serum bilirubin ≦1.5×ULN; (vii) Estimated serum creatinine clearance of ≧50 mL/min using the Cockcroft-Gault equation; (8) Females of childbearing potential (FCBP) (A female of childbearing potential is a sexually mature woman who 1) has not undergone a hysterectomy (the surgical removal of the uterus) or bilateral oophorectomy (the surgical removal of both ovaries) or 2) has not been naturally postmenopausal for at least 24 consecutive months (ie, has had menses at any time during the preceding 24 consecutive months) must: (i) Agree to use at least two effective contraceptive methods (oral, injectable, or implantable hormonal contraceptive; tubal ligation; intra-uterine device; barrier contraceptive with spermicide; or vasectomized partner), one of which must be barrier, throughout the study, and for up to 28 days following the last dose of study drug; (ii) Have a negative serum pregnancy test (sensitivity of at least 25 mIU/mL) at Screening; (iii) Have a negative serum or urine pregnancy test (investigator's discretion) within 72 hours prior to Cycle 1 Day −1 of study treatment (note that the Screening serum pregnancy test can be used as the test prior to Day −1 study treatment if it is performed within the prior 72 hours); (iv) Avoid conceiving for 28 days after the last dose of any study drug; (v) Agree to ongoing pregnancy testing during the course of the study; (9) Males must practice complete abstinence or agree to use a condom (a latex condom is recommended) during sexual contact with a pregnant female or a female of childbearing potential and will avoid conceiving while participating in the study, during dose interruptions, and for at least 28 days following study drug discontinuation, even if he has undergone a successful vasectomy; (10) All subjects enrolled into treatment arms receiving Compound A must: (i) Understand that the (investigational product) IP could have a potential teratogenic risk; (ii) Agree to abstain from donating blood or sperm while taking IP and for at least 28 days following discontinuation of IP; (iii) Agree not to share IP with another person; (iv) Be counseled about pregnancy precautions and risks of fetal exposure and agree to requirements of PPRMP; (11) Able to adhere to the study visit schedule and other protocol requirements.

Exclusion Criteria:

The presence of any of the following will exclude a subject from enrollment: (1) Symptomatic central nervous system involvement; (2) Known symptomatic acute or chronic pancreatitis; (3) Persistent diarrhea or malabsorption ≧NCI CTCAE grade 2, despite medical management; (4) Peripheral neuropathy ≧NCI CTCAE grade 2; (5) Impaired cardiac function or clinically significant cardiac diseases, including any of the following: (i) LVEF<45% as determined by MUGA or ECHO; (ii) Complete left bundle branch or bifascicular block (iii) Congenital long QT syndrome; (iv) Persistent or clinically meaningful ventricular arrhythmias; (v) QTcF>460 msec on Screening ECG (mean of triplicate recordings); (vi) Unstable angina pectoris or myocardial infarction ≦3 months prior to starting study drugs; (vii) Troponin-T value >0.4 ng/ml or BNP>300 pg/mL (Subjects with baseline troponin-T>ULN or BNP>100 pg/mL are eligible but must have cardiologist evaluation prior to enrollment in the trial for baseline assessment and optimization of cardioprotective therapy); (6) Subjects with diabetes on active treatment or subjects with either of the following (for subjects treated on Compound 1 containing arms only): (i) Fasting blood glucose (FBG)≧126 mg/dL (7.0 mmol/L); (ii) HbA1c≧6.5%; (7) Prior ASCT≦3 months before first dose; (8) Prior allogeneic stem cell transplant with either standard or reduced intensity conditioning; (9) Prior systemic cancer-directed treatments or investigational modalities ≦5 half lives or 4 weeks prior to starting study drugs, whichever is shorter; (10) Prior treatment with a dual mTORC1/mTORC2 inhibitor (Compound 1 only) or BTK inhibitor (Compound AA arms only) (Prior treatment with rapamycin analogues, PI3K or AKT inhibitors, lenalidomide and rituximab are allowed); (11) Subjects who have undergone major surgery ≦2 weeks prior to starting study drugs (subjects must have recovered from any effects of recent surgery or therapy that might confound the safety evaluation of study drug; no specific washout is required for radiotherapy); (12) Women who are pregnant or breast feeding (adults of reproductive potential not employing two forms of birth control); (13) Subjects with known HIV infection; (14) Subjects with known chronic active hepatitis B or C virus (HBV/HCV) infection; (15) Subjects with treatment-related myelodysplastic syndrome; (16) Chronic use of proton pump inhibitors or H2 antagonists or their use within 7 days of first dose for subjects treated on Compound AA-containing arms (B and C). Subjects with chronic gastroesophageal reflux disease, dyspepsia, and peptic ulcer disease, should be carefully evaluated for their suitability for this treatment prior to enrollment in this study (these medications are prohibited concomitant medications throughout the study); (17) Any other significant medical condition, laboratory abnormality, or psychiatric illness which places the subject at unacceptable risk or that would prevent the subject from complying with the study; (18) History of concurrent second cancers requiring active, ongoing systemic treatment.

Enrollment is expected to take approximately 24 months to complete (18 months for dose escalation, and 6 months for expansion). Completion of active treatment and post-treatment follow-up is expected to take—an additional 6-12 months. The entire study is expected to last approximately 3 years.

The End of Trial is defined as either the date of the last visit of the last subject to complete the study, or the date of receipt of the last data point from the last subject that is required for primary, secondary and/or exploratory analysis, as pre-specified in the protocol and/or the Statistical Analysis Plan, whichever is the later date.

Dose levels to be explored in this Phase 1b study are shown below:

| | | | | | | | | Arms |
|---|---|---|---|---|---|---|---|---|
| | | | | | | | | A, B, C, D |
| | Arm A | | Arm B | | Arm C | | Arm D | Ritux |
| Dose | Cmpd A | Cmpd 1 | Cmpd A | Cmpd AA | Cmpd 1 | Cmpd AA | Cmpd A | (mg/m²) |
| Level | (mg QD) | (mg QD) | (mg QD) | (mg BID) | (mg QD) | (mg BID) | (mg QD) | (q 28) |
| 1 | 2 | 20 | — | — | — | — | | — |
| 2 | 2 | 30 | 2 | 500 | 20 | 500 | | — |
| 3 | 2 | 30 | 2 | 500 | 20 | 500 | 2 | 375 |
| 4 | 3 | 30 | 3 | 500 | 30 | 500 | 3 | 375 |

BID = twice a day;
QD = once a day;
q 28 = once every 28 days (Day 8 in Cycle 1; Day 1 in subsequent cycles);
Ritux = rituximab

All treatment cycles are 28 days in length. Dosing will start at Dose Level 1 for Arm A, Dose Level 2 for Arms B and C and Dose Level 3 for Arm D. Each dose level must clear before initiating the next higher dose level. If unacceptable toxicity occurs at the initial dose level, dose reductions for Compound A (1.5 mg QD and 1 mg QD) and Compound 1 (15 mg QD) are allowed. Additionally, exploration of an alternative schedule of Compound A (daily for 5 out of 7 days) is allowed based on SRC review. No starting dose reductions for Compound AA are planned.

For Arms B and D, the Compound A dose will be reduced; for Arm C, the Compound 1 dose will be reduced. For Arm A, the SRC will determine which of the two drugs in the doublet to dose reduce.

Compound A, Compound 1 and Compound AA will be dosed daily on a continuous basis in 28-day cycles. Compound A dosing may be modified to 5 out of 7 days based on SRC review (the cycle length will remain 28 days). To minimize the risk of tumor lysis syndrome, rituximab, when administered, will be dosed on Day 8 of Cycle 1, then on Day 1 of each subsequent cycle.

After the first dose is administered on Day 1 in any cohort, subjects will be observed for at least 28 days before the next higher protocol-specified dose cohort can begin. Intra-subject dose escalation of study drugs is not permitted during Cycle 1 but may be permitted in later cycles if approved by the SRC. Dose reduction and temporary interruption of one or both drugs due to toxicity is allowed, but dose reduction during Cycle 1 will constitute DLT.

Study treatment may be discontinued if there is evidence of disease progression, unacceptable toxicity or subject/physician decision to withdraw. Subjects may continue to receive study drugs beyond disease progression at the discretion of the Investigator.

The estimated total number of subjects to be enrolled during dose escalation is approximately 36 to 72, depending on cohort size. Approximately 40 to 80 additional subjects (10 to 20 per selected regimen) will be evaluated for safety, PK, PD, and preliminary antitumor effects during the expansion phase.

Subjects will be evaluated for efficacy after every 2 cycles through Cycle 6, every 3 cycles through Cycle 12 and every 6 months thereafter. All treated subjects will be included in the efficacy analyses. The primary efficacy variable is tumor response rate and duration. Tumor response will be determined by the Investigator, based on International Workshop Criteria (IWC) for Malignant Lymphoma (Cheson et al, J Clin Oncol, 2007, 25 (5): 579-586).

Secondary and exploratory endpoints include evaluation of Compound A, Compound 1, and Compound AA pharmacodynamic and predictive biomarkers in blood and/or tumor and exploration of PK, PD, toxicity, and activity relationships

The safety variables for this study include adverse events (AEs), safety clinical laboratory variables, 12-lead electrocardiograms (ECGs), Eastern Cooperative Oncology Group performance status (ECOG-PS), left ventricular ejection fraction (LVEF) assessments, physical examinations, vital signs, exposure to study treatment, assessment of concomitant medications, and pregnancy testing for females of child bearing potential (FCBP).

During dose escalation, the decision to either evaluate a higher dose level or declare an MTD will be determined by the SRC, based on their review of all available clinical and laboratory safety data for a given dose cohort.

The SRC will also select the dose and schedule and treatment regimens of interest for cohort expansion. One or more regimens may be selected for cohort expansion. The SRC will continue to review safety data regularly throughout the study and make recommendations about study continuation and dose modification, as appropriate.

The steady-state plasma pharmacokinetics of Compound A, Compound 1, the M1 metabolite of Compound 1, and Compound AA will be determined in Arm C. Sparse plasma concentrations of Compound A, Compound 1, and Compound AA will be evaluated after single dose administration of drug combinations and at steady state in all arms (except dose level 2 in Arm C, which will undergo intensive PK monitoring at steady state). Correlations of drug exposure with safety, PD and clinical endpoints may also be explored as an exploratory endpoint.

Pharmacodynamic biomarkers of each novel agent at baseline and on study treatment will be explored, including: 1) Compound A, modulation of CRBN substrates in B and T cells; 2) Compound 1, mTOR signaling pathway biomarkers (p4E-BP1, pAKT, and possibly others); 3) Compound AA, B-cell receptor signaling pathway biomarkers (pBTK, pERK, and possibly others).

Overview of Statistical Methodology.

Statistical analyses will be performed by study phase, treatment arm, and dose level as needed or applicable. All analyses will be descriptive in nature. The efficacy variable of primary interest is tumor response and duration. Other preliminary efficacy variables, including (FDG)-PET outcomes will be summarized using frequency tabulations for categorical variables or descriptive statistics for continuous variables. Efficacy analysis will be repeated for enrolled, treated and efficacy evaluable populations, with the result using treated population considered primary. All summaries of safety data will be conducted using subjects receiving at least one dose of Study Drug (the Safety Population).

All biomarker-related data presentations will be based on treated subjects with at least one baseline and one on-study evaluation (the biomarker evaluable population), unless specified otherwise. Descriptive statistics will be presented for baseline and change from baseline of continuous biomarker endpoints, by treatment arm and overall.

During the dose escalation phase, approximately 36 to 72 subjects will be enrolled. After that, up to 20 subjects may be enrolled in each of the selected cohorts during the dose expansion phase. Since the primary objective of this study is to determine safety/tolerability and MTD/RP2D, an exact sample size for either phase will not be stated in advance.

### 6.6 Compound Formulations

Illustrative formulations of Compound 1 useful in the methods provided herein are set forth in Tables 10-13, below.

TABLE 10

| | Amounts | |
|---|---|---|
| Ingredients | mg | % w/w |
| Compound 1 | 20.0 | 15.38 |
| Lactose monohydrate, NF (Fast Flo 316) | 63.98 | 49.22 |
| Microcrystalline cellulose, NF (Avicel pH 102) | 40.30 | 31.00 |
| Croscarmellose sodium, NF (Ac-Di-Sol) | 3.90 | 3.00 |
| Stearic acid, NF | 0.52 | 0.40 |
| Magnesium Stearate, NF | 1.30 | 1.00 |
| Total | 130.0 | 100 |
| Opadry yellow 03K12429 | 5.2 | 4.0 |

TABLE 11

| | Amounts | |
|---|---|---|
| Ingredients | mg | % w/w |
| Compound 1 | 5.0 | 3.80 |
| Lactose monohydrate, NF (Fast Flo 316) | 78.98 | 60.70 |
| Microcrystalline cellulose, NF (Avicel pH 102) | 40.30 | 31.00 |
| Croscarmellose sodium, NF (Ac-Di-Sol) | 3.90 | 3.00 |
| Stearic acid, NF | 0.52 | 0.40 |
| Magnesium Stearate, NF | 1.30 | 1.00 |
| Total | 130.0 | 100 |
| Opadry II pink 85F94211 | 5.2 | 4% weight gain |

TABLE 12

| | Amounts | | | |
|---|---|---|---|---|
| Ingredients | mg | | | % w/w |
| Compound 1 | 15.0 | 20.0 | 30.0 | 15.38 |
| Lactose monohydrate, NF (Fast Flo | 48.37 | 64.50 | 96.75 | 49.62 |
| 316) | | | | |
| Microcrystalline cellulose, NF | 30.23 | 40.30 | 60.45 | 31.00 |
| (Avicel pH 112) | | | | |
| Croscarmellose sodium, NF | 2.925 | 3.90 | 5.85 | 3.00 |
| (Ac-Di-Sol) | | | | |
| Magnesium Stearate, NF | 0.975 | 1.30 | 1.95 | 1.00 |
| Total | 97.50 | 130.0 | 195.00 | 100 |
| Opadry yellow 03K12429 | 3.9 | | | 4.0 |
| Opadry II Pink 85F94211 | | 5.2 | | 4.0 |
| Opadry Pink 03K140004 | | | 7.8 | 4.0 |

TABLE 13

| | Amounts | |
|---|---|---|
| Ingredients | mg | % w/w |
| Compound 1 | 45.00 | 15.38 |
| Lactose monohydrate, NF (Fast Flo 316) | 143.955 | 49.22 |
| Microcrystalline cellulose, NF (Avicel pH 102) | 90.675 | 31.00 |
| Croscarmellose sodium, NF (Ac-Di-Sol) | 8.775 | 3.00 |
| Stearic acid, NF | 1.170 | 0.40 |
| Magnesium Stearate, NF | 2.925 | 1.00 |
| Total | 292.50 | 100 |
| Opadry pink 03K140004 | 11.70 | 4.0 |

Illustrative formulations of Compound 2 useful in the methods provided herein are set forth in Table 14, below.

TABLE 14
Exemplary Tablet Formulations

| | % w/w (mg) | | | |
|---|---|---|---|---|
| | Batch # | | | |
| Ingredients | 1 | 2 | 3 | 4 |
| Compound 2 (active ingredient) | 10 | 10 | 10 | 10 |
| Mannitol (Mannogem EZ) | qs | qs | qs | qs |
| Microcrystalline Cellulose | 25 | 25 | 25 | 25 |
| (PH 112) | | | | |
| Sodium Starch Glycolate | 3 | 3 | 3 | 3 |
| Silicon dioxide | 1 | 1 | 1 | 1 |
| Stearic acid | 0.5 | 0.5 | 0.5 | 0.5 |
| Disodium EDTA | | | 0.5 | 0.5 |
| BHT | | 0.4 | | 0.4 |
| Magnesium Stearate | 0.65 | 0.65 | 0.65 | 0.65 |
| Total | 100 | 100 | 100 | 100 |
| Color | Yellow | Yellow | Yellow | Yellow |

A number of references have been cited, the disclosures of which are incorporated herein by reference in their entirety. The embodiments disclosed herein are not to be limited in scope by the specific embodiments disclosed in the examples which are intended as illustrations of a few aspects of the disclosed embodiments and any embodiments that are functionally equivalent are encompassed by the present disclosure. Indeed, various modifications of the embodiments disclosed herein are in addition to those shown and described herein will become apparent to those skilled in the art and are intended to fall within the scope of the appended claims.

What is claimed is:

1. A method for treating a cancer, comprising administering an effective amount of a TOR kinase inhibitor in combination with an effective amount of an IMiD® immunomodulatory drug to a patient having a cancer, wherein the TOR kinase inhibitor is a compound of formula (I): 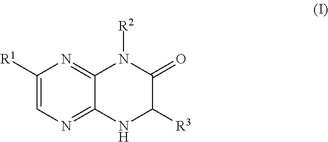
and pharmaceutically acceptable salts, clathrates, solvates, stereoisomers, tautomers, metabolites, isotopologues and prodrugs thereof, wherein:
R¹ is substituted or unsubstituted C₁₋₈ alkyl, substituted or unsubstituted aryl, substituted or unsubstituted cycloalkyl, substituted or unsubstituted heterocyclyl, or substituted or unsubstituted heterocyclylalkyl;
R² is H, substituted or unsubstituted C₁₋₈ alkyl, substituted or unsubstituted cycloalkyl, substituted or unsubstituted heterocyclyl, substituted or unsubstituted heterocyclylalkyl, substituted or unsubstituted aralkyl, or substituted or unsubstituted cycloalkylalkyl;
R³ is H, or a substituted or unsubstituted C₁₋₈ alkyl,
provided the TOR kinase inhibitor is not 7-(4-hydroxyphenyl)-1-(3-methoxybenzyl)-3,4-dihydropyrazino[2,3-b]pyrazin-2(1H)-one

2. The method of claim 1, wherein the cancer is a blood borne cancer.

3. The method of claim 2, wherein the blood borne cancer is a lymphoma, a leukemia or a multiple myeloma.

4. The method of claim 3, wherein the lymphoma is non-Hodgkin's lymphoma.

5. The method of claim 4, wherein the non-Hodgkin's lymphoma is diffuse large B-cell lymphoma (DLBCL), follicular lymphoma (FL), acute myeloid leukemia (AML), mantle cell lymphoma (MCL), or ALK+ anaplastic large cell lymphoma.

6. The method of claim 4, wherein the non-Hodgkin's lymphoma is diffuse large B-cell lymphoma (DLBCL).

7. The method of claim 3, wherein the lymphoma is a B-cell lymphoma.

8. The method of claim 7, wherein the B-cell lymphoma is a B-cell non-Hodgkin's lymphoma selected from diffuse large B-cell lymphoma, Burkitt's lymphoma/leukemia, mantle cell lymphoma, mediastinal (thymic) large B-cell lymphoma, follicular lymphoma, marginal zone lymphoma, and lymphoplamacytic lymphoma/Waldenstrom macroglobulinemia.

9. The method of claim 8, wherein the B-cell non-Hodgkin's lymphoma is refractory B-cell non-Hodgkin's lymphoma.

10. The method of claim 8, wherein the B-cell non-Hodgkin's lymphoma is relapsed B-cell non-Hodgkin's lymphoma.

11. The method of claim 7, wherein the B-cell lymphoma is chronic lymphocytic leukemia or small lymphocytic lymphoma.

12. The method of claim 3, wherein the lymphoma is a T-cell lymphoma.

13. The method of claim 1, wherein the cancer is a cancer of the head, neck, eye, mouth, throat, esophagus, bronchus, larynx, pharynx, chest, bone, lung, colon, rectum, stomach, prostate, urinary bladder, uterine, cervix, breast, ovaries, testicles or other reproductive organs, skin, thyroid, blood, lymph nodes, kidney, liver, pancreas, and brain or central nervous system.

14. The method of claim 1, wherein the cancer is a cancer associated with the pathways involving mTOR, PI3K, or Akt kinases and mutants or isoforms thereof.

15. The method of claim 1, wherein the IMiD® immunomodulatory drug is lenalidomide.

16. The method of claim 1, wherein the IMiD® immunomodulatory drug is pomalidomide.

17. The method of claim 1, wherein the IMiD® immunomodulatory drug is (S)-3-(4-(4-(morpholinomethyl)benzyloxy)-1-oxoisoindolin-2-yl)piperidine-2,6-dione, N-[2-(2,6-Dioxo-piperidin-3-yl)-1-oxo-2,3-dihydro-1H-isoindol-4-ylmethyl]-2-phenyl-acetamide, 2-(2,6-Dioxopiperidin-3-yl)-4-phenylaminoisoindole-1,3-dione, 2-[2-(2,6-Dioxopiperidin-3-yl)-1,3-dioxo-2,3-dihydro-1H-isoindol-4-ylamino]-N-methylacetamide, 1-[2-(2,6-Dioxo-piperidin-3-yl)-1,3-dioxo-2,3-dihydro-1H-isoindol-4-ylmethyl]-3-p-tolyl-urea, or N-[2-(2,6-Dioxo-piperidin-3-yl)-1,3-dioxo-2,3-dihydro-1H-isoindol-4-ylmethyl]-2-pyridin-4-yl-acetamide.

18. The method of claim 1, wherein the TOR kinase inhibitor is a compound from Table A.

19. The method of claim 1, further comprising the administration of an anti-CD20 antibody.

20. The method of claim 19, wherein anti-CD20 antibody is rituximab.
